(19) United States
(12) Patent Application Publication
(10) Pub. No.: US 2026/0041723 A1
(43) Pub. Date: Feb. 12, 2026

(54) BACTERIOPHAGE COMPOSITIONS FOR TREATING STAPHYLOCOCCUS INFECTION

(71) Applicant: Armata Pharmaceuticals, Inc., Marina del Rey, CA (US)

(72) Inventors: Stacey Lynn KOLAR, Los Angeles, CA (US); Brian C. VARNUM, Santa Monica, CA (US)

(21) Appl. No.: 19/309,246

(22) Filed: Aug. 25, 2025

Related U.S. Application Data
(62) Division of application No. 17/179,295, filed on Feb. 18, 2021.
(60) Provisional application No. 62/978,006, filed on Feb. 18, 2020.

(51) Int. Cl. A61K 35/76; A61K 9/00; A61K 45/06; A61P 31/04; C12N 7/00

(52) U.S. Cl. CPC A61K 35/76; A61K 9/0019; A61K 9/0078; A61K 45/06; A61P 31/04; C12N 7/00

(57) ABSTRACT

The present disclosure relates to bacteriophages and compositions capable of infecting and killing Staphylococcus, and use of the same for treating Staphylococcus, e.g. Staphylococcus aureus, bacterial infections.

### CROSS-REFERENCES TO RELATED APPLICATIONS

This application is a divisional of U.S. patent application Ser. No. 17/179,295 filed Feb. 18, 2021, which claims the benefit of U.S. Provisional Application No. 62/978,006, filed Feb. 18, 2020, which is incorporated herein by reference in its entirety and for all purposes.

### SEQUENCE LISTING

The instant application contains a Sequence Listing, which has been submitted electronically in ASCII format and is hereby incorporated by reference in its entirety. Said ASCII copy, created on Feb. 18, 2020 is named 054249-519P01US_SEQUENCE_LISTING_ST25.txt and is 377,606 bytes in size.

### BACKGROUND

There is an increasing demand for alternative antibiotics as the number of bacterial strains resistant to traditional, small molecule antibiotic treatment regimens are becoming more numerous. Bacteriophage therapy uses bacterial viruses, or phages, to target and destroy bacteria at various sites of infection. Recent advances in biotechnology have allowed for the fast expansion of naturally existing phage libraries in order to generate potent and specific bacteriophages that can target and destroy a bacterium of interest. Antibiotic-resistant Staphylococcus aureus (SA) is typically found in hospitals and in areas where immune-compromised patients reside. Secondary SA infection is a potentially lethal infection (>20%) that is a common threat to these patients. Indeed, there are approximately 50/100,000 population SA bacteremia cases diagnosed each year in the US alone. Bacteriophage treatment approaches that can circumvent traditional mechanisms of antibiotic resistance, avoid the toxic side effects of traditional small molecule therapies, can be effective against biofilms, and avoid disruption of the native gut flora are especially attractive.

Thus, there is a large unmet need for a more efficient, potent, and specific anti-SA therapy to replace or augment the traditional small molecule antibiotics currently used to treat SA infections today.

### SUMMARY

Described herein are bacteriophages, compositions of bacteriophages, and use of the same for medical and non-medical applications.

In an aspect, provided herein is an isolated, purified bacteriophage that includes a polynucleotide sequence of SEQ ID NO: 1.

In an aspect, provided herein is an isolated, purified bacteriophage that includes a polynucleotide sequence with at least 93% identity to SEQ ID NO: 1. In an aspect, provided herein is an isolated, purified bacteriophage that includes a polynucleotide sequence with at least 90% identity to SEQ ID NO: 1.

In an aspect, provided herein is an isolated, purified bacteriophage that includes a polynucleotide sequence of SEQ ID NO: 2.

In an aspect, provided herein is an isolated, purified bacteriophage that includes a polynucleotide sequence with at least 93% identity to SEQ ID NO: 2. In an aspect, provided herein is an isolated, purified bacteriophage that includes a polynucleotide sequence with at least 90% identity to SEQ ID NO: 2.

In an aspect, provided herein are bacteriophage compositions that include one or more bacteriophages selected from a bacteriophage including a polynucleotide sequence of SEQ ID NO: 1, SEQ ID NO: 2, a polynucleotide sequence with at least 93% identity to SEQ ID NO: 1, and a polynucleotide sequence with at least 93% identity to SEQ ID NO: 2. In an aspect, provided herein are bacteriophage compositions that include one or more bacteriophages selected from a bacteriophage including a polynucleotide sequence of SEQ ID NO: 1, SEQ ID NO: 2, a polynucleotide sequence with at least 90% identity to SEQ ID NO: 1, and a polynucleotide sequence with at least 90% identity to SEQ ID NO: 2.

In an aspect, provided herein are uses of a composition including one or more distinct bacteriophages that target Staphylococcus aureus in the treatment of subject with a Staphylococcus aureus bacterial infection. The uses include administering the composition to a subject; where at least one of the one or more bacteriophages is selected from a bacteriophage including a polynucleotide sequence of SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, at least one of the one or more bacteriophages is selected from a bacteriophage including a polynucleotide sequence of SEQ ID NO: 1, SEQ ID NO: 2, a polynucleotide sequence with at least 90% identity to SEQ ID NO: 1, and a polynucleotide sequence with at least 90% identity to SEQ ID NO: 2.

In an aspect, provided herein is a bacterial host manufacturing strain including a bacteriophage where the bacteriophage includes a polynucleotide sequence selected from SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, the bacteriophage includes a polynucleotide sequence selected from SEQ ID NO: 1, SEQ ID NO: 2, a polynucleotide sequence with at least 90% identity to SEQ ID NO: 1, and a polynucleotide sequence with at least 90% identity to SEQ ID NO: 2.

In an aspect, provided herein are methods of treating a subject with a bacterial infection including selecting a bacteriophage based upon resistance to blood complement inactivation and administering the bacteriophage to the subject.

In an aspect, provided herein are methods of treating a subject with a bacterial infection including administering to the subject one or more distinct bacteriophages selected from a bacteriophage including a polynucleotide sequence selected from SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, the methods of treatment include administering to the subject one or more distinct bacteriophages selected from a bacteriophage including a polynucleotide sequence selected from SEQ ID NO: 1, SEQ ID NO: 2, a polynucleotide sequence with at least 90% identity to SEQ ID NO: 1, and a polynucleotide sequence with at least 90% identity to SEQ ID NO: 2.

In an aspect, provided herein are methods of modifying the microbial flora in a subject including administering to the subject at least one bacteriophage that targets Staphylococcus bacteria, where the bacteriophage includes a polynucleotide sequence selected from SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, the bacteriophage includes a polynucleotide sequence selected from SEQ ID NO: 1, SEQ ID NO: 2, a polynucleotide sequence with at least 90% identity to SEQ ID NO: 1, and a polynucleotide sequence with at least 90% identity to SEQ ID NO: 2.

### BRIEF DESCRIPTION OF THE DRAWINGS

Embodiments will now be described, by way of example only, with reference to the accompanying drawings, in which:

FIG. 1 shows efficiency of plating and plaque morphology. Equal titers of phage were serially diluted and 2 μL were plated on a lawn of S. aureus. Phage potency was assessed by plaques clarity and efficiency of plating.

FIG. 2 shows compstatin effect on Sa87 activity after exposure to plasma. Sa87 was exposed to fresh plasma isolated from three donors, in the presence or absence of complement inhibitor compstatin, for 60 minutes; phage were titered using the double-layer agar method. D indicates donor.

FIG. 3 shows lead phage activity after exposure to plasma. Lead phage candidates were diluted in fresh plasma from six donors and the infectivity was monitored for 90 minutes.

FIG. 4 shows AP-SA02 markedly reduces SA biofilm mass. Biofilms were treated with AP-SA02 (10⁷ phage/well of a 96 well plate) for 5 hours and the percentage of biofilm that was eradicated calculated is reported as percent relative to the same strain treated with vehicle. NRS100 is the negative control.

FIGS. 5A-5B show AP-SA02 activity in the presence of vancomycin. FIG. 5A shows bacterial growth measured by turbidity (absorbance at 600 nm) in the presence of AP-SA02 and vancomycin in a checkerboard assay. Grey boxes indicate lines on graph depicted in FIG. 5B. AP-SA02 is active against a VRSA strain. Vancomycin, 16 μg/mL; AP-SA02, 0.1 pg/mL.

FIGS. 6A-6B show the synergistic activity of vancomycin and AP-SA02. FIG. 6A is data showing bacterial growth measured by turbidity (absorbance at 600 nm) in the presence of AP-SA02 and vancomycin in a checkerboard assay. Grey boxes show lines on graph depicted in FIG. 6B. AP-SA02 and vancomycin show synergistic activity against a VRSA strain. Vancomycin, 2 μg/mL.

### DETAILED DESCRIPTION

It is to be understood that the present disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The detailed description of the present disclosure is divided into various sections only for the reader's convenience and disclosure found in any section may be combined with that in another section. Unless defined otherwise, all technical and scientific terms used herein have the same meaning as commonly understood by one of ordinary skill in the art to which the present disclosure belongs.

### Definitions

It must be noted that as used herein and in the appended claims, the singular forms “a,” “an,” and “the” include plural referents unless the context clearly dictates otherwise. Thus, for example, reference to “a bacteriophage composition” includes a plurality of such candidate agents and reference to “the bacteriophage” includes reference to one or more bacteriophages and equivalents thereof known to those skilled in the art, and so forth.

The term “consists essentially of” as used herein means that only the bacteriophage(s) explicitly indicated are present in the bacteriophage composition, but that said composition may also contain a further non-bacteriophage constituent, such as a pharmaceutically appropriate carrier, diluent, excipient, antibiotic (e.g., chemical antibiotic), etc., or combinations thereof.

As used herein, the term “about” when used before a numerical designation, e.g., temperature, time, amount, concentration, and such other, including a range, indicates approximations which may vary by (+) or (−) 10%, 5%, or 1%.

When a range (e.g., dosage range) is listed herein, it is to be understood that the value may include any individual value or range within the recited range(s), including endpoints.

As used herein, the term “mutant” refers to a bacteriophage differing genetically from ARSA0001 or ARSA0002 but still retaining the ability to infect and kill Staphylococcus aureus target bacteria. Mutants typically comprise bacteriophages with, e.g., silent mutations, conservative mutations, minor deletions, and/or minor replications of genetic material, and retain phenotypic characteristics of the reference bacteriophage. In an embodiment, the mutants retain any observable characteristic or property that is dependent upon the genome of the bacteriophage as described herein, i.e. phenotypic characteristics of said bacteriophage and/or lytic activity against Staphylococcus species. Preferred mutants retain the ability to infect and kill Staphylococcus aureus target bacteria and have less than 10% nucleic acid variation as compared to the genome of the reference bacteriophage, even more preferably less than 7%, more preferably less than 1%. Alternatively, or in combination, mutants have preferably less than 7% amino acid variation in a coded polypeptide sequence as compared to a polypeptide of the reference bacteriophage.

As used herein, the terms “% identity”, “% sequence identity” and “percent identity” in relation to nucleic acid or amino acid sequences designates the level of identity or homology between said sequences and may be determined by techniques known in the art. Any of a variety of sequence alignment methods can be used to determine percent identity, including, without limitation, global methods, local methods and hybrid methods, such as segment approach methods. Protocols to determine percent identity are routine procedures within the scope of one skilled in the art. Global methods align sequences from the beginning to the end of the molecule and determine the best alignment by adding up scores of individual nucleotide pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22 (22) Nucleic Acids Research 4673-4680 (1994); and iterative refinement. Non-limiting methods include, e.g., BLAST, Match-box, see, e.g., Align-M, see, e.g., Ivo Van Walle et al., Align-M-A New Algorithm for Multiple Alignment of Highly Divergent Sequences, Bioinformatics 20 (9): 1428-1435 (2004). This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 100 nucleotides in length, or more preferably over a region that is 100-1000 or more nucleotides in length.

As used herein, the term “bacterial complementation” refers to the ability of a bacteriophage with a particular genome to compensate for a different, distinct bacteriophage with a different genome. More specifically, bacteriophage insensitive mutant colonies (of target bacteria) may arise to a particular bacteriophage but may still be sensitive to a different bacteriophage. In other words, bacteriophage resistant mutant bacteria arising to one phage are still sensitive to another phage.

As used herein, the term “generalized transduction” refers to a process by which any bacterial DNA may be transferred to another bacterium via a bacteriophage. It is a rare event; a very small percentage of phage particles happen to carry a donor bacterium's DNA, on the order of 1 phage in 10,000. In essence, this is the packaging of bacterial DNA into a viral envelope.

As used herein, the term “treat” or “treating” is intended to encompass prophylactic treatment as well as corrective treatment (treatment of a subject already suffering from a disease). This may include the medical management of a subject with the intent to cure, ameliorate, stabilize, or prevent a disease, pathological condition, or disorder. This term includes active treatment, that is, treatment directed specifically toward the improvement of a disease, pathological condition, or disorder; and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization, or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, the term “lytic” or “lytic activity” designates the property of a bacteriophage to cause lysis of a bacterial cell. The lytic activity of a bacteriophage can be tested on a bacterium (e.g., S. aureus strains) according to techniques known in the art. The lytic cycle is named for the process that occurs when a phage has infected a cell, replicated new phage particles, and bursts through the host cell membrane. Some phage exhibit a lysogenic cycle during which the bacteriophage DNA remains practically dormant due to active repression of bacteriophage processes. Whenever the bacteria divides, the DNA of the phage is copied as well. In this way, the virus can continue replicating within its host without lysing the host. At a certain point, conditions may change and the phage enters a lytic cycle. “Obligately lytic” refers to phage that are unable to undergo a lysogenic cycle.

As used herein, the term “complement system” or “blood complement system” refers the part of the innate immune system that triggers phagocytosis, inflammation, and membrane disruption of foreign bodies, including bacteriophages. The complement systems consists of a classical pathway, an alternative pathway, and a lectin pathway. The classical pathway initiates when a multi-protein complex called C1 is formed upon antigen recognition by IgG or IgM. C1 multi-protein complex is composed of one molecule of C1q, two molecules of C1r, and two molecules of C1s. After undergoing several internal proteolysis steps, the subunits C4 and C2 are generated, and further processed to form C2a, C2b, C4a, and C4b. C2b and C4b combine to form classical-form C3-convertase. C3 is further processed to form C3a and C3b. C3b can then join C3 to form a C5-convertase. Low levels of C3 can be converted to C3b without the presence of an antigen, and can combine with other proteases to form alternative complexes in the alternative complement pathway. The lectin pathway replaces the C1q protein from C1 with lectin-binding proteins, including mannose binding lectin (MBL).

As used herein, the term “C3” or “complement component C3” refers to any of the proteins involved in the complement pathways: classical, alternative, or lectin. One form of activated C3 is a heterodimer of activated C2 and C4 proteins, C4b2a, also known as C3-convertase. One form activated C3 is a heterodimer of activated C3b and activated factor B, Bb, to form C3bBb.

As used herein, the term “bacteriophage target” refers to any bacteria species that can be infected by a particular bacteriophage. A bacteriophage recognizes the target bacterial cell surface, binds, and injects its genetic material inside the bacterial host. The genetic material from the infecting phage can be incorporated into the bacterial genome. The bacteriophage may become lysogenic, where the viral genome remains dormant in the bacterial host genome until a triggering event. The bacteriophage may also become lytic, wherein many copies of the infecting phage are produced by the machinery of the infected bacteria, and the copies are subsequently released by bacterial lysis, extrusion, or by budding.

As used herein, the term “bacterial host manufacturing strain” or “manufacturing strain” refers to the bacteria used to grow bacteriophage. A method for bacteriophage production may require a production process involving at least two operating units, growth of the host bacteria and bacteriophage propagation (or infection). It is important to consider basic parameters for bacterial growth and phage infection, such as the selected substrates for the bacterium and the optimal temperature, both for bacterial growth and phage infection, since these factors may influence the infectivity of phages.

As used herein, the term “bacteremia” refers to the presence of bacteria in the bloodstream. Bacteria may be introduced into the bloodstream by ordinary activities including brushing teeth, or may be introduced by surgical procedures, implantation of temporary medical devices, urinary tract infections, at the site of a severe injury, or at the site of long-term device implantation.

As used herein, the term “septicemia” refers to a bacterial infection elsewhere in the body which enters the bloodstream; also known as blood poisoning by bacteria.

As used herein, the term “Silviavirus” refers to a virus that belongs to Regum: virus, Group 1: dsDNA, Ordo: Caudovirales, Familia: Herelleviridae, Subfamilia: Twortvirinae, Genus: Silviavirus. Species of Genus Silviavirus include, without limitation, Staphylococcus virus Remus, Staphylococcus virus SA11, Staphylococcus virus Romulus, Staphylococcus virus Qdsa001, Staphylococcus virus MR003, Staphylococcus virus StAP1, and Staphylococcus virus Stsau2.

A use or method typically comprises administering a bacteriophage or bacteriophage composition described herein to a subject. As used herein, a “subject” is a mammal, such as a human or other animal. Preferably, the subject is a human.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a caregiver's expertise, but that include the knowledge that the subject is ill, or will be ill, as the result of a condition that is treatable by the compositions of the invention.

As used herein, the term “isolated” indicates that the bacteriophage are removed from its original environment in which it naturally occurs. In particular, an isolated bacteriophage is, e.g., cultivated, cultured separately from the environment in which it is naturally located.

As used herein, the term “purified” indicates that the bacteriophage are removed from manufacturing host bacteria. In particular, a purified bacteriophage has production impurities, such as bacterial components, removed from its manufacturing or production environment. Bacterial components include but are not limited to bacterial host proteins, lipids, and/or bacterial endotoxin. The term “purified” may also refer to genetic purification in which the strain of bacteriophage is genetically homogenous.

As used herein, the term “substantially purified” refers to a composition containing less than 1%, less than 0.1%, less than 0.001%, or no detectable amount of contaminants such as host bacterial proteins or endotoxin. Also, as used herein, the term “substantially pure” when used to describe a bacteriophage strain refers to the genetic purity of the composition such that the strain is greater than 99%, greater than 99.9%, greater than 99.999%, or 100% of one particular genome sequence.

Typically, a composition is substantially pure when at least 80%, at least 85%, at least 90%, at least 95%, at least 98%, or at least 99% of the total material (by volume, by wet or dry weight, or by mole percent or mole fraction) in a sample is free of impurities or genetic variants.

As used herein, the term “subject” or “patient” refers to a human or non-human animal. Preferably, the subject or patient is in need of treatment with the composition as described herein, e.g., has a bacterial infection susceptible to treatment with the composition.

As used herein, the “synergistic amount” refers to the sum of a first amount (e.g., a bacteriophage) and a second amount (e.g., a different bacteriophage) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

As used herein, the term “substantially free” refers to something having less than 10% of the substance that it is to be free from. For example, 0.01% to 10% free of the substance, including any subvalue and subrange therein, including endpoints. For example, 0.01, 0.02, 0.03, 0.04, 0.05, 0.06, 0.07, 0.08, 0.09, 1, 2, 3, 4, 5, 6, 7, 8, 9, or 10%.

As used herein, the term “obtainable” as used herein also encompasses the term “obtained.” In one embodiment, the term “obtainable” means obtained.

Additional terms and phrases are defined below.

### Bacteriophage Compositions

In an aspect, provided herein is an isolated, purified bacteriophage that includes a polynucleotide sequence of SEQ ID NO: 1.

In an aspect, provided herein is an isolated, purified bacteriophage that includes a polynucleotide sequence with at least 90% identity to SEQ ID NO: 1.

In embodiments, provided herein is an isolated, purified bacteriophage that includes a polynucleotide sequence with at least 99.9%, 99.8%, 99.7%, 99.6%, 99.5%, 99.4%, 99.3%, 99.2%, 99.1%, 99.0%, or 99% identity to SEQ ID NO: 1. In embodiments, provided herein is an isolated, purified bacteriophage that includes a polynucleotide sequence with at least 98.9%, 98.8%, 98.7%, 98.6%, 98.5%, 98.4%, 98.3%, 98.2%, 98.1%, 98.0%, or 98% identity to SEQ ID NO: 1. In embodiments, provided herein is an isolated, purified bacteriophage that includes a polynucleotide sequence with at least 97.9%, 97.8%, 97.7%, 97.6%, 97.5%, 97.4%, 97.3%, 97.2%, 97.1%, 97.0% or 97% identity to SEQ ID NO: 1. In embodiments, provided herein is an isolated, purified bacteriophage that includes a polynucleotide sequence with at least 96.9%, 96.8%, 96.7%, 96.6%, 96.5%, 96.4%, 96.3%, 96.2%, 96.1%, 96.0%, or 96% identity to SEQ ID NO: 1. In embodiments, provided herein is an isolated, purified bacteriophage that includes a polynucleotide sequence with at least 95.9%, 95.8%, 95.7%, 95.6%, 95.5%, 95.4%, 95.3%, 95.2%, 95.1%, 95.0%, or 95% identity to SEQ ID NO: 1. In embodiments, provided herein is an isolated, purified bacteriophage that includes a polynucleotide sequence with at least 94.9%, 94.8%, 94.7%, 94.6%, 94.5%, 94.4%, 94.3%, 94.2%, 94.1%, 94.0%, or 94% identity to SEQ ID NO: 1. In embodiments, provided herein is an isolated, purified bacteriophage that includes a polynucleotide sequence with at least 93.9%, 93.8%, 93.7%, 93.6%, 93.5%, 93.4%, 93.3%, 93.2%, 93.1%, 93.0%, or 93% identity to SEQ ID NO: 1. In embodiments, provided herein is an isolated, purified bacteriophage that includes a polynucleotide sequence with at least 92.9%, 92.8%, 92.7%, 92.6%, 92.5%, 92.4%, 92.3%, 92.2%, 92.1%, 92.0%, or 92% identity to SEQ ID NO: 1. In embodiments, provided herein is an isolated, purified bacteriophage that includes a polynucleotide sequence with at least 91.9%, 91.8%, 91.7%, 91.6%, 91.5%, 91.4%, 91.3%, 91.2%, 91.1%, 91.0%, or 91% identity to SEQ ID NO: 1. In embodiments, provided herein is an isolated, purified bacteriophage that includes a polynucleotide sequence with at least 90.9%, 90.8%, 90.7%, 90.6%, 90.5%, 90.4%, 90.3%, 90.2%, 90.1%, 90.0%, or 90% identity to SEQ ID NO: 1. In embodiments, provided herein is an isolated, purified bacteriophage that includes a polynucleotide sequence with at least 89.9%, 89.8%, 89.7%, 89.6%, 89.5%, 89.4%, 89.3%, 89.2%, 89.1%, 89.0%, or 89% identity to SEQ ID NO: 1. In embodiments, provided herein is an isolated, purified bacteriophage that includes a polynucleotide sequence with at least 88.9%, 88.8%, 88.7%, 88.6%, 88.5%, 88.4%, 88.3%, 88.2%, 88.1%, 88.0%, or 88% identity to SEQ ID NO: 1. In embodiments, provided herein is an isolated, purified bacteriophage that includes a polynucleotide sequence with at least 87.9%, 87.8%, 87.7%, 87.6%, 87.5%, 87.4%, 87.3%, 87.2%, 87.1%, 87.0%, or 87% identity to SEQ ID NO: 1. In embodiments, provided herein is an isolated, purified bacteriophage that includes a polynucleotide sequence with at least 86.9%, 86.8%, 86.7%, 86.6%, 86.5%, 86.4%, 86.3%, 86.2%, 86.1%, 86.0%, or 86% identity to SEQ ID NO: 1. In embodiments, provided herein is an isolated, purified bacteriophage that includes a polynucleotide sequence with at least 85.9%, 85.8%, 85.7%, 85.6%, 85.5%, 85.4%, 85.3%, 85.2%, 85.1%, 85.0%, or 85% identity to SEQ ID NO: 1. In embodiments, provided herein is an isolated, purified bacteriophage that includes a polynucleotide sequence with at least 84.9%, 84.8%, 84.7%, 84.6%, 84.5%, 84.4%, 84.3%, 84.2%, 84.1%, 84.0%, or 84% identity to SEQ ID NO: 1. In embodiments, provided herein is an isolated, purified bacteriophage that includes a polynucleotide sequence with at least 83.9%, 83.8%, 83.7%, 83.6%, 83.5%, 83.4%, 83.3%, 83.2%, 83.1%, 83.0%, or 83% identity to SEQ ID NO: 1. In embodiments, provided herein is an isolated, purified bacteriophage that includes a polynucleotide sequence with at least 82.9%, 82.8%, 82.7%, 82.6%, 82.5%, 82.4%, 82.3%, 82.2%, 82.1%, 82.0%, or 82% identity to SEQ ID NO: 1. In embodiments, provided herein is an isolated, purified bacteriophage that includes a polynucleotide sequence with at least 81.9%, 81.8%, 81.7%, 81.6%, 81.5%, 81.4%, 81.3%, 81.2%, 81.1%, 81.0%, or 81% identity to SEQ ID NO: 1. In embodiments, provided herein is an isolated, purified bacteriophage that includes a polynucleotide sequence with at least 80.9%, 80.8%, 80.7%, 80.6%, 80.5%, 80.4%, 80.3%, 80.2%, 80.1%, 80.0%, or 80% identity to SEQ ID NO: 1. In embodiments, the bacteriophage genome comprises the polynucleotide.

In an aspect, provided herein is an isolated, purified bacteriophage that includes a polynucleotide sequence identified as SEQ ID NO: 2.

In an aspect, provided herein is an isolated, purified bacteriophage that includes a polynucleotide sequence with at least 93% identity to SEQ ID NO: 2. In another aspect, the purified bacteriophage includes a polynucleotide sequence with at least 90% identity to SEQ ID NO: 2.

In embodiments, provided herein is an isolated, purified bacteriophage that includes a polynucleotide sequence with at least 99.9%, 99.8%, 99.7%, 99.6%, 99.5%, 99.4%, 99.3%, 99.2%, 99.1%, 99.0% or 99% identity to SEQ ID NO: 2. In embodiments, provided herein is an isolated, purified bacteriophage that includes a bacteriophage that includes a polynucleotide sequence with at least 98.9%, 98.8%, 98.7%, 98.6%, 98.5%, 98.4%, 98.3%, 98.2%, 98.1%, 98.0%, or 98% identity to SEQ ID NO: 2. In embodiments, provided herein is an isolated, purified bacteriophage that includes a polynucleotide sequence with at least 97.9%, 97.8%, 97.7%, 97.6%, 97.5%, 97.4%, 97.3%, 97.2%, 97.1%, 97.0%, or 97% identity to SEQ ID NO: 2. In embodiments, provided herein is an isolated, purified bacteriophage that includes a bacteriophage that includes a polynucleotide sequence with at least 96.9%, 96.8%, 96.7%, 96.6%, 96.5%, 96.4%, 96.3%, 96.2%, 96.1%, 96.0%, or 96% identity to SEQ ID NO: 2. In embodiments, provided herein is an isolated, purified bacteriophage that includes a polynucleotide sequence with at least 95.9%, 95.8%, 95.7%, 95.6%, 95.5%, 95.4%, 95.3%, 95.2%, 95.1%, 95.0%, or 95% identity to SEQ ID NO: 2. In embodiments, provided herein is an isolated, purified bacteriophage that includes a bacteriophage that includes a polynucleotide sequence with at least 94.9%, 94.8%, 94.7%, 94.6%, 94.5%, 94.4%, 94.3%, 94.2%, 94.1%, 94.0%, or 94% identity to SEQ ID NO: 2. In embodiments, provided herein is an isolated, purified bacteriophage that includes a polynucleotide sequence with at least 93.9%, 93.8%, 93.7%, 93.6%, 93.5%, 93.4%, 93.3%, 93.2%, 93.1%, 93.0%, or 93% identity to SEQ ID NO: 2. In embodiments, provided herein is an isolated, purified bacteriophage that includes a polynucleotide sequence with at least 92.9%, 92.8%, 92.7%, 92.6%, 92.5%, 92.4%, 92.3%, 92.2%, 92.1%, 92.0%, or 92% identity to SEQ ID NO: 2. In embodiments, provided herein is an isolated, purified bacteriophage that includes a polynucleotide sequence with at least 91.9%, 91.8%, 91.7%, 91.6%, 91.5%, 91.4%, 91.3%, 91.2%, 91.1%, 91.0%, or 91% identity to SEQ ID NO: 2. In embodiments, provided herein is an isolated, purified bacteriophage that includes a polynucleotide sequence with at least 90.9%, 90.8%, 90.7%, 90.6%, 90.5%, 90.4%, 90.3%, 90.2%, 90.1%, 90.0%, or 90% identity to SEQ ID NO: 2. In embodiments, provided herein is an isolated, purified bacteriophage that includes a polynucleotide sequence with at least 89.9%, 89.8%, 89.7%, 89.6%, 89.5%, 89.4%, 89.3%, 89.2%, 89.1%, 89.0%, or 89% identity to SEQ ID NO: 2. In embodiments, provided herein is an isolated, purified bacteriophage that includes a polynucleotide sequence with at least 88.9%, 88.8%, 88.7%, 88.6%, 88.5%, 88.4%, 88.3%, 88.2%, 88.1%, 88.0%, or 88% identity to SEQ ID NO: 2. In embodiments, provided herein is an isolated, purified bacteriophage that includes a polynucleotide sequence with at least 87.9%, 87.8%, 87.7%, 87.6%, 87.5%, 87.4%, 87.3%, 87.2%, 87.1%, 87.0%, or 87% identity to SEQ ID NO: 2. In embodiments, provided herein is an isolated, purified bacteriophage that includes a polynucleotide sequence with at least 86.9%, 86.8%, 86.7%, 86.6%, 86.5%, 86.4%, 86.3%, 86.2%, 86.1%, 86.0%, or 86% identity to SEQ ID NO: 2. In embodiments, provided herein is an isolated, purified bacteriophage that includes a polynucleotide sequence with at least 85.9%, 85.8%, 85.7%, 85.6%, 85.5%, 85.4%, 85.3%, 85.2%, 85.1%, 85.0%, or 85% identity to SEQ ID NO: 2. In embodiments, provided herein is an isolated, purified bacteriophage that includes a polynucleotide sequence with at least 84.9%, 84.8%, 84.7%, 84.6%, 84.5%, 84.4%, 84.3%, 84.2%, 84.1%, 84.0%, or 84% identity to SEQ ID NO: 2. In embodiments, provided herein is an isolated, purified bacteriophage that includes a polynucleotide sequence with at least 83.9%, 83.8%, 83.7%, 83.6%, 83.5%, 83.4%, 83.3%, 83.2%, 83.1%, 83.0%, or 83% identity to SEQ ID NO: 2. In embodiments, provided herein is an isolated, purified bacteriophage that includes a polynucleotide sequence with at least 82.9%, 82.8%, 82.7%, 82.6%, 82.5%, 82.4%, 82.3%, 82.2%, 82.1%, 82.0%, or 82% identity to SEQ ID NO: 2. In embodiments, provided herein is an isolated, purified bacteriophage that includes a polynucleotide sequence with at least 81.9%, 81.8%, 81.7%, 81.6%, 81.5%, 81.4%, 81.3%, 81.2%, 81.1%, 81.0%, or 81% identity to SEQ ID NO:2. In embodiments, provided herein is an isolated, purified bacteriophage that includes a polynucleotide sequence with at least 80.9%, 80.8%, 80.7%, 80.6%, 80.5%, 80.4%, 80.3%, 80.2%, 80.1%, 80.0%, or 80% identity to SEQ ID NO: 2. In embodiments, the bacteriophage genome comprises the polynucleotide.

In an aspect, provided herein are bacteriophage compositions that include one or more bacteriophages.

In embodiments, provided herein are bacteriophage compositions that include two bacteriophages according to any embodiment described herein. In embodiments, provided herein are bacteriophage compositions that include two or more bacteriophages according to any embodiment described herein. In embodiments, provided herein are bacteriophage compositions that include three or more bacteriophages according to any embodiment described herein. In embodiments, provided herein are bacteriophage compositions include four or more bacteriophages according to any embodiment described herein. In embodiments, provided herein are bacteriophage compositions that include five or more bacteriophages according to any embodiment described herein.

In embodiments, the composition includes one or more bacteriophages that are resistant to inactivation in the blood. In embodiments, the composition includes two or more bacteriophages that are resistant to inactivation in the blood. In embodiments, the composition includes three or more bacteriophages that are resistant to blood complement inactivation in the blood. In embodiments, the composition includes one or more bacteriophages that are resistant to blood complement C3 protein-mediated inactivation in the blood. In embodiments, the composition includes two or more bacteriophages that are resistant to blood complement C3 protein-mediated inactivation in the blood. In embodiments, the composition includes three or more bacteriophages that are resistant to blood complement C3 protein-mediated inactivation in the blood.

In embodiments, the composition includes bacteriophages that target Staphylococcus bacteria. In embodiments, the composition includes bacteriophage that target Staphylococcus aureus. In embodiments, the composition includes bacteriophage that target one or more of vancomycin-intermediate Staphylococcus aureus (VISA), vancomycin-resistant Staphylococcus aureus (VRSA), and/or methicillin-resistant Staphylococcus aureus (MRSA). In embodiments, the composition includes bacteriophage that target vancomycin-intermediate Staphylococcus aureus (VISA). In embodiments, the composition includes bacteriophage that target vancomycin-resistant Staphylococcus aureus (VRSA). In embodiments, the composition includes bacteriophage that target methicillin-resistant Staphylococcus aureus (MRSA).

In embodiments, the composition includes bacteriophage that infect and kill Staphylococcus bacteria. In embodiments, the composition includes bacteriophage that infect and kill Staphylococcus aureus. In embodiments, the composition includes bacteriophage that infect and kill one or more of VISA, VRSA, and/or MRSA. In embodiments, the composition includes bacteriophage that infect and kill VISA. In embodiments, the composition includes bacteriophage that infect and kill VRSA. In embodiments, the composition includes bacteriophage that infect and kill MRSA.

In embodiments, provided herein are bacteriophage compositions that include one or more bacteriophages that belong to the genus Silviavirus. In embodiments, the bacteriophage composition includes a strain from bacteriophage genus Silviavirus. In embodiments, the bacteriophage composition includes a bacteriophage selected from MR003 (Accession No. AP019522.1), Qdsa001 (Accession No. KY779848.1), Remus (Accession No. NC_022090.1), Romulus (Accession No. NC_020877.1), SA11 (Accession No. NC_019511.12), StAP1 (Accession No. KC532239.1), and Stsau2 (Accession No. NC_030933.1). In some embodiments of the bacteriophage composition, the bacteriophage is Silviavirus Staphylococcus virus Remus. In some embodiments of the bacteriophage composition, the bacteriophage is Silviavirus Staphylococcus virus SA11. In some embodiments of the bacteriophage composition, the bacteriophage is Silviavirus Staphylococcus virus Romulus. In some embodiments of the bacteriophage composition, the bacteriophage is Silviavirus Staphylococcus virus Qdsa001. In some embodiments of the bacteriophage composition, the bacteriophage is Silviavirus Staphylococcus virus MR003. In some embodiments of the bacteriophage composition, the bacteriophage is Silviavirus Staphylococcus virus StAP1. In some embodiments of the bacteriophage composition, the bacteriophage is Silviavirus Staphylococcus virus Stsau2.

In an aspect, provided herein are bacteriophage compositions that include one or more bacteriophages selected from a bacteriophage including a polynucleotide sequence of SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, the compositions include one or more bacteriophages selected from a bacteriophage including a polynucleotide sequence of SEQ ID NO: 1, SEQ ID NO: 2, a polynucleotide sequence with at least 90% identity to SEQ ID NO: 1, and a polynucleotide sequence with at least 9% identity to SEQ ID NO: 2.

In embodiments, the bacteriophage composition includes a bacteriophage that includes a polynucleotide sequence of SEQ ID NO: 1. In embodiments, the bacteriophage composition includes a bacteriophage that includes a polynucleotide sequence of SEQ ID NO: 2. In embodiments, the bacteriophage composition includes a bacteriophage that includes a polynucleotide sequence with at least 99.9%, 99.8%, 99.7%, 99.6%, 99.5%, 99.4%, 99.3%, 99.2%, 99.1%, 99.0%, or 99% identity to SEQ ID NO: 1. In embodiments, the bacteriophage composition includes a bacteriophage that includes a polynucleotide sequence with at least 98.9%, 98.8%, 98.7%, 98.6%, 98.5%, 98.4%, 98.3%, 98.2%, 98.1%, 98.0%, or 98% identity to SEQ ID NO: 1. In embodiments, the bacteriophage composition includes a bacteriophage that includes a polynucleotide sequence with at least 97.9%, 97.8%, 97.7%, 97.6%, 97.5%, 97.4%, 97.3%, 97.2%, 97.1%, 97.0%, or 97% identity to SEQ ID NO: 1. In embodiments, the bacteriophage composition includes a bacteriophage that includes a polynucleotide sequence with at least 96.9%, 96.8%, 96.7%, 96.6%, 96.5%, 96.4%, 96.3%, 96.2%, 96.1%, 96.0%, or 96% identity to SEQ ID NO: 1. In embodiments, the bacteriophage composition includes a bacteriophage that includes a polynucleotide sequence with at least 95.9%, 95.8%, 95.7%, 95.6%, 95.5%, 95.4%, 95.3%, 95.2%, 95.1%, 95.0%, or 95% identity to SEQ ID NO: 1. In embodiments, the bacteriophage composition includes a bacteriophage that includes a polynucleotide sequence with at least 94.9%, 94.8%, 94.7%, 94.6%, 94.5%, 94.4%, 94.3%, 94.2%, 94.1%, 94.0%, or 94% identity to SEQ ID NO: 1. In embodiments, the bacteriophage composition includes a bacteriophage that includes a polynucleotide sequence with at least 93.9%, 93.8%, 93.7%, 93.6%, 93.5%, 93.4%, 93.3%, 93.2%, 93.1%, 93.0%, or 93% identity to SEQ ID NO: 1. In embodiments, the bacteriophage composition includes a bacteriophage that includes a polynucleotide sequence with at least 92.9%, 92.8%, 92.7%, 92.6%, 92.5%, 92.4%, 92.3%, 92.2%, 92.1%, 92.0%, or 92% identity to SEQ ID NO: 1. In embodiments, the bacteriophage composition includes a bacteriophage that includes a polynucleotide sequence with at least 91.9%, 91.8%, 91.7%, 91.6%, 91.5%, 91.4%, 91.3%, 91.2%, 91.1%, 91.0%, or 91% identity to SEQ ID NO: 1. In embodiments, the bacteriophage composition includes a bacteriophage that includes a polynucleotide sequence with at least 90.9%, 90.8%, 90.7%, 90.6%, 90.5%, 90.4%, 90.3%, 90.2%, 90.1%, 90.0%, or 90% identity to SEQ ID NO: 1. In embodiments, the bacteriophage composition includes a bacteriophage that includes a polynucleotide sequence with at least 89.9%, 89.8%, 89.7%, 89.6%, 89.5%, 89.4%, 89.3%, 89.2%, 89.1%, 89.0%, or 89% identity to SEQ ID NO: 1. In embodiments, the bacteriophage composition includes a bacteriophage that includes a polynucleotide sequence with at least 88.9%, 88.8%, 88.7%, 88.6%, 88.5%, 88.4%, 88.3%, 88.2%, 88.1%, 88.0%, or 88% identity to SEQ ID NO: 1. In embodiments, the bacteriophage composition includes a bacteriophage that includes a polynucleotide sequence with at least 87.9%, 87.8%, 87.7%, 87.6%, 87.5%, 87.4%, 87.3%, 87.2%, 87.1%, 87.0%, or 87% identity to SEQ ID NO: 1. In embodiments, the bacteriophage composition includes a bacteriophage that includes a polynucleotide sequence with at least 86.9%, 86.8%, 86.7%, 86.6%, 86.5%, 86.4%, 86.3%, 86.2%, 86.1%, 86.0%, or 86% identity to SEQ ID NO: 1. In embodiments, the bacteriophage composition includes a bacteriophage that includes a polynucleotide sequence with at least 85.9%, 85.8%, 85.7%, 85.6%, 85.5%, 85.4%, 85.3%, 85.2%, 85.1%, 85.0%, or 85% identity to SEQ ID NO: 1. In embodiments, the bacteriophage composition includes a bacteriophage that includes a polynucleotide sequence with at least 84.9%, 84.8%, 84.7%, 84.6%, 84.5%, 84.4%, 84.3%, 84.2%, 84.1%, 84.0%, or 84% identity to SEQ ID NO: 1. In embodiments, the bacteriophage composition includes a bacteriophage that includes a polynucleotide sequence with at least 83.9%, 83.8%, 83.7%, 83.6%, 83.5%, 83.4%, 83.3%, 83.2%, 83.1%, 83.0%, or 83% identity to SEQ ID NO: 1. In embodiments, the bacteriophage composition includes a bacteriophage that includes a polynucleotide sequence with at least 82.9%, 82.8%, 82.7%, 82.6%, 82.5%, 82.4%, 82.3%, 82.2%, 82.1%, 82.0%, or 82% identity to SEQ ID NO: 1. In embodiments, the bacteriophage composition includes a bacteriophage that includes a polynucleotide sequence with at least 81.9%, 81.8%, 81.7%, 81.6%, 81.5%, 81.4%, 81.3%, 81.2%, 81.1%, 81.0%, or 81% identity to SEQ ID NO: 1. In embodiments, the bacteriophage composition includes a bacteriophage that includes a polynucleotide sequence with at least 80.9%, 80.8%, 80.7%, 80.6%, 80.5%, 80.4%, 80.3%, 80.2%, 80.1%, 80.0%, or 80% identity to SEQ ID NO: 1. In embodiments, the bacteriophage genome comprises the polynucleotide.

In embodiments, the bacteriophage composition includes a bacteriophage that includes a polynucleotide sequence with at least 99.9%, 99.8%, 99.7%, 99.6%, 99.5%, 99.4%, 99.3%, 99.2%, 99.1%, 99.0%, or 99% identity to SEQ ID NO: 2. I In embodiments, the bacteriophage composition includes a bacteriophage that includes a polynucleotide sequence with at least 98.9%, 98.8%, 98.7%, 98.6%, 98.5%, 98.4%, 98.3%, 98.2%, 98.1%, 98.0%, or 98% identity to SEQ ID NO: 2. In embodiments, the bacteriophage composition includes a bacteriophage that includes a polynucleotide sequence with at least 97.9%, 97.8%, 97.7%, 97.6%, 97.5%, 97.4%, 97.3%, 97.2%, 97.1%, 97.0%, or 97% identity to SEQ ID NO: 2. In embodiments, the bacteriophage composition includes a bacteriophage that includes a polynucleotide sequence with at least 96.9%, 96.8%, 96.7%, 96.6%, 96.5%, 96.4%, 96.3%, 96.2%, 96.1%, 96.0%, or 96% identity to SEQ ID NO: 2. In embodiments, the bacteriophage composition includes a bacteriophage that includes a polynucleotide sequence with at least 95.9%, 95.8%, 95.7%, 95.6%, 95.5%, 95.4%, 95.3%, 95.2%, 95.1%, 95.0%, or 95% identity to SEQ ID NO: 2. In embodiments, the bacteriophage composition includes a bacteriophage that includes a polynucleotide sequence with at least 94.9%, 94.8%, 94.7%, 94.6%, 94.5%, 94.4%, 94.3%, 94.2%, 94.1%, 94.0%, or 94% identity to SEQ ID NO: 2. In embodiments, the bacteriophage composition includes a bacteriophage that includes a polynucleotide sequence with at least 93.9%, 93.8%, 93.7%, 93.6%, 93.5%, 93.4%, 93.3%, 93.2%, 93.1%, 93.0%, or 93% identity to SEQ ID NO: 2. In embodiments, the bacteriophage composition includes a bacteriophage that includes a polynucleotide sequence with at least 92.9%, 92.8%, 92.7%, 92.6%, 92.5%, 92.4%, 92.3%, 92.2%, 92.1%, 92.0%, or 92% identity to SEQ ID NO: 2. In embodiments, the bacteriophage composition includes a bacteriophage that includes a polynucleotide sequence with at least 91.9%, 91.8%, 91.7%, 91.6%, 91.5%, 91.4%, 91.3%, 91.2%, 91.1%, 91.0%, or 91% identity to SEQ ID NO: 2. In embodiments, the bacteriophage composition includes a bacteriophage that includes a polynucleotide sequence with at least 90.9%, 90.8%, 90.7%, 90.6%, 90.5%, 90.4%, 90.3%, 90.2%, 90.1%, 90.0%, or 90% identity to SEQ ID NO: 2. In embodiments, the bacteriophage composition includes a bacteriophage that includes a polynucleotide sequence with at least 89.9%, 89.8%, 89.7%, 89.6%, 89.5%, 89.4%, 89.3%, 89.2%, 89.1%, 89.0%, or 89% identity to SEQ ID NO: 2. In embodiments, the bacteriophage composition includes a bacteriophage that includes a polynucleotide sequence with at least 88.9%, 88.8%, 88.7%, 88.6%, 88.5%, 88.4%, 88.3%, 88.2%, 88.1%, 88.0%, or 88% identity to SEQ ID NO: 2. In embodiments, the bacteriophage composition includes a bacteriophage that includes a polynucleotide sequence with at least 87.9%, 87.8%, 87.7%, 87.6%, 87.5%, 87.4%, 87.3%, 87.2%, 87.1%, 87.0%, or 87% identity to SEQ ID NO: 2. In embodiments, the bacteriophage composition includes a bacteriophage that includes a polynucleotide sequence with at least 86.9%, 86.8%, 86.7%, 86.6%, 86.5%, 86.4%, 86.3%, 86.2%, 86.1%, 86.0%, or 86% identity to SEQ ID NO: 2. In embodiments, the bacteriophage composition includes a bacteriophage that includes a polynucleotide sequence with at least 85.9%, 85.8%, 85.7%, 85.6%, 85.5%, 85.4%, 85.3%, 85.2%, 85.1%, 85.0%, or 85% identity to SEQ ID NO: 2. In embodiments, the bacteriophage composition includes a bacteriophage that includes a polynucleotide sequence with at least 84.9%, 84.8%, 84.7%, 84.6%, 84.5%, 84.4%, 84.3%, 84.2%, 84.1%, 84.0%, or 84% identity to SEQ ID NO: 2. In embodiments, the bacteriophage composition includes a bacteriophage that includes a polynucleotide sequence with at least 83.9%, 83.8%, 83.7%, 83.6%, 83.5%, 83.4%, 83.3%, 83.2%, 83.1%, 83.0%, or 83% identity to SEQ ID NO: 2. In embodiments, the bacteriophage composition includes a bacteriophage that includes a polynucleotide sequence with at least 82.9%, 82.8%, 82.7%, 82.6%, 82.5%, 82.4%, 82.3%, 82.2%, 82.1%, 82.0%, or 82% identity to SEQ ID NO: 2. In embodiments, the bacteriophage composition includes a bacteriophage that includes a polynucleotide sequence with at least 81.9%, 81.8%, 81.7%, 81.6%, 81.5%, 81.4%, 81.3%, 81.2%, 81.1%, 81.0%, or 81% identity to SEQ ID NO: 2. In embodiments, the bacteriophage composition includes a bacteriophage that includes a polynucleotide sequence with at least 80.9%, 80.8%, 80.7%, 80.6%, 80.5%, 80.4%, 80.3%, 80.2%, 80.1%, 80.0%, or 80% identity to SEQ ID NO: 2. In embodiments, the bacteriophage genome comprises the polynucleotide.

In embodiments, provided herein are bacteriophage compositions that include two or more bacteriophages selected from bacteriophage including a polynucleotide sequence of SEQ ID NO: 1, SEQ ID NO: 2, a polynucleotide sequence with at least 93% identity to SEQ ID NO: 1, and a polynucleotide sequence with at least 93% identity to SEQ ID NO: 2; and where the composition's target bacteria range is broader than the cumulative range of the individual bacteriophage in the composition. In another aspect, the bacteriophages compositions may include two or more bacteriophages selected from bacteriophages including a polynucleotide sequence of SEQ ID NO: 1, SEQ ID NO: 2, a polynucleotide sequence with at least 90% identity to SEQ ID NO: 1, and a polynucleotide sequence with at least 90% identity to SEQ ID NO: 2; and where the composition's target bacteria range is broader than the cumulative range of the individual bacteriophage in the composition.

In embodiments, the bacteriophage composition includes one or more additional bacteriophages. In some embodiments, the one or more additional bacteriophages are suitable for treating a bacterial infection, in particular a Staphylococcus infection. In embodiments, the additional one or more phage can be natural or non-naturally occurring. In embodiments, the one or more additional phage can be a phage with at least 80% nucleic acid sequence identity to any of the phage described herein. In embodiments, the one or more additional phage can be a phage with at least 80% nucleic acid sequence identity to SEQ ID NO: 1. In embodiments, the one or more additional phage can be a phage with at least 80% nucleic acid sequence identity to SEQ ID NO: 2. In embodiments, the bacteriophage include a polynucleotide sequence with at least 80% but not 100% identity to any one of SEQ ID NO: 1 or SEQ ID NO: 2.

In embodiments, the range of target bacteria of the bacteriophage composition is broader than the range of target bacteria of any single bacteriophage included within the composition. Such activity can be considered synergistic as the effect of the composition (target killing range) is greater than the sum of individual effects (target killing range) of each component bacteriophage. In embodiments, provided herein are bacteriophage compositions where the composition's target bacteria range can have an effectiveness that is greater than the sum of effectiveness of the individual bacteriophage.

In embodiments, provided herein are bacteriophage compositions that include two or more bacteriophages, where the composition includes a bacteriophage with polynucleotide sequence of SEQ ID NO: 1 and the composition targets more Staphylococcus aureus strains than a bacteriophage with polynucleotide sequence of SEQ ID NO: 1. In embodiments, provided herein are bacteriophage compositions that include two or more bacteriophage, where the composition includes a bacteriophage with a polynucleotide sequence with at least 93% identity with SEQ ID NO: 1 and the composition targets more Staphylococcus aureus strains than a bacteriophage with a polynucleotide sequence with at least 93% identity with SEQ ID NO: 1. In embodiments, the composition includes a bacteriophage with a polynucleotide sequence with at least 90% identity with SEQ ID NO: 1 and the composition targets more Staphylococcus aureus strains than a bacteriophage with a polynucleotide sequence with at least 90% identity with SEQ ID NO: 1. In embodiments, provided herein are bacteriophage compositions that include two or more bacteriophages, where the composition includes a bacteriophage with polynucleotide sequence of SEQ ID NO: 2 and the composition targets more Staphylococcus aureus strains than a bacteriophage with polynucleotide sequence of SEQ ID NO: 2. In embodiments, provided herein are bacteriophage compositions that include two or more bacteriophage, where the composition includes a bacteriophage with a polynucleotide sequence with at least 93% identity with SEQ ID NO: 2 and the composition targets more Staphylococcus aureus strains than a bacteriophage with a polynucleotide sequence with at least 93% identity with SEQ ID NO: 2. In embodiments, provided herein are bacteriophage compositions that include two or more bacteriophage, where the composition includes a bacteriophage with a polynucleotide sequence with at least 90% identity with SEQ ID NO: 2 and the composition targets more Staphylococcus aureus strains than a bacteriophage with a polynucleotide sequence with at least 90% identity with SEQ ID NO: 2. In embodiments, the compositions can have any combination polynucleotide sequences of SEQ ID NO: 1 and SEQ ID NO: 2.

Provided herein are bacteriophage compositions that include two or more bacteriophage, where the composition includes at least one bacteriophage that is genetically modified. Provided herein are bacteriophage compositions that include two or more bacteriophage, where the composition includes at least one naturally occurring phage. Provided herein are bacteriophage compositions that include two or more bacteriophage, where the composition excludes naturally occurring phage. Provided herein are bacteriophage compositions that include two or more bacteriophage, where the composition includes one or more bacteriophage that is resistant to inactivation by the blood complement system.

In embodiments, the bacteriophage composition includes an additional component selected from a pharmaceutically acceptable carrier, diluent, excipient and combinations thereof. In embodiments, the bacteriophage composition includes a pharmaceutically acceptable carrier. In embodiments, the bacteriophage composition includes a diluent. In embodiments, the bacteriophage composition includes an excipient. In embodiments, the bacteriophage composition includes a combination of a pharmaceutically acceptable carrier, diluent, and an excipient. In embodiments, the bacteriophage composition includes a combination of a pharmaceutically acceptable carrier and diluent. In embodiments, the bacteriophage composition includes a combination of a pharmaceutically acceptable carrier and an excipient. In embodiments, the bacteriophage composition includes a combination of a diluent and an excipient.

In embodiments, the bacteriophage composition includes a storage media for storage at a temperature at or below 8° C. In embodiments, the bacteriophage composition includes a storage media for storage at a temperature at or below 7° C. In embodiments, the bacteriophage composition includes a storage media for storage at a temperature at or below 6° C. In embodiments, the bacteriophage composition includes a storage media for storage at a temperature at or below 5° C. In embodiments, the bacteriophage composition includes a storage media for storage at a temperature at or below 4° C. In embodiments, the bacteriophage composition includes a storage media for storage at a temperature at or below 3° C. In embodiments, the bacteriophage composition includes a storage media for storage at a temperature at or below 2° C. In embodiments, the bacteriophage composition includes a storage media for storage at a temperature at or below 1° C. In embodiments, the bacteriophage composition includes a storage media for storage at a temperature at or below 0° C.

In embodiments, the bacteriophage composition is in a liquid, semi-liquid, solid, frozen, or lyophilized formulation. In embodiments, the bacteriophage composition is in a liquid formulation. In embodiments, the bacteriophage composition is in a semi-liquid formulation. In embodiments, the bacteriophage composition is in a solid formulation. In embodiments, the bacteriophage composition is in a frozen formulation. In embodiments, the bacteriophage composition is in a lyophilized formulation.

In embodiments, the bacteriophage composition is stored in containers containing from about 1 ml to about 5 ml injectable solution. In some embodiments, this includes single dose containers for single use. In other embodiments, this may include multi-dose containers containing injection solutions that provide multiple doses of the bacteriophage composition described herein. The composition may also be stored in various suitable containers, including, but not necessarily limited to, vials, cuvettes, cartridges, prefilled syringes, plastic bags, ampoules, bottles, pouches, pumps, sprayers, stoppers, needles, plungers, caps, stents, catheters, implants, or blister packages. In some embodiments, the aforementioned containers may be made of glass or plastic, or they may be plastic-coated, or may be made of various other suitable materials. Containers may be clear, or may provide protection from light by being amber colored or of various other colors, or may be wrapped in aluminum foil.

In some embodiments (alternatively or additionally), a “mutant” bacteriophage is capable of lysing some or all the same target bacterial strains as one or more of ARSA0001 and/or ARSA0002, and/or further capable of lysing one or more additional bacterial strains. In one embodiment, a mutant may have at least 90, 91, 92, 93%, 94%, 95%, 96%, 97%, 98%, or 99% sequence identity to a nucleic acid sequence of one or more of ARSA0001 and ARSA0002. In some embodiments, a mutant or variant may have at least 90, 91, 92, 93%, 94%, 95%, 96%, 97%, 98%, or 99% sequence identity across its entire genome sequence when compared to one or more of the genome sequence of ARSA0001 and ARSA0002. In one embodiment, a mutant may have at least 90, 91, 92, 93%, 94%, 95%, 96%, 97%, 98%, or 99% sequence identity across its entire genome sequence when compared to SEQ ID NO.: 1. In one embodiment, a mutant may have at least 90, 91, 92, 93%, 94%, 95%, 96%, 97%, 98%, or 99% sequence identity across its entire genome sequence when compared to SEQ ID NO.: 2.

In embodiments, a “mutant” may be a bacteriophage progeny. A bacteriophage progeny may be a bacteriophage obtainable after lysing Staphylococcus (e.g., S. aureus) target bacteria using a bacteriophage as described herein (i.e., the “parent bacteriophage”). In other words, the bacteriophage progeny may be a second (or further) generation bacteriophage.

In embodiments, “genetically modified” may be a bacteriophage whose polynucleotide sequence has been altered by genetic engineering techniques. Genetic engineering of polynucleotide sequences can be achieved by any modern molecular biology technique well known in the art, including but not limited to homologous recombination, bacteriophage engineering, CRISPR-Cas based manipulation, transformation of full-length naked phage into a host bacteria, and any combinations of techniques thereof.

In embodiments, a bacteriophage progeny is obtainable by contacting one or more bacteriophage(s) described herein, including for example, one selected from ARSA0001 or ARSA0002, with a Staphylococcus target bacteria such that the one or more bacteriophage(s) infects and lyses the target bacteria; and obtaining a bacteriophage released following lysis of the target bacteria. The bacteriophage progeny will typically comprise one or more nucleotide(s) mutation(s) when compared to the relevant parent bacteriophage.

In embodiments, the bacteriophage may be provided in the form of a single therapeutic composition or as a number of separate compositions each comprising one or more bacteriophage components of the composition. In embodiments where the bacteriophages are provided in a number of separate compositions, the bacteriophages may be administered to a subject sequentially or simultaneously (suitably simultaneously).

In embodiments, the bacteriophage composition includes bacteriophage concentrations between 1×10⁵ and 1×10¹¹ PFU per ml of each bacteriophage. In embodiments, the bacteriophage composition includes bacteriophage concentrations between 1×10⁶ and 1×10¹¹ PFU per ml of each bacteriophage. In embodiments, the bacteriophage composition includes bacteriophage concentrations between 1×10⁷ and 1×10¹¹ PFU per ml of each bacteriophage. In some embodiments, the bacteriophage composition includes bacteriophage concentrations between 1×10⁸ and 1×10¹¹ PFU per ml of each bacteriophage. In some embodiments, the composition includes 1×10⁸ to 1×109 PFU, 1×10⁸ to 1×1010 PFU, 1×10⁸ to 1×10¹¹ PFU, 1×109 to 1×1010 PFU, 1×109 to 1×10¹¹ PFU, or 1×1010 to 1×10¹¹ PFU of each phage per ml of composition. In some embodiments, a bacteriophage composition is administered to a subject at a dosage of at least about 1×10⁸ PFU of each phage, at least about 1×109 PFU of each phage, at least about 1×1010 PFU of each phage, or at least about 1×10¹¹ PFU of each phage per ml of composition. In embodiments, one or more bacteriophage(s) may be combined to form a composition that includes 1×10⁸, 1×109 or 1×1010, or 1×10¹¹ PFU of each phage per ml of composition. Concentrations include any value or range within the recited ranges, including endpoints. In specific embodiments, the composition may be administered at a dosage of at least 1×10⁸ PFU of total bacteriophages per milliliter. In specific embodiments, the composition may be administered at a dosage of at least 1×109 PFU of total bacteriophages per milliliter.

In some embodiments, the total dosage may vary depending on the formulation and storage container. In some embodiments, the total dosage may be lower or higher when administered to the patient, depending on whether it is delivered from a single-dose pre-filled syringe, or a multi-dose container. In some embodiments, the bacteriophage composition may be administered at a basal dose rate or a bolus rate. In some embodiments, the bacteriophage composition may be administered by an IV fluid drip.

In embodiments, the bacteriophage composition is stored at a range between 2-8° C. In some embodiments, the bacteriophage composition is stored between 2 and 3° C. In some embodiments, the bacteriophage composition is stored between 2 and 4° C. In some embodiments, the bacteriophage composition is stored between 2 and 5° C. In some embodiments, the bacteriophage composition is stored between 2 and 6° C. In some embodiments, the bacteriophage composition is stored between 2 and 7° C. In some embodiments, the bacteriophage composition is stored between 3 and 4° C. In some embodiments, the bacteriophage composition is stored between 3 and 5° C. In some embodiments, the bacteriophage composition is stored between 3 and 6° C. In some embodiments, the bacteriophage composition is stored between 3 and 7° C. In some embodiments, the bacteriophage composition is stored between 3 and 8° C. In some embodiments, the bacteriophage composition is stored between 4 and 5° C. In some embodiments, the bacteriophage composition is stored between 4 and 6° C. In some embodiments, the bacteriophage composition is stored between 4 and 7° C. In some embodiments, the bacteriophage composition is stored between 4 and 8° C. In some embodiments, the bacteriophage composition is stored between 5 and 8° C. In some embodiments, the bacteriophage composition is stored between 5 and 6° C. In some embodiments, the bacteriophage composition is stored between 5 and 7° C. In some embodiments, the bacteriophage composition is stored between 6 and 8° C. In some embodiments, the bacteriophage composition is stored between 6 and 7° C. In some embodiments, the bacteriophage composition is stored between 7 and 8° C. In some embodiments, the bacteriophage composition is stored at 2, 3, 4, 5, 6, 7, or 8° C. The temperature may be any value or subrange within the recited ranges, including endpoints.

In embodiments, the bacteriophage composition is stored at room temperature. In embodiments, the bacteriophage composition is stored at about 20-30° C. In embodiments, the bacteriophage composition is stored at about 20-25° C. In embodiments, the bacteriophage composition is stored at about 20-22° C. In embodiments, the bacteriophage composition is stored at about 20° C. In embodiments, the bacteriophage composition is stored at about 21° C. In embodiments, the bacteriophage composition is stored at about 22° C. In embodiments, the bacteriophage composition is stored at 23° C. In embodiments, the bacteriophage composition is stored at 24° C. In embodiments, the bacteriophage composition is stored at 25° C. The temperature may be any value or subrange within the recited ranges, including endpoints.

In embodiments, the bacteriophage composition is resistant to inactivation by the immune system of a subject. In some embodiments, the bacteriophage is resistant to inactivation by the mononuclear phagocytosis system of a subject. In some embodiments, the bacteriophage is resistant to inactivation by the complement system of a subject. In some embodiments, the bacteriophage is resistant to inactivation by the blood complement system of a subject. In some embodiments, the bacteriophage is resistant to inactivation by the blood complement C3-complex of proteins of a subject.

In embodiments, the bacteriophage composition includes at least one lytic bacteriophage. In some embodiments, the bacteriophage includes at least one lytic phage that can kill a bacterium and release phage progeny through cell lysis.

In an aspect, provided herein is a bacterial host manufacturing strain including a bacteriophage where the bacteriophage includes a polynucleotide sequence selected from SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, provided herein is a bacterial host manufacturing strain including a bacteriophage where the bacteriophage includes a polynucleotide sequence selected from SEQ ID NO: 1, SEQ ID NO: 2, a polynucleotide sequence with at least 90% identity to SEQ ID NO: 1, and a polynucleotide sequence with at least 90% identity to SEQ ID NO: 2.

In embodiments, provided herein is a bacterial host manufacturing strain including a bacteriophage that includes a polynucleotide sequence of SEQ ID NO: 1. In embodiments, provided herein is a bacterial host manufacturing strain including a bacteriophage that includes a polynucleotide sequence of SEQ ID NO: 2. In embodiments, provided herein is a bacterial host manufacturing strain including a bacteriophage that includes a polynucleotide sequence of with at least 93% identity to SEQ ID NO: 1. In embodiments, provided herein is a bacterial host manufacturing strain including a bacteriophage that includes a polynucleotide sequence of polynucleotide sequence with at least 93% identity to SEQ ID NO: 2. In other embodiments, provided herein is a bacterial host manufacturing strain including a bacteriophage that includes a polynucleotide sequence of with at least 90% identity to SEQ ID NO: 1. In embodiments, provided herein is a bacterial host manufacturing strain including a bacteriophage that includes a polynucleotide sequence of polynucleotide sequence with at least 90% identity to SEQ ID NO: 2. In embodiments, provided herein is a bacterial host manufacturing strain including a bacteriophage according to any of the various embodiments described herein.

In embodiments, provided herein are uses of a composition according to any of the various embodiments described herein in the treatment of a Staphylococcus aureus infection in a subject. In embodiments, the use includes administering a composition according to any of the embodiments described herein to a subject suffering from a Staphylococcus aureus infection.

In embodiments, provided herein are uses of a composition including one or more distinct bacteriophages that target Staphylococcus aureus in the treatment of subject with a Staphylococcus aureus bacterial infection. The uses include administering the composition to said subject; wherein at least one of the bacteriophage is selected from a bacteriophage comprising a polynucleotide sequence of SEQ ID NO: 1, SEQ ID NO: 2, a polynucleotide sequence with at least 93% identity to SEQ ID NO: 1, and a polynucleotide sequence with at least 93% identity to SEQ ID NO: 2. The uses may include administering the composition to said subject; wherein at least one of the bacteriophage is selected from a bacteriophage comprising a polynucleotide sequence of SEQ ID NO: 1, SEQ ID NO: 2, a polynucleotide sequence with at least 90% identity to SEQ ID NO: 1, and a polynucleotide sequence with at least 90% identity to SEQ ID NO: 2.

### Methods of Use

In an aspect, provided herein are methods of treating a subject with a bacterial infection including selecting a bacteriophage based upon resistance to blood complement inactivation and administering the bacteriophage to the subject.

In embodiments, selecting a bacteriophage includes performing a blood/serum resistance assay and selecting bacteriophage based on survival in the blood/serum for a pre-determined amount of time.

In an aspect, provided herein are methods of treating a subject with a bacterial infection including administering to the subject one or more distinct bacteriophage selected from a bacteriophage including a polynucleotide sequence selected from SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, provided herein are methods of treating a subject with a bacterial infection including administering to the subject one or more distinct bacteriophage selected from a bacteriophage including a polynucleotide sequence selected from SEQ ID NO: 1, SEQ ID NO: 2, a polynucleotide sequence with at least 90% identity to SEQ ID NO: 1, and a polynucleotide sequence with at least 90% identity to SEQ ID NO: 2. In an aspect, provided herein are methods of treating a subject with a bacterial infection including administering to the subject one or more distinct bacteriophage as described in any embodiment herein.

In embodiments, provided herein are methods of treating a subject with a bacterial infection. In some embodiments, the bacterial infection at least partially includes Staphylococcus. In some embodiments, the bacterial infection at least partially includes Staphylococcus aureus. In some embodiments, the bacterial infection includes Staphylococcus aureus strains resistant to chemical antibiotics. In some embodiments, the bacterial strains include drug resistant and/or multi-drug resistant Staphylococcus aureus strains. In some embodiments, the bacterial strain is the drug-resistant strain vancomycin-intermediate Staphylococcus aureus (VISA). In some embodiments, the bacterial strain is the drug-resistant strain vancomycin-resistant Staphylococcus aureus (VRSA). In some embodiments, the bacterial strain is the drug-resistant strain methicillin-resistant Staphylococcus aureus (MRSA).

In embodiments, provided herein are methods of treating a subject with a bacterial infection including administering to a subject a bacteriophage that is resistant to inactivation in the blood. In embodiments, provided herein are methods of treating a subject with a bacterial infection including administering to a subject a bacteriophage that is resistant to inactivation by a blood complement system. In embodiments, the bacteriophage can be any bacteriophage as described herein.

In embodiments, provided herein are methods of treating a subject with a bacterial infection including administering to a subject a bacteriophage that includes a polynucleotide sequence of SEQ ID NO: 1. In embodiments, provided herein are methods of treating a subject with a bacterial infection including administering to a subject a bacteriophage that includes a polynucleotide sequence of SEQ ID NO: 2. In embodiments, provided herein are methods of treating a subject with a bacterial infection including administering to a subject a bacteriophage that includes a polynucleotide sequence with at least 93% identity to a SEQ ID NO: 1. In embodiments, provided herein are methods of treating a subject with a bacterial infection including administering to a subject a bacteriophage that includes a polynucleotide sequence with at least 90% identity to a SEQ ID NO: 1. In embodiments, provided herein are methods of treating a subject with a bacterial infection including administering to a subject a bacteriophage that includes a polynucleotide sequence with at least 93% identity to SEQ ID NO: 2. In embodiments, provided herein are methods of treating a subject with a bacterial infection including administering to a subject a bacteriophage that includes a polynucleotide sequence with at least 90% identity to SEQ ID NO: 2.

In embodiments, provided herein are methods of treating a subject with a bacterial infection including administering to a subject a bacteriophage that infects and kills one or more of vancomycin-intermediate Staphylococcus aureus (VISA), vancomycin-resistant Staphylococcus aureus (VRSA), methicillin-resistant Staphylococcus aureus (MRSA). In embodiments, provided herein are methods of treating a subject with a bacterial infection including administering to a subject a bacteriophage that infects and kills vancomycin-intermediate Staphylococcus aureus (VISA). In embodiments, the bacteriophage composition includes bacteriophage that infects and kills vancomycin-resistant Staphylococcus aureus (VRSA). In embodiments, provided herein are methods of treating a subject with a bacterial infection including administering to a subject a bacteriophage that infects and kills methicillin-resistant Staphylococcus aureus (MRSA).

In embodiments, provided herein are methods of treating a subject with a bacterial infection including administering to a subject one or more bacteriophages. In embodiments, the one or more bacteriophages are suitable for treating a bacterial infection, in particular a Staphylococcus infection. In embodiments, the bacteriophage includes one or more additional phages and can be a phage with 93%-100% nucleic acid sequence identity to any of the phage described herein. The bacteriophage can have a polynucleotide sequence, that includes a polynucleotide sequence having at least 93% but not 100% identity to any one of SEQ ID NO: 1 or SEQ ID NO: 2. In embodiments, the bacteriophage includes one or more additional phages and can be a phage with 90%-100% nucleic acid sequence identity to any of the phage described herein. The bacteriophage can have a polynucleotide sequence, that includes a polynucleotide sequence having at least 90% but not 100% identity to any one of SEQ ID NO: 1 or SEQ ID NO: 2. Percent identity may be any value or subrange within the recited ranges, including endpoints.

In embodiments, provided herein are methods of treating a subject with a bacterial infection including administering to a subject a bacteriophage composition that includes one or more bacteriophage and the composition's target bacteria range can be broader than the range of any individual bacteriophage or the phage collectively in the composition, or have an effectiveness that is greater than the sum of effectiveness of the individual bacteriophage. In embodiments, provided herein are methods of treating a subject with a bacterial infection including administering to a subject a bacteriophage composition that includes a bacteriophage with polynucleotide sequence SEQ ID NO: 1. In embodiments, provided herein are methods of treating a subject with a bacterial infection including administering to a subject a bacteriophage composition that includes a bacteriophage with a polynucleotide sequence at least 90% identity with SEQ ID NO: 1. In embodiments, provided herein are methods of treating a subject with a bacterial infection including administering to a subject a bacteriophage composition that includes a bacteriophage with a polynucleotide sequence at least 91% identity with SEQ ID NO: 1. In embodiments, provided herein are methods of treating a subject with a bacterial infection including administering to a subject a bacteriophage composition that includes a bacteriophage with a polynucleotide sequence at least 92% identity with SEQ ID NO: 1. In embodiments, provided herein are methods of treating a subject with a bacterial infection including administering to a subject a bacteriophage composition that includes a bacteriophage with a polynucleotide sequence at least 93% identity with SEQ ID NO: 1. In embodiments, provided herein are methods of treating a subject with a bacterial infection including administering to a subject a bacteriophage composition that includes a bacteriophage with a polynucleotide sequence at least 94% identity with SEQ ID NO: 1. In embodiments, provided herein are methods of treating a subject with a bacterial infection including administering to a subject a bacteriophage composition that includes a bacteriophage with a polynucleotide sequence at least 95% identity with SEQ ID NO: 1. In embodiments, provided herein are methods of treating a subject with a bacterial infection including administering to a subject a bacteriophage composition that includes a bacteriophage with a polynucleotide sequence at least 96% identity with SEQ ID NO: 1. In embodiments, provided herein are methods of treating a subject with a bacterial infection including administering to a subject a bacteriophage composition that includes a bacteriophage with a polynucleotide sequence at least 97% identity with SEQ ID NO: 1. In embodiments, provided herein are methods of treating a subject with a bacterial infection including administering to a subject a bacteriophage composition that includes a bacteriophage with a polynucleotide sequence at least 98% identity with SEQ ID NO: 1. In embodiments, provided herein are methods of treating a subject with a bacterial infection including administering to a subject a bacteriophage composition that includes a bacteriophage with a polynucleotide sequence at least 99% identity with SEQ ID NO: 1.

In embodiments, provided herein are methods of treating a subject with a bacterial infection including administering to a subject a bacteriophage composition that includes a bacteriophage with polynucleotide sequence SEQ ID NO: 2. In embodiments, provided herein are methods of treating a subject with a bacterial infection including administering to a subject a bacteriophage composition that includes a bacteriophage with a polynucleotide sequence at least 90% identity with SEQ ID NO: 2. In embodiments, provided herein are methods of treating a subject with a bacterial infection including administering to a subject a bacteriophage composition that includes a bacteriophage with a polynucleotide sequence at least 91% identity with SEQ ID NO: 2. In embodiments, provided herein are methods of treating a subject with a bacterial infection including administering to a subject a bacteriophage composition that includes a bacteriophage with a polynucleotide sequence at least 92% identity with SEQ ID NO: 2. In embodiments, provided herein are methods of treating a subject with a bacterial infection including administering to a subject a bacteriophage composition that includes a bacteriophage with a polynucleotide sequence at least 93% identity with SEQ ID NO: 2. In embodiments, provided herein are methods of treating a subject with a bacterial infection including administering to a subject a bacteriophage composition that includes a bacteriophage with a polynucleotide sequence at least 94% identity with SEQ ID NO: 2. In embodiments, provided herein are methods of treating a subject with a bacterial infection including administering to a subject a bacteriophage composition that includes a bacteriophage with a polynucleotide sequence at least 95% identity with SEQ ID NO: 2. In embodiments, provided herein are methods of treating a subject with a bacterial infection including administering to a subject a bacteriophage composition that includes a bacteriophage with a polynucleotide sequence at least 96% identity with SEQ ID NO: 2. In embodiments, provided herein are methods of treating a subject with a bacterial infection including administering to a subject a bacteriophage composition that includes a bacteriophage with a polynucleotide sequence at least 97% identity with SEQ ID NO: 2. In embodiments, provided herein are methods of treating a subject with a bacterial infection including administering to a subject a bacteriophage composition that includes a bacteriophage with a polynucleotide sequence at least 98% identity with SEQ ID NO: 2. In embodiments, provided herein are methods of treating a subject with a bacterial infection including administering to a subject a bacteriophage composition that includes a bacteriophage with a polynucleotide sequence at least 99% identity with SEQ ID NO: 2. In embodiments, provided herein are methods of treating a subject with a bacterial infection including administering to a subject a bacteriophage composition that have any combination polynucleotide sequences of SEQ ID NO: 1 and SEQ ID NO: 2.

In embodiments, provided herein are methods of treating a subject with a bacterial infection including administering to a subject a bacteriophage composition that includes at least one bacteriophage that is genetically modified. In embodiments, provided herein are methods of treating a subject with a bacterial infection including administering to a subject a bacteriophage composition that includes at least one naturally occurring phage or can exclude naturally occurring phage. In embodiments, provided herein are methods of treating a subject with a bacterial infection including administering to a subject a bacteriophage composition that includes at least one lytic phage. In embodiments, provided herein are methods of treating a subject with a bacterial infection including administering to a subject a bacteriophage composition that includes at least one bacteriophage resistant to inactivation by the blood complement system.

In an aspect, provided herein are methods of modifying the microbial flora in a subject including administering to the subject at least one bacteriophage that targets Staphylococcus bacteria, where the bacteriophage includes a polynucleotide sequence selected from SEQ ID NO: 1, SEQ ID NO: 2, a polynucleotide sequence with at least 93% identity to SEQ ID NO: 1, and a polynucleotide sequence with at least 93% identity to SEQ ID NO: 2. In another aspect, the bacteriophage may include a polynucleotide sequence selected from SEQ ID NO: 1, SEQ ID NO: 2, a polynucleotide sequence with at least 90% identity to SEQ ID NO: 1, and a polynucleotide sequence with at least 90% identity to SEQ ID NO: 2.

In embodiments, modifying the microbial flora includes killing the majority of a particular bacterial strain in the microbial flora, limiting growth a particular bacterial strain in the microbial flora, and/or slowing the growth of a particular bacterial strain in the microbial flora. In embodiments, modifying the microbial flora includes killing the majority of a particular bacterial strain in the microbial flora. In embodiments, modifying the microbial flora includes limiting growth a particular bacterial strain in the microbial flora. In embodiments, modifying the microbial flora includes slowing the growth of a particular bacterial strain in the microbial flora.

In embodiments, provided herein are methods of modifying the microbial flora in a subject including administering to the subject a bacteriophage that targets Staphylococcus aureus bacteria, where the bacteriophage includes a polynucleotide sequence of SEQ ID NO: 1. In embodiments, provided herein are methods of modifying the microbial flora in a subject including administering to the subject at least one bacteriophage that targets Staphylococcus aureus bacteria, where the bacteriophage includes a polynucleotide sequence of SEQ ID NO: 2. In embodiments, provided herein are methods of modifying the microbial flora in a subject including administering to the subject at least one bacteriophage that targets Staphylococcus aureus bacteria, where the bacteriophage includes a polynucleotide sequence with at least 93% identity to SEQ ID NO: 1. In embodiments, provided herein are methods of modifying the microbial flora in a subject including administering to the subject at least one bacteriophage that targets Staphylococcus aureus bacteria, where the bacteriophage includes a polynucleotide sequence with at least 93% identity to SEQ ID NO: 2. In embodiments, provided herein are methods of modifying the microbial flora in a subject including administering to the subject at least one bacteriophage that targets Staphylococcus aureus bacteria, where the bacteriophage includes a polynucleotide sequence with at least 90% identity to SEQ ID NO: 1. In embodiments, provided herein are methods of modifying the microbial flora in a subject including administering to the subject at least one bacteriophage that targets Staphylococcus aureus bacteria, where the bacteriophage includes a polynucleotide sequence with at least 90% identity to SEQ ID NO: 2.

In embodiments, provided herein are methods of modifying the microbial flora in a subject including administering to the subject a bacteriophage that infects and kills Staphylococcus aureus bacteria, where the bacteriophage includes a polynucleotide sequence of SEQ ID NO: 1. In embodiments, provided herein are methods of modifying the microbial flora in a subject including administering to the subject at least one bacteriophage that infects and kills Staphylococcus aureus bacteria, where the bacteriophage includes a polynucleotide sequence of SEQ ID NO: 2. In embodiments, provided herein are methods of modifying the microbial flora in a subject including administering to the subject at least one bacteriophage that infects and kills Staphylococcus aureus bacteria, where the bacteriophage includes a polynucleotide sequence with at least 93% identity to SEQ ID NO: 1. In embodiments, provided herein are methods of modifying the microbial flora in a subject including administering to the subject at least one bacteriophage that infects and kills Staphylococcus aureus bacteria, where the bacteriophage includes a polynucleotide sequence with at least 93% identity to SEQ ID NO: 2. In embodiments, provided herein are methods of modifying the microbial flora in a subject including administering to the subject at least one bacteriophage that infects and kills Staphylococcus aureus bacteria, where the bacteriophage includes a polynucleotide sequence with at least 90% identity to SEQ ID NO: 1. In embodiments, provided herein are methods of modifying the microbial flora in a subject including administering to the subject at least one bacteriophage that infects and kills Staphylococcus aureus bacteria, where the bacteriophage includes a polynucleotide sequence with at least 90% identity to SEQ ID NO: 2.

In embodiments, provided herein are methods of modifying the microbial flora in a subject including administering to the subject a bacteriophage that that is genetically modified. In embodiments, provided herein are methods of modifying the microbial flora in a subject including administering to the subject at least one naturally occurring bacteriophage or can exclude naturally occurring phage. In embodiments, provided herein are methods of modifying the microbial flora in a subject including administering to the subject at least one lytic phage. In embodiments, provided herein are methods of modifying the microbial flora in a subject including administering to the subject at least one bacteriophage resistant to inactivation by the blood complement system.

In embodiments, provided herein are methods for administering a bacteriophage to a subject, where the bacteriophage includes a bacteriophage concentration range between 1×10⁸ and 1×10¹¹ PFU per ml of each bacteriophage. In some embodiments, the bacteriophage concentration is 1×10⁸ to 1×109 PFU, 1×10⁸ to 1×1010 PFU, or 1×10⁸ to 1×10¹¹ PFU of each phage per ml of composition. In some embodiments, the bacteriophage concentration is 1×109 to 1×1010 PFU, or 1×109 to 1×10¹¹ PFU of each phage per ml of composition. In some embodiments, the bacteriophage concentration is 1×1010 to 1×10¹¹ PFU of each phage per ml of composition. In some embodiments, the bacteriophage is administered to a subject at a dosage of at least about 1×10⁸ PFU of each phage, at least about 1×109 PFU of each phage, at least about 1×1010 PFU of each phage, or at least about 1×10¹¹ PFU of each phage per ml of composition. In embodiments, one or more bacteriophage(s) may be combined to form a total concentration of 1×10⁸, 1×109, 1×1010, or 1×10¹¹ PFU of each phage per ml of composition. Concentrations include any value or range within the recited ranges, including endpoints.

In embodiments, the methods provided herein include administering a bacteriophage, where at least about 80% of bacteriophage retain lytic activity after exposure to human plasma for a time period between about 10 minutes and about 120 minutes. In embodiments, the methods provided herein include administering a bacteriophage, where at least about 81% of bacteriophage retain lytic activity after exposure to human plasma for a time period between about 10 minutes and about 120 minutes. In embodiments, the methods provided herein include administering a bacteriophage, where at least about 82% of bacteriophage retain lytic activity after exposure to human plasma for a time period between about 10 minutes and about 120 minutes. In embodiments, the methods provided herein include administering a bacteriophage, where at least about 83% of bacteriophage retain lytic activity after exposure to human plasma for a time period between about 10 minutes and about 120 minutes. In embodiments, the methods provided herein include administering a bacteriophage, where at least about 84% of bacteriophage retain lytic activity after exposure to human plasma for a time period between about 10 minutes and about 120 minutes. In embodiments, the methods provided herein include administering a bacteriophage, where at least about 85% of bacteriophage retain lytic activity after exposure to human plasma for a time period between about 10 minutes and about 120 minutes. In embodiments, the methods provided herein include administering a bacteriophage, where at least about 86% of bacteriophage retain lytic activity after exposure to human plasma for a time period between about 10 minutes and about 120 minutes. In embodiments, the methods provided herein include administering a bacteriophage, where at least about 87% of bacteriophage retain lytic activity after exposure to human plasma for a time period between about 10 minutes and about 120 minutes. In embodiments, the methods provided herein include administering a bacteriophage, where at least about 88% of bacteriophage retain lytic activity after exposure to human plasma for a time period between about 10 minutes and about 120 minutes. In embodiments, the methods provided herein include administering a bacteriophage, where at least about 89% of bacteriophage retain lytic activity after exposure to human plasma for a time period between about 10 minutes and about 120 minutes. In embodiments, the methods provided herein include administering a bacteriophage, where at least about 90% of bacteriophage retain lytic activity after exposure to human plasma for a time period between about 10 minutes and about 120 minutes. In embodiments, the methods provided herein include administering a bacteriophage, where at least about 90% of bacteriophage retain lytic activity after exposure to human plasma for a time period between about 10 minutes and about 120 minutes. In embodiments, the methods provided herein include administering a bacteriophage, where at least about 92% of bacteriophage retain lytic activity after exposure to human plasma for a time period between about 10 minutes and about 120 minutes. In embodiments, the methods provided herein include administering a bacteriophage, where at least about 93% of bacteriophage retain lytic activity after exposure to human plasma for a time period between about 10 minutes and about 120 minutes. In embodiments, the methods provided herein include administering a bacteriophage, where at least about 94% of bacteriophage retain lytic activity after exposure to human plasma for a time period between about 10 minutes and about 120 minutes. In embodiments, the methods provided herein include administering a bacteriophage, where at least about 95% of bacteriophage retain lytic activity after exposure to human plasma for a time period between about 10 minutes and about 120 minutes. In embodiments, the methods provided herein include administering a bacteriophage, where at least about 96% of bacteriophage retain lytic activity after exposure to human plasma for a time period between about 10 minutes and about 120 minutes. In embodiments, the methods provided herein include administering a bacteriophage, where at least about 97% of bacteriophage retain lytic activity after exposure to human plasma for a time period between about 10 minutes and about 120 minutes. In embodiments, the methods provided herein include administering a bacteriophage, where at least about 98% of bacteriophage retain lytic activity after exposure to human plasma for a time period between about 10 minutes and about 120 minutes. In embodiments, the methods provided herein include administering a bacteriophage, where at least about 99% of bacteriophage retain lytic activity after exposure to human plasma for a time period between about 10 minutes and about 120 minutes. In specific embodiments, the time period is between about 10 minutes and about 90 minutes.

In embodiments, the methods provided herein include administering a bacteriophage, where at least about 80% of bacteriophage retain lytic activity after exposure to human plasma for a time period between 10 and 20 minutes. In some embodiments, at least about 80% of bacteriophage retain lytic activity after exposure to human plasma for a time period between 10 and 30 minutes. In some embodiments, at least about 80% of bacteriophage retain lytic activity after exposure to human plasma for a time period between 10 and 40 minutes. In some embodiments, at least about 80% of bacteriophage retain lytic activity after exposure to human plasma for a time period between 10 and 50 minutes. In some embodiments, at least about 80% of bacteriophage retain lytic activity after exposure to human plasma for a time period between 10 and 60 minutes. In some embodiments, at least about 80% of bacteriophage retain lytic activity after exposure to human plasma for a time period between 10 and 70 minutes. In some embodiments, at least about 80% of bacteriophage retain lytic activity after exposure to human plasma for a time period between 10 and 80 minutes. In some embodiments, at least about 80% of bacteriophage retain lytic activity after exposure to human plasma for a time period between 10 and 90 minutes. In some embodiments, at least about 80% of bacteriophage retain lytic activity after exposure to human plasma for a time period between 10 and 100 minutes. In some embodiments, at least about 80% of bacteriophage retain lytic activity after exposure to human plasma for a time period between 10 and 110 minutes. In some embodiments, at least about 80% of bacteriophage retain lytic activity after exposure to human plasma for a time period between 10 and 120 minutes. In some embodiments, at least about 80% of bacteriophage retain lytic activity after exposure to human plasma for a time period between 20 and 30 minutes. In some embodiments, at least about 80% of bacteriophage retain lytic activity after exposure to human plasma for a time period between 20 and 40 minutes. In some embodiments, at least about 80% of bacteriophage retain lytic activity after exposure to human plasma for a time period between 20 and 50 minutes. In some embodiments, at least about 80% of bacteriophage retain lytic activity after exposure to human plasma for a time period between 20 and 60 minutes. In some embodiments, at least about 80% of bacteriophage retain lytic activity after exposure to human plasma for a time period between 20 and 70 minutes. In some embodiments, at least about 80% of bacteriophage retain lytic activity after exposure to human plasma for a time period between 20 and 80 minutes. In some embodiments, at least about 80% of bacteriophage retain lytic activity after exposure to human plasma for a time period between 20 and 90 minutes. In some embodiments, at least about 80% of bacteriophage retain lytic activity after exposure to human plasma for a time period between 20 and 100 minutes. In some embodiments, at least about 80% of bacteriophage retain lytic activity after exposure to human plasma for a time period between 20 and 110 minutes. In some embodiments, at least about 80% of bacteriophage retain lytic activity after exposure to human plasma for a time period between 20 and 120 minutes. In some embodiments, at least about 80% of bacteriophage retain lytic activity after exposure to human plasma for a time period between 30 and 40 minutes. In some embodiments, at least about 80% of bacteriophage retain lytic activity after exposure to human plasma for a time period between 30 and 50 minutes. In some embodiments, at least about 80% of bacteriophage retain lytic activity after exposure to human plasma for a time period between 30 and 60 minutes. In some embodiments, at least about 80% of bacteriophage retain lytic activity after exposure to human plasma for a time period between 30 and 70 minutes. In some embodiments, at least about 80% of bacteriophage retain lytic activity after exposure to human plasma for a time period between 30 and 80 minutes. In some embodiments, at least about 80% of bacteriophage retain lytic activity after exposure to human plasma for a time period between 30 and 90 minutes. In some embodiments, at least about 80% of bacteriophage retain lytic activity after exposure to human plasma for a time period between 30 and 100 minutes. In some embodiments, at least about 80% of bacteriophage retain lytic activity after exposure to human plasma for a time period between 30 and 110 minutes. In some embodiments, at least about 80% of bacteriophage retain lytic activity after exposure to human plasma for a time period between 30 and 120 minutes. In some embodiments, at least about 80% of bacteriophage retain lytic activity after exposure to human plasma for a time period between 40 and 50 minutes. In some embodiments, at least about 80% of bacteriophage retain lytic activity after exposure to human plasma for a time period between 40 and 60 minutes. In some embodiments, at least about 80% of bacteriophage retain lytic activity after exposure to human plasma for a time period between 40 and 70 minutes. In some embodiments, at least about 80% of bacteriophage retain lytic activity after exposure to human plasma for a time period between 40 and 80 minutes. In some embodiments, at least about 80% of bacteriophage retain lytic activity after exposure to human plasma for a time period between 40 and 90 minutes. In some embodiments, the bacteriophage is administered to a subject for a time between 40 and 100 minutes. In some embodiments, at least about 80% of bacteriophage retain lytic activity after exposure to human plasma for a time period between 40 and 110 minutes. In some embodiments, at least about 80% of bacteriophage retain lytic activity after exposure to human plasma for a time period between 40 and 120 minutes. In some embodiments, at least about 80% of bacteriophage retain lytic activity after exposure to human plasma for a time period between 50 and 60 minutes. In some embodiments, at least about 80% of bacteriophage retain lytic activity after exposure to human plasma for a time period between 50 and 70 minutes. In some embodiments, at least about 80% of bacteriophage retain lytic activity after exposure to human plasma for a time period between 50 and 80 minutes. In some embodiments, at least about 80% of bacteriophage retain lytic activity after exposure to human plasma for a time period between 50 and 90 minutes. In some embodiments, at least about 80% of bacteriophage retain lytic activity after exposure to human plasma for a time period between 50 and 100 minutes. In some embodiments, at least about 80% of bacteriophage retain lytic activity after exposure to human plasma for a time period between 50 and 110 minutes. In some embodiments, at least about 80% of bacteriophage retain lytic activity after exposure to human plasma for a time period between 50 and 120 minutes. In some embodiments, at least about 80% of bacteriophage retain lytic activity after exposure to human plasma for a time period between 60 and 70 minutes. In some embodiments, at least about 80% of bacteriophage retain lytic activity after exposure to human plasma for a time period between 60 and 80 minutes. In some embodiments, at least about 80% of bacteriophage retain lytic activity after exposure to human plasma for a time period between 60 and 90 minutes. In some embodiments, at least about 80% of bacteriophage retain lytic activity after exposure to human plasma for a time period between 60 and 100 minutes. In some embodiments, at least about 80% of bacteriophage retain lytic activity after exposure to human plasma for a time period between 60 and 110 minutes. In some embodiments, at least about 80% of bacteriophage retain lytic activity after exposure to human plasma for a time period between 60 and 120 minutes. In some embodiments, at least about 80% of bacteriophage retain lytic activity after exposure to human plasma for a time period between 70 and 80 minutes. In some embodiments, at least about 80% of bacteriophage retain lytic activity after exposure to human plasma for a time period between 70 and 90 minutes. In some embodiments, at least about 80% of bacteriophage retain lytic activity after exposure to human plasma for a time period between 70 and 100 minutes. In some embodiments, at least about 80% of bacteriophage retain lytic activity after exposure to human plasma for a time period between 70 and 110 minutes. In some embodiments, at least about 80% of bacteriophage retain lytic activity after exposure to human plasma for a time period between 70 and 120 minutes. In some embodiments, at least about 80% of bacteriophage retain lytic activity after exposure to human plasma for a time period between 80 and 90 minutes. In some embodiments, at least about 80% of bacteriophage retain lytic activity after exposure to human plasma for a time period between 80 and 100 minutes. In some embodiments, at least about 80% of bacteriophage retain lytic activity after exposure to human plasma for a time period between 80 and 110 minutes. In some embodiments, at least about 80% of bacteriophage retain lytic activity after exposure to human plasma for a time period between 80 and 120 minutes. In some embodiments, at least about 80% of bacteriophage retain lytic activity after exposure to human plasma for a time period between 90 and 100 minutes. In some embodiments, at least about 80% of bacteriophage retain lytic activity after exposure to human plasma for a time period between 90 and 110 minutes. In some embodiments, at least about 80% of bacteriophage retain lytic activity after exposure to human plasma for a time period between 90 and 120 minutes. In some embodiments, at least about 80% of bacteriophage retain lytic activity after exposure to human plasma for a time period between 100 and 110 minutes. In some embodiments, at least about 80% of bacteriophage retain lytic activity after exposure to human plasma for a time period between 100 and 120 minutes. In some embodiments, at least about 80% of bacteriophage retain lytic activity after exposure to human plasma for a time period between 110 and 120 minutes. The amount of time may be any value or subrange within the recited ranges, including endpoints. The bacteriophage composition retains at least greater than 80% of its lytic activity in human plasma at the end of the time point.

In embodiments, provided herein are methods of treating a subject with a bacterial infection including administering to a subject a bacteriophage that is at least partially resistant to inactivation by immune system of the administered subject. In embodiments, provided herein are methods of treating a subject with a bacterial infection including administering to a subject a bacteriophage that is resistant to inactivation by the mononuclear phagocytosis system of the administered subject. In embodiments, provided herein are methods of treating a subject with a bacterial infection including administering to a subject a bacteriophage that is resistant to inactivation by the complement system of the administered subject. In embodiments, provided herein are methods of treating a subject with a bacterial infection including administering to a subject a bacteriophage that is resistant to inactivation by the blood complement system of the administered subject. In embodiments, provided herein are methods of treating a subject with a bacterial infection including administering to a subject a bacteriophage that is resistant to inactivation by the blood complement C3-complex of proteins of the administered subject.

In embodiments, provided herein are methods of treating a subject with a bacterial infection including administering to a subject one or more bacteriophages that belong to the genus Silviavirus. In embodiments, provided herein are methods of treating a subject with a bacterial infection including administering to a subject a bacteriophage strain from the bacteriophage genus Silviavirus. In embodiments, the bacteriophage includes a bacteriophage selected from MR003 (Accession No. AP019522.1), Qdsa001 (Accession No. KY779848.1), Remus (Accession No. NC_022090.1), Romulus (Accession No. NC_020877.1), SA1l (Accession No. NC_019511.12), StAP1 (Accession No. KC532239.1), and Stsau2 (Accession No. NC_030933.1). In embodiments, the bacteriophage is Silviavirus Staphylococcus virus Remus. In embodiments, the bacteriophage is Silviavirus Staphylococcus virus SA11. In embodiments, the bacteriophage is Silviavirus Staphylococcus virus Romulus. In embodiments, the bacteriophage is Silviavirus Staphylococcus virus Qdsa001. In embodiments, the bacteriophage is Silviavirus Staphylococcus virus MR003. In embodiments, the bacteriophage is Silviavirus Staphylococcus virus StAP1. In embodiments, the bacteriophage is Silviavirus Staphylococcus virus Stsau2.

In embodiments, provided herein are methods of treating a subject with a bacterial infection including administering to a subject one or more bacteriophage administered intravenously. In embodiments, provided herein are methods of treating a subject with a bacterial infection including administering to a subject one or more bacteriophage administered via an intra-articular injection. In embodiments, provided herein are methods of treating a subject with a bacterial infection including administering to a subject one or more bacteriophage administered via inhalation. In embodiments, provided herein are methods of treating a subject with a bacterial infection including administering to a subject one or more bacteriophages administered via nebulization. In embodiments, provided herein are methods of treating a subject with a bacterial infection including administering to a subject one or more bacteriophages administered intranasally, orally, by inhalation, vaginally, rectally, or parenterally, for example by intradermal, subcutaneous, intramuscular, intraperitoneal, intrarectal, intraarterial, intralymphatic, intravenous, intra-articular, intrathecal, and intratracheal routes. Parenteral administration, if used, is generally characterized by injection.

In embodiments, provided herein are methods of treating a subject with a bacterial infection including administering to the subject one or more distinct bacteriophage. In embodiments, the bacterial infection is selected from bacteremia, septicemia, pulmonary infection, rhinosinusitis, urinary tract infection, intra-abdominal infection, skin infection, skin structure infection, endocarditis, and an implant infection. In embodiments, provided herein are methods of treating a subject with a bacterial infection including administering to the subject one or more distinct bacteriophage, where the bacterial infection is bacteremia. In embodiments, provided herein are methods of treating a subject with a bacterial infection including administering to the subject one or more distinct bacteriophage, where the bacterial infection is septicemia. In embodiments, provided herein are methods of treating a subject with a bacterial infection including administering to the subject one or more distinct bacteriophage, where the bacterial infection is pulmonary infection. In embodiments, provided herein are methods of treating a subject with a bacterial infection including administering to the subject one or more distinct bacteriophage, where the bacterial infection is rhinosinusitis. In embodiments, provided herein are methods of treating a subject with a bacterial infection including administering to the subject one or more distinct bacteriophage, where the bacterial infection is urinary tract infection. In embodiments, provided herein are methods of treating a subject with a bacterial infection including administering to the subject one or more distinct bacteriophage, where the bacterial infection is intra-abdominal infection. In embodiments, provided herein are methods of treating a subject with a bacterial infection including administering to the subject one or more distinct bacteriophage, where the bacterial infection is skin infection. In embodiments, provided herein are methods of treating a subject with a bacterial infection including administering to the subject one or more distinct bacteriophage, where the bacterial infection is skin structure infection. In embodiments, provided herein are methods of treating a subject with a bacterial infection including administering to the subject one or more distinct bacteriophage, where the bacterial infection is endocarditis. In embodiments, provided herein are methods of treating a subject with a bacterial infection including administering to the subject one or more distinct bacteriophage, where the bacterial infection is an implant infection. In embodiments, provided herein are methods of treating a subject with a bacterial infection including administering to the subject one or more distinct bacteriophage, where the bacterial infection is a cardiac implant infection. In embodiments, provided herein are methods of treating a subject with a bacterial infection including administering to the subject one or more distinct bacteriophage, where the bacterial infection is a cardiac implant infection caused by a ventricular assist device. In embodiments, provided herein are methods of treating a subject with a bacterial infection including administering to the subject one or more distinct bacteriophage, where the bacterial infection is a cardiac implant infection caused by a pacemaker. In embodiments, provided herein are methods of treating a subject with a bacterial infection including administering to the subject one or more distinct bacteriophage, where the bacterial infection is a prosthetic joint infection. In embodiments, provided herein are methods of treating a subject with a bacterial infection including administering to the subject one or more distinct bacteriophage, where the bacterial infection is prosthetic valve endocarditis. In embodiments, the bacterial infection is resistant to one or more antibiotics.

In embodiments, provided herein are methods for treating bacterial infection by administering any bacteriophage composition described herein in combination with an antibiotic. In embodiments, the antibiotic is selected from the group consisting of a fluoroquinolone, carbapenem, aminoglycoside, ansamycin, cephalosporin, penicillin, beta lactam, beta lactamase inhibitor, folate pathway inhibitor, fucidane, glycopeptide, glycylcycline, lincosamide, lipopeptide, macrolide, oxazolidinone, phenicol phosphonic acid, streptogramin, and tetracycline.

In embodiments, provided herein are methods of administering to a subject any of the bacteriophage described herein, where administration is over a range of about 6 to about 24 hours. In embodiments, the bacteriophage is administered to a subject every 6 hours. In some embodiments, the bacteriophage is administered to a subject every 12 hours. In embodiments, the bacteriophage is administered to a subject every 18 hours. In embodiments, the bacteriophage is administered to a subject every 24 hours.

In embodiments, provided herein are methods of administering to a subject any of the bacteriophage described herein for up to 7 days. In some embodiments, the bacteriophage is administered to a subject every 6 hours for a maximum of one dose. In some embodiments, the bacteriophage is administered to a subject every 6 hours for a maximum of two doses. In some embodiments, the bacteriophage is administered to a subject every 6 hours for a maximum of three doses. In some embodiments, the bacteriophage is administered to a subject every 6 hours for a maximum of four doses (1 day). In some embodiments, the bacteriophage is administered to a subject every 6 hours for a maximum of five doses. In some embodiments, the bacteriophage is administered to a subject every 6 hours for a maximum of six doses. In some embodiments, the bacteriophage is administered to a subject every 6 hours for a maximum of seven doses. In some embodiments, the bacteriophage is administered to a subject every 6 hours for a maximum of eight doses (2 days). In some embodiments, the bacteriophage is administered to a subject every 6 hours for a maximum of nine doses. In some embodiments, the bacteriophage is administered to a subject every 6 hours for a maximum of ten doses. In some embodiments, the bacteriophage is administered to a subject every 6 hours for a maximum of eleven doses. In some embodiments, the bacteriophage is administered to a subject every 6 hours for a maximum of twelve doses (3 days). In some embodiments, the bacteriophage is administered to a subject every 6 hours for a maximum of thirteen doses. In some embodiments, the bacteriophage is administered to a subject every 6 hours for a maximum of fourteen doses. In some embodiments, the bacteriophage is administered to a subject every 6 hours for a maximum of fifteen doses. In some embodiments, the bacteriophage is administered to a subject every 6 hours for a maximum of sixteen doses (4 days). In some embodiments, the bacteriophage is administered to a subject every 6 hours for a maximum of seventeen doses. In some embodiments, the bacteriophage is administered to a subject every 6 hours for a maximum of eighteen doses. In some embodiments, the bacteriophage is administered to a subject every 6 hours for a maximum of nineteen doses. In some embodiments, the bacteriophage is administered to a subject every 6 hours for a maximum of twenty doses (5 days). In some embodiments, the bacteriophage is administered to a subject every 6 hours for a maximum of twenty-one doses. In some embodiments, the bacteriophage is administered to a subject every 6 hours for a maximum of twenty-two doses. In some embodiments, the bacteriophage is administered to a subject every 6 hours for a maximum of twenty-three doses. In some embodiments, the bacteriophage is administered to a subject every 6 hours for a maximum of twenty-four doses (6 days). In some embodiments, the bacteriophage is administered to a subject every 6 hours for a maximum of twenty-five doses. In some embodiments, the bacteriophage is administered to a subject every 6 hours for a maximum of twenty-six doses. In some embodiments, the bacteriophage is administered to a subject every 6 hours for a maximum of twenty-seven doses. In some embodiments, the bacteriophage is administered to a subject every 6 hours for a maximum of twenty-eight doses (7 days). In some embodiments, the bacteriophage is administered to a subject every 12 hours. In some embodiments, the bacteriophage is administered to a subject every 12 hours for a maximum of one dose. In some embodiments, the bacteriophage is administered to a subject every 12 hours for a maximum of two doses (1 day). In some embodiments, the bacteriophage is administered to a subject every 12 hours for a maximum of three doses. In some embodiments, the bacteriophage is administered to a subject every 12 hours for a maximum of four doses (2 days). In some embodiments, the bacteriophage is administered to a subject every 12 hours for a maximum of five doses. In some embodiments, the bacteriophage is administered to a subject every 12 hours for a maximum of six doses (3 days). In some embodiments, the bacteriophage is administered to a subject every 12 hours for a maximum of seven doses. In some embodiments, the bacteriophage is administered to a subject every 12 hours for a maximum of eight doses (4 days). In some embodiments, the bacteriophage is administered to a subject every 12 hours for a maximum of nine doses. In some embodiments, the bacteriophage is administered to a subject every 12 hours for a maximum of ten doses (5 days). In some embodiments, the bacteriophage is administered to a subject every 12 hours for a maximum of eleven doses. In some embodiments, the bacteriophage is administered to a subject every 12 hours for a maximum of twelve doses (6 days). In some embodiments, the bacteriophage is administered to a subject every 12 hours for a maximum of thirteen doses. In some embodiments, the bacteriophage is administered to a subject every 12 hours for a maximum of fourteen doses (7 days). In some embodiments, the bacteriophage is administered to a subject every 18 hours. In some embodiments, the bacteriophage is administered to a subject every 18 hours for a maximum of one dose. In some embodiments, the bacteriophage is administered to a subject every 18 hours for a maximum of two doses. In some embodiments, the bacteriophage is administered to a subject every 18 hours for a maximum of three doses. In some embodiments, the bacteriophage is administered to a subject every 18 hours for a maximum of four doses (3 days). In some embodiments, the bacteriophage is administered to a subject every 18 hours for a maximum of five doses. In some embodiments, the bacteriophage is administered to a subject every 18 hours for a maximum of six doses. In some embodiments, the bacteriophage is administered to a subject every 18 hours for a maximum of seven doses. In some embodiments, the bacteriophage is administered to a subject every 18 hours for a maximum of eight doses (6 days). In some embodiments, the bacteriophage is administered to a subject every 18 hours for a maximum of nine doses. In some embodiments, the bacteriophage is administered to a subject every 24 hours. In some embodiments, the bacteriophage is administered to a subject every 24 hours for a maximum dose of one (1 day). In some embodiments, the bacteriophage is administered to a subject every 24 hours for a maximum dose of two (2 days). In some embodiments, the bacteriophage is administered to a subject every 24 hours for a maximum dose of three (3 days). In some embodiments, the bacteriophage is administered to a subject every 24 hours for a maximum dose of four (4 days). In some embodiments, the bacteriophage is administered to a subject every 24 hours for a maximum dose of five (5 days). In some embodiments, the bacteriophage is administered to a subject every 24 hours for a maximum dose of six (6 days). In some embodiments, the bacteriophage is administered to a subject every 24 hours for a maximum duration of seven (7 days). In some embodiments, the bacteriophage is administered for at least 7 days. In some embodiments, the bacteriophage is administered for at least 14 days. In some embodiments, the bacteriophage is administered for at least 21 days. In some embodiments, the bacteriophage is administered for at least 28 days. In some embodiments, the bacteriophage is administered for at least one month. In some embodiments, the bacteriophage is administered for at least 2 months.

In embodiments, is a method of administration of a bacteriophage composition to a subject where the subject is human.

### EXAMPLES

### Example 1: AP-SA02 Product Optimization

Experiments were conducted to create a bacteriophage therapy that met the following criteria: 1) Obligately lytic, to avoid specialized transduction of bacterial genes; 2) Not known, by empirical testing and/or inference from genomics, to be prone to generalized transduction, and 3) Fully sequenced, to avoid phages with genes known to carry antibiotic resistance or bacterial virulence genes.

Collectively, the phages used together to treat a subject should: 1) Have broad activity against the target pathogen but not other species, to maximize potential utility and minimize off-target effects, and 2) Be capable of complementation, in which resistant mutants arising to one phage are sensitive to another phage.

In addition to characteristics of the phages themselves, material for clinical use should be produced in such a way as to give confidence that the final product retains these characteristics (i.e. are still the same phages) and does not contain potentially harmful (or harmful amounts) of impurities such as endotoxin or host cell proteins.

Each of the phages were identified by acquiring a diverse panel of MRSA isolates and several VRSA strains that were then screened against a variety of phages to identify phages that exhibited both broad host range coverage and robust potency. Similar to antibiotics, bacteria are considered susceptible to phage if the minimum inhibitory concentration is less or equal to the susceptibility breakpoint of 103 phage/mL which corresponds to 0.1 pg/mL of protein.

Each of the phage candidates was then advanced through a series of selection criteria and different methods, including sequencing, bioinformatics and comparative genomics, in order to identify potential receptors, phage identity and confirm lytic activity. This process yielded a smaller candidate phage pool with the desired attributes for a product candidate, namely, broad host range, complementarity, compatibility and targeting different bacterial receptors which are essential intrinsic attributes that contribute to the robustness and potency of the therapeutic cocktail. Complementarity aims to ensure that a clinical isolate is targeted by more than one phage which limits the emergence of resistance. Targeting different receptors on the surface of bacteria also contributes to resistance prevention and also has the potential to decrease bacterial virulence and fitness. Compatibility between different components of the multi-phage product ensures that the activity of one phage does not interfere with the infectivity of another phage.

Phage candidates were then validated for efficacy and potency. Specifically, this included performing killing kinetic assays to demonstrate cooperativity, activity in bodily fluids and in the presence of current anti-Staphylococcal therapies in vitro and biofilm inactivation.

Phages were also selected based on manufacturing feasibility and process optimization efforts with the goal of achieving high-quality phage product free host cell proteins and other contaminants whilst maintaining adequate phage titers. Equally important, the ability to formulate phage components in the same diluent suitable for inhalation and intravenous (IV) delivery that would allow long term stability is an important consideration in the selection process.

### Example 2: Selection of Phage Components for Optimized Product AP-SA02

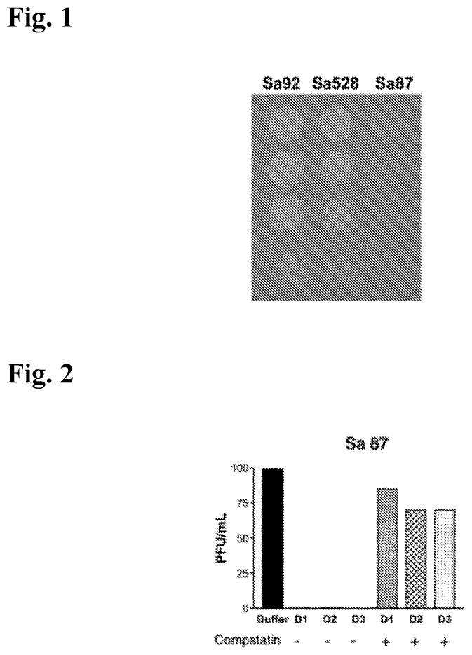

Host range and potency. The host range of SA phage is generally above 80% (Table 1), yet their potency is variable. While phages belonging to the family Kay virus, such as Sa87, have a broad host range, their potency is not ideal. By contrast, as exemplified by plaque turbidity, Silvia viruses such as ARSA0001 are extremely potent and completely eliminate bacteria, which results in the formation of clear plaques and very high efficiencies of plating (FIG. 1). ARSA0001 potency was further demonstrated by the failure of attempts to obtain phage-resistant bacteria.

TABLE 1

| | | % S. aureus |
|---|---|---|
| Phage | Family | Isolates Sensitive |
| J-Sa36 | Kayvirus | 83 |
| Sa83 | Kayvirus | 86 |
| Sa87 | Kayvirus | 92 |
| ARSA0001 | Silviavirus | 83 |
| ARSA0002 | Silviavirus | 91 |

Compatibility with relevant biological fluids. A criterion for lead candidate selection is phage activity in bodily fluids to ensure that infectivity is not locally or systemically inhibited. To ensure in vivo efficacy, the viability of individual phage after exposure to human blood and plasma was evaluated. Phages were diluted to a concentration of 10⁷ phage/mL into plasma obtained from three healthy volunteers or phage buffer and incubated at 37° C. The activity of each phage component was assessed by using a standard double-agar layer plaque assay. Ten (10) S. aureus phages were screened and most were partially inactivated within 60 minutes of exposure to blood or plasma compared to phage diluent (Table 2). The lack of recovery after exposure to blood was abolished by heat inactivation of serum and the complement inhibitor compstatin, suggesting that these phages are inhibited by complement (FIG. 2). No significant loss in phage titer was observed for 2 lead candidates, namely ARSA0001 and ARSA0002.

TABLE 2

| Phage | % Recovery |
|---|---|
| J-Sa36 | 0.06 |
| Sa83 | 0.95 |
| Sa87 | 6.33 |
| ARSA0003 | 1.07 |
| Sa474 | 0.03 |
| ARSA0001 | 93.94 |
| ARSA0004 | 1.76 |
| Sa101 | 0.05 |
| Sa68 | 0.71 |
| ARSA0002 | 94.44 |

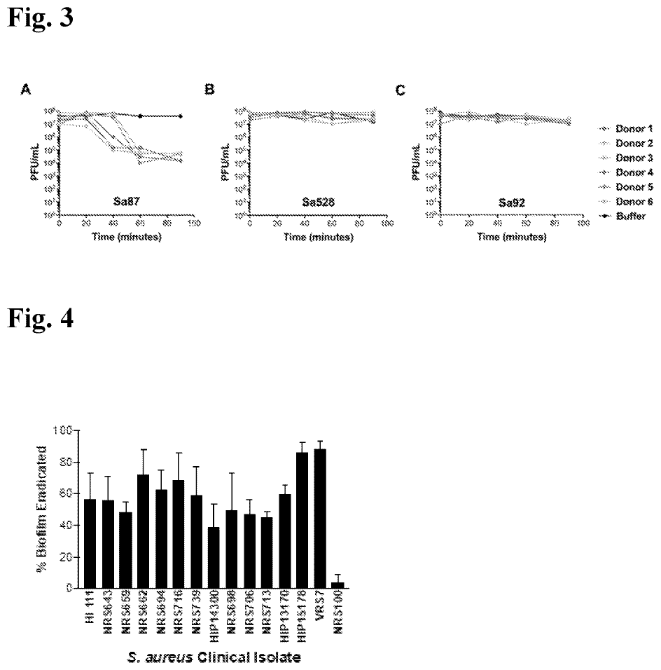

Next, the activity of Sa87, ARSA0001 and ARSA0002 was assessed at 90 minutes after exposure to plasma obtained from 6 subjects to ensure that the inhibition is not donor specific (FIG. 3). Since all phages from AP-SA01 are highly related, Sa87 was selected as an example of a Kay virus. A significant loss of activity was observed only in the case of Sa87. Recently published data also suggested that S. aureus phage may not retain full activity in human blood. The data herein demonstrate that antimicrobial activity of ARSA0001 and ARSA0002 is not inhibited by blood components and these viruses are suitable therapeutic candidates.

### Example 3: AP-SA02 Characterization: Biofilm Activity

AP-SA02 characterization: biofilm compatibility. Biofilms formed by several different S. aureus clinical isolates were treated for 5 hours with AP-SA02. The remaining biofilm biomass was stained with crystal violet and quantified (FIG. 4). NRS100 is not infected by any of the component phages of AP-SA02 and serves as a negative control. The extent of biofilm eradication achieved with one concentration of AP-SA02 ranged between 60%-90%. These data show that AP-SA02 can penetrate pre-existing biofilms and decrease attached biomass.

### Example 4: AP-SA02 Characterization: Compatibility with Current Anti-Staphylococcal Therapies

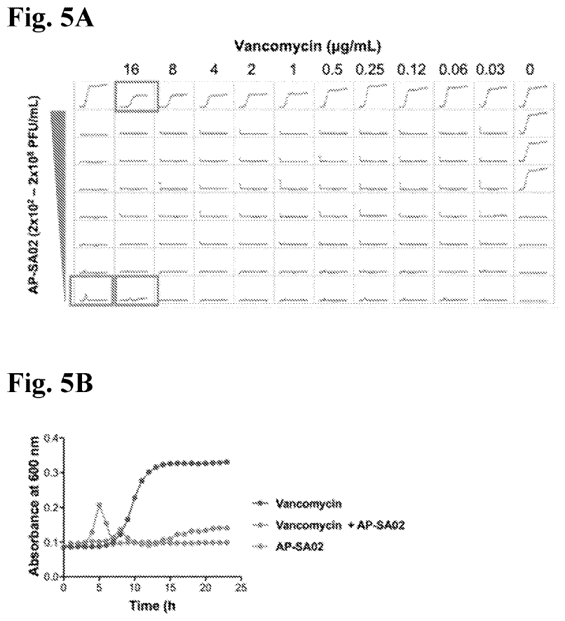

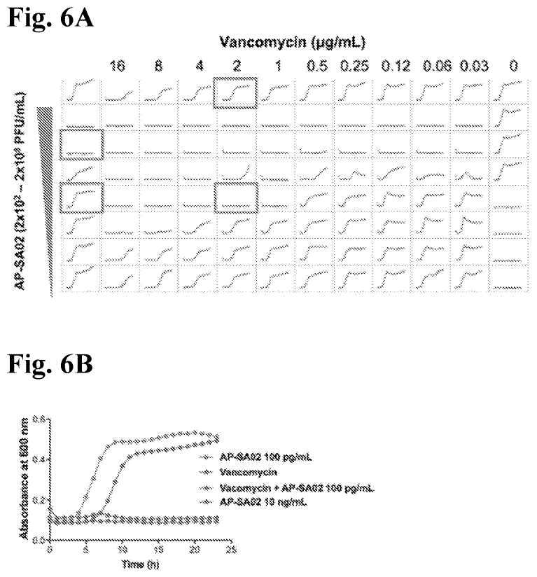

Standard therapy for subjects with a S. aureus infection includes vancomycin and daptomycin. Since subjects who receive AP-SA02 are likely to be on these therapies, the effect of AP-SA02 on these antibiotics and vice versa was assessed. Using the standard checkerboard assay, the minimum inhibitory concentration of each antimicrobial alone or in combination with each other, was used to calculate whether two antimicrobials are synergistic, antagonistic, or indifferent (Table 3). AP-SA02 showed additive, indifferent, or synergistic effects in combination with vancomycin in killing all S. aureus strains tested. No antagonistic effects of AP-SA02 in combination with vancomycin were noted. Several strains resistant to vancomycin were also tested (MIC of >16 μg/mL). While vancomycin does not have an antimicrobial effect on these strains, less than 1 μg/mL of phage was able to inhibit growth (FIGS. 5A and B). It was observed that in the presence of phage, the breakpoint for vancomycin changed from resistant or intermediate to the sensitive range (0.5-2 μg/mL) (FIGS. 6A and B). In addition, these data indicate that vancomycin and AP-SA02 act synergistically since addition of AP-SA02 at subinhibitory concentration (100 μg/mL) to a subinhibitory concentration of vancomycin (2 μg/mL) results in complete inhibitions of SA growth. Inhibition of VRSA can also be achieved with AP-SA02 alone at a higher concentration (10 ng/mL). AP-SA02 clinical candidate demonstrates robust activity against both MRSA and VRSA strains and maintains its activity in the presence of current standard anti-staphylococcal therapy.

TABLE 3

| | |
|---|---|
| Vancomycin | Daptomycin |

| S. aureus | FIC value | S. aureus | FIC value |
|---|---|---|---|
| HFH-29753 | 1.01 | HFH-29753 | 1.00 |
| F339777 | 1.01 | F336222 | 1.00 |
| NRS35 | 2.00 | NRS35 | 1.00 |
| NRS696 | 1.10 | NRS696 | 1.00 |
| HIP13170 | 0.07 | HIP13170 | 1.00 |
| NRS719 | 1.01 | NRS719 | 1.00 |
| NRS698 | 1.01 | NRS698 | 1.01 |
| NRS706 | 0.51 | NRS706 | 1.10 |
| NRS713 | 1.01 | NRS713 | 1.01 |
| HIP15178 | 1.00 | HFH 30364 | 1.00 |

### Example 5: Manufacturing of AP-SA02

AP-SA02 manufacturing includes phage and host bacteria selection, and increased robustness in the purification process. Additional information and protocols for bacteriophage growth and manufacturing can be found in for example, PCT/US19/12113 (WO 2019/136108), PCT/US19/12114 (WO 2019/136109), PCT/GB17/50376 (WO 2019/136109), and U.S. Pat. No. 10,517,908, each of which is incorporated herein by reference in its entirety for everything disclosed therein.

All processes are conducted under cGMP conditions including manufacturing, product storage, Quality Control (QC) raw materials, analytical and stability testing, and product release by Quality Assurance (QA). Bacterial fermentation, filtration and chromatography are performed in ISO 8 certified cleanrooms. Aseptic filling is performed in an ISO 5 certified isolator located within an ISO 7 certified cleanroom. QC analytical and stability testing are performed in a controlled environment. Where possible, single-use disposable parts (filters, tubing, vessels, etc.) are used to reduce the risk of contamination during the manufacturing process.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the claims.

1. A method of treating a Staphylococcus aureus infection comprising administering a therapeutic composition to a subject in need of treatment, wherein the therapeutic composition comprises two or more bacteriophage comprising a bacteriophage comprising a polynucleotide sequence with at least 97% identity to SEQ ID NO:2.

2. The method of claim 1, wherein the two or more bacteriophage comprise a bacteriophage comprising a polynucleotide sequence with at least 97% identity to SEQ ID NO: 2, and a bacteriophage comprising a polynucleotide sequence with at least 90% identity to SEQ ID NO:1.

3. The method of claim 1, wherein the two or more bacteriophage comprise a bacteriophage comprising a polynucleotide sequence with at least 98% identity to SEQ ID NO: 2, and a bacteriophage comprising a polynucleotide sequence with at least 90% identity to SEQ ID NO:1.

4. The method of claim 1, wherein the two or more bacteriophage comprise a bacteriophage comprising a polynucleotide sequence with at least 99% identity to SEQ ID NO: 2, and a bacteriophage comprising a polynucleotide sequence with at least 90% identity to SEQ ID NO:1.

5. The method of claim 1, wherein the two or more bacteriophage comprise a bacteriophage comprising a polynucleotide sequence with at least 97% identity to SEQ ID NO: 2, and a polynucleotide sequence with at least 95% identity to SEQ ID NO:1.

6. The method of claim 1, wherein the two or more bacteriophage comprise a bacteriophage comprising the polynucleotide sequence of SEQ ID NO:2, and a bacteriophage comprising the polynucleotide sequence of SEQ ID NO:1.

7. The method of claim 1, wherein the two or more bacteriophage infect and kill Staphylococcus aureus.

8. The method of claim 1, wherein the two or more bacteriophage are resistant to blood complement inactivation.

9. The method of claim 8, wherein the bacteriophage are resistant to complement C3 protein-mediated inactivation.

10. The method of claim 1, wherein the two or more bacteriophage belong to the genus Silviavirus.

11. The method of claim 1, wherein the composition is substantially free of a bacterial component, wherein the bacterial component optionally comprises bacterial host protein.

12. The method of claim 1, wherein the composition further comprises a pharmaceutically acceptable carrier, diluent, excipient or combinations thereof.

13. The method of claim 1, wherein the composition is a liquid, semi-liquid, solid, frozen, or lyophilized formulation.

14. The method of claim 1, wherein the two or more bacteriophage of the composition target one or more of vancomycin-intermediate Staphylococcus aureus (VISA), vancomycin-resistant Staphylococcus aureus (VRSA), methicillin-resistant (MRSA) Staphylococcus aureus.

15. The method of claim 1, wherein the composition comprises between 1×10⁸ and 1×10¹¹ PFU of at least one of the bacteriophage in the composition.

16. The method of claim 1, wherein the composition administered at a dosage of at least 1×109 PFU of total bacteriophages per milliliter.

17. The method of claim 1, wherein at least one bacteriophage is obligately lytic.

18. The method of claim 1, wherein the Staphylococcus aureus infection comprises a pulmonary infection, rhinosinusitis, urinary tract infection, intra-abdominal infection, skin infection, skin structure infection, bacteremia, septicemia, endocarditis, wound infection, or an implant infection.

19. The method of claim 18, wherein the implant infection comprises a cardiac implant infection (e.g., ventricular assist device infection, pacemaker infection), prosthetic joint infection, or prosthetic valve endocarditis.

20. The method of claim 1, wherein the Staphylococcus aureus infection is bacteremia.

21. The method of claim 1, wherein the Staphylococcus aureus infection is resistant to an antibiotic.

22. The method of claim 1, wherein the composition is administered intravenously, via intra-articular injection, via inhalation, or via nebulization.

23. The method of claim 1, wherein the bacteriophage is administered at least every 6 hours, at least every 12 hours, or at least every 24 hours.

24. The method of claim 1, wherein the bacteriophage is administered for at least 5 days.

25. The method of claim 1, wherein the subject is human.

26. A method of treating a Staphylococcus aureus infection comprising administering a therapeutic composition to a subject in need of treatment, wherein the therapeutic composition comprises one or more bacteriophage comprising a bacteriophage comprising a polynucleotide sequence with at least 97% identity to SEQ ID NO:2.

27. The method of claim 26, wherein the composition comprises between 1×10⁸ and 1×10¹¹ PFU of the bacteriophage.

28. An injectable therapeutic composition formulated for treating a Staphylococcus aureus infection in a human subject in need thereof comprising one or more bacteriophage comprising a bacteriophage comprising a polynucleotide sequence with at least 97% identity to SEQ ID NO: 2, wherein the composition comprises a single dosage between 1×10⁸ and 1×10¹¹ PFU of each bacteriophage, and the composition comprises one or more pharmaceutically acceptable carriers, excipients, or diluents.

29. The injectable therapeutic composition of claim 28, further comprising a storage medium for storage at a temperature at or below 8° C.

30. A bacterial manufacturing strain comprising a bacteriophage wherein said bacteriophage comprises a polynucleotide sequence selected from a polynucleotide sequence with at least 97% identity to SEQ ID NO:2, and a polynucleotide sequence with at least 90% identity to SEQ ID NO:1.


SEQUENCE LISTING (2 sequences)

SEQ ID NO: 1 - DNA, 144299 bp, Bacteriophage ARSA0001
  source 1..144299: mol_type = genomic DNA
ctwcatagtgtataaatagttaggtwtctaactgttagatacctaactatttatttttatatagttttaactactgtgaactatgcttatgtagtattaaactatgctgattatatgctaaactattctaatgtatagttaaactaagctgaagtagtatttactgtgtataaaggtatatttagtattagatattgtgtatatggtatactataattctgaacattacttatttaatatatatatatagtttgtttctatggtagtattgacttaattttccctagagtaaaagtttaatatatttagtaatttccgaacaataaagaaattttaactattttacacatatgtgttgacagtatattacttatggtttatactaagtatataaagttaaaggagatgatttatcatgaagattaaaagagaagtttcgtttatagaattaatgtatatgtattataaaggtgatgtaggtaaaggtcagtactattctactaatagtcagtttattgttgttataactgttgatgatatatcgttttataatgcattgtaccctaataaatctattactattactatacctaatgacactaagtttattgtagaagcggttatagatgcttctactgagttaagtagtatagtagttacttataagcaaggtaacagtgtattaaactcagtatggtataggattagtattaatgaagttattgactactatgtcgaaggtaaaaatacatggttactatctgttagtactgttgtagataatgagttagttactatatggaaagatggtaacgtattgtgatttggtatatagtaatattcagtatacttatatatgtacatcacttgtattattatgtgtagtactagtatatagaggggttaaataaccgcttcaataaagacttagtaacgaggagttgtattatgagtttatcagatatttatgtacatttaaatcagttaggatataaaatagaagttttaggggacatgagtacattaaaagttaatgactactacatcgaaaatagatgtatagatagtggttttagggttacaggggataatgtatttttatctgtatattatggtgaagacgtagtaagattctttaaacaaaggttattaaaatagtattttgttaagttatacttgagaataattataaaggagggattagtttatgagtatatttataagtatagtttctatatttttagctactattagtattgtactaagtataaggtttggtaagaagtattataagcatgtacttgataattatgatagtgttagtgatttagaatcagctagtgtatttggtttattatttatgagtatttgtattggtactatttctattgttttgagtttaggttacatagtaagtagagtattttattatataggagggttgtagatgatgagttcaactttaatgtatattgcggttagtattttaggtattagtattatagttggtatgcttagtgctttaattactgatatagttaaatctattaagcagaaagaagtgttttttagtatatatcttattgtttcttttgttttaactgttttagtttttgcttctataatactacatatgttaggtatgtaatagaaagagggtattagtataggttactttgtagtaagtttatcattaatgtatttagttatagatatagatatatgttatagaatatatagtaaagaaggtaatagtaatagatttaagttagcagtgtttttaactatacttatattgattatgtttatatatggagtaggtgttttattaacacaatagaaagaggcttattatggaagaattagatatactagatagagtgttacataataaatcaggtataggtgagtttacgtatacaaattgtataagaacaagtgatgataagtatatttataggttctttctggaagatacttattttggttgtgctattatggtatatgatgatatgagtgagtatgaagtagtagtacaaggtacaggtaaggtgtatgtttccttattgaaagatgtagttagtaatatgtataagatactagtagatgagtatgataagtatttaaaagatagtgcatatcaaaaatatatgtataatatatttgaagaacaagtagaacagtataactaaagtttttctctagggaaaagcagtcaaaggtaaaaatagggcttgaacccgcattctatggttgacatttaggtaaaattagataggttttccctagaagaaaagtttaaaaggtaaaaaaggagttggttactatgtcaaaattagctaagttttgtaggaagtatggagaagttactggcataatggagagtattacagattatgaggtgtacttaggtaggggttataaagtaaggtgtagagtaggtaatagccttatttatattcttgtatataaagacaatcatatcattgctaggtttgaggaagcggataatatagtatttcctttgagtgatacgcttgaggtgggaacaattaaagacttagaggagctagaaggtttgtatactaatatctatatattactagggtatttatatacagaatatgaaaataaagttatgtatgaatatgggaggttagaataatggatattatggtaagtgaattatttgagtatatattaggtcataacactcacattcttgggaattggcatacttattacttagataaaggttactgtattaaagttaatgatgataaacattataataacatgaagtttatcaaaaatggtagagtagtaaaagaggttattaacagtgacaccttactacctaaaggattcagtagtatctatagtcttattgattactatgaggaagctttagatgaaaagtgcagaaaagaaaaagagcgtctgaaagaaattcgtacgttcctaggtaagtaagtattatatataaccgcttctatatgaaactttatacctatagctattataactataggtatttttttatatttcttacataaatgtgttgattttatgtgtgttaaggtttatacttaatatataattaaaggagtgaatataatatggatatgaacaaggaagtattagaatgattagatagtttaggtggtaagttagaacaagcaggtacacatagttatgaatctttaattaaatacacaattactcaaggtattatagatttatgttcaataagtatattattatctataacagctatcttatgggtggctttgtataagtcagataaaaagtctaatgaaggtcatagtaccctattatttgagtttgtttataagaatgaaaaagagctttctgctgcaggatgcacagtaattatatttgctgttactctgtctattatttcttttttagtgctattaataggcttgcctattgctattcaagagatttttaatccagaaggatatttgattaagagtacgatagataatttaaaataattgtaaaaataaggaggaaaatataatgttagaacaagctaaaggcataactacagattatattatggaatttattgatgaacatgagcattatttctatgaagacttacacaagttagcaggggagttagtgttgaaagatacacatgacacaaaccctattgttattaatgatggtgatggttactcaggtactattatatctaaatttaaatcaaaggtagagggtgttccagatatcactgttagtgatttttacggttcatgttctttctgtgatactttagctaatatctatgaaggtagtgatgaacttgagataattagagatttagctactatggtactacatacaattcaatctatggaagagaaagtagaataatgtataagggagtgatatagtgtttagtaagagaaaagaaattaaagagtataagaaacaaggttactctgttgattatatgggtggttttttaggattatttggtcttggcgatgttttatactatgtagaaactaataagaaatacttaaagtcaattagcgtaggtaaagatgacaaccaatatgtatatgactttaatggttatgaatacttatcaagacataagttaaatatcaaagaagaagaactaatgtctaaagctattgctcagttaagtatcaaaggtttaaagaattttaatggagttaagcatatttgggatgtaggtttcttagagcaatcttttaatatacatcgtaaagatattgatacggttaaacttaaactaagttttgatgaagagggtaacctatattatgaattagaattaggttggattaatatttataaagagtatacaggtgtaaaagactttcttaatacagtagataactatctcaacttcttctataaagatatagtcactagtggtcttgagttaaaacaagtactatataatacttttatgggaggagattatgagtttgtagtatctccatataatgttgtagtatatagggaagttgagttaattgatataaagaaattttactctgatacttatggaatttattcctatggttatggcactaatgtacataaacctttacaaacaattaagtattatctggaataccctatgaatgaagtataaccacttcaaatattatataaaaggagtgcttaatatgagtaacggaaaagtagcgttaattggtagtaaggtattagaagattataatatttatacaatcatgagtgagaaacatatatacttaagagatagtgttgatgtgacttttaatgatgggaaggtattacctatatacattgatagtgtattaaaacaattaagtaaagatgataaaaatctatatttttctgaagaagatatagatattagtaaacttactaatgacgagtattatacagttactaaaaaataaaaggagaatagataatgagtagtaataaaaaaatagcagaaagagttattgagaaatttttagatgagactaataaaggtgtagattttactaatcaagatgaatttaaccgcttcttagataatacagaagacttgtatgaagtatctggtactcaaagagataagattattaagtgtttacaaaacattgtagttactgtatttatggataaggggaaataacaatgaaaacaataaaaagaaaaatagtacaagaaggtatgaacaaagaagaaactatgatggaagacgattctgttgtaataaaaaaaattcaaaaaataacaggcggagctattcttcctttagccaatggcaaggaatatgtagtagtgtttagtaaagtagaccctactaaattaattgtatttaatgtggaacgtaatcaaatctatttaagtggttctggtttcgatagtgaactgtcttatgtagacagtaaaagcaaccctattaccttagaaacagttaggttaatcactaatttattataaaggttattaggcttttaaagattaagttattttatagaataaatactactagaaaatgattactgtttaatacagaattttaacgtacctagcttaagggtattaaatataaaggagggtattagaatgactaaagttgtaagtagaggatatagttttgaagaacaagaagtaagtatgaactataatcaaggacaatgggtgatttatgcaagtagaaaaccttatattactgatattatgaaaaagtaccctgataaggtggaagtattagaacaattagaaaatgaaacaccagtattggttaaagttgtattggatgaagatttaattacattgagaaagccaataagtaacgagcgtaaagaacaaatgaaaaagattgctaaagaaagatttggtaaataatagagaggctgactttaatgaacatcagtatgtacactaacacctgtagggactagattaatattacaagatatcttatagggttatagtgtcattgagaatgagtatgctttattaaggttagttaaagaagataaacagtatttataatatcatattgaagaagtagaagcggtattaaaccgcttcttttttatattatatttatattctgagtaattatgttgtatttaatcttggctaataattgtatatcaatttgagcatagtatgcccgtttcaacatagttaatctaataatttgtatatttatctgttagttatgtataaatatttgttattacttatttatatatttgtatttcgtactaaccgtacgacttatctttttaaaaagataagaacaaaacagcgtataccttgttgttttacgctacgctacaacaaggtaacgcctgtaataagttattcgtattaaaatactaacaactaattacttgttgtttagattataacatatgtttatgagaattagatgagaatttagttacaacagcattataataatattacagttatgttacaagttgctaagtatgttttgttactacaatatattgaatatgaaagctttagaccttttctgcagacaagtaattattacaggtatgtatgataaaagataagtatcagggattaaattaaggttggattaatgaagcggataagttagatactggttaaaataaaatactttacattacctaaaacttatgatacacttaggataggataaaggaggaatatcaatgaatattcaagaagcaaaggataaattatacgatgaattactttatacagtaagtctttgtgagggaacagaagaagatttactagaacttattgagaatttattttatatttacaatgacgagcctgcatttttaagtaaagacggtatgcaatatactattaaagattttatggagagaagaaaaacattagaacctatcttaactgaattaggtatagggtacgcaatgatagaatatcacacacatacagaatacgagttagttacttattcagtacttaaagttatacagtatgatgtagataactatactgtaattattgatactaatagtgataagaatattaagttatcagatttaacattagaagctgttaaggatattttgaatcaaattaagtagctagggttgttttactctagctttttcatatgcttgtgttgcctatttaactaccttatataagcaagtaagtattataggataaatactacctaaaaagtattaccgttgaatatagacctataagcttaatgctctcttactgttataaacatcataagtataaatctatgattttcctaagtagcaggcgctgttttccctttatattaaccgcttcaaacaacataaaaaaagattaggttaatagcctaatctataactcttttatataattcttgtaattctttttctaacttttttattgtttctatatcattatttcttttaatagactgacatataaatgcaatacctttaatcaaattaaatggttagttttacactgcattgtatacaattaaatcgatcgattataataattaatttaatctgtactttctagtttcttctatataatagtaagaaatcatttcttttgaaaaactttcgtataacctattgacattaaatctaggatagtgtaatgtaataagcgtagtaaacaaaccgaaaggaattgataagatatgaacgtaaatcaaattggagatatactaatggaattattagatatagatggttacaaaagcaataactactatagaaatgacttaaaatactcaacacattatcaagttaaatatttagcttacattgatggtagttatgaaacattagaactatttaaagaaacgaaagtctttagcgtatcacattcaaatagattaccaattaacattaatgttaaggtagtgaacaaacacttatttatagagctagataatacgttatacctagtgaatgagaatgcagaaattattaaagaaaaaaagttaaaataatagttgacactatcattaagatagtgtaatataatagatgtagtaaacaaacagaaaaggaattgattaattatgaaaacactaattactaacagtaaaccatataagaactacggctatgaaatggaacaattcgagagagaaaacaacgtaattatcacacgagcataccacgaaccaacaaagcaaggttatagtcttgtaatacactatacggaggttaaataatatgaaatatttatattacattaaaggaacttataacagaattaaagcacaaggtacaatatcaaatgactatagattatggtcaggtattgatgaatgtgatgacttatactaccatattaataaagcttgtcatagtctttataataatggtgtaaaggaaattacagttacagacaaatgcgttaaactaacacttaataataataaaacaaaacttattaaaaaagtttaagaaaagtgttgacacttactaaaatacatggtaatatataaatataaagaaaacacaataaacgaaaaggattgattaattatgaaaaacacagtaaaagaaatggcaaacggaaacactaaacttgaaagattattagttttagcaattgaggaagcggttatgacagagcttgttgaatatgatgcattagataaatacaaccacttagaacaacagttattaaacaatgaagcattaacacaagcagaacgtaaaccatctaacattgattatagcacttacgagggagctatattagcacaagacaaagcagaacactttactaacgttatcttaaaccacatgaacaaagaacaatttgaacaaatgattatggatattgaaacactagaatttaatgcagaacaaatgctattctaaggagacattacacatgaacttatctgataaagaacttatagatataatggacaacgttaatagcttcactgatatcagtgaagctattaacgaaacagtaagcaaacaaacaattaaaggcagtgacaaagacagcggaacgattaaacagtacaatagtttagatgagttaagacaagactatgacataacaacactagcaccagacgtaataaaagaattactgaataatcattagtgtaaaacttcccaaaataattaactaaaactattgcaattaactgtatatttgatataatgtaattaaggaaatacaaataaaagaaaagggattgataaactatgaaattacttaaccaaatggaacaaacagcagaacttgaaggaattgaatttgacttattactaacagctatctatgaaacaatagaggtagatgttgctgaacacgatttactatgcaaatacattcatattgtaaaccaattaaaagagaataaagacataacaccattagaatatataccgtctaacctagactttacagacgatacaacaataagactagttgataataaagtgtttgagttagcagattatatattggaacatatgcgtgttgaagagtttaataaacacattaaagactatgaacaaattaaagcagaagctatagaaatgatgtaaaacaaattgaaagggaataaactccctttctttttttttattatttattaggattgtatacaataggttaatataaatatattatacgggggcaggggtaaaatgccactaaagtgggtgggtctgacagggctatggggctacctaaggaacctaattagtgagtaatattatataacttattattaaaagtcaacattaaccattactttattaaactaggtcaacattactaataactaattgtattcattttacaataaagttagtaatatatccccctcttcccttacacttaattagagtattagtatattacagcatataagtagtttagtatataggagcctttccttatatatagttttctatggtattctttagtataccattttaataactagttatactatgtactattagtagtgagtaatattatataacttattattaaaagtcaacattataaagtatataataataaaaagtcaacatagttttattattgttattaaagtaaacatggtaagatttcttttactgttattattgtcctgtctatctctactgcctttactattatagtagtagtgttactctatttcttacaatataaaaaacacctaaattaataggtatttaatgtgacatatcatatctatattcaaataattctctatctacttgttcgaaacctttctttttatcttttataatatagttaccatgtaaaactttttcaatacctgaaggtgcattaataaacaggctattgtttccttgtttgtctactgctctaaatgtctgaccttctgtccactgattgatgtcattcttattatagcctgtatattgaatatattctactacgtattccatgtcaattaagacttttagcatatactctcttactttagcataatcatctgtaagggtatctaagtctagaggttttacttttgctatattgcttatactacgttcttttacaggtgtttttgcatattctaccattaaatcaaataactcactatctgtgcctaagcttcgaaaagaatatggttcatttagaaatacccaagcaataggttgttggttttcttttttaataagtattttttcttttctcttggtagtatttttatttgcgaggctggtttcatatcctaactgatttaccttcttaataaattcatttgtatacattttttaatacctccttatttattgtccacgatttctatttcatgtattgtaatcatgtcatctctaaaatcatctatatgtttaaggtaaacaataccatcatgatttctagacatgccttgtaattctttgtatccttcagatttaatgtctttaataagctgttcttgactagtatatattttatcttctctataagagtagctgtcttcgtatagctcacaattatcatgctctacttggtaatatttcattcttttttactccttttttcatagatatttcctacaacttcagtattaccaatctctgaaatgcacatcatagagaatggtatgtaatctcccccttcaacccatccaaaagaagcttctttaaatattacaggtctacaatccccccaatatctaagtatatcaccttcaaatatttcagtaccattcttatcttcaagtcctgtagattgcataattacaaggttttctcctaagtaaccatcacaagcaaataggtgtgaatatactgtatggtctgcaaagtcaaatctagcaatattaaacattctttgtgattctttatcccatgctctaaatttcggtatcattctattacctccaataaatgagatgattcaaatacgttgcctttaacctcacagtcgtacataaggaaagattttttgtctgtatactcaaagtaatcattttcagagattgcgctctcaaacataaagtcttttaagtgaatgccacttacaacatcaatagatatcactgctctgttaattgtttctactgcagattcaaaatcacctggtattgctaacacttcatcttcgaactcaataatatcaccctcatatattttagtaccgtttttgtctttaagtcctgtgtattgtagtaactgtatatccttagaaaaaataaaattaccaccttctaacattattttctcatcttttttccatgctctaaacctaagtgtcatactgctaccttcactttttcgacccctatgcttgcagttttgaatgggagttttttacgagtcagttttaatgtcctatttttggcttcttcctcattcgtactttgcacaaaataatgcttttttattttataatcacatttaaacgctaagaacttaatacaaagactcactttataggtttgcatcattctaccaactccccatctttccagattaacgtcatagttaggtcatcgtttaagatgtagaatgctttggtaggcacacatctgccatataaacattcttttatactagtgttctcatatagtgtagagttatagtctccttcttgaatctcgaataattcaatcaacctatcaaccttagcctcttctgtgatttcctcttcagcttcgacttcaaaagtgtcgtcagctgatataaattcactaacaagacattttcttccgtcttccgaagaaaaataaacaaagttctcattatcttgattttctgtgtaaattctctttccttttgttaaatcaggatattcccaagcccattccatgagttcggctagcgtcattacttttttagtttttattttcatttctatatctcctaattatttaatttttctatcattttcacaatctcttttgattttctaatgtcagcattttcacagttttttaatatgaagttaattttgttttttacatcattaacgatactgtctacttctttttctgttggcttagtagggaaaagcaaccctacaactacctgaatttgatttttatttagtgagtaaacactaatgttacatttctcattaaagtcaatatgtggtttacttgaattaaatacctcatctacaattgtatttaattcttgtataggtttaaaataatgttgttttctttgtattattacctgtgacattagtttatatagtttctgttctttatcaagaacttcttttgtaagccttttaacttctcctatccttatagtattaccccatgcatcataattatttttaacatactcagcttcttctatgttagtagtatatcctattacttttgaggtgggttcctcatcataataacagtcctcttctacatatatagcatatatcttattcatatctatcaaacctttctttaagttaaattaactataacatataaacattaggatgtcaactactaatatacacttttttcagtaagcaaaaaaaaagaacctttttacaggttctttactgggttattaattttttaaatcttggtttccttttattaattttttatcatcaataggatgaccattgtataaatattttacttccatatatacatgattatccgtagccgtaagggcatctaggatatccggatttgtgttaattgcatttgtatcaggaaacgaaaattttcttttatccgactggattatatctacatgaaatttttcttgttttggtaaagcgtaattaaggtcttctaaattttgtaagaatttagtaagagaaaagggtttaaactttccttgtaaattattatctatatagtcatttaatagtcttgtcatttcctctttatttaaagtcattgtttcataatcgttaacatcataaaaaacaacaacattaggttgctcaatacttgtgtctccatctttgtacttagcaatgtaatagcctatttcacctttgtgagctaaataatcttctttcatgatattaaatacttctctagcaataggttttgtatatagttcttcccatttatttgtttctgtgttgtaaaaatgtggtaataagtttgccattctgagttatcctttcttattctgatttaagccatatcttattagtatttttaggcggttcattacttattgtgtatccatcagtacccatatctagtgtgtttacaatgacaggctgagctacagcttctttttcactctctaatgttttgaaactaggcacgtctgttttattagattcttgtccatcgactaaccaagagactttaaagtcacctttattataagtagtattaggttttaaatctgttaatgttacttttacagaactacctgtaacttcatcagaagttaaaataagctgttcctctttgttgtagacctttaattttttagacataatgttatccctttctgttgtgtaatacactaataatataacatataaaaaacaggtaaggtctttcaaaacctttacctgcttattctttactgataatcataccaatcaagaaaatcatctgaacagcttaactctatagttatatcatttataagtttagtagcttcatctttacttaatgactctatatcaaaagtaaactcatcatataaacaatctatatgcttataaataaaatctttttgtttttccgtagctttgtttaattcattgctttttttacttataaattgactaaagttatcactcatttaaatcttcctctttaataaaggtaccattaactgttcttcctttacgacctttaatttcattataagcaaactctaaacactcttgtaatgacatgtcgtgttgctgagctaaaataattaaagtaactacagtgtccccaataccatctttaagagcttctaggttaccacgtgataatgccgatgctacttcacctgcttcttcgtagaatttaagagcttgtctatcagggttgttattgttcaatcctttatctttactccattcctctacttgactaactaaaccttctagcgtgcggtcttctgtttcaccttcattaaagttaggtttttcatcgtttggtatatggtcttcttgttctctatcctctattgtaataaataaagtgtctatttctttttcgtacttatttctctgcatataattatactgctcatactcaggataaggtacatatacagtatcgtctgtaccatcctcataattcaaacaaacacaaacaatatctttgccacttagtctatcatataatgtcatgttgtagggctcatctaagaaaccatgaaactcttcaaagtattcatccttcataccacttacctcaatagaaactttttttgctgatttacttgtatctacttcactatttccatatatcatagttttttcttcactaatgccttctagtatcaactctacattttctggttctaacattgcgctatctaagttttcaaaaacaatttcaatatttttaatttttttagtcattaataatcattccttttttttagtatatagtaaatataacacataagtaaatagaagtcaataaaaagacacctactttaaaaataagtaggtgtaagatattttgacaggtagcttgtggaaaaaaatcctgagagaggactcgaaccccaacgactaggtagctacaactagccaataccattactcaggattgatagtaacgctaaatagagtcataacgttaccatagaccttttctacgcttggtatataggtagaatataatgattttacagtacccatatagttagactgttactctcattataaggttaaaaaatgctaactttgtttcgcattgtcaagaggcgttagttaactactataataatatagcatatattttattgtttgtcaagttctttattccatttacttttttcttttagtaatccttttttaattagttcttcactaatatttttatactctttattttctttgtagaaaactttagctaaatatctaccaaaagaatcatctttgtatgtttgaattaaaacatcttttccttctagcatagttgttgtaaattccttagcttcttggtaattattttgtttcttttctggtgtgtcgacacctagtaatcttattctttgctcagaatatgttctcattccgtggtcaatagttacatgtatagtatctccatccactactttgtccacatgtgctttaaataaatataaattgtcttctatcatcattaaatcccctttttactaatttaaaattatccaatcagtattgcttgatgtacccttaactacatatacagatgaacctattatagcaatttgacctttatttacagatatcatttagatgaatatggtcttgtactgaaaagtctacaatgacatgtttatgctttttcttttttgttaatactttatatgtgataaagaatgatataatagttaaacatatatttaattttttcataaaatttctcctttaaagaaggtatttaccatctcagtagttaattctttgcttttaaagatacaatattctgtatttttattcaatatttctaacatattttgcatactacttgctttttgtaaatccgttaagtctataacaactgttatttctgcgttatctattagaatattacctatgtaataatttttaactccattctccgtagcatgagaggtaacattatgatttaaaacaactataccttttgatggtaagtactctgaaaagtataagtaacaaaagtatttgtgtatcttttttttattctcttccattactattaccatattctatgaagtttaatagcctttcggtataggttttaattttctctacttcttttctttcatcgtcttttcttcctaatcttgttgtatatttaacaatattaaatagcatgcagtctataaagccaataatgtagaaatattcttctaaaaatgatataacatcttttttacctttataatggttcggtgtattagtggtagaattgacattatagtataactccagttgttctacatttttcataagctttttagcataatataatgtgtcatctaaagaatgtgtcacatctaaagatgcttcaacaatgtttatctttaattcttttaattctgttgcctctttgttacttccaatttgtgaatcaataaattctctagtatccatattggtgtcctttcgtatctaaaataaaaaccctatctagaatatagatagggaaaaggaaagatattatggaagtgtactataaagtacacatataatataacagttcctttgccttttaatctatatcatttattttaacttcttgtactttaacataatctgataaactattaacgacgatattatcttctactaattcgtttctattatttgtaacatcatatgagtctctataaggtgtttgtactaatgctctaactgtaatgtctatatctacatattctttactatacctttccattactcaacgtagtttcctttagcaagctgatactttttgtgtaattcttcatataatttctccatatctttatattttcgtacatcttcttcaaaatgacttaaatactctattaatgtcttaacttctttaggttctaaagttataactggttttaccattacgcaacatcctcactaaattgtttatttatatttttaatatcttttattaattcgtcttttaattctttaggtaactcatgactttttaaagattgttgttgacctaacgtatatccatattttgttatccttacttctacagtataccatgatttttctaaatcgtcaacttttctagctagtaatattaaacatctgtcatttataatattcgaagcataactgccgacacaatgagatagctgttcaccttctctttttaatttttcagtagtatccgcaggtaaaaatttaactttagagccatcgtttaacttatattctttatttgtgatattatttaatttattatcatatttttgttgtaactttaagtcatcgacttcatctttaactgttaaatactcatcagtaacaatatcatgctctagttttaaagaatatggtgttaagttaatactttctttagttctatatccaagaagtactaggtctgaagcaaagtctatataatctctatataaattattaacagcgtatgaatgtatacgttgtctgtttctgacatcatagtttatataattcaccatttttctaaagtcagaaaatgaaaagttaagaacttctttcgtagtacctttaaaagataaagcatttcctattgaatatgaattagtcaccattataaagtcgtcatcgaatagtttgtgtaagttagactcattatataaactaggaaatttatttatgataactcgaagtatattaaggtaattttcttgctcttctttactaagagtaataaacttattatatgtttgtttacttaaatttaaagcttcatgtaatttccatttatttttgtttggtattactagaaactctttgtgttctttaaatatatcacaatcttcgttgttataaactttatcaatattagacacaactaattttaacatattgtttaaattatcagtagcattttcacctttactaactgtgcgataaccatcaggtctatctatcatagagtatacatcaaagttttttacattaagtctacttagatttaagtaataatcattattctttagtgtttggattattatgttaaaagctaaattttcctttccatatttacttaaaataatatttaaaaggacaggtaaagtaatgttattttttagtttatccttattaaaatcatcttcatacatattaaatttaaattctttatgttgtagtgtatttgataggtgtaggtatacatttccatttttattattaaagtcaccgtttactatactccaatctgttgttactatacttatgtcttttaaataacaagtcttaattaaaaacggagtattattatcttttataggtgttaatttgttaagccctatttttatgttttttaaaacttcattgtagtattcgtagttattatcataagatttaccaaatgcataagggtcaatattaataccaaatatactatagtcacccattaataatacatcaagtatatcattagttgtagtatttatttgtctttttaagtctcttattttttttgtaaggtaattattactattcttattttgcgtattgtcaaactgttttagttcattttcatagtttgcacggcagtatatattattacgatagtgtcttttgttatataaaggaaattcagacctatcgtcactttctatatactcactatatacagtatttacatctgtaactgttttacttaactcttgtacttttttatatgggtctcccttaaacgtagtaagttttttatttccacataatgctataaataaaagtaaatttaaagcgctagaataacaaaaggtattattaatatctcttaataattctttagagccaatatctacaatatttttattatttatttttaaatatactctttcttcacctataaatatactatttctatttactttagacaatttaagaatttcttcttccgttaatcctgtttctttatattctactgacatgtgtcatcacctcatttattattatataagtatgataacatagttaaatttaatagtcaacaaaaaagaagaacttttttacagttcttctagataagtttcatatttaatttttttagttcttcttacgggtacaatatcgggattactcattaagtctttgtacttcttagaatttttaaagttaatagtattaggcattgctaaaacttcctctaactcatctgtgtacatagtaactttagatattatatctatgtcactaatatataaatttttagatttacttttaaaaataatctcatcggtactagttacttctttattatctattaaatccatatagaagtcctttctagtctacataaacaagtatacaatctttgtctgataaagacaccgaggtcatatcattaagttcttgtagttgtttaatagacacatcatatttcttacttatagtccataaggtatcaccatttctaaccttataataaaatttattacctttttgtattaagtctttcattgttatctacgcctttatattcataataagatgcctgtaggtaacatatcgtaataagaataaagttagctacttctgtatatattacatgtggtgtcgtatttgttattaacatactaataattaaacaagctaatgcaatacctataataagaaataaatatttgctagttccatttgtacttttagttttataaaatttatttatttgggataaataagctacgataatactaatagtagccattatctgtccatattcaacactatagttagctactgaatagccaatataaataacaattataaacataaccatttctaaaatactaacttctgttgttctaaataagtatataataaaacaaataatacctagtaataaattaatacttactgatataatttgaaataggggtgcatgtgttacaaataagcttgttaagctaatacttactgtaacaacaatataaaaccaaaaatagctatctacacctttaacattcttatctcttatcaaacctactaaagcaggtatatatcctacagtaactaatatagcatataggatgcttaatatatgagctaaattattcatatgaccctccgaaattctctatctcatttaaaacttcttcccatgaaataaaacctttaccgttggttaatgataaaatgtgcttggtaagattattccatgttttattatttttaatatttgtaattgtagtataagtaactttaaattttttagataaagtagatgccttctctttgcttgtgtaaatcaagtagacatcatagtctgttaattttgaatttataccaaaaatattgtcgttccttagactggtttttattgcatgtaaagtgttctctctaggagtaacccattctaagttttcaactctattatcatcttttatcccatttttatgattcacttggtttctatcaatatttttatttaagtctagcaaaaagcattctgctataatcctgtgtactttgcattgaatacgtttgttattttctccaaataacgttactattatatacccatctttatcttttcttgtctttaatacatttttacgcttacctttgatgatacctgctatacttatagaatattcttttgcaaatggaatagtagaccataaaagaccatcttcaaaataaatatttctaggagtgtatttcaaagggtaatacttcatagaatctttgtagcttctaggagaatagttatattttttaaatatagttctcagttccgtataactaactccatactttgtagcaactttcctcatactgagaccactctcataatctcttattgcattatcaatcttgctgtcccaatctttcaagttctaataatacctcctcccacgaaatgaaacctttgccatcggttaatgacagaatcataccaaaaataaactggttcgttgaaaactccgctctgtctgggtcagtgtatggttttccgtgcccttgacgaatatcagagcaataaattaaaacaggtttattgacgacatgatacgcttctaatatatcatcttgtgtgtttttatctaactctctaaaatctacatcttcatatttgttgataatttgttgagcttggtgtttcattcctaataaaatactaagttctgctattgtacctaatccctcattcaatacatcaaatacaaatatatctgactcttgcatagccttaaagtcattatttagaatacgctcagccaaaccctcctgtctagcatttgctttgtcgttaattgagccatcttgatgaggagagtatggagatacacctacaattccttctacttgattgtgttgtttttctctatattctatcattgcttgattaagtagatgacctcccatatagcaaatcttatctttgattttatttaccattgtttttatctcctttttatttatttaaaagttactttacccatttccaatacttgtattctcttatttctctgattgtagttctatttacaccatacttttttgctaattctgtatgacttaaatcactcactcttacttcttttattttttcttcatttaacttagaaacagggttttttcttttaccacttatataagaattaagtttatgctctgtattatactcattagtacaccactctagattagaagcatagttgttacttttatcaaaatccttatgattgacttggggtaggttgtaagggtttaaaataaaaacactagcaactaatctatgcactatttgagtgttactcttatctgagtagtttcttaaagacacaatatgatacccttttttgtttatcgtatgttttagtatctttttggttttatcactgtatacatttcctttttcgtttatataataaccgtagaagttaaataaagggtaaaatacctttccatcaacatttttaatatttaaaggctcttgatttctattaacaaaaccatatttatttttcttcttgtatgctgagtaacaaccacaacttctaatagttctatttttatactggtcttttcttacctctactatattaccacacttacataagcatttaacatatttacgctttctatttttacattgtttaaatagtgtttttattgggattaaccatgtttcttgtaattccatcatacaactcccttatatcttataatttctccttttctttttcaaactcatctcttagtgtgatgatatctttattaagttcatctgaatctttcattttagcccaatagtaaaagtctctaagttcaggatttcttgctaaaatatattgcttaggtcttacatctcttccttgtgataagaagtaagctatatctctcatatatttccagtctttgtaatagtcagacttatatttaaacataaatcctttcaaatcttcaaatacatacccttcatgcaatatattataagagccattattatccttataccatttataaaattcttcccaactagcaaactccaactctttttcttttagttgtaatcctacttcatcagcaacatatttcaaataatcataatctttctttttcatatttaacgtatttgaaataatatcgagtaagaatatatcgctttttcgatattttatgatatgagggtcttcttcgatatcaactacttcaaacactaaagtaaaactatcgtcagcaatataatgattttcaaggaatgttgttaatgtctgtatctgtcttttagttaaagttttttctaccagctctttaaataataatgcgtatttattcccatgtccacttaaatgtgtttttgatttacttgcatatacaattccttctaacgggtcatagaacattaatcctaaaaatccattttctttcttatatccagttaaagggaattggatagtgtctggcatattctctaatttggtttcttctacttcattgatattaaagaacttattatatcctcttcctaatactgtatttctaccttcatcaatataaagacctctagagctcactgttaattgattccatttctttttagaaaaagctctttttgtaaagttaactgagaacacatctctatattgtttattggttttgatgattttattttcttttctagctatatctaagtattgattaggagtgaggtctttgtcaatactatcatttcgtctgtttgataaaaggaatttagggtcgtagacagtgttttttatttcatgtttctcaatgttgttgtttttgtctattgttactgctcttaaacaaccacctttttctacgcgcccttctaaattaattgattttttattattatttaaaggttctctatataaatttctatacccatgtatttggattacgtcagaatgttcccattggtcatcaacattaaaatcatatcccccgattccattaataagctgacttgttgaaatcttgtttaaattaccaagaacttcaggaaggactccgccatgactaacaatatagtcctgacctttaaaactaaagtaagccacctgttgtaattttctaagaatagactttactctattttgagttattccgttctctatgaaggatttcaaggtaggcataaatcctcttgcatgaaacagatttagagcatgtttataaaaataacttttcccaaaatccacctttttgaaacttctttgataattttttcatcataatatttctccttatattccaattgctttttttgctaatgcatctacatattcattccatttgttattatcatgagactttacttttataaaatttatatctataaactttgagtattcccttagcatattaatatatgttttacttaaattgtttttagttttccacgaaccttcataccattgaataagtcctatataatctacatgaataatagcctgcttataacctagttttatagcctcttcaataccataacaacaagctagtatttcacctgcaacattattatacttaattaaattactattttcaataggtttactgatttcttttacaatgttctcatttttatctaagataatagctcctgctccaacaagacctttattataagatgagctaccatctgtatatattaatgcagtcttatgcatatttatgcctttctacaaagcaagggaacatctcacttgtataaatctctttgtagtcttgtaattttatagtagagaaatcaaaaaacttataacaatgttcttctgcttcgatatagtcatctgcaataacattaacttcaaaaccatcaactaaaataacgaacaatttcttaaacatttaaaaaacccctttataatttaataaggtacttaattaaatcaatttcagtgtacttttctgagtgttctagctcttctaatttttcttttacttcttcaagtctgtatgactcttctaaaagttcaaactgctctaataatgtgatactatcaaattcactactattaataatgttgtgtatatattctagatgtgtgtctttaataacgtttgttaagtcatcaccagtataataatcctcgatatacgataggtctcctaataactcagccgtacttctaataatactatatttagatatattagggtaagattgatataaacaattatctaatttattacttaaatgctctactactttttgcattgtattacctttttcttcagtactatatgaaattctaatattagggtttttagaatcataatcaactaaagaactcctattagtgtttaataacttatctaaagaagttaaaataatagctctatcttctatattatctaaggttaagatagtatctataatagtataaaatgctttgtctcctattgctttattaacatcttgaagttcaaaaatattaatcatatcctcaatctcatgacgctcaacaaagttaataatagattttgtaaatttattcatcaacgtcaacatctttaaataattctacactattagtatttaaagctttttctaatttcataagattagcactcttaggttttcttttaccactctcccaataagaaataagtgagtagtgaactccaatctcttgtgctagacttctaactgtatgacctttttcttctcttactttttttaaatttaatactttattctcttccataataaaccactacctatctttcttcaatttttaaaatactatagttatttttattataagcatacttatttaaaacgtcaagcgttttttctttagcctctactttacctttagctttcatattaacatagaaaacattacttttatttgaacgtgtatgcttaatatatactttgtactgcgtaacctcactttttaatacattacgtttaaaccatttctttaatttttccatttattataaccacctttaaaacttatatatgtattgtagcatatgataaatacattgtcaacttaaaaataaaaaaatactagggaattttccctagtaaattatttccaattaatacgaccccaatacttctcttttagcatacgttcccacttgtcagtgattttacatactgccatatagaagttcccagctgacgggtttgtaggatatttaaatctaatccaccaatagccatctgctttaataacttggtcaaagtttacatattgaccactataaataaatgattcagctcctacgattgaaccacttaaacctggtgttcgtcttaccttaatcgttctatcagcagtaaatttacctctccaattccaagttgtttgtacctcgtctttgaaattaggttctataaaccacataggaaagtcgtaaccatgggctctaatagtagctttttcccagccaccgcctccttgagcatctccccaagtccaacctccacctaaccaattattttcaattactataatttggttttggtttgcacttagtacccatgcgacatgaccatgaccacacccgtatgaattattaaagataaccatagtgcctggtttaggaatgtaggatagtgtattttctactactctagctttatcttttaacatagctttattatcccatggaatattacatgcacttgctccacctaaagaataacctgggaagagcttgttaaatccatagttagctaagtcgtaacattgctgaccataccaaccatcaaagtctattgctttgccttctagactttttaaaaatcgaacaaattcacttctagtcatcgatgctttcaaaagttttcactccttatttccaatctatttttccaaaatatttttctttcttaatacgctcttgtttatctgttatctcacatacagcacaatagaaataatttttactagagcctggttttggatatttaaatcgaatccaccaatagccatctgctttaataacttggtcaaactcaacccaatcatccttatcgtatagccatgaatctctatttactatagtgcctttaagtcctggagattttctaactttaatagctctatctggataaaaagtacctttccagctccacatagttttagtagattgtgtttttactctaggctcagcattacctgatttctttacatctactgacttaataactttcttgtaatacttagctacttccttatctaagttactagtgtttctacctaatccacatgcttctaataagttacctgggtcttgtttgtcgttttggatgtcttggtgacctggcatatttgttttgtaatcaacaccccagtattcagctaaataagctaagacacgagctccattgtctaatgattttttagaacgttctttatcactaaagtaacaaatttctacaccaatagcaccatcattagcgttaattccgtaccataggtcatccataggagcgttatacaacacatgccaagctttttctgtagtaggaacacatatgatagcttctttatcatctacaaagatatgtgcggatgcagtggaagcccaatcaatgttatatgtatttttgtaataatctacattattttgagctgtagtattaatattccctgtatcatgagctacaataaaaacgggttttttaaccgccatagcttgacctgtgcgtcttgttccaataggcagtaaatcatatctaacatttacacctttccaaatttctatcattactgttcacctacttcttttgggttatcttctttatctaattcagacactaagtctttttctactttaccatgttctaatggttctgcatgacttgtatctatgtcttcattattaactccttcaggagccatcggcatctttgtaggaggtaactctgaggtactattcattgcttcactcttgtatgttaaagctttatctgggtcattataatctctaggtttgttaaagtcctgtacaatagggctatccgctatacctttagtgtcatgacttacaagaacacctaaaccagtcaagaaactaataactacacctgcaattgcagtgccttggtttacaatatcttctgcacctttgattcctagtaaactacatacactaacaataaacgtagctaaaacaggtacaaaacctgtccaaaatgtagcactttttgcttttgcagtaatattgttacctgctattaataatggttttttatctttttttgacataaaaaaataccatcctttctgttattgtaaagctaataataatataacaaaaagaatggttttagttattaaattttaagttattgttcgttaatttgagaaagtgttttgattacttctgacatttgtttattatctaagtcttctaacttagtgtgtggaagtaaatctaatttatcccaaatatcttttttagttgtgaatttttcattaataattgctctaccgattaaactttcggtataataaatttgttttgcagatgccatattagtatctaccgttgtcaaaaatttataaaactttataaatcctattcctttttcaacgtatccccttttgagtaccctcatcattactgattactcttatatactctactcggatttgtatgatactccaaaggcttaaattcccatacaacgaatggtacacatatttagtatcttatttgtgataacttaaatattatagttagctaaattaatactagcattaaggtctctatcaatactaaaaccacatttacatttaaatactctgtctgatagttttaggtttttatggatactaccacatgtagaacatgttttactagaagggaaaaatctatctacaataacaagttcaattccttgtctttcacatttgtattctaattgccttctaaactcataaaatccttgtttttgtattgctttagctaaatgtctattcttcatcatcccttttacattcaaatcttcaataacaattcttctaggtttggttttcacgatagaagatgttacttgatgtaaatagtttttacgaatattagacaatcttcgttgtaggagtttaatttgtttttctagttttatgatattgcaagttttgacaaacgtactcccttctttatttttttggtatttgaaagataccttacgttgcaacctacgtaatcgtttttctaattttttaacctttattgttttattaatattaccataaatagtaccatctgaacatacagctaactcttttatccctaaatcaatacctaaggactcgttggtcaaatcttcatatttattttcttcttctatagagatacttatataccagtatttaccatcaaaagtaattctaggattgtagtacttagtgtctataggtagttgttcagaagttctaacccaacctaccttttctattagtacttttttctcttttactactagtgcatacgtgtcattatagaaactaggtcttgttttctttttagatttgaacttagggaaaccagatttctttttaaaaaattttttatatgcattgcatgcgtctttaactgcttgtttaggaacattgttacttacttcatgtaaccaactaaactctttagttttctttagttgtgtaatttcttttcttaatactgtgtcttgtataaattttttaccttgttcgtaattttcttgctgttttgctatagtccaattatatataaatcgtgctataccagctgatttacataattttaatttttgttgcttggtaggtttaagtctgacttttttcgttatcactattcaattcacctactatttttttagcgttgttcttgtgtcttatttgatttacttctaagaacttatttggttttatttctgttttttacggagcatataggatttgaacctatgagtgaaataaatcacctattgagttagcaacccaacctcttaagccacttgagtaatactccttagtataattaaacatacatacataaaccgtctcactggtaagtaacatattagacctattgttaagttaatcactacgcttgactctattaggtagctaacctaagtccatctcacgtaacctactaaagcagtacaaccatagggttgagtccgtttaatagtgtaatgtgatactaacatattaattagtaactttcacatggtatgaagaattttaacggcatctttatatttttaccaattattattttgcaggtttatgctactcgtactagtatcgtatgttatcaaacatttttcaatgctccaactgcatgtacttgttaaggtgtatttgtaaaggataccaccaaaaccccctacaaaaattaagcccatcggatactatactttataaacctaatacattactgctaagcattccgattgtaataaacattactattgacgaattaccttaccgacgtataccgataagttttaattcgttcgacaatcttacgccttacaggtaccaccctgtttaaatcagccattatgtatgtatgtttaattatactacatttaaagtataacatattatcattcatttgtcaattaaaaaatgtgttacgttttatagtttttttatactagggtaaaaacgtaataaaagacctagtttcgacaaataaaaataaggagaagaatgtttattatgcaaaatgaaattatagctctttatgagcttatatataatataacatactattttgagtttgtcaagttattttttacttttttatcttgataattcttcttgtactctatcccaatatttttctgattctgttctattatcctcaatgtgagcagtagtacatttacatttgatacagtataaatgctttatatgtccctcttctcttttctgagctctttttcttggtactgtaaaagtattaccacattttgaacaaactaaattactgtggaacattttctgtctttttttcatgattatcaatccttttcttttaatttgttttatatttagattatatcaggtgttgagtattaggtcaacacctttatttaatgtttctagttaatttttaatactcaagctttaagaaacaagatgggtattcttctgatactggagtagtatatttatcttctaagttatctagggctttgaataatttatcaacgtactcaatatcagttcctatagtgtcaataatttttgtattatctattgtactttgaggaacgttattacttttcattttctttaatcttcgagtatgttttgatttaatattgagattttgagaatatagcatttctttataaacctcattccatgttttaccaaagttggggttgataatactactaattttaggtgttaattttctaacccttttatttgcttcttgtttccaatcattattttcaggtcttttttgtaaaaactctagtttattttctaattccataacttcgttctctactgaagataacctatcattttctttttgtaaatcagtcaccaatttatttaatctttctgactcctcaaagtacttaagcatttcttttttagcttcttgaggattatctataatagctttagcaaaatcatcattcatgtacattccatactttctaatatcaggtagaacttctgaagttacccaacgcttgaatcgtttagcttattctagttttgatgaaaagattaaactgtataaacctgattcgttgattaatgtcacattccttaattgacctgcgtacctaatttgggtacgtagcttatcttcatcctctgcatggttatttatagcgtttcgataatttgaatatcctaatatcttagctacttcgttacctacaaagtaagtttgattgtctttaactactgttcttactgattctccttcaaaattaaacattaattggttttccatgttacatcagtccttttcttttaattgatactcttattctaacgtaagacttaataaatgtcaatgctttttgttaaattttatgtaattattttgagaaatccttttatatcaaggtttttaaagatttttttacctatttggaaaataaggaggattccttatatatataataaataattataaatactaaaataataatactagttataataattaataatatatataggaaagtcaacaaagagtaataaaaaagtgacttacttaaaagtcactaattttcaaatactaatttaaatgattctatgaataaagggttaatgaatactgtatcagaattaatatcctcagagactctaactctaactgatgcttgtccatgtcctctaattgttccatttatctctctttgtgtatactcaatactatcgtcagagattaaataatatttttttccactaatcattgttacctcagcatgttttaccatatcaaaactcctaactaaatttattgaaaatgaaagttataatagaacctaacactagtaatagtactttctcggtagtatccttctttcttgtgtccttaacaactgtattgttagtgagttcttttatcttttcgtctaatctatctagctgtatgtagatagacgattgtttctcactattaacagctacttctttgtctaaactaactaccatttttcttagttcatcaacttcattttctaattccttatacgtatttttatcaatatagctaccatcttgtattttatcttttatcttatctacttgtcctacaaggttttttagttcttcattcaactaaagtcacctcttaatgtctaacttttcagattctgaatggacgtcatagttttcgaaaaagttattaataatttgcatataatcctcagtaactatctcaccgtatgacgttttagtatttatttcaaacctaagcttaccaaactcatggaggtctatttctttaataacggtattaggattatcagatggtatgtaataataatcaaaatatataatagagccgttgataagcaacctatcaatcctatagacgtggaagaattttctatctcgtctaaagtgagctaaggcatctttaaactctaattttaatatatctttatacttagtcaaatcaacttacccccttataattaattttgagctacactagttttgtgttataatagttattgcagtagcagatataagtattatgtttaataaaattagcaaccaccaaccacctgtacttattaaccataatgaaaaacaagatagtatataaacaaccaccaagtagcctatatatttcataatgtttttacactcttttaatactactacgtatgctagtgatttagataagctaaacagaaaataaaaataaaaaataaaaagaggtatagctataatgcctgctagtataaccactaattacacctaatttcttttagctagataataaaatcttatataatacaactttaatataacattgaaacctgtttataaatcaacaatgttacattaatagtgtacggtatattttaaattaggaggactgaactaatagggtgaataataacatagctgtatttatatttaaaactctaataattattatttttattttgtttattttgtctattattaactcactgtcattaatatactctattcgacctagtacagttatgacttactttatattcggtgggatagtttctaatgtggcattaacaataactgataaattattattacacaaagaagaccccttacccgattatattctaaaagaagtagagctaaataacaatgaattacaagtactaaagaaaattgtggagtcacaaactaatgtttctgctgaggaagtaagagtaagagcaaaagcacaacgtcgaatactaaaagataaaaagagagatgctgtcgatgaaaacgaagaaggacattaaggaacaacgtaaagagctaaaagataatgcaaccgctgtttccttaacaaaaaaaggagataaacgtattgcaagtcctagtagggtgtgttgtttatgtggtcaatctctttcagggatgaactatactaaaggtaaatctatagctaaagttaatcattttcatttacagtattctaagtacttatattttgacatatgcgcagatattaataactgttacaagaatttaaagaaagtaggtgaattgatagagtgagcgctgaaagtttaagggatatgttatctaagaagaaaattgaagacgaagataaacgtaaatatatagctgatggatttatgtcaggtattactaaacttatgtatgactttaataaaaagattgatagaggggaaatagaagtaagagaccctaatgatttgtacaaagtatttgttatcttccaacaaatgcaaaacttaattacagatggttctgatggtggtggtgctataccgcaattatcaagaccacaacaagagctctttgatgaaataacaaatgagaatagtaaaggtgagaaagaagtagatttagagaagttatctcaactatctgctgatgacattactgctatgattattgataaagaaaaggttatgaacgaagagaactcaaatacattctaaagaaaggtgtatatgaatggacggtaaagaattattaaaaataacacaagaaacattcggtacacaaaatgttacgcaagaacaagttgaccatattatcaacatgttaaaccctagtacttatatgctaaaatatcatacgttacgaggtcacccgataacttttagtattccaaacagggacagaagtaaagcacaagctcatagaccttggcaagtccgtaaacatactgtgcggactataaaccttttctaaacttggaaactctaagtgcttaaacaagcatatgacaatcaagtgctaaatttagtagtgatactaataaatgcctaacgactaaacttactggtaaacttataaatggataagtgtagaacagtataaaagggaaaccgtaacgaaagagggcgtatacacctaaatgtatacgtatgatatagtctaatcccctaataaatatcgggaaaccgagggtaataaatgatagtaaatgacaaccatcctaataaagttgtgattaaatcacgacagcttggataaagctttgttcaagtaaaactttcactaaaaggaaaaactctaagggataatatcctatgacaattccttgctaaaaaaatgcctaacgactaaatttctaggtagataaacgaataggtttattgagaactagataaaagggttaatgacccgtaacgtgaaagagttaatgtcttttcattagctaagatatagtctaaaccgattcttaaatgaatgttaaagtattacgaaagtaacggtagtaattgtaagtgagatgggtgtaatggaaatggtacattttgctgatatgcatagttatgcaagtgtaaagtgtctatacacattccctagattataagttggggaattaaaactctgttaaacggtcataggaaaataaagaatatcctggtaagagagactaagtcctaatggatagagttaataccgtgctaaatcaacctaataagttgtaaatgccgaacgactaaatttctagatagcttattaaaaggaataagtgaaaactagataagaaagtcttgacaaaaataccaaagtaagttaatatattaaataggaggtggaaaaacaaatgcctagaaaaaaacacatgaagaatttgtaaaacaagtagaagacatagcaggaaatgaatacgctgtaatagggcattatgaatacgcaaatcaaaatataaaaatgtttcataacgaatgtaaggaagaatttgaaataagaccaaataatttcctacaaggtaaaagatgttctgcttgtagaagtaaaagaataagtaggatacaagataaaccacttagtgaagttaaaagacaactaaaagagaaacataagggtactatagagattataggtgagtatacaaatactcacactaaaacagattttaaatgtttaacatgcaaccatactttctcttcagaacctaattctgttttaagactgagtggatgtcctaaatgtaaagtttctaaaagggaaaaattaatagctgaattccttaaagaaaataatatacgttttaaagaacagaaaagatttaaagattgtgttcataaaagacctctaatatttgatttctacctattagatttaaatactttaatagaatatgatggattacagcacagtaagccaatagattattttggcggtgttaaagcttttgaagaattgaagtatagagatagcttaaaagatgattacgcaaaaagaaataatattaggatggtaagagtgtcctataaacaaaaagagaaagagataatagaagaattagaaagtgttttgggtgtggtaacccgtcaagaaagtaaagcagagagctcaacaccaaaaagttgagttaagatatagtctagtccgtatgtaaatatcacgaaagtgacggtagtaacgactaatgaacagatgaagaaattcgtacaatcaagacttaatccagtattagaaaaaccttattttagagatattgttaattgggataaggactcattaggttataaacaaataagaaattctagtttatttttcagaactagtagtaaagctagtactgtcgaaggtgtggataggtaacaagtccacattaaactctgttaaacggggaaacccctaacgtaaagacgagggcaatcccgtgctaaatgttgactttataaaaagaatatgatataatgtatttataaggttagctaaatgcctaacgactaaatttctaggtatctatacaaaagggtatagtgagaactagataagaaaaccgccaagattatttttaataagtaaataattttatgaaaaaaagcgtgtaaagcatattactaatgataagaataaagtacaagccttaattaaagattatcaatatatggacttacatggatgagttatttgaaggggaatataaatttttagaaccttatataaatagtgacacagctatcaaaggaactaatattagaacttcttggcggaagtaaagcagagaacctttcacctaatagaaaggttatgatatagtctaaacccactcttaagtaagtgttaaagtactaggaaactaggggtacaaattgattgactatctttcacttgacgagtacgaccgtgtaaataatttagctgagagctcagctttagaatctatgtcaagttcaccttttaaaatagtaagaaggtggtctacaccaactgtaagagattatggtatagataaactgtataaacaatcagaccaatggttttatggttatgattgtgaacactgtggtcattttaatgaaatgaagtatgctgattataaacctgatgacttagaaaatagtggtaatgtattactagtaaatccagatggaatagatgagatagctaaaactgtccaagatggtacttaccaatatgtttgtcaaaaatgtggaaagcctttagataggtggtactctggacgttgggcttgtaaatatgccgagagaacagaaaataataaaggtattcgtggatactatataagtcaattaaacgcaacgtggataagctgtgatgagattaaagaaaaagaaatgaatacagaatctaaacaaagcttttataactacatcatcggagccccttttgacgatgtaaaaatgaaagtaacagaagatgatgtattcaataataaatctgaaattgcaaaaacacaattatttaatagagataaatacaaatttatttcttgcagtgttgactggggtaattttcactggtgtgctgtacacggtatgacagaagatggtcatgtagatttaatacgtttattttctgttaagaagaatagtagacctgatatggtagaagcagacctagaaagaattatattggagttctctaagtacgaccctgatattattgttgctgataatggtgattctggtaataatatccttaaattgattaatttttttggtaaagataaagtatttggttgcacttataagtccagcccacgttcatcaggtcaattgtacgcacagtttaatgagaatagtaatacagtaactgtagataaattaatgcataataaacgttatatacaggatttaaaagctaaacgtattagtgtataccaacaagtagatgaagagttaaaaacttatcttaaacattggcaaaacgttattataatggatgaagaagatgataagacaggagaaatgtaccaaattattaaacgcagaggagacgattttgtaatatggtcgtctataaacagtgttaaacgggcatagtaaaataatgtatagaaaaacaatatactggtaagaaaccctaaatctgactatacacagacagtgggaaccaaccgtgctaaattcttaatttaaatattaacattatattttttaatgttattattaaattaagataaaagcctaacgactaaattcgttactattaacgtaagagattaaaataatttattgagtatactaaaaaggagggtagttaagtggtttatgataacgagtggtttattaatagaattaaagatgttagaccagaggactatgaagaatatgaatttttagatgactatgttaatcagtacacaaaactaagagctattcataaaaaatgtggtaacaaaatacttataacaccatcaacatttatgagtaaacgtaagacaggttgtaatacatgttctcaaaaaagagtccatagtaagcaaagaaaaacacatgaagaataccaaaaggaattacctgaaggtataatggctataacaaaatatgaaggtgcatttaagaagataactttacactgtttcttttgtgatagtacttatactattactgcaagagatattttaaagtataaacaatgttcgagatgtaacaggagatttaataggacattggaagatattaaatttgaagtacgtaaagaaacgaaaggaaactatgaagtagtttcaaatcattataaaaatgcacacacccccatagaaataaagcataatgagtgcggaaaaacatatcggggaactatggcaaattttagaaaaggtagaagatgctcacactgtagaaattccataggtgagaaattagtagaaagttatctagagcaaaaaggtataacatatgaaaaacaaaaaacttttgaaggcttaaagttaataaataacttatcatatgatttttattttcctgaatataacttacttatcgaatatcaaggtgaacaacacgttaaacctgtcaagcattttggaggagaagaacaatttaagctacaaaaaaagatagatagtataaaaagttcttatgcaaaagataacggttatgagctttttgagatattctatacaactaaggattacaaaagtatagagaaattgttaaacaataaattattttattcgtaaagcactgagttatcaatctagataactaagatatagtctaatcccctaataaatatcgggaaaccgagggtatgaaatgcattacgcacagtcttcagtaattggttacattggtcttactcgtttaaaagaattgtatgataaaggcaacggtacagcattcacatccacttttataccaacggattataataatacacaaaacaactcgtctaatgagttctattttgatgaataagaaaggactgacgacttgacaaatatattaaattatgatacaatacaagaagatgatataagcaaggagacatttaatttattaattgaagagcctttaacattatcatatgtttctatacatagtgattttttagataataaatacgattacatatataaatccttaggctttgataatttcatggactgttatttatacgtaacaagagaaccagaacgtatatataaaggcggagctaaagatataggctcattgaataaagtaaaaagaactgtagtacgtaatgggaaagaaattgaaatgacaatctatgaagataataaaaacgatgataaacctaaagagagtaatagtgaagaaccaaaagaacctaaaactgctaaaggttctagggttattactcacggtgatgatgagaaacctaatcctaaaaagattgccagttctctatctaccttaaagaataaaggtgtgaacactgaccacatagatggcaacgcatcattatataaagaatttgtagatgataataataatacaataggactagcttcctttaaagagacagattctgatattatattacatggatatgtatcagactttgaaactactggcgtaggtgctaggtctataatagagttaatttatttagcaataaagaagaacaagaacgctgttgtctatgctatagaattacctcaagctatagagttcttaaaattgttagggtttattaaaaataaagattactatgtattaaaaaaacaagatataaagttatttttaggtgagtacagtgagcttatttagcactgtactcatttatacaattatatatataagtattattatattagtagttgtaaatgtaatagtaaaagaaaaaagattgaataatgctaagcagttacttatacaagtaaaaaaggatattataaacaacttaaaaaagggagatatgaatgattaaagataataatacaaaaacattggaagagttaattacacctatttctacagaagaaaaagaaaataaagttaaagaatttagtaaaacattatgtgagatgattaataagctatatagttcttacaatgtattaaggcaagaccctattgatgaaacacaaagattagacggttctctaatggtgtttcaaagtagattagatgatacattaacaggtgaagaacaccaaaaactttttaaattagcttttgattataatattgatatttttgaagctaacaagcagtttaaagaagatgtacaaaatggagaagctattaagttaggtgatgttgttgtaaccgacacagctttaagtaatgttttatctagtaatgagcttaatgttagtattacatttatgttaagcaaagactatgaagaaaagaaacgtattgaagaagaagaaaaggaaaaactagataagttataatagatattatgagacctttatggataggtaggaataaataatatgaaacgtaagtttagtagtaatgagattactataacacttataactattgtagttgctctatttatagtattaattacagtagcatttaacaagtaccaaattgctaaagaagataaggacagatatcaaaagctagtagaaatttatcaaaaagcagatgatgatgacggtaaaactaaaaaaaggtatgttgagaagttaaatagggctgaggaagaacttaaaaaggttaaagaagaaactaattataaaggttataatgataaaacagaaaaagaaagagataaagaggataagagtgtaagagaacgtatttatggtaatgataaggataaagatttagtattagtaaataataaggttgttgtaagtaatgaagttactagacctaagataatagaagataatggtgtcagtacagttgtagtacctcctgttgtaagtcctgttgagccaccaagtgaacctagtttatcttctcctactatacctaaacctattattcctaatcctattaaccctaaacctattattcctaatcctattaaccctaatcctattctacctaaacctaacatccctacccctaacccactaccccctatagaacctgaaaaacctatagaacctgaaaaacctatagaacctgaaaaacctatagaacctgaaaaacctatagaacctgaaaaacctagctcagatactacaatacctttagttccattggaacctagcacacctgcagatgaaagtaatgatactaatggagagggataacctctccttttttttgctatattaaaagagtaataaaataaacattatagttggtgattttttgataggattaaacgtacttacagctatatccgtagtttttgcttgtgtaagtctactagcactaatggtatttgcgtatattaaaactgaaaaaagtagtaagttggttctatatttgatatatgctataattgctataggttcttatgtagtattaacaatgtttcaagccacgtcaatcattataaaaaatgatgtcatagactctattgagaatacggagcagtatgtagggtttaataaccctttaattgtttttggtataagtttctttggagctgtaataggcagtttttggtttttagttatgaagattgtaaatactaggaatttaaaaaataaaaaattaaaggattgaagcatttgctattttcaaaagacgataaatgggatgaagcaaaagactttataaaaggtcaaggattagcagataattggatagaagtagtggattattatagacagataggtggtaaacatgtagctatgtttattgctattgataaagttaaatatatggtactagaagctacaaaggataataaagtaatattggtagataaggataataatattgtgttagaggattatgatattgttatggaaagtaagaaaatgttttactatatagaagaaccattcgaagtaaagataaatattcctaaacatatccaagacattacctataataacactgttatactaactactattcgaggaggtaaataaaaagttgcctgatttatttaaaagtctaaggttaggtagtatgtataaagaagacactgaagaccttatggttcctatagatgacggaatacaagctaacattagacaaatagaacaagatacaaaagagatgcaagaaattactaaatctctatatggtaaacagcaagcttacgcagaaccatttttagaaatgatggatacaaatcctgattatagagataagaagagctacatgaggaatgctcataacttacatgaagtgttaaagaaatttggtaataactctatactaaatgctataattattacaagagctaaccaagtatctacatactgtaaacctgccagatattcagaaaaaggcgtaggttttgaagttaaattaaaagacttagacgctacacctggtattaaagaaaaagaacagatgaaacgtattgaagaatttattcttaatacaggtacagataaagatatagacagggattcattccaagagttttgtaaaaaaatagtaagagatacttacacgtacgaccaagttaacttcgaaaaagtttttagtcctaaaaacaaaactaagatggagaagtttattgcagtagaccctagcaccattttttatgctactgataaaaacggtaagataattaaaggtggcaatcgctttgtacaggtcatagataagcaagtagtagctagttttacaagcagagaacttgtcatgggtattaggaaccctaggtcagacttaaattctagtgggtatggattgtcagaagtagagattgcaatgaaagaatttatagcttacaataataccgaaagttttaacgataggttctttagccatggcggtaacaataagttacaattaaattaaaataaaaaaatatttaatataaactttgccgtgcatgtttagtaataagcatgtatcaagggttaaatgctatgaaatcctaaagctcaattaactaaaacagaaagagaaatcttagacggaaaagttacgaaagtagaaaaaataattgagatacgttaaggttaaatcctaagacgaagaacaatggaagtatagcagggaaagtcctaagttcaaatgaataaggaagaccctcaacgactatctcctgacgggagagtaaagccacaagctattggtggaagaaaaatccttatcctacgaaaagtaggaatgacaaatagtctccactcgtatcgaaagatagagctatagattaactataggagttataagtagcgttataactcaaagaaagttaaaatttatagataattacaattttattccaaataactgttgacattatttcctttatgtagtatattatatactataaggaggtgaatataatgtcggaagagaaatataaacgtattaatattagattactaccaacaaaagaacaagaagaattaatgtggaagtccgtaaataatgttagatttattaagaattattttattgctcgatgttacgaggaaaaagataaaggtacttttataaatttgtctttggaaaaagactttaaaaaagaattaacatatattactaaactaaacgaatataaatggttaaccgaatccgatagacattctaaagtagtagcaataaaagatgttttgaatggttttgttagatactataagggcatagcaggaaaacctaaatttagaaagagagggctaacacctaataccttttctgttagaaatgatgtaatgcctagtggattatgtaggtttagaagagatggtaatacatttaaagtatctgtaataggatatattaagtattctaaaagaagctttaataatttcaaacatatatttgatattatagaggataaagataaattttattctcctgtaataagacatgatggtaagtactggtatatttcattgttatataaagataacttagttacaaatcaaaaaccaagagagttaactgatgaaattataggtatagatttaggattaagaacattaatgacttgttcaaatggtattaaagtcagaaatattaatttagatagaaaagttttaatactagaaaaaagagaacgacgattacaaaaacaggtttttagaaaatatgacatgaataaacaaggtaataaatatgttaaaactaaaaatattattaagttagaacgtaaaataagacacataagaagaaaacttaaaagtataagaaataatcatatacacacagaaactaaaaaaatagttgaaatgttaccgaaagaaatagttatagaagatttacatgttaaagaaatgttaaaagataaatggagaagacatgaaatacaatatgctaagttctttgctataagacaaatactttcatataagtgtaaagatagaggtattctactaactgttgctaatagatggtacccttctagtcagttatgtagtaattgtgaaactagagtaactggtaaacaaaaaacaactattaagtcgggttatcatacttttaaatgtaaaaactgtaatcataatctagatagagatttaaacgctagtattaacttaaagaactatagatactccaattggtatcgaaataatattattgagaaacaacaatagtaatattatatgtaccgttccatgaacgggagtttaagcctttggagtatctagttagtagaatattaaaagtaactaacttagttagtgaatgaatgaatggattattcaaaaacagttacagaataaggaataaaatatataattatttataaattttaactcatggtactactcggggcatattacagattagagctgaccaacagcaatctcaacatgcgttagaaaattttaaaagagaatggaaatctagtttttcaggtataaatggtagctggcaggtgcctgttatgatggctgatgatgttaagtttgttaatatgacaccaacagcaaatgatatgcaatttgagaaatggttaaactttttaatcaatattatttctgctttgtatggtatagacccttctgaaataggattccctaatagaggaggaggagccacagggtcaaaaggcggttctacattgaatgagtcagaccctgctaagaaacaacaacagtctatgaataaaggtttacagcctctattaagatttatagaagaattaattaatagacacattatatctgaatttggtgataagtatactttccaatttgttggaggagatactaagtctgaattagataagcttagtatcttacaaaaagaagtacaagtatataaaactgtaaatgaagcaagggaagaacaaggattgaaaccaatagatggtggagatgtaattcttgattcagcatttttacaaagtacagcacaagaaatacaaaaagaccaatatttagatactaaacaaaaagaaagacttcaattacttatgagctatacacaaccacaaactgatgagcctactgatgatagtaaacaagaaacaactaatcaagaggtaggaacagataatcagttaaaaggtgatgataacgtatatagaacacaaacatcaaataaaggacaaggaagaaaaggtgagaaatcttccgactttatgtaaataatactaacaacctaattaatacttaggttgttattttttaatttggagctataaatattttatgctatactatgtgtgtagtaatgaataatgttgatgatatactttttgttatattatgtatgtacatataaaataacaattaagtgaggtgtataatcactttggaagaaatgaaatttaatgcttttgttcctatggacttaaagaaatccatagatacggaagatgatacaaataaatattctatcgtatctggttgggcttccacaccagctgttgatttgcagaatgacattataaatccaaaaggtattgatatagaatactttaaaaaaaatggatatattaactacgaacatcaacaagataatgtagtaggtatacctacagataactgttatgttgatttagaaaaaggtttatttatagaagctaagctatggaaagatgatgaaaatgttataaaaatgttagacctagctgagaaactagaaaagtcaggtagtggtagacgtttagggttttctatagaaggcgctgttaagaaacgtaacataaatgacaatagaattgttgatgaacttatgattacaggggtagcactagtaaaaaacccagctaatcctgaagctacatgggagagttttgttaaatcattccttactggaacggaaacaacaccagacacacaagtagatgcaggagccttacgtaaagagtcattagcaagctctattactaatttaacctatgtaacaaaaattaaagatgttaaagagtataatgatatttggaataatgtagtagaggacttaactaaatctaataatatgggttatgaagaatcagttattacattacaattagccaaaggactatcaagaaaagatgctgagatagcagttatgagtattaataaaaaaaatttagaataggggattatattatatgcctacggaattacaaaatattctacaagaatatgataacttagacaaagaagaagtatcaaaatctgtagaagagtctaaagaagaagtaaaagaagataaaacagaagacactaaaacagaagacactaaaacagaagaaaaagaagaaattaaaaatgaagtagaagctacagaagagcaagtagcagaagctaaagaagaaccacaagaacctgcaaaggttactgaagaagatgctaaagaagcacatgaacaagaagaaaatctagaagaagaagtatctaagtcagctaaggagtcgaaagaccctgtagataaaaaagatactaaaacagaagacaaagacaacgagaaacgaaaaaataaaaaagacaaaaaagaagaaaaagatgaagaagaggaagaaaaaacatctaaatctatttctgataaagatattgtagatggtttccgtactatcttgaaatcattccaagacattaaaaaagaaaatgaacaatatgtgactaaatcagatttagaagaagtaactaaatctattcaagctttaactgataaaattaattctaatgaagaagtttctaaatcagttgagagcaaccaagaagataaagaagaagcagtagaaaaatccgtaacgactaataacacttcgaatgaacaagatgtagttggttatgtagctaaatcagtacaaacagaagaacaagcagaaactacagaagataaagaagaacaagaagtagaagttaaagaagaaagtagagacttatctgtagaagaccgtgaaaaattcatgagtacctataaagaaaaatctcaaaacccacatacgaatcgttcagaagtattgaatgcttatcaagcttacttaaatatccgcaacaatccagagcaagcaagtgaaaaagactttaatattgtaaaagaatttgctaatctttaattaagaaacaaagttgtgttatattatagtgtgtaaaataaaacaataaaaaaaaagagcatatatgaaaggtgatatatataaatgaaccaaaatgaactttcaaaaaagcaacatgcagttgctgatgaattacaagaaaaattatcgaaatccttccaaactggatatggtatcactcctgaaacacaagtggatgcaggtgctttacgtcgtgaaattttagacgaccaaatcactatgcttacatggggaaataatgatttagtattttaccgtgatgttgcacgtagaccagctgaatcaacagtaattaaatatgatgttttcttacgtcacggtaaagtaggtcattctcgttttgtacgtgaaatcggagtagcgtcagtttcagaccctaatatccgtcaaaagaccgttacaatgaaatacatttctgatactaaaaatatgtcattggcatctagtttagtaaataacattgctgaccctggtcaaatcttgactgaagatgctatctcagttgttgctaaaacaattgaatgggcttcattctatggtgatgcttcattaacttctgaagttggtggagaaggactagaatttgatggactagctaaattaattgatgctgataacgttatcgacgctaaaggcgctcacttagatgagaaattattaaacctagcttcagttaaaatcggtaaaggttttggtacagctacagatgcttacatgcctatcggtgtacattctgactttgttactaatattttaggtcgacaaatgcaattaatgcaagacaatagtggtaatgtaaatactggtttcagtgttaacggattctattcatctcgtggattcattagattacatggttctactgtaatggaaaatgaattaatcctagatgaaactttaatcccacaaccaaatgcaccacaaccagctacagtaaaagctgaagtaaaaacaaaccaaaaaggtaaatttactaaagaagaagaccgtgcaggtttatcttataaagtagttgttcactctgatgaagctgaatcagcaccatcagaagcacaagttgctacagtaacaaatgctacagatggagtagaattaaagattactgttaactctatgtaccaacaaagcccacaattcgtatctatttaccgtcaaggtaaagaaacaggtatgtactacttaattaaacgtgtagcacttaaagatgcacaagaagatggttctttagtattcgttgataaaaacgaaacattacctgaaacagcagacgtattcgttggtgaaatgtcaccacaagtattacacttgttcgagttattaccaatgatgaaattaccattagctcaaattaatgctagtattacatttgcagtattatggtatggtgctttagcgttacgtgctcctaaaaaatgggctcgtattaaaaacgtaagttaccttgcattaaaatagtaaatacaattaaaaattgaatacaatagaataaggagtatacctaagggttgctccttttttattactacttatagtatacaagaaaggaatttaaaaatcaatgttaacttataaattaaaaaatattaaaatggctactgtacatggtcaagtagaagtagacgataaaggcgtagttaaaggactaactgctaaacaagaaaaagattttgctaatttaccaggttttacacatatggaagacaagaaaacaacaaagaaagaagtaaagcctaaggaagaaaaagaagagaagaaaccaactaaaaaaacaactaaaaaagaagataagtaggttgataaaatggtaaacagtatgtttggtggtagtttagacccttacgaaagctctttatcatatgagtatccttatcacccatctggaaacccaaaacatatagataaaagagaattagatagtattactctagctgattatggttggtcagcagatgctgtgaaagcctatatgtttggtataacagtacaaaaccccgatacaggacaacctatgggtgatgatttttataatcatatattagaaagagctataggtaaagcggaaagagctttagatatatctattttacctgacttacaacatgaaatgagagattactacgaaacagaatttaatagttatatgtttgtacacgcatataaaaaacctatattacaggtagaaaatttacaactacaatttaatggtagacctatgtatgattatccagctaactggtggaaagtggagcatttagcaggtcacgtacagttattccctacagctttaatgcagacgggtcaatctatgtcttacgatgctgtattcagtggataccctcaattagcaggaatgtacccaccaagtggagcaacttttgcacctcaaatgattacattagattatattagtggtatgttacctagacaaagagcaggtagaactaaaccatgggaagtaccaccagaattagagcaattagttattaaatatgcactaaaagaaatttaccaaatatggggttacgatgatattaccctgtgtgtagcgtaagttgcacattttcaaaggttaactgctaagaaatcctaaagctaactaaactacaaagtaagtcgaaagactaagcttgaatgttgagaaatcagaaaaaattagttagatactctaaggttaaatcctaaagagaagagcaatggaagtttagcatcgaaagtcctaagtcaatagataaggaatacgttcaacgactattctgtatagacgacagaagtaaagctccaagtgattgggagaagaaaaatctttaccctagtcttagggataacaaatagtctgcacacgtcttgaaaaggagtgctaggaattgacctagacttatttagtagcgtaagtaaggaaacaatgttaattaaatgaaaacttcctaggaagttaatttaattaacatatggtaatctcattatcggtgcaggaatagcaaacaaaactctagatgtagatggtattacagaaacaataggtactacacaatctgctatgtatggtggagcaagtgctcaaatactacaaatcaatgaagatataaaagagctattagctgggttaaaatcttactttggctccaatatgataggcatatagtaggaaggagatattaaatgcaaaaaccttttatgataggtactaataataatacttctaatataattaataagtctactatgtatacatctacaacacaagcagatgaacaagaacaaaagttatctacagctagactagagtttgacactaaggatatgagaagatttgtcaatgatagaggtataaaggtactgtgggaaagagcttacttttgtccttgccttaaccctgacacaggacatcctagagtagattgtcctaggtgtcacggtaaaggtattgcctatttacctcctaaagagacaataatggctattcaatctcaagaaaaaggaactaacaatttagatataggaattttagatactggtacagctataggaactacacaactagaaaaacgagtatcttatagagacaggtttactgttcctgaagtattgatgccacaacaacttatataccatgttactaaagagcgtatacaaaaaggtattcctttgtattatgatgtcaaagaagtaacttttattacatctaacgatggtaatgtttatgaagatgactataatatagagaataatagattattcatggacgctaagtttgaaaacaaaactatatccttaaatattttaatggtacttagatatgtagtatcagatatattaaaagaaagtcgataccaatatactaaatttaatcaacctaatacaaagtttgaaaatttacctcagaagctattattaaaaagggaagacgttattgtactacctgagccatttaaagttaatgatggtatagaagaggacttagagatacaggtagaagaccctaaaaaagaatctaaagataatcattcaggtggtttctttggaggaatgctataatgcctataaaagcaactagacctaagttatttagagacaagaactataaaaatataggtaagagaacagtagatgctatgcgttctgatattttagatagattacaagctacagcattacaagtagataatgtaagtgtcaaacgcatgcctacttatctacaaataactgagaaaaagctagagaagcaaggtgtaatagaccttaagaaagcttttgctcattcatctaaaaagaaaaggactaaagatggtggttggtatctaacggtgcctataagaataaaaacaagtaagatgaataataaaacataccaagacttaaggtctttaaaaatagacagttcaagtaattcagtatctactcttactgattatctagaaggtagacgaagtaatattagtcacccatcactaaagcctgctagtaaatcaagtagaattactaaagtaagaaatggtaaaaaatctagttactttatgtttagaacagtatctagtaaatcccctgcaagttcatggatattaaatagagataaggttaatgaaaacaacttctctaaaacaacattaaagtatgttagaaatctgatgaactggaagattaagaatttaatgtagaaaggttatggaagtaggatggcaataacatcagtagactcatatttattagagcaaataaagcccagattacatacagtactaagtaattgttatattatagatgaagttttaaaagactttgattatcaaactagggaaatgttcaaagaggctttttgtggtaagaacgcaaagcacgaagtaacagtagggttcaatttccctagctttaaaaacaactacgaagctcattacctaatacaactagggcaaggtcaagaggttaaaaactctttaggtagtatacaagggtcttatctagaagcttcgggtaacacatatagtgaacaatcagtagctaaaagagaaggtaatcgtttagtatttcatgtatctaaacctatttctaatgtaataagagtagaagacatagcgtttgattcttacgatgatgttaaagtatcagataacactgtatcttttaattatactaacaatgagacttatgaaggctacaacgcaaatattgtatatgtagaaaaaacaaatgatagtaaagggttagtaaaaggtattaccatagaagaacaagtaacaattgtgggtatgtcgtataatgtagatgttgctagatgtttagatgccgtattaaaaatgatattgatatctatgagagacagtatagaagaacaacaaacattccagctacaaaatcttgcatttggagatatcgcacctatattccaagacggtgagtcatctatctttggtagacctactataattaaatacacaagttcattagatttagattatacaataacacaagatattaataagattactttcaagaataaaggagcaaaaagataatggttaagaaaacacctaaaggtaaaacctttacaggttatgtacatatagatacatttttaaaaacagcacagaccttgtttaatatgaaagagtcacaggtagcaggttttaaagcctacatggaaggtaaacactacctgttcaatgaaaaagactttattccatttttagaaaagtatttaggtagagaattagaaatttaatatatagataggagaattatacatggcagttgaaccgtttccacgaagacctgtcactcgtccacatgcagtcattaatgtagacagtacaggtatcggtaaatcagctagctccagtgaaaagattttatgtttaattggacaagctgaaggtggtaaacctgataccgtatatgaattaagaaactacgcacaagctaaaagactattccgctcaggtgaattacttgatgcaattgaattagcttggggttctaatccacaatacacagcaggtaagatttttgctatgcgtgtagaggatgctaaaccagctacagctgaagtaggcggattgaaagttacttcacagatttatggtaacgtagctaacaacattcaagtaggattagaaaaaaatacaatcagtaattcattacgtttacatttaatcttccaagatgatagatttaatgaaacatatgataatattggtaatatcttcactatcaaatataaaggtagtgaaagtacagctactttttctgtagagcacgataaagaaacacaaaaagcaaaaagattagtattaaaagctaatagcacagaagttaagtcttatgacttaacaggaggagcttacgctactactaatgccatcattaacgatattaaccaattacctgattttgaagctaagttatcaccttttggagataagaacttagagtcttacttattagaccctatgacagatgttaacattaaggataaagctgagtatgtaaaagcagtatttggtgatttagagaaacaaacagcctataacggacttgtgtcatttgaacgattagtatctgaacaagcacctaaaaacgtagaagtagacgcacatgaagaatctgctactgtaacagctgtgtccccaattcaagaaatcacaccttttgaattaacaaaactttcaggtggtacaaatggtgaacctcctgctacatgggcggataaaatcgaaaaatttgcacatgaaggtggatactacatggtaccactatcttctaaacaatctgttcatgcagaagtagcttcttttgttaaagaacgttctgatgcaggtgaacctatgcgtgctattctaggtggaggatttaatgaaacgaaagaacagttgtttggtcgtcaatcatcattatctagcccacgagttgctttagtagctaactcaggtatattcacaatggacaatggtcgtaagttacatgtaccagcttatatggtagcttctgctatcggaggtttggtaagtggtttagctataggtgagtctattacctttaagcaactacgtattaataatgtagaccaattatatgaatcattagacttagatgagttaaatgaaaacggtattatcaccgtacaatatgttcgtaatagagctaatacattctttagattaacagacgatgttacgacatttaatgataaaaatgaccctgttaagtcagaaatggctgttggggaagcaaatgacttcttatgttctgagttaaaacttgagcttgaaaataactttattggtactcgtactatcaatacaagtgcttcaattatcaaagactttgtacagtcattcttaggacgcaagaaacgagataacgaaattcaagacttcccacctgaagacgtacaagttattgtagaaggtaatgaggctagaatttcaatggttgtataccctatcagaagcttcaagaaaatttctgttagcttggtataccaacaacaaacattaagagcctaaactagggcaaggagtacctagtttaggtactcctatttaatttaataggagagtgaattaatatggcatcggaggctaagcaaagtgtccatactggtaataccgttatgttaatggtaaaaggtaaaccagttggaagagcacaatctgctaaaggtcaacgtgaatacggaactactggtgtatacgaaattggtagtattatgccacaagagcatgtttacttaaaatatgctggtacaattacactagaacgtttacgtatgaaaaaagaaaacttcgcagatttaggatatgcttcacttggtgaagagattcttaagaaagatattattgatatcttagtagtagataacttaacgaaacaagttattattagttaccatggtttaactaaatagaccccttgtacagtgatgtgcattgaataataccttaaattgctagaacaccgtaaagctaactaaactacaaagtaagtcgaaagactaggcttgaatgttgagaaatcagaaaaaattagttagatggtataaggttaaatcctaagtactacataattggtaattagcatccaagctcctgtaaaatggagaaggttcaacgactataataggtaagtcttagttacatattaagattatggtatagtctagtccctttaaaatatatcgaaagatagggtataaacggttcagcaaacaactacaacgagacttggcagacaaatgaaattgtaacagaagaaattgagtttagctacctttaactaatagaggctatgtttggtgacaagcatagaaaacactttaaattgcgtgaaagtcttaaagactagataactacaaagtaacctgaaagggtaagcttgaatgttgagaaatcagaaaaaatatctagtatagtataaggttaaaccctaagtacagtaaaatagatgatacgcaggcaagcctacgagagtgggaagcttcaacgactataataagtgagtcttagttacacattaagattatggtatagtctactccctttaaaatatatcgaaagatagggtatagaggacagcatcagataaggctagaacttaaatttcgtattaagacctaacaataaaagttaggtctttttttatacttgttttattagaagtactgtattataattaaaaataaaaggaggtgtactaaatgacaaacaggaaaacaataggtaaagtaagtaattcaagggcaacatggaaaattaaaccaacaacaagaattaaaaaagataaaacaaaatattctagaaaaaataagcataaaggtattgacaattataaataactgtaatactatagttatataaggagatgatgaactgtgaaaactttttattcaggcagaagaacaacaagtaaaaacaggcaagtaaagaagcattataaacaaatgactaatcaagaaataattatatgtaggaacttactaaaagaggcttataataaaaataattactttaagttaacaaatcacttcaaaggtaaatgtaagaaaccagtaaattttaaagcgtttgttcattatatacataataagaacttagatattatagaatataatgaaacattatacaataacaaaatacaacgtagggtagttgttagacacccttatgtagtaaaagtaaataataaattatcttaccaatatttagttatagaagttgaaacaggtgaagttgtaactatgtactataacagagtcacagataaccacaaaacattaaatttaaatgattattatgataaaaatttaaaaataaagtattgacataagtaaatacataatatatactaaggttacattaataaagaggagaataaaaaatgacaaataaattaagagctatccatgaagaaatgaaaattgagttacataaattcccacagaatgtagatttaacaagtaagacaactgctattgcaatcaatcaaattcttgacaagtttaaaaccttaacggaacaagcaggtaaaatcactaaaaagtattttgagggtcaagagattttgacgattgattttgaatattatgactcacttcaagattactacatttacttacttaaaaatagtgaaacaattgaacaaaggttacaagaaattgaaaaaagagtagctgaatatgttaagtaatttggagttataaccacattatgttatactgtatgtgtataaaaaacataagttagctgttatattaattatgagaaaataataaaggatggataaagaattggaaaaagaacaaaaagatattaaagatatgacacctgaagaaattgatgagttaaaatatcagcaacgaaaagataaagagcgtgttattaacaaagtaattaaaggtgttaatgatgtatgggagaaagaatacaactttgaagaattagatttaaaattcaaagtaaaaattagattacctaatgctagagaacaaggtaatatcatggctttacgttctgcatacttaggtggaatggatgcttatcaatctgaccaagttattaatgcttatcagatgttagctactctacaagaagtaggggtagaagtacctaaagaatttagagaccctgaagagatttataacctaactccacttgctattatgtgggatgattggttagactttatgacgtcctttcgttactagtaacgtaaagaagttaaatgaagatatagaacggttaggtggactttcacatatagctagagagcctttatcaagaaacttatgggctatcatgcgtgagtttaatgtgctacctaccgaacaacgttttaaagatttagatgactatcaaatagagtttattatagctaacatgaatttagatacaaaagaaatgatggaagctagagacggtaaaaactatgatatgaaagcggaagacgtagacacatcatggttcgaagcgccgacagatgaatttgatgttgttcctgatttcttagacttagatgatttaaatacacaagtagataaaagtttaagtgagaaagaaaagatagaaagagatagacgagttgaagctgagttagaagatgaaaccgagggattaacaactcaacatttagctatgatggaacacataagacaaaaacaaaaagagcttgatgaaagtttaggtattgataacaaagaaacaacacaagaagatataaacaaagcaatagaagatgtagatgattggtacatgtagggtaatggtataacaccaatacccttattttttgattaggtggtgaatactgtttggcaatgaatgacgattataggttggtcttgactggtgatagttctagcttagaacaaagtttaaaagctatagagatgtacatggatgcgttagagtctaaaaacatagatgcaccattaacaaacttcttagagaaattaaaagtaatagcaaaagaagtaaaatctgtacaaaacattatggataaacaaagtgataagtctttaatatcaccaaaagctatggatgaggctattagttctacacagaatgtaactaagaatataaacgacctaaagaaagctttaaatgacatacaaacagataatattacaaagggtatagctcctgaccctgaagtagagaaagtatatagtaaacttaataagacactaaataatacacaaacagaactagagaaagtagcatctcaaaagattggttcagactcagatataacaaacagaataaaagaaatgaaaacactgaaccaagttacagaagagtataataaattagtaaaagatgccagttcagctaaggagtacacaaagcaactaagagctaatcgtaatatggttagaggtcatatatctaggtctgaaggctctaatagaatgtcctatgaccaaggtgctagagtacgttctgaattaggtaaagtagacacttttgaaaagcaacgagaagctaacaatagaagaataaaagaaaaccaagatagatatagaggttatagacaacaacaacaagatttagtaggcaaaagagctcgtggtgaaataagttctgaggattataaaaaacaatcagcttctattaaaatgatgattgatgaaagtgaaaagctttctgaagtttatagaaaaacaggtgctgaattagacaagtcaatcaactactataaaaatagtgctaaaaaacagtttgctcagcgtgagatagaacagcaacgaggaacaatgggtaaattgttccaagatagattaccatctattggtgcacatgctactatggcagtaactgctcttgcaggtggtatgtatatgaaaggtgcctccttatccgaggcaaatagacctatggtaacatcattaggacaaaattcagataatatggatatagacgcagtaagaaatacatatggagatttgtcaatagataataagcttggttataattcaactgacatgctaaaaatggctacgtcatatgaaagttctataggacataaaagcgatgcagatacgtataaaggagctaaacagttagctgtaggtggtcgttcattaggtatacaagaccaagaagcttatcagcaatccatgtcacaacttatgcatacaggtggcgtaaactcagataatatgaaagctatgcaagatgctttcttaggtggtatacgtgaatctgatatggttggtagacaagacgagcaattaagagctttaagtactattgccgagcagtcaggacaaggtagaacattatctaaaggggaaatgtctaatcttacttctttacaagctcaagtagcgggtacaggtagtaaaggtctacaaggtgcacaaggagctcaagccttaagtagtattgaccaaggtattaaaaatggtatgggtaattcttatactagattagcaatgggatggggtactaagtaccaaggattagaaggtatgcatgacttacaagcacaaatggataaaggtatatcagaccctaataacttagttaatatatttgaccaagctaataatataggaagtacaacaaaagaaaaacaagccatagctaaaaaaggctttgaaagcatgggagctaacttaacacaagagcaaacagatgagttgtacaatctttatgcctcaggtaaactatctaaagaagagttagctagtaaagctaagagcatggaacaagaaggctctaaagcaggagataaaaataaggataagtattccgagtctaaagcaggtagaaatgaccagaacaaagctaagacagatgataaagctgaggatatttatgacttagctcaacctattagagatgttcatagtgctatggcaagcttgccagctcctttatacctagcttcaggagcagtacttgcctttgtagcatctttagctaaatctactgccatgatggctggtggttctttattaggtaaaggtcaaaaaggactaaaagattggtttaataaccgtaaaggaggttcaacaggtaagactggtggtggtaatcctaacggtggtggacgctttggaggacttaaaaacttagctgacactatactaggtgataaccctagaggtggagaccgaccatttaaagaccaagcaaaaggagcaggacaaactgctaaaggcatgggtaaaaccttatttgatacctttggaaaaagagattttacttttggtgaatatatgggaagaaccaaagacctaggtaaaggtgtatggaacaaaggtaaaggagcttttggtaaagctaaaggtaaatttagtggtaaagactctaacttaggttttatgtcacaagcacctactgctaatgcaggcggtcttggtaaactaaaaggtcttacaagtaagctaggttggattggcgcaggtctatcagcctttgacataggtagttctctaatacaaggagatactaaagaagcttcttctaaatttggtaatactataggaagtgtcatagacccattaaacctaggttatggagacggcttaagagattatgctacgaaaacagcggaaggttctatgacaagtgatggttggaaattatggtctaatggagataaagatggtaagaataaattccaagactctcctgtaggtaaactatggggtggcataacagatttcttcacaccagataacccttctaaattagacgagacagtgaaaaataaaaaaggtaacataattagttatatactaattgccttgcttatagagcaatctgtaagtaccgagtgttaactgctatgaaactactaaagctaactaaactacaaagtaagtcgaaagactaagcttgaatgttgagaaatcagaaaaaattagttagatactctaaggttaaatcctaaggagaagtacaatgggtgtatagcatcgaaagtcctaaagacctgaaaggtctatggaacacgttcaacgactattccgtataggtgacggaagtaaagctccaagctattgggagaagaaaaatactcaccctatactaatagggataacaaatagtctgcgcacgtaccgaaaggtagtgtctagaaataactagagaatatacagtagcgtgtatatttaaacattgttaaaatatataaataattaaaatttgtattctacttctgcatataaacctttcttattttgtaaggtaaacttgttattaaataaatatacataactaatacgataggttatatatgaaagtatactattgtttatatgttttaacttatggtattaatgataaatcacaaagtaaagacaggcaaaaagcagaagaaaaaagagaaaagaataatgattctagagaaggaaatgtaacagtatactcacatttcttagaccatttagcagataacctaacaaataaaggtagtagttctaacgacagttcatcaggaggaacacttgactaccttgatggtaaaaactttacggataaagacgtaactaagcatgacctaggtaaaactgctaaaggtgtaaacgccgaaatgttaaataaatggattgagtctcaagcacctagtaattctaaaatgcgtggtttaggtgagctttatatgaaagcaggtaaagagtcaggacttgacccaaggtatttagtagcacatagtgctgttgagacaggctggggaacatcaaatctttccaaaggcggagaccctaacaaaggtaactggttcggtataggtgcttttgataacaatcctaataatggttttaactatggtttgggtattgttggtggtgctaaatggattagagacaacttctacaacaaaggacaaaagaacttacacgatatgagacacaacaatggtgttcatgaatatgcaacagcaggtaattgggatactatgattgccagtattatgaaaggttcagacaagttcataggtggcggaggtggaggcagtgatgttatgcctagacatgctctaaaaaacgaccatcaaccaaagaatcttaaatataacatagaagtacctaaaagtaaaaaccctaatgaaacagggaaagcaataggtagaaaattaaaacaattattagaatctaatttttagaatacaattataatacttatatactttaacaaatggtacaaataataaaaaacagaaaatgtctacagaacaactaagagaaaagaataataaatcagaaactaaaaacttaaaaacttacagtaacctattagatagagcacaacaaattatagaagatgctaaagcgctagactcaggaggttcagacagtagttctgactcagggggttcagcttccgatgtagatggagaaggtgcgggtaagatttacaaattccttaaaggtaaaggtttatccgataaccaagtaggtgccgttatgggtaaccttaagcaagaatcagggcttgaccctaatgcaaagaacgcctctagtggtgcttttggcattgcacaatggttaggtagtaggaaatcagaccttgatagctttgctaaatctaaaggtaagaagtctaatgacttagacgcacagttagatttcttatggaaagaaatgagttctggtcaaaatagtgatatgcttaaaaatgcaggatggagtaaaagtggtagtttagaaaaaaatacaaaagcttttgcacaaggttttgagcgtatgggtgctggagaggctatgatgagtactcgagtaaataacgctaaaagttatgtaagcaaatatggtaagtcaggtggcggtggcggtggcggtggagctataccatcatcatatacgtccgtattaagaaacccgatacttagtagtgcttcaggaagtactgcatctcaaaataatgtatctgtgaatgttagcattacaggaacaaatgacaaaagggatgctgaagaaataggtaaaggcattaagtctacttttggtgacgatttagacatcttctcaaacacgtataaacgtaattattagggtagtattattaagtactaccctttatataaaaataaaggaagtacatatatgtatagaattagacgacctaagataagaatagagatagtaacagatgacaatacatttacattaaggtttgaagatactaaagattacacaggaaatcagtttgcttcaaaattaataaacttccaaactaaaaatgcaatggaagatgacagtgcagtattccaaataacaatggcaggggatacatactgggataagttagtaatggcaaatgatattgttaagatatatattacacctaacaatgaccctaaagataaggaaggaaataaagagaaacttatacaagtaggtatggtatctcaagtttctaaagtaggaagctatagtaatgaccaggttcagtttagaataacaggtcaatcttttgtaaaaccttttatgaaatttggtttaggtgttatacaagaagtacaagctgtattaccaactgttggttggttagttgatggtgatggagagaaccaagttaagtttacaggtagttcagctaaagatgtaatgcaaggtattatacaacgatttatcccatacatgaaatataattatacagataaaacgtataacacactagagagttatttagattatgatgatttatctagttgggatgagtacgaaaagttaactgaagtttccgcttttactaacttcgatggtacattaaaacagctaatggacttagtaactgctagaccttttaatgagttattttttagaaattcagataaacataaaggtaaagcacagctagtattaagaaaaactccttttaatccaactgaatggaaagcattagaatatatagttgtacctacggaagattttatagaagaagatgttggtaagagtgacgtagaaacatactctatatttacagtaaaaccagcaggtatgttaaaagaattaacaggggatgtattctctaaacctcaattccatcaagaattagtagatagatatgggtattctaaatatgaaacagaaaacctatacataggaactaagagtggtagtgctacagaagatagtgaaactacaggtggagataacggtcatgaacgagtaacttatgacagactacttaaagatttaaataactatggtagagagtctatatctaaaggattagataaatactctagtaagatatcatcaaaatacaacaatattactaaacctgaagtaaagagtattttagaatcttatataaaaaaaggtaatctaagtaaggaagaatatgaaaaaataacaaagaacaatacttcaagagagtcagtatctgatactagaccaaaactaacaaaagaaaaattaaagtctatattagcggaaaagtttaataagaaagaaacttttgatgataaagatttaaataagaaaacaacaaaagctgtaatagatgatattacaaagaattataagtatggtaatcctacacatgctaagaaactactagaggattacactagatttaaaggtaacccacctacaagtaccacaggtgatatttatgataagtatcttaaagccgtagaaggagtagctaatgttgctatagatacaggttctgacgcaagtgatgaccctttaattatatttactaaaatgctatttaattggtatcactcaaaccctaatttttatgcaggtaatattgttgtattaggtgaccctaagtatgacctaggtaaacggttatttataaaagataaacaacgaggggatatatgggagttttacatagagtccgtagaacataagtttgattataaacaaggatactttactacaataggagtaacaagaggactaaaagatgctattataaaagatggtgaaggaagcaaacataggtttaaaggactatggaatcagtcatctgactttatgggtggtcttatgggtgaagagacatctaaagaattaaaagaaaaaggtgtagccgagaagaaaacaaaaggaaataaagatggagattctgacggaggcgcacaagatggaggtaccttagcttcactagaaaaatataatggtaagttgcctaaacatgaccccaactttgttcaaccaggcaataggcattataagtaccaatgtacatggtatgcctataatagacgtggtgagctaggtattcctgtgcctttatggggagatgccgccgactggattggcagtgctaaaagtgcaggttatggtgttggaagaactcctaaacaaggggcttgtgccatatggcaaagaggtgctcctgggggaagcttacaatatggacatgtcgcttttgtagaaaaagtattagacggtggagctagtatatttatatcagaacataattatgcaactcctaacggttacggcacaagaacaatagacatgagttcagcaataggtaaaggtgctcaatttatatatgataaaggataattgatatggcaacggataaacaagttaaagatgttatagataaatttatagataatcttttcaattttgaagtattaaccaaagaaaaggttgaagaaaaagataaggaagttaaaaagataactacagaagacgtatacaataaagttgtttatattagaccatatgtaggtgttatacaaagtataacacctagacaggtaacatatgatagttttactaataatggtacagatatagaagctaaactcacacttagaaaaataagttatactactgataatctcacagtacctactgatgcattatcaacttgtatggtacatatggtagaaagaaatggtgtacttgttatagattactttgatgagttacagaacatactgtatggtagttacatggataatgagcatatatttgatgaagatgttccagttagtacattagtaacattagacttgaaagagaatttaaataattataaacatatacagtatatgtttaaagggaatacggataaaaacccttttgaaagtaaggatatagtacctttagatacatacaacttattatattggatgtataaaaagagaaatgtagagctaaaaaatccattaacagttaactacttcttcacaggtaaaagcttttatactatatttggtaaaggacacaaatataaagttgatattgacaaattcaaaataggtgatatactattctttggacgtagtgatgataatatagggatatacataggagataagaagtttatttctcttatgggtacatttcctaaagataacacctctattagtacctataagttagacgattattgggatgactttaatggtagggttatgaggtttgatgaggatgatatagcatggtggtaagattacaatcttctttaggtagaagtttaaaaaagatagactcagaagaacttaatgttaaaggtctattattaggtacagtaagcaaaataaattataagtaccaaactgtagaagttaaaataaatagcttaacattaggacataaccctagtgatgatggcaagttagctgttccttatcctaaagattatgtaggaagaacacctgaaggtagcgtgtatggcagtaatactttaataacggaaggaactactgtactgttaggatttattaatgatgacattaattaccctattatactagctatctatggtaatacagaagcaaaccaactcataaatacgaatcctttagagggtggtaaaatgagtaatgatgatgtttataaatatagtagtgctttgtattctatattacctgacctaacttatacatataaagatggtgaaggtacatctataaaaacattcaatggtaaatctttcttatccattacttcaggtgataaagaaaaacctttatcttcagacttttatacaggtactgaataccaagacttatttccatcatactatagtaacaaaaatttaatagaacctagaatacaaaaggcacctaacatgttatttaaacaccaaggtctatactttgatgatggttctgaagataatcatgttacaactttatttatttccgaaaaaggtgatgtaagagcttcagtattagacaaaagcacacaaaaaagaacaacacaagagatgtctcacgatggttcttatagagttataaagcaagatgatgacctattattagactcagcgcaagtatgggtagagtttggtataaatgataataatgtgttttacataaaaaaccctaagcatatgtttgaatttaatgataaaggtatatatttagatgataagccaattttagatacgttagatgctagcattacagagtctatagatagacttaaggaagtacagaaagaattagatgacattaactacttacttgagggtgtaggtaaagaaaacttagaggagttaatccaagagactaaaaaatctatagaagtttctagtaaggcaagtagcgatgttacgactatgaaatctcaaatatctaatgtaagtagtagagctgagggtattattacacagtttcaagagtttagagacaaaacatttaaaggcttatatgaagatgcttctactatgataaataaatacaaccaagaaatacctgagttaaaaagtagattgaatactctagatacaattacattacctaacattaataataccttattagagaaatcaagtaagacctatgttaataatagttctcaaagtgttagtagtaattatctaggttcattactgcaattagatagtaaccttactatttatccatacacacctacacgtattacatggaataagatagtttttaataattctgagtttgtaaataagactaaggatgcttttgttattccgttaggtgtatctaaagttaaagtttctattaatcttaaatgggatatattacaagataaagtaaaaactattatagtcaagaagaatagtaatacatacaaaactataactaatggtaatacacacgataatataattacaaatgttatacctgttaaacaaggagatacattcacagtagaagtacaacaagataatgatattaaccttacattactacaagataatacaatgtttagtatagaagtattagaaactactttagcacctactaacactgagttattaggtacagcatccgaacaaacagataagcaactacaaacacttgtaaattctaatgttgatttattgagtttgacaacacaagttactaaagacaatcagttagtctgtcttaatgattctaaattagatactataacaacaggtaaaggtaatgtgtctgattacactctacaacaaataaagtcattttcatctaagaatggtgagagtattttatctttagaagatgtattagctaaatataaaaattctattagatatagtattaatttaccaactactgcaaacatagtagaactactgaaaaaatataatttaattggtataagagcacctaggaatcaagtgtatttgaaagttaaagatttaacaatcacacaagaagtattaaataatttttctaacttaccagtattatataatacttcctatctaacaagtacagatattaacaaaggtatatatacaaatatatatggagtactactaccatatactagtatcactcaagatacagtaaatacagcacataataataacctatctatatatgctagtggtgatatgacaagtgacattataaataagcttattttatatggtatagatggtgtagaaacaaataaaatattagagtttaaaaaagtttaaaagtcctattaatagggcttttttatatatgtcatgttatattaaacttgagaaagtagttgtatttaaatactatatatgatataatacgatatagaaaggtggtaagttatgcctcaatcagatggaaagaatacaataagacgtattgcattacgttttccaacagcaagcggtaaatataatatgtaccgtttcaaagtaaaccctgaaagttatgaaatagatgcaccacaaagaacatctataactaaaactaaatcagacattattgttgaagactatggtaaagatatagagacaataactttttctggtactactggatttagacctgtaaaagaagtagatggtactaaaacaggaaaacaaaaaatagaggaactacaagaacaagtagaagcatacgctaagcagggtggagatggtagcgtagcaggagcttatatagagttctataactttactgatgataaatattacaaagtacatctagcaccgcaaggtttaaaaataacaaggtctaaagatgaatccttgttatatagatatgaaataactttagttgtgataggtaaccttgctgaagcagatagaggttccgtaacatctgctgagtttggtaatgtaaaacctaatgctaaacaaagagttgatgagggtgttaatgatttagatagtaacgcacgaaaaacaagaaacaagaataatcaaatagtatcacaatttgaaaataaaagtagtcaaagctcttctaattctagtagtagcggttcacgatatggaacggcaggaacattaagaggaggtagtggcataagaacaggtggttctgaaggtaatagagacggtatgagtaatataggtatatataaccctagacagaatactaatggtcttagtagtactgttgatgatatggcacttaaaataggatacggtgatggaggtgtatctaggtaatgaataattatatacctcaaccacaaggattattaaggtttttaaatggattagagttagacatgtcttacacacatatgaacttattagatgaggaagtaccttttgtttctaaattctatacaccacaaatacaactaagtgaacttgcaagaataaaattaaaagagattaagtctactgatatccctactttagaaagaacatttaacaacaatacaatagttaataaagtagaacaaacaagtttaaagttacaatcacctagaatgtatttaattatgcaatctatcgtaatggaagcttacgctattgtaaactgctttgtagaaagtcctgaatacttaaaatatttaacagaaaaagatgtatctatagtaagaagtaatgttaactacgtagctgactatttaagtgattatgaagaatacaatagtattgttctcgatttaagagacttagacttatgttttggctctttggaattacaactaccactaattaagagtgaggtggtataatgagatttagaaagcatgaagtacaatcagaagaaaccttacaagctatatcacaaaggtattatggtgatgttagttactggttagatttaatagaacataacaatctacaatatccttatattgtagatacagatgaagaaaagaaacagaaccccgagcacttaataactaaaggtgatattattatcatacctttagattctgagttaacagactcggtttctaaagaaataacatcaagagacaaagatgtattagtagaattagctttaggtagagacctaaatatgacggataataacgagtatatagacgctcatggaacagatgatgaaatattagccctatcttcagatggtagaggagaccttgatatagttaaaggcatagataacattaagcaatctttaagagctagactattgacacctaaaggttctttattattacatcctgaatatggttctaacattcataatttatttggtttaaatatacctgaacaagctacgttaattgaagtagatgtattaaaaaccctaacatcagatagtagagttaaatcagctaacttaaaaggttgggagataaaaggtaatgaatactatgggtcatttaatgtagaaatcaagtccgtagaagaatctatagactttgtattaggtcaagatgattcaggtgtctttgctttatttgactaagaaaggaaatttatatgagaacaagaaagttaacacagatattatctaggttaatggataagacgatacaaggtacaagcaaaataacagactttacacccggttcagctgtacgctctcttttagaagcagtagctttagaaatagaacagtattatatattaacaagagaaaacattaattggggcattcaagaagggattattgaagcttttgattttcaaaaaagaaaagctaagcgttcttatggtaatgtagatattcaattttaccaacctttagacatgagaatgtatatacctgcaggtactacattctcatctactaggcaagaataccctcaacaatttgaaacattagttgattactatgctgaagaaggaaccactgaaattacagtagaagtttactgtaaagaagttggtattgtaggtaatatacctgaagatatcattaatacaatagcctcaggctctagtttaattcgtacaattacaaacccgacttcctttaatacaggaacaaaagaagaatctcaagaggattttaaacgtaggttccatatgtttgtagaatcgagaggaagagctactaataaatctattagatatggtacattacaagtacctgatgttgatggtgtttatgtttatgaagaagtaggtcatgttactgtatatgcgcatgataaaaacggaaatttatctgctactctaaaaaatgacataataacggcattagaagattacagacctagtggtataaagttagatgttgtaggcgtagagaaagaggaaatagatgtgtctgcagtagtaacaatatctaataaagctaggataggtgatacattacaaagacatattgaaagtgttattagaggatacctaaatagtctgactacttctgatgacttaattattaacgatttaattcaagctattatgaatatagatgataatttaatttatgatgtagcatttaagaatttatctacaaatatagaagtatcttcgaaaggtattattagagcaggggatattaaggtagaattaatataagggggtaaacaatggctaattttttaagaaacttacaccctttattaagaagaaataagaataataaagataaccaagaccctaactatgctttaataacagcacttaatgatgagatgaacaacatagagtcagacaccattaaaagcaaacttcaatcatcattaaaaactaccacaggagattacttagataagtttggggattggtttggtgtatacagaagaataaatgagaatgatgacagctatagaaaaagaattataaaatacttattattaaaaagaggaactaacaacgctattattagtgctattaaagattacttagataatgagaatataaatgttagtgtatatgaaccttttaaaaatatattctataccaataaatcaaaattaaatggtgaagattatttaatgggttattattatagatttgctgtgataaatgtttctgttgatggttactttcctttagagattatagatgtaataaatgaatttaaacccgcgggtgttaagttatatttaacttatgatggtgctactaccataaagggtgatggtgttgttaaatggcttaaaaatctacctaaaattgaaacatatacggatttagatagattattaggttatgatgaaactttctatgggcatttaaacatgagtgaagcaacagctgaacaatctgataaaacaaggttcagaacaaataagagtacattaaatagtgaagatattctatcaggctcttctaatattggacgtagatttataaattatgcttatgttacgacatatgcatatgaacctactagtacatcagccgtcacacatatagcttctttaaatgataaaggaaaagaagcacctttagattattacttatatacatccttaaagaatactaatgatataaatatatcaatggaaactgcttatggtgtgtcttatttatacaataactttaatgtaggtgagtttatcagtagatacaaacctaatatagatataactagagataatgctaaaaaacaaatatccgattttgtaggggaactaacaattgatttctttgcaaaagctttagtttcacctgatgaatcattacctattaagttacaaatatttgattttcgtactaatgaatggttaactgtatcagaaacagaactatctttttacgagaaaaacataggtgtaaacataggttatataagagattacttaaatgatgaccttaatttatttacaagattagaggttaatataggtaaagatgagcaaaaagatataagtattaactacatggatttacatttttataattataaaaaagatgtgtatactatcaaaccttttaaggcactagttgaaaactacatagatttgactaaagatacttatattgaaggctttaaagtagcctcattattaaatggagacataattaataaaaatggatatcaacccataaaatatttaaggttacatgctacttatgattcaactaaacctattgtaagtattacaggtaaaaacagcaataatgaggtacaaaataatacacctatagatttatataaaaatacaactaatagaaacttattgcaatcatataaaggtgatagtagtatattcaagggtgttgtaagcacaaaagagttttatatctcatcatggtctgacaccttatataactcaacgtatttatctaaagtactagaaccgcaaaaagtatatactttgtcattcgagatggaaatcacagatgctaatatggacttaaaaacatattctgacaatcatggtatatatttatatagtagtactaaaggtattgttgctaatggtgttaaaagcatgcaaagaaagataggtaataagattgatgtatctaccacatttacagcacctacaattacagaccatagacttgttgtttacacaggtagatatacagcagatggtactgcctctaaaacacctattgcttctaatacggtaaaagtaacaaaccttcaactaaaagaaggagctactaagaaggactacacggtagcacctgaagatgtaactaatgttatagacaaagctattgtgtttgataacatattaacagatatacaaacaataggaataaatacaacaacacctcttaaaaatattgagctaagttattcttattatggggataattggacaaacctaaaaacaataagtaatttaagtcaaggtgaaacaataacacctaataacctagtagatttatatggcttagaaacaatagattactcaagtattacacctttctctagggttatacttaaaactatatggaatgctaacctgaataatttaacaaataaaaaaggtagtatttctaacatgagtaataactatttcaatgctgtatggcaagaggtagatatgatatataacactacattaagtagcatgagcatacttactgatagtgaaggtaatgtatttgatagttcaacaggacaggtaataaaattagcacctaagatataccaaggatatactgatgtagatagattgctaacgtctacgactctatataacgagaatattacattaggttcaaatagatttatttatgacttacgggatatgttactatcaagtgatacattcacagtagataataaaattaaagtaatagatacatacacagaagtacagtaaagatattgacaataaagtattaatatgatatagtaaggtatgcgtattatagtataccttgttatattaatacagaatactagacatgaaaggaataaaaaatatggctatagcaacaaatgcttcacatgtagaattagctaaatacattgtaagtaaggctgattctatttatttaacaattggtaaaagtacagcgtggtctaatgaaacaagtccacctcaacctgaagaaacagcaacatcattacaagaagttatcggatataaaaaagcaactaaggtagcattagttagacctgcaaaaacaacgggtgatgaaagtaaaaaagaaattacatatggtaataaacgatgggtagaaattgactcagctaatgcagttgctgaaggtgctaagtgggtgtacttagaaagtagtattgttggagatgagttacccttaggtacgtatagacaagtaggatttgttatggatttgttagctaaagatagtatttctaaatttaacttattacctagtgaagtgcaatctacaggaactttattattttatgataataaacaattccaaaatagaagtgaacaaacaactgtaaaagagcgctttatcattgaagtttaaaaaggagaattgaatatatatggcaattaattttaaaagttctccatacttagatagatttgaccctacaaaagatagaacaagagtactatttaatccagatagaccactacaacaatcagaactaaatgaaatgcagtctattgaacaatattacttaaaaaatttaggggattctatttttaaagatggggataaacaatcaggattaggttttacattaactaaagataatgtactacaagttaacccaggttatgtgtatataaatggtaagatacgttactatagtagtgaagacactgttaaaattacgggtgtaggtaaagaaacagtaggtattaaactaacagaaagaattattacacctgatgaagataatagcttattagaccaaacaagcggagttcctagttatttttctaagggtgcagaccgtctagaagagaagttatctttaacagtcaatgaccctacttctgctactatttatacttttatggatggagacctctatatccaatctactaatgctgagatggataaaatcaacaaagtattagcagagcgtacttatgatgagtcgggttcatataaagttagtggatttgagctattttcagaaggaaacgccgaagatgataatcatgtatctgttgttgtagatgctggtaaggcatatgtaaaaggttataaagtagataaaccagtttctactagaattagtgtagagaagtcaagggaactaggtaaagcagaaaatgaaagtactgtatttaataaatcatcaaataatattactttagctaatacccctgtaaaagatgttcaacgtgtaacagcgcaagtattagtagataaagagcgtgttacaagaagttcaacaggagattctcaagactatttatctaataaaacagcttttgaagtagttagagtatggacagaaacaagtccaggtcagacaacaaaagaatacaaacaaggagaagactatagacttactgatggtcaaacaatagactggtctccttcaggtcaagaacctaatggaggtacatcttattatgtttcgtataagtacaacagaaagatggaagtaggtaaagattatgaagtatctacagtaggtgagggagtaggtaaacgttggtctattaatttcacacctgttaatggtactaagcctattgaccaaagtgttgtacttgtagactatacttactacttagctcgtaaagatgctgtttttattaataagtttggtgatattgctattcttaaaggtgaacctaatattatgagattagtaacaccacctttaaatacagaccctgagaacctacaattaggtactgtaacagtattaccagactctaatgaagcagtatgtatttcatatgctattactagattatctatggaagacttacaaaaagtaaaaactagagtagataatttagagtataatcaagctgttaatgctttggatgatggtgcaatggaaaaacaaaacccattaacattacgttctgtatttagtgaaggttttatatctttagataaagccgacattacacatcctgactttggtattgtatttagttttgatgatgcagaggctactctagcgtataaagaagctgtgaaccaacctaagattattcaaggagacactacagctcatatatggggtcgactaatttccgcaccatttacagaagagcgtacaatttatcaaggacaagcatccgaaactttaaatgtaaacccttataatataccaaataaacaaggtgtgttaaaattaactcctagtgaagataactggatagacactcagaatgtaacaatcacagaacagaaaactaaaaaagtaacaatgaaaagattctggagacataatgaaagttattatggagaaacagaacattacttatattctaacttacagttagataaaggacaagagtggaaaggtaagtcatacgcatatgatagagaacatggtagaacaggtactctattagaggctggaggacagcgtacacttgaagaaatgattgaatttattcgtgttcgtgacgtatctttcgaagttaatggtttaaatcctaatgataataatttgtatattttatttgatggtgttcgttgtgctattacaccagcaacaggttatagaaaaggttctgaagatggtacaattatgtctgatgctaaaggcacagctaaaggtacatttacaattccagcaggtattcgttgcggtaatagagaagtaacattgaaaaatgctaactctacaagtgctacaacatatacagctcaaggacgtaagaagactgtacaagatgttattatcaaaacacgtgttacagtaaacttagtagacccattagcacaatcattccaatatgatgaaagtagaactattagttctttaggtttatactttgctagtaaaggagataagtctagtaatgtaactgtacaaattcgtggtatgggagatacaggttaccctaacaagacggtatatgctgaaacagtaatcaactcagatgatattaaagtatctaataatgctagtgctgaaactagagtatactttgatgaccctatgatggctgaagcaggtaaagagtatgctattgtcattattacagaaaatagtgattatactatgtgggtcggaacaagaacaaaacctaagattgataaacctaatgaagttattagcggtaatccatatgtacaaggcgttctatttagttcatctaacgctagtacttggactccacaccaaaattcagatttaaaatttggtgtatatacctctaagtttaatgaaacagctacaattgaatttgaacctattaagaatgtatcagcagatagattagttttaatgtctacgtacttaactcctgaaagaacaggttgtacttgggaaattaaaatgattttagatgacatggctacatctactacatttgaccaattaaaatgggaacctattggtaactaccaagatttagaggtattaggtttagctaaacaagttaaattaaaagctacatttgagtctaataagtatatttcacctttattaagttctaatgacttaacgtttactacattcttaactgagttaaaaggttcatatatcggtagagctattgatatgacagaagctccatacaacacaataagatttagttatgaagcattcttacctaagggcactaatgttattcctaagtattcagacgatgatggtaaaacttggaaaacatttactaagtctgctcaggtagttaaggctaatagagactttaatagatatgttattgatgagaaagtaaatcaatctactaagaataacaaattacaagtaagattagacttatcaactgaaaatagtttcttaagacctcgtgtacgtagacttatggttacaacaagagatgagtaagtttaaagagggtttcataccctctttttatttaactaaggaagtgatattatgcctacagaatatagagacccttactcacaagctaaactctttattcctactgtagaagaaaaagctataaaagaaatggaacaaacatataaagataaactacaagaagttaatgagcttattgatgagcttaaaaaattgaaaggtgattacacacatgacgtttaactacacacctctaacagaatcacaaagattaaaggatatgtaccctaaagtaaatgaaatgggtaaatacttaaaagaagaagttaacttatctgatttaaaaacgattagttatcctgatttaaataatgttttaaatttcatacaagactcaggtgactactttgtaacacaagcaagaaacaccccgcaaggtgtaagtaatatagggtttttacacttagataaaaagggtagtacttattataagttatattatgccccgtctaacacaaataaattgtttgtgaaaacttattataatggtactgtctatgattgggttagttttaaactagatgaaggtacattatacgattcaggaaatacaattaatgtaaaaaatttaacagagtccacaactcaatttgcaacactcataaatccccctaaaagtaatttaaacacaggatggataaattataaggaaagtaaaaatggtacctcagctttagctgaatttaatcctattaattctacttcaacatttaaaatgattagaaagttaccacgccaagaccaacaacctaacttattaagagatagtttattcacattaccaatgacggatgtagctaatataagaacggattatatagataatacatctttttggggttttactaataatgcatctacctcaacagttagatatatgggtgaaaatactattcaattagatgatggaggggaatcatatcctacagttatgtcaaaacgatttagaataggagacgaactatatgtaggggatactgttacaatgtctgtttatattaaagtaaatgaccctaatttgttaaatggtaatttcccctactttgagattgctgggtataattatgtttatcaagacaataacccatacacaggaggtaggagagaggttaactattatgacttatctacggagtggaaaaagttctcctttacttttactttacaatcatataaccaagagtctagtaatgatagggttaattatatatcagccctactaagatttaactgctctaattctactaataatggagctattgtgtattatgcactaccaaaattagaaaaatcatactcggtaactccttttattactcatacttatgataaaagaaaatatgatgaaatatggagtaattggactgagacaacaagtaaagatgagcttcaatcacacagtagcgtagatacatatagtaatgactactataaatacgtatgggattaccaaagctcgaatggtagaagtctgcaacaattagcaatgtctgtacctcaaggtttttataccttctactgtgatggttctataagtggaactcctagtaataaaacaatacaaggcacaatacaagtagcgtataaaaataataatataaacactacaaataaagtaatacaaatggattttagtgatactaaaggcaagaaatattctttaacatataataaagataataatggttggcaagccatgaaagttcaaatggctagtaatagcctttgggaaggaacgcttgaccttgcttcaggaaacacagtaaatctatcagattcgttatataactatgattttattgacgtagtatattggactagaagttctggacataatgcaactagaagattccctgctactacatctagtattgtaattagagactttaacttagtaaatgatggctctaattctagcgtagatttctttgaaggatattgtgacattactgataagataaacgtcactgctaaaatgtcaaaaacaattaacctagatggaaatagaaataccacaacagtcgcacaattcaatcaaacaggtcacattgaagtactaagagtaatagggttaacatatatttaagagaagaggttttatagatgataaagtatgacattggtaaagacgaaattgtgttacacttaaaagataataaatatattatgggatatactattgtaggtggatatgaagagaacaatggtgatgtaaaagtatcaaggtctattctacctgatggattctttgttgattttgttagtaataagtatgcgtattataagaacactaacgaagtaatatataataaaagctttgaagataactcgaagagtgtagaggatatagaataccaaccaccagaagagtttgtacctaaagaagactacaaaaaattagagagtgaagtagaggaactaaaaagaatgttacaagaattgttatctaagaaaggataaatagtatggcactaaattttacacctattacggaaaacagtacaatagcagacctaacaaaacaagttaataacataggtagtgtattaactgaggatagaaatatctttgaagtaactacagacttacaattagacgtaagtaaagcacagaaagttaagttaacaacggatagaggattagctaaagatatagattataccaattacttaagagacattaaaaaagtaggtttatattacataggagcacgtacattggctactatgtatgataaaccagatatggaaaatatagatgtattactgcaagtattaacattagatactgaggatagagtaatacaaatattgtatacattgtcaacggaggacagtaaatcaaaagtgatgcaccgattcgttactaactaccaaacatcacaatggcaagtagtacaaagtttacctaataataaatatacatcacatataggtggaagtccttttgatattaatacacaaggtacatattacgtgtctaatatgtcggatatgcctagtggtgtttatgaaggattcttacaagtttccatagacagtaatgataatagaatgttaagattaacggatagtaacacgggaaaagaatatcttaatgttaagaaatcgtatgacaattggggtgattggagaactgatttagacgtacaaaagataagcaagtatcttgtatctaatgtaaatggagatgcggaaaacaataattactcattatccgtatatacatcagacaatataacattccaacaagctatagctaaacatattgaagaaacaagtaaaactgtttttactttctatatacaaggtggtgtttcaggctctccgtctacggttagttgcagaggtatatttatatcagactcaaacaactatactaatttatatggtgtatataccgcaataggtactgacggtagaactattaatggttccgtaaatggtaatacatggaacacaacaaaaacattacctggttttaaagaactatggaatggagcacataactttaaagatacaaataaaaaagaaactatgtctgattctatatctaactaccaatacgtagagatatacacaagatacagagccttacaaaacacaaaaggtacggataaaacaggtactttatgtcataagttttatatagatggagatggtatttacacatgttcaggagcttatgtatcaggagactctaatattggagtagagtactatcgagtaactttatctattactggtgatacatggacaattaaagatagtgcagtaaataataataaagaccaatatgttacacgtgttgtaggtatctctttaccatagactaggataattcctagtcttttttttttcttgactaactttaataaatatggtaagataataaaggactaaaaagaaaggagaatatatgcagttaagacaacagaatatatatacatatgtagattttgatgaaagtgacgggtatattaaagatatcatacttaagcgtgttcataaaacgttaggagctaagaaagagggctatcaacatagcttagcttttaaacgaggtgtgtgggatggttatgtggatttctacgattatgctgagaacaaattccctagcgggttgttacctaagatgataacattactaggtgagttacaatcaagacataactttcaatacgaaattattgatgaaaaatctgaaagctttttagctgaagaagatattgatgatgaaatacaactactagataataacgtaggtaagattacattacgtgactatcagtatgaagctgtgtttaatagtcttgtcaattataatggtatatgcaagcaagctaccaatgcaggtaaattttgccccttatagtagaaatattataagtgtaactcctttaattcatgggaactctctcatttgagacaatcatgagccaagcttaataaattactaggaaggtgcaacgactattatgtacaatcaagtgattggaaacagggagaacctaagtctcattagatatggttaagatatagtctgaactttgtaggagactacaagaaggttaggagtaacgaacctaatcgtaacataattgaaaacagaggttgctagcggtatcatagaccaactattaccacaactagaaaaaggagagcgtgtagctttttttacaagttctactgagatttttaatcaatctgctgaccgtttaagtgaacgattgaatataccagtaggtaaagtaggttctggtaagtttgatgttaaacaagtaactgtagttatgattcctactgttaactctaacttaaaagaccctacagaaggagtaaaagtatctgctaaagtaaacttaagtaaaaagatagctaaagagatacttcctaaatttgaaggtggtaaaaatcaaaaaaggttacttaagttattattagaaagtacaacacctaaaactaaagtagagcagaatgtgcttgatatacttcaagatatttatcataattcaaagacggatgctgaagtattaatggcattaaacaatcataatgttattttccaaaaagaagtaagaaagaaaaatcaaaagagctatgataaatatcataaaatgcgtgagttcctagactctatagctgtaatgatagttgatgaagcacatcacagtaaatcagattcatggtatacaagtttaatgtcctgtgagaatgcactatacaggatagcactaacaggctccatagacatgaaagatgagctactatggatgcgtatgcaagcattattcggagatattattgtaagaacatctaatgagtttttaatcgagaatggctattcagctagacctacaataaatattatccctattgctaaccctgatgatatagataatattaaggattacagggatgcatataataaaggtattgtacataacgagtttagaaataaacttattgcaaaactaacagctaagtggtataatcaagataaagctacgcttattattattaattttgttgagcatggtgaaatactatccactatgttgaatgaaatgggtgtagagaatttctttttacatggtgaaatagagtcggatatgcgtaaacaaaagttagatgaaatgcgtaatggtaaacttaaagttatgattgctacaagccttattgatgagggtgttgatatcagtggtattcgttcattaatcctaggtgcaggtggtaaatctttacgacaaacactacaacgtgtaggtcgtgcgttacgtaaaaagaaagaagataatacaacacagatatttgattttaatgacatgacacataagtttttatataaacattctaatgaacgtagaaaaatatatgaagaagaaaaatttgaaattaaagaattaggaaagtaggtatgagtagtgactaagacagaaagaagattatatgattacattgaaaaaaaatcaaaaaataatacttatcaaatagctactaaaaaagagttagcagaagaactagatgtctcagtctctactttatctaaaaaccttaaaagattagagcaggataacaaaattaatgttgtctctaaaagaggaaataaaggtggtattgttatatcactagtaagagattatgatacagatagtctcttacattttaatgacacaaatgataatgttattacttctaatctagaatacgctactgaattacgaaatagattttttcctagctacgtatatgaaagaaaagaaaataaacgtagaacaaaattagaaatggttcaatataatgctagtaaagataaaaacaggaaaataatatcagatatgaatttttataatagtagcttaccttaccctactaaagatatttttaatatgtcttatgaccctgagggtttttataaagcttatatcttatgtaagttatatgaccaatatgctatagcacacatgaatgctaaatatatacaacatttaacattatcaaatggtaatgaagacgtagatagacataagcatttatctgagtactacagaaagaaaatgttacagaatttatcacgagatagtatttgtaaagacttctttggtagtaagacatttaatacattctacaacttttacttaaagataaaagataagaacattaatgtttttaaatacatgcaaaatgtttttaagaataccacatttgggtttgagaataaaacacaaccttaccctatacctgcaccaaatttttttagttccgataaatacttaactaattatgaaaattatgtaaaaggtattaaacgaggtgtaaataaaacaaatagacaaattggggaagtagagtctttaattaagtcatctgattacttattaaatcctgctgtagcacaattacatcaactatatacaacacctttaaatgaagagatacatgatattgatacaatgtttaaaacagctttagatatagaagatactatttatggtatatttaatggcatgaaacatattatacttttatcatataataaatatatagaacatctaatagaaaaattacctagtaaacataaaaacttattaaataaatttattaaacaatgtattgtaaatgagtattcacctactacaatacctaacaatgctagactatctatgtttttaatgcaaaaagagtataaagcaaatatcgcagaattaaatggtggtatagatagaagagacttaatagatataagtttagtaaacactacagacttaagtaaacaagatattgtaaatatagaacaaacaacaatgaattatctacatatgagaagatttacttcaacatcatatattctacaaatgtatagtaattatttaggatatgaggttaatttaacagaagttaagactattattgaaaagtataatttatttaatgaaataccattgacaaaagaaggtatgatagaatacaataaggttattgatatagtaaaggatgacacatatgagtaaacgaattaaggagattatcctacataagtctatgaaggatatacattttgcaagagaagtactagataagttacctaaaagtctatttgctacagaatcagaagacatgggttacttattcacagctataaaaagaacagcacacattgctgataaaatgtcaaacgaatctttagctattaaagtagagcaattaatgggtagtgataaacaagatgatgaaaaagtaacacaaacattaaaatatttagatagtttatatacagtagacatgaataataaagacgattctgtaaactatgaagtagaaaaatatattaaaacagaaatgtctaaagaagtattagttaagttcatagctgagaataagcaagaggactcagataacttacctgaactagtagagaaactaaaacaaatagaagttagtgacattaatggtactgatggtgaatttattgatttctttgaagatatagacaagaagagagaactattaagtaacttaagtattaataaatattctacaggtttccattctatagaccaacaaattgaaggtggtattggtagaggggaagtaggactagttattgcacctacaggtagaggtaagtctcttatggcaagtaatttaggtaagaactatgttaaacaaggtttaaatgttttatatattgccctagaggaaaagatggatagaatggtattaagagcggaacaacaaatggtaggtgtacaaaagaatcaattacttaatggtgacttatctttaaacacagatgcttataataagatacaagaacactataaaaagaatagacaactgctaggtaacttttatatcgttaaacatatgcctggtgaggtaacaccaaaccaattagaacaaattataattaatacaacaattaagaaagataagcatatagatgttgttattattgattatcctaaacttatgcgtaacccttaccttaagtatcaaagtgaatcagatgctggtggtcgtatctttgaagatattcgaaaattatcacaagaatacaattttgtttgttggacattagcacaaacaaacagaacagcttatggttcagaaattattacaagtgaacatgttgagggaagtagaaagattgtcaatgctgtagaggttgcttttgccgttaaccaaaaagatgaagaatttaaaaatggttttttaagattgtacttagacaaagtaagaaacagttcaaacacaggtgagagatttgttaatcttaaagtagagcctactaaaatggttgttagagatgaaacaccagaagaggcagaagaacataagcaaatactagcggaacttaatggtgaagataaaagtagatttaaagaaaaacctaataaagctgaagctattaataattcttttggagggctgagtttttaatggatgactttactaagctttataaaaaagtacgtagaacatttacacgtaataaagcagaatgggattatacaaatacaggattaagaaccttccaattaaaaggaactaatgtatgtgttacattactagactctatagcaaggaataatacaacaggatataagtacaacgttatgtctaataaagtacctgctataccttgcaatacacatcaagaagtaattaaagcaattgaatataagttaaaaaattgagaaaaagccttgacaatgtatttgttaaggattatacttttattataaacaataaagaggtggtaagatttgaacttaaatgaagcaaaagtaaaattaaaaaactttaaagtcattgtagtgagggagatatagtatttgatggtttcatgggtagtggtaccactgctattgcctgtatgaatactaatagaaattatataggttttgaattagatgaagaatactataatttagccagtgaaagaatagaaaaacataaggagatactaaacaatgaaaatgacaatacaggaactacaagataacactgaatttttaagacatgtaaaggtagatacggaagaagcaatggatatccttgaagatatattaagaggagagccttacacagatgaggtatgtgaattgatacatttttattcaagtaagctactaaatgagattaatgacaatcatatgtttaaattacaggagtttgctaatcatgatgttataggaattagtgaagagggagtaaaataatgataaaattattacagggggattgcctagagtcactagatacagttgaagataatagtatcgatgctattgtaacagaccctccatataacattgcaaaagagaacaggttcacaactatgggtagagctggtattgattttggggaatgggataaaaattttgaagctactttatggattaataaagatataccaaaagttaaaaaaggtggtaatatcgttgtgttttgtgacatgagacaatttacacctattattgatactatggaagcattaggctgtgaatttaaagatgttatacgtatggaaaaatctaatcctatgcctcgtaatagagatagacgatttatttttgatactgaatttgctctttattttgttaaaaaaggagctaaatggacttttaaccgtttagatgaaaaatatgaaagaccttgtattaaaacaagtgtaacacctagaagtgaaaaattaggtaaaggacacccaacacagaaacctttatatgctatggaatggttgttagagcgcttaactaatgaaggagacattgttctagactgcttcatgggaagcggaacaacaggtgtcgcatgtaaaaagttaaataggaattttattggttgtgaattatcagaagaatacttcagtatggcacaagatagattactaaataatacaaattaaaacaaaacccttgacaacatgtttgttaagggttatacttttatcataacgtactaaaggaggacactaatgaaatttatattttttacagatagccattttcatatgtttacgaattatagtaaacctgatgaagaatatggtaatgatagatttaaagaacaaattgagacattacaaaaagtatttgatttagctagagaaaataaagctaaagttatatttgggggtgatttgtaccacaaacgtaatgcagttgatactagggtatataacaaagtgtttgaaacatttgctaataatcaagatattcaagtttacatggtacgtggaaatcatgatgctatttcaaatagtttgtatactcaatctagtattgacatttttgaaacgttacctaatgttgaagttactaagtctttgcgtagtgaatcattaagttcaaaagtacagttaactatgtgtgcatatggtgatgaggtacaggaaattaaagactttattagtaagtcatatgttaaagataaggtaaatattttagtagcacacttaggcgtagagaatagtttaacaggtaaaggctcccatagattagaaggtgcatttggttaccaagacttattaccagaacaatatgattttattcttcttggacattatcatagacgccaatatctaaacaataatcctaaccatatgtatggtggttctttaatgcaacaatccttttcagatgaacaagaagctaacggcgtacacttaatagacacagacaagctaacatcaacgttcatttcattagatactagaaagtttattacagtacaaggtgacaatgtaccagataacatgaatgatttagtaaaccaaggtcactttattaggtttattggtactccagaacaagctaaagtaattgaattagataaaacagaagatgaatctaatgtacaagtacaaatgaagaaagaatacacagtagaaaagagagtcgacgctgacgtgtctgacagccctataacgattacttctagttatgcagacaaatactatccagaagcaaaagatgagcttctagagtgtttaaaggcggtgttgtagtatgatagatattattataaagaatattaaaccaagtatagataaatctaaacctattttacagttcttacataataaaagaacatatgatgtatgtaatgttaaaaatattgatgatgcgatgtaccttattaacaaagtattagaggaagctaactacgctcattattatacgagggttattgaacaagaagattgttatattctagattatggttcacatcaatgtttctttaaattgttaaaagaaggtggtacaaatggttaagtttaattatgtagagatgaataactttttagctattgagcatatcaagctaaatctggataagcaaggtcttgtacttattgaaggtattaataaaacaaatgatagttttgaaagtaatggaacaagtaaaacaagcatgatatcctctatcacatatgcactgttcggaaagactgagaaaggtttaaaggcagatgatgttgttaataagtataagaagaaagacacctacgttaagctttctttcaacataggtaaagacgagtatttaatcgaaagatatcgtaaacacaaggaacacaagaacaaggttaagctgttttgtaataacaaagagataacaggctctacaaatgatgttacggatactcaaatacaagagttatttggtattccttttaatacatatgtaaatgctattatatatgggcaaggtgacatccctatgttttcacaagctaccgacaaaggtaaaaaagaaatattagaatctattactaagactgatatttataaacaagcccaagaagtagctaaagaaaaagttaaagaagtagaggagaaacaaagtaaagaacaacaagaaatagaaaaactagaatataaaagaaacctaaaacaagaacaatataataatgaagtaaataaatataataacttattagagcgtaagaagcaagaagaggaacagtttaatcaaaagaaacaagagtacgagaataaattaaaagatttagacaatcagattggtgtatgtaaaggtagtattcctaaagttgaagactttgaatttgtatttagtgaaaattatacaaaagctaaccaaggtattgaaaagattaacacaaatattaatgataaattattgccattattgagtcaagaaacaacaaataagaatacaactctaaatataattaatcaattaaaacaatctattaataaactagatacaaatgaccattgtcctgtttgtggttctcctatagataatacacacaaaataaaagaaaaagaaaacttagaattacaaattaaagaaaaacaagctaaaatatctcagtacgaacataatgagcaggctataattaataagaaagaagagctactagctaaatctaaagaattacaacaatttatacagcaagaagatatagaaaagaaaaaacatgatgatgatattcaacggcaatacagagaacagcaagaagtatatgatgaaattagccaattagaaaacactaaagctaatttaaaagaaccaatattaaatgattactcttatatagaagaacctaatgaagaattacacaataaagaactaaaagatattgacaatactattgacaaacataaggaaaatatagtacaattagagagtaagaaaacaaaatataaacaagctgtagatgcatttagtaataaaggattacgttctgtaatattagactttattacaccgttcttaaatgagaaagctaatgagtatttacaaatattagcgggttcggatatcgaaattgaatttcaaacacaagtagaaaatgctaagggagaacttaaagataagttcgatgttattgttaagaataacaatggaggagagtcttacaaatctaattcagcaggagagcaaaaacgtattgacctagctattagttttgctatccaagacttaattatgagtaaagaagatatatctacaaatatagccctttacgatgaatgctttgatggtctagacactattggttgtgagaacgtagttaaactactaaaagatagactaaaaacagtaggtactatttttgttattactcataataagagtctagcacctttatttgagaatacaattacggtagtaaaagaaaacggtgtagctacactaagaaaggaattaaataatgaaactaaagattaaagataaccagtttgcatctttaacagtaaattatacaaacaatgcaaagttacatatagataatattcctgtttctactttagtagactggtaccctttaagtaatgcatacgagtataaagctagtaatgactttggatatattgaactaaagagattacgctctagcttacctgtgtcttatgggttaactcaccaaaccttatataaatgtgaaactgttaaatgtaaattaggattatggtataacgaaaaaataaaagaagataatgagaaaattattgagaaagctaagttgtacggtttacctactatagatgaacccttcacaagtaaagatgtaaagcaaggatttagtgacttaggtgttattttccaaacactaaaaacaatcagtacaaacgagtacttgaaagataaaacaattgaagagattaacacctttagtaagaaatcagaggattatcaattaaatgaagtactcaagtacagtacaacactggtagatgatacttatagtgatttaagtcaaatctataatatgttactattaatgaagaaaattgtttctatataggaagtgactgaaaatgaggtttgaagactttttaagtcaagagattggaacacccaaagagaatactgtaggagaattacgatattgttgtcctttttgtggagaacagaaatacaagttttatgttaagcagtctttagactctactaatggtatgtatcattgtaagaagtgtggagaacatggtaatcctattacttttatgaaatcttattatagtattacaggtagacaagcttttgagttattagaaacaaagaatattgatatacaattctcaccaacattagaaatatataacacagatttaactgacagtgagaaactattactaaggcttaatggatatacaaaagataatagtagtataaaatcaaaagcacctgaactacctatagggtttaaattgattaaagataacttaaataataaagaagtagtaccgtatttaagatatctcaagaatagaggtattacactacagcaaatcctagactatagtatagggtatattataaatggttattgctattcttatggtattaataatgagaaaaagaaaatagtacttagaaatagtgttatattttttacttatgataatgaaggtaagtatatatactggaatacaagaagtattgaaacaaatccatatattaaatctataaatgcaccttctaaacctaatgagtatggtaaaagcgatgttatatttaatttaaatatagctagtaaacaaaagtttgttgttataactgagggtgtttttgacgcccttacatttgataagtacggtatagctacgtttggtaaacaagtttcaaagatacaagttaataacctcatttcaagtattcctaaagaaacacctatatatattatgttagacacagacgctacagactttagtattaatttagctaataagcttatctcacatttcaatgtagtgtatttagtaccacatggtaatgaagatgctaatgatatgggtatggaaaaagcttttagtactttgaaacacaatagatttttagttacacctgaaagtattcaaagttacaagttacaacaaaagttaaaactttaatcttgcattataatgtatagtatgttacaatacatatataaaaataaaggaagtgttattttggaagagaaagatattctacattttgtagatgagtatgttacagcattacgtgtaggtaatgaacagcgtatgcaccaattagaagagttaggtaaagaacaaacagcaactttaacagatgtaatgaaggctattactaacttattgttaggtgttaataaacaaatggttgacctagagcaaaatacagagttaaacttaaacattctaattgatgctttatacaaagctgagttagttaacgatgacgtattaaaatatattgaggaagcaattgaagaatcaaaagaaaaggatgaagaataatggaaaaaaatattagtacacatacaaaaggtatttcacaaaaagatatggacatgtggttagaagctattacacaaggaacagtagaaggcaaggaattaactgaggacactgctaaacaattacatcgtattggagcacgtagtgtatcattagatgaggctactagaattgcacgagcaattaatgccgttacagttcaagagtatgcatctgcctttaatgatgcttttaatgctattgatttgttaatgattgttatggaagataacctagatgtaactactgagcaagtagagaaggcgcaacttaaattaaaagaaaaacgtgaaaagtatttagaagaaaaacaagaagaagtaagaaaaaaacaagaagagcataaagcaaaagaagataatgaaaaagtagtgcagttgaagaaacatgacaaatagtaaaaaaaaaggtgatgtattcgaaagaaagattgccaaagaactttctgattggtggggttatccttttagtaggtctccacaatcaggaggtgcatcatggggagctaacaataatgctgtcggtgacattgtagcacctttagagtctaattttccattagttgtagagtgtaaacatagagaaaattggattatggataatgttctacttaataataaagaacctcatacatggtgggaacaagttataggagatagtttgcaagttaataaaactccttgtttaatatttacaagaaatagagcacagtcatatgtagcattaccttatatagaagatgtatatgttaagttaagagatgaagaataccctattatgagaacggattttattgtagaaaatgttagaaaagataaacatttttatgatgttcttataactacgataggtgggttgactaacttaacaccttcttatattatatcttgctacaacaaaaaagagattaaaccatatagagttgtcaagtctaaaatatctaaagttagtaaaaaagaagataagttgattgatgatttacttgatggtatataaggaagggatactagttgattacacaaataaaaaaacgtgatggttccgttgtagattatgatttaagtaaagttactaatgctattttaagagccaactcagaaacagaagaatatattgaagatgttattgattctcttgtagatgacgtagactctttactagaagaacaagacactatcattactgtagaattaatacaagatatagtagaagaagttttatttagtagtatgtataaagatactgctaaagcttttattttatatagggataaaaagaaaagagagcgtaaaagagacttattcaaaccaagaaaagaattaaaaccctatgaataccctgaattattagaatataaagatgctatacgccaaagttactgggtacacaccgagtttaactatacatcagatattcaagactataagaataacgttaaatacaatgagcaaatgaccattaaaaaagctatgctagcaattgcccaagtagaagtagcagtaaagactttctggggtgacctatatcatagaatgcctaaatgggaaactggagctgtgggtgcaacttttagtgagagtgaagcacgtcatgccgacgcttattcacatttattagagattttaggtttgaatgaggagtttaaaacaattgacgatattcctgctcttaaagaacgtgttgatgagttagcgatgcatgtaaaactaagtaagagtgaggatgatagagattacgtactatctgttttattattttctatatttattgaacatgtatctttatttagtcaatttttaattatgatgagttttaataaatataaaaacttatttagaggtttatcaaatgctattgaagctacatctaaagaagagcaaatccatggattatttggaacagaaatcattaatattcttagagaagaaagacctgagtggttcaacaaagaaatggaagatgcagtttacacagcctgtaaaaaatcttatgaatcagaaacacggttacttgattggatgtatgaagatggggaactagagttcttacctaagaaagtagtacaagaatttattaaaaataggcttaataactctctagtagctgttgggtatactaaactttttgatgttgatgaagagttactaagtgaagtgtcatggtttgatgatgaagttattagtactaagttaacggactttttatctaaacgttctgtaaactatacaaaatttagtaattctgtaacggaagatactattttttcaacggaatctgattttgaaaatactggtaaacgagttactaaagataaagtaaacgctattttaagactaaaaatgatataaaggcagtgtatatagatgagtaattttgaatggttaaataatgattctagagtttttctagaacgtggttatttaagtgatggtgaaacaccagaacaaagaattaaagatattgcagattatgctgaagatattttagatattgatgggttttctgacaagttttatgattatatgagtaaaggttattattccttatccagtcctatatggtctaatttcgggaaagatagaggatttagtattagttgttttggtagttgggtagatgatactataccatcgatacttaatactgctagtgaagtaggtatgatgagtaaatacggtggtggtacaagtggttacttcggtaacatcagacctagaggctcagaaatcactgataatggtgtaacaagcggttccgttcactttatgaagttatttgagcaaatgacggacacaataagccaaggatgttatgatgataaaactgaaatactcacagagaatgggtatgaattattatctaatgtagttagtagaaaagatggaacaaaaattgcacaggtaacagataatgatgaaatagagtttgtagagcctacagattatatggaatttgtacctgaagataatgaattagttcattttaaagatagtaaaaatattgacttattagtgactaagaaccataatatggtttttaaatacagaactaaaaggactaataaagaaacaggaaaaagagaaagtgttttagtacctgagtatagaaatagattagcagaagatatgccattacatagagacgtttatttagcacattcttcatttgctaaagaaggcagaggactcacatttaaagaacgtcttttggtagccttgcaagcagatggtagtattattaaaaaataccctaaatctttgaagtttagatttagtaaagaacgtaaaaaagatagactattatggatattagatggtctaggagttgaatacacatataatgtagacaaagacaataatcataatatttatgttaatatgggtgaagaattacctaaagaatttagtaaatgggtaaatttacaaaatgtcacacaacaatgggcttatgattttattgatgagttgtctcattgggatgctagtagaagaacggataaatcatttatctacatgagtgtggttgaacataatgtagacattgttcaagccatagcttctatggtaggttataaatctcgtaaatcagttgatttaagggaaagagaacctaataaagccactatgtataaaatatatctatctgaaggtcaattatttggaggctcaaatattactgtggaaacggtacaatataaaggtaaagtatattgtgttgaagtacctactcataaacttgttgtaagaaggaaagggcatacattagtttgtggtaattctacacgcagagggcgttttagcccttatctacctatagaacacaaagatattgacgagtttttagagattggtaccgaaggtaatccaattcaaaacttaacacatgcagtaacagttactgatgattggttattagacatgattaatgggaacaaagaaaagagaaaaatatgggctaaagtattaacaagaagaacacaaataggatacccttatatcttctttcatgataatgcaaatagtaatacagtagatgtatataaagacaaagggttaactattaataactcaaatttgtgtaaaaatttgcacctttagtaagtaattactatcgaaaaatccatctaaacggtgaatatctaagttaaaatatacgacaacaccgtgctaaatcagattataaatctgtaaatgcctaacgactatccgtaacctattcacataagtgaagggataaggttagagccaagtggtataagtattgagtaataatcaagtaaatcttatttaaatcgaaatggtggacttccttattaaggaagatgatatagtctgctctatatggaaacatatagaagttcatattaacaataataattaagagggtcgaaaatggagtaccatatttataaaattacaaacaaaaacaataataaaatatacatcggaattacatcaaaaacagttcaacatagatttaaaaatcatattagaaaagctaagataggctcaacaactaatttacaccaatcaatgatgaaacatggtgaagagaatttttatgtagaagaattatacacttttagtacagaggataaaaagtttgcttacatggtagagcaaatttttatagacatgtacgaatctgtaaaaaaaggttataatatggagataggttttgggtggaatataatggatagacgaggaaaaaacaatccgatgtatggtaaagaaagcgctaatgctaaaaaagttattgttgatggcgtagtttataaaaatatgggtatatgctctgaagagctaggaataaacagaaatacattaaccagaagatgtaattctgaaaaattccctaactataaatattgttaattagagaactggtagtgattaacgaccactattgaacatgtaggagtgaaattatgttacctaataaagaagattggtcatttgtatgtgacctatctagtatgaaccttgttaagtatgatgaatggaaacacacggatgctgtagagacacttgtttactttttagatgctgttatgtctgagtttattagagacttagaatatctaagagactcagctgaaaaagataaacaagaagcatttaaatttatggagagagcttataacttttctgttgagaacagagccttaggaataggcgttcttggatatcattcatacttacaatcaaaactcataccttttgaaagtattaaagctagtcaaattaatgaagagatatttaaattattaaaagaaaaaacttataaagctagtgagcagttagctaaactttatggagagccttctattttaaaaggttatggtagaagaaattcatgtttgatggcggtagcaccttaatttatagggcgtatatggattaacatatatgataacactcttgaatttctgaaaagcctaagtgtgtaaacataaggtaatcagaagctaagatttatttatagctaacttgacaaaaacaaatatatacattatactagttactaagaggtgataataaatgtggaaaaatagtattagttttcctaggctcgaagtaactaaggatggaagagtaagaatgtggaataaatcatggaaaagatatgtagaaaaaagacataggtatgataaagatggttactcaataatttctacaagaaaagaagatggtacatccacatcagcaagagttcatagattagtagcagaagcgtatataccaaacccatacaataaacctgttgtaaatcataagaatgggattaaagatgataatagggttgaaaatttagaatgggctactatatcagaaaatactaaacatggttatgatgttttaggtgttaaacacgctaacgctcaactaattttagtctacttagatggcaaacctttttctagttatgactccttaactttaatgggtaaaacattaggtataaatagaaatcttatacgtagagtagaaaaagaaactgatgggtacatctcatttaaatggatagaaaaaccatataaagtacacaataaagatttttggaaaacatcaagaaaattaaatacgagaggctcttgttatgttgtaaataatgtatactataatagtgcaaagaaagctatggaggctcttaaagtaagacaagatgttttctataatagacttaaaaaagataaaaatgtatatagtgtaagtattaaagagtacttaaaaaactgtcaagatataaatagataaagttcaacgactatcctgttgcggggagtacactcaagtgagtggaagtgggagtggtcttacatttagtaagatgtgatatagtctgctctctatggaaacatagagctgttattaaattaacgcatgtaaagtaacgaactacatggaacataaaggacaacaagttcgtcatttatcctaggtcaagtttctaaatcaattgaaccttttatgagtaattattacgttgtagacactgctaaagtaaagaaaactatgattaatccttaccttaaaagactattaaaagaaaaaggaaaggacaataaaaaagttattgagagtattagggacaatgatggttccgtacaacacctaactttcttaagtgattatgaaaaagaagtatttaaaacatatggtgaaattaatcaatataatatattagaccaagcttctacaagacaaaaatatattgaccaaggacaatctataaacattatgattaaccctaaacatgttacagcagaggagttaaatgaactttatctctttgcatggtctaatggtattaagtcattgtattatcaacatggtacaaatgcatcacaacaattcaatttatctaaaatttgtattaattgtgaagcctaaagaggtgactaaatgaataaaaaagaagcgtttggattactaagtagaactgaagatatattcaaatcaaatgatattttctcacaagtaagtaatgtacaagaccctattaagatgtttaatagggaagatgatactaaagcagatagtaaatcaagtaaatttcagctagagtttatttataatacatatgtgtacattcttatatatgaaggtacattaaagctatttaaagctgatggtacggagcagttaacacatgtagctgatattgaaacatttaaagatattattgacacactagaaaaggatgatacagaaaatggaaaaagtgaatagtttactagaattaaatacaacaattagacaaaagaaagatgttattgtcatggttacacaagatgagtgtgctaaatgtgaaattttaaaaagtgtgattcctcttttcgaagcagaaggagatattactaaacctgtttatgttattaacttagatgataaagatgtagatagagaaaaagcggtcaagttatttgacattatgagtacacctgtacttattggttataaagatggggaacttgctaagaagttcgaagaccaagtaacacctaaaaacttaatggaattagatttactttaatttgtaattacctactacttatgctatacttatattagtaaaaggtagtaggttttttcatgaaaggatgaatagttatcgcaaaaaataagacattaactatatataatagtgatagatattttaatatacatgtaaaaaacaaagatgaattatctaaagctattaaagttacaactttaaatgaagatgatatagaaaaagatatggatatcttagctaataaatcaactaggtatatactaagagacaataaacattacatgctatttaatgagaagtataacaatgataagttgattgaaaaaatatgtaagcatggtggacaagtaacatactataccgattcagtagttccttactatgtgcttaaagacttatcagcccaccctaaatcctcagttgtatacagaatgcgtagtaaattcaatagcaaagagatggataatatagccttgagtttcatgggaacaaaagtcattattgatatttcagttgtatttccttacgtcaatccttacgatattattagaagtttatatgatgttaaaacaaatgtagacgaggttcatctatcattccctcgtattagttctatagacgctaaacaaggtaagtactatacgtatgataaagaagcttataaactaaatcctgagtatacacttgattttgcagataaattaagagtttctttgtccgtgtggaaaatgtatatctatattcttgctagtaaagaaaataaagattatgaaataatagaagacctacttactgaattaaaaagacaaagaaagattaagatttaggtgattttgtgagtacagcaaatagaagagatatagcaagaaagatatctgagagtacagggtactatatccaagacgtagaagagatactagaagcagaaacaaaagctatttctgaattgttacttgaagattatgataaggtaaagaatcataagtactttcaaatagaagtagtagagcgtaaaggtaaaaaagcatgggatggactaaataaaacatacttcactttacctaatagaaaggcactaaagtttaaacctttgtctgaattagataaagttatagatgtattaaacaaagaggattaagttcctcttttttttattgactactatgattagatatgctatactgtatttaagttaaagaagggattggtaataacatgaaagttttaattttatacgactatatcagagaggaacacttttcagttagtaatgatggtagtgttaagaatgtactactaaatacacctaatggtagagtattaaaacaactattatctaatatttctggtattaatcgagatagaagtacaaaagattatgatattgagtttttatatcctatggtgcctacacctattaagaataattatggtaaaacaattaagtaccaagacgttaagttatctgaagttaaaccttactatgaaagaatgaatcaaataatcataaacaataagtacgatattattattcctttaggtaagctaggggttaaatatttattaaatgtatcagctattggtaaagttagaggtgtacctaataaagtaactattactagtgaagataaaaaacatgatgtttgggtattacctacttatagcatagagtacacaaacgttaataagaatagtgagcgtcatgtagtagcagaccttaagttattaggtaagtttgtagaacaaggtgaagacgtatttaaacctaaagaagttaagtatgaacttgttacaagtattgagcgtgtaagagaaatatttaataaagaagtaaagaatgataatcatgacggtgtagacattaccgcatgggatttagaaactaactcacttagtcctgatagagaaggaagtaaacccttagtattatcaatgagttgggaaaatggtcaaggtgttacaatacctttatataaatcagactttacttgggagaatggacaacaagatattgatgaaattctttccttactaaaagaatgggtagctagcaaagaagacattaaggtactacataatgctacttatgatataaattttctaatgacaacacaaggtttcacagactttgaaaataatcaagacactaaggtaggttggtatttagctgtaacacaagaacaagcagagtctctaagattatcagaccttgcttacgaagtaacagatgtaggaggatatgacaaaccccttgaagattttaaagagtggtatgtattgaagctactaagattcttatcagataaactaaaagagattaaaaaagaaaataaaaaagttgttaagaaagaatataacataaaagcaaatgagtatgatacttggttgaaaaataaactcgataacattgatatagaactaacagatgaagataagtattatggtatcacagaagagcaaaaaagatatttagagttaaaactaacacctgaggttattacaaagaatatgcttatggattctgaatttaaagaagtagccgaatcatcacctgagtatatgagtttatctaacaaagctaaagattatgtattaggtacatctattaatttaattaacacgtataaagacaatactaaagttattaatgaggtagatggaggtgactttaattatgattggattccattagagcttatgcatccttatgccagtggtgacaccgatgtctgtagaagaatatactgtgatgttattgaaaagttaaaagaacaaaatagacctaaagcattggacttaatgtcaataagttaccctagattaattagaacattagctagaatacaatcaaatggtttacattgtgacctagaatacatgcataaaaatgatgaattttatattaatgagatggaaaaaacccaccaagaaattagagaacattgggctatccaagaatttgaggagactaggtataatttataccaattagccttagcagaacatgagaagaaaccatctgatagaaataaagaaatacatgagtatagagctaaatttaaagatgaaggttggaagtttaaaccaagctcaggtgaccataaaggtgaagtattgtatagcattttaggtatacagttaccttatagtaaagagacagttaaggataaacctttcagtaatggtactaaagaagatgagttatcatggaaagattacaaaacagatactaagtctataaagatggcattatctttggttgagaatgaagataataaaaagctactagacttacttatttattatgcctctttgcaaactaaaagaaattcttttactaagaaattacctaaaagagtaaataagaatacacataatttacatggtaactataatagtacaggaactgcatgtattacaggtgattctttagttattactgataaaggtataaaaaaaataaaagacttatcaaataatagaaaagaaaaagtatttagtagtattgatgtaggtattgttaatagacagggaaatttagaaaaagcttcacacttttattatagtggtgtaagaaatggattaaagattactttagaagatggtactacattaactactactttaaaccaccccttattacgaaataattattatagtaatactggtagggttctaaataaaacaaaacaaacaactaagcatttactagataatgattgggttgtagctgaagatttaaaaataggtgattatataaaaatgtcttataattctaatctttataataatagttatattgatttagatactaaagattttatatatagtaaagaaaaaagtattacaaacacaaaaagctatacactacctaattatgtatctgaagattttgctgaatggtacggtatgtatacagctgatgggtctttttctacaaataacggttcattcagtataagattaactaatagtaatcttgaagttaggaatagattctttaacctaactaaagatttatttgatattacaccatactacattagtaataaagatagaagtgattctattgaattttcttcaattggtttaggtagatggttagaacatatttttaatatgcaaagtaaagcattaaataaagaaatcccacaacaaatattagatagccctaaaagtgtacaacaagcatttcttaagggtttaagtttagatactgcaacagaaaagaaaaagtatcctagtttgtactataatactgtatctaataaaatggctttacaaataagaacaatgctaatgaatatgggtatctattgtaggtattctataggtaaagtttataaaaatattaatagaaaagtacaaaatgaatgctattctatacaaataacttatgatgcattagataaattttacgatgaaatagggtttattgagagtattaagcgtgatagagtaaagtataaatcagaaaatctaaaggcattaggcagaagaaacggtattcttttatatgataatgttttattaggtaaaataaagaaaattgaaaaaataaaagatatggaattttttgatttacatgttcctagtagccattcatttgtagctaataatattgtaaatcataatactagcaggctttctagtaacaaccctaaccttcagaatcttcctgctcatacatctgatgtaaataagtttgattatcaccaccctattaaacgttcttttatctctagatttaaagatggtgtaattttacaagccgattacagtgccttagagatgcgtattacggcactatatacagatgataaagaaatgttagagatgtttttaacaggacaagatattcataaaaatacggcaagtattatgtatggtaaaagtatgaaggacgtaacagcagaagaaagacaagccagtaaagctgtagcctttggtttaatttacggttaacaaaatatagccgtagtaaaactaggtaaacctattgagtatagggtgtatagttcacgttaggagctacaggaaatggtagttaagaactatgctaacagggaaggctttaaaatgctaatcctgtgtagaataatttaagtgtaatttaataagtaaggatgatttaaatagaatttaaaataggagaaagatataatcacaataagctatattctaaattagctcatgtatggtctcatatgatagatagatgttataatcctaaatgtaaggaatataaatattatggattactaggaataacagtagacgatagatggcataattttaataactttcttaaggatgtagagttaattgatggttggagtttagagaagtataaaaatggtgagttagtgttagataaggatattaaagaactaggtaataaagtatatagcttagataaatgtaaatgggctactagagaagagaacttaggatttgtaccttcaagaagtaaaaagatattaggtatagaccctacaggtaaagaatatacattcttcaatcagtctaaatttgctaaggaacataacttaattcaaagccatatttctgcagttcttagaggggaaagaaaacatcataagaactgggtttttaaaacactacaagaatataattatcaacctgtagaaaacccttctaaactttatgcacctcactatgttgcttataagaatcatgtggaaattgattatgatagagtaaaaactaagctactaaagagactaaatatacctacgtcaggcactaactttagaaaaatggaaaccggtgaagattgttatgggattacattcaaaatagagaaaagagaacgatttgattacacttaaacacttaaattattttatcgaacgactatcaaaaggtgagctttacgccgagagggcttattgcaatataaccgagtagagtacatgtagctcatacctacatggaatagcctagaagtagatagtaacgtatttacttaagatatagtctagtcccctaataaatatcgggaaaccgagggtataaacggagtcagagttttcgtttgcaggtaaaaataatatgacagttgatgaagctactgaaatatttaataagttttattctaataaacctgctattaagaagtctatagatgaaactcatgagtttgttcaaaaacatggatatgtagaaacaatgaatggacacagaagatatatacactcagcacaatctagagataaaaagattaaaaatgagggtcttagacagtcctttaacacgattattcaaggtacaggtggttaccttactaacatggctattacttatattgacgactttattcaaaataagaatatgaaatcgaagttagtagctactgttcatgatagtattgtagtagactcacctcctgaagaagttaatattatggctaaagttattgtccatgtaatggagaaccttccttatgatttcctaaaaataaaaattaatggtgagttaaggcagtatcctatagatgctgatatagaaatagggctaacatataatgatatggtagaatacaacgaagagttaataaataaatttaattcatataaaggatatattaaatataaactagccttacagcaaattaaggactactatgaatcaggtaaactaactgaagagcagtacaaacaaaaaacggaatatattaaaaataatattgacagtttcaaagttatatagtatagtaacctatatcataataaaatagaggatggtattatggagatacatttagatacattagatttcaatgagttaaccttaaaagataacaatggaaacacacagacttttaatatccatgatgaactaaaacttagtgagtatacaattcaagacgaaatgtaccaacagtcgtctaaatttgcatggtgggcttccttaaaagaacgtgtgagtaattatgccgaagcagagcaacgtaagttagaaaaaataggagctcaacttaatctacaaattagagctcagtacgaacaacagggaaagaaacctacaaaagaccaagtagagtctgctgtatttctttctgatgaatatcaacagcaagcaaaggttgtagaagcatggaattatagagaaaaacaattacattatatcgtaaaagcttttgaaacacgtacaactatgttagtacagattagtgcggaactaagacaaacaaataaaaatggtggtgttactaacccatttacacattaggtattgacaaacaaaagaaaacatgttatactaattaagtaatttttaaaataaaaggagaaaagatattatggattttaatcaatttattaatcagcaatcagaaaagctacaatcaagtggttttgacaatgaggtagagacgtacaaaccaaagaatccggttttgcgattaggaaagattaaggacgttaacaataatcaaattaataaagagtctgcacttgtacgtgtactaccgcctgtagcaggttctaatgagttctttaaggaatttagaacattaggtattaactatgttaaaaaagacggggcacaaaagttctctatgcttactttacccgagaaagttaactcatctgttgtagacccttatgtaaatacatggttaaagcaaggtgtacaatttagtaacttccctaataaacctgcacgacgagcatatattcatgttattgaatactttaatcaaaatggtcaattagttcctaacacagatgaacaaggtaatgttgttattcaacctatggagctatctaacacgggtctgtctcaattgattgaccgattaaaagataaaatgttatcaccatcacctactgctacacacagctttatttcagccgatgatgcattccctattaatattgctaaagctaaaaaaggtgaaaaatcatggaatgtaactgtttatcctacagtaaaattaggtgccttacctcaaggttgggaacagcagttatcggatttagataagttagctacaccaacagaagaaaataaccctaactttgtaaactggttgattaacaatgttaataacacagaagtttctcatgataattttaaattcagtagagaaacaaacacattaggtgaagaatcatcaacacaagagcaaacacaagctcctacgcaacaaagtgtagagcaacaattacctagtaatttaggtggacaaaataacacacaaccaaactttaataatgtacaacaaccaacaccacaagttcccccacaacaaaatacacagtttggtcaaggaacacctgtgcaacaagcaccacagcaaccaacacaacaagaacaaagtaatccatttgagaactttgatgctaataacattgacgattctcaaatcccattcaacacaaatgaatcaacaccagaaccacctaaacaaaatcaacctaagagtgtagatgatgtattagcaggtttagatttataataactttatagagtgctatcttagcactcttttatttaaatttcatataaaaggatgatataatggctagagcaaaaaaaggtaaagaagtagatactacaaatttaaatactattgacttaggtaaagaattaggtttaactttactatcagatagtaatagagcagatattaagaatatcattcctactatgattccacaatatgatagaattttaggtggaggtattcctctaggaagacttacagaagtttacggattaacaggtagtggttaccttaaatagccacaatggcttataatgagttattacaaatccacgtgaattgctgggaagccttaaagacttgttaactacaacgtaactggaaacggtaagcgtgaatgttaaaaataacaagtatatgagttatatggtataacgagttattaaactgttatattatatagtgtaggtaatcagcatccaagctcctgtaaaatggagaaggttcaacgactatcgaaaaggtagctaatattgagaagtattagtgagtaactgagtagagtagggaaaccgaagtacgtgggaattgcctacgaaatctatttaaaatggagagtagtataatatggtaaaacgagtatggtctaatgaagaaaaacaagatatagtgaaatcctttcaagaaggtaagaaatttagtgagatacaaaaaaagtataatgcacattactttacaattaaaaaagtattagatgagtttaatattgacacaaatagcaagagaagatggactagtaaacaaaaacaagatattttaagaatgtacactgaagagtctatgtctattgcagagataaaagcagtctatacaacacatgcaagagagataagtaaaatattaagagactttggtattgatacatcttactatcaacctaggagagttaatagaaatattaataaagatttctttgaagtaattgatactgaagagaaagcatatattttaggtttattgatggctgacggttgtgttagatatagaagagaaggtcaatgttacttaacactagagttaattgataaagaaatagtagaaagagttcaaaaagaattgaattcagacagtaaaatttatgaatctcatagaaaaagagattatataaaaaatgaaaattctacgtatacttttagtgttaccgatgaaaaactatgtaatgatttagctaaatatggtataatacctatgaaaactaaaaaaacagattggttaacacaagacataccttatgatttaaggaaacattacttaagaggattgtttgatggtgatggttctattggatgttataataataataataataatagatggtcaataactttagttaacaatcatcctgaatttttaaaagaagtaggtagttggattgaagacttattaggttttaaatgtcctacaatatctaaaacttctacctcttatagaataggttatacaggtaaaaaagctaaaaaattaatgaaaacattatatcgagataataatattcatatagaccgaaaacaaaaacttgcagaccaagcaattaaagatatagtctaagcccactattaagttagtgttaaagtactaggaaactaggggtagtaactgaaatcgacctttgcagtacacttatctagagtagcaacacaattaggagttattacagtttggattgacattgaaggaacagcggataaccaacgtatggaacagttaggtgtagacgtatctaaattgttctcaatacagtcaggtgaaggtagacttaaaaatgttactgaactatcagtagaaaccgtaggtaaagagttagaatactggattgatacatttaatgagaaagcaccaggcgtacctattttgttcatttgggattccctaggagctacacgcacacaagatgaaattgatgcaggagtagaccataaaaaacttggaacgaaagcgacaagtacacaaaaagtagtaaatgctatttcacctaagcttaatgatacaaatacaggattaattgttattaaccaagcacgtgataatttaaatatgagtaatccttatgatgaccctattaagtctacaggaggaagagcttttgaacatggtgctagtttacgtattaaagtatctaaaggtaaagaatcagaattaaaacagaatgatgctatgacaggtaaacctacgtataaaggtcatgttatgcgtattgaaactaagaagtctaagctatctagacctggtcaaaaagcggaagttgatttattatcaggttatgaagtaggttctgatgaagatactattcaattaaacggtgttgacccttatcatactatctataaagaagccgttgagcgtggtttaattactaaaggaacatggcgtaattatgttacgttaaatggtgaagaaattaaaaaatatgataaagattgggtaccgacattaatggacaaccatgaattatacttagaattatttagtagagtttaccacgagaatttccctcataagtatgcacctttaaacaatacaaaagtagacactactcaattagaagagtttaaagcattacaagattactacgaagagcttagctctactaataaagagactaaggaagatgtaaatgaataatctaatagatagaaatattaatagtgttaaagaagcattaggtagggctaatacaaatgatgtattgcccttaccttatattgaaatagcagaacgttttaaaaaagctagagataataaggagcctatcattgtagaagagtcgggatttccttacactgattctacagttatgtatattgagcatgtagagtcaagatgggcaggaggttactctttagtacgttataatagttctgaagtcaaagtacctaaaacaatacattattccgatatctatgtatcaaatgatgtacacaggattaagattgtatttgagggagcacatccttatgaagaaagttaataatgggaatagatatattatagacttagatggtattcctattgactttggtagagacttagacatgcttttaaagagatataaaaaccttagatggtcattatttcataggtactctagaggtatgagtcatgactttgagaaacaagagctacgggaatacatagatgagcagtttattaagttagttaaggagtatgatatacaaagtaaagtagattttccagggtatattaaaactaaactaacactacgagtacagaacagttatgttaagaagaacaataaatataaacgtactgaacttgtaggtaaaacagattatacagtagagtccttaacacaggagttaaatgtgggattagaagaaaatgaattacttaattatgtctttgatgatacacaatttactgaagtacaaagtgaactgctaaaagagctccttattaatacggataaagaagacaacgcatttattgtgtctactgtagctaataggttagatgtagaacgttcagaagtatctagagagcttacagaacttaaagattatgttaaatttaaaataaatgcttatcacgaacaaaacagtagaagatacattagagacaacaaaatagatacacaaaatcatgtatgggaataacacaaacaagccttccttttgttatattattaatgaaaataataacatagggaggttttttctatgttataatgtaaaaaataatgattggagattaattattatggctaagaaaaatattaatgaagtattaaaacaagataccgctactgtagcagataagtatttacaagttcaagtagagcaagatggttatacacgtacacaccgtggtcagtataactacaaagtagtagataataagggagaattattcttatacccgattgaaacagatggtagaggaaatattaaaattatgaaaaaggctcctgttgcttatacagatggagaccaaattcattttgtagttaatactgttaaagacccttacaaccatgcttttattagaactgaaaatattaaaggtaaagataaaggtaaacaattaattcaagcgtttttagcattcgttgaagaccgtttcagctttggagtatacaatgtattccttgctaataacaagagggatgtttttgctttagttgatgctgaaagtaaagatgctaaaaaagtagtagatagtaatgagaaacctcatgaagacgtgagcgctgagtttcctgctagtccactacgtaaagacgttaaaggagtagactcaggagaaggtcaaggagatacttcagaaccgtcagtacctaaaaatgtaacagtaacagctaaagaaacaacagctgatattacagctgaataattatataaaggatgacttagtttggataaattaaatttatataaaggtgaccagctattaaaaagtgaagagaaacaagcaggtaaaacatcaattacaattgataacttaactgctaacacagactacccacaaggtacatataaagtatctttctctaatgaatcaggagaatcagaaaaagtagatgttcctgcgtttaagactaaggacattaaagtagtatccgttactttagatgtagaaacactagatttaaaagtaggagaaacacataatttagtagctacgattgaacctagtaacgcaacaaaagctacttatacatttacttcagaacatgatgatattgctagtgtatcaagtaaaggtttagtagaagctaaagcaaaaggtcagactacaatcactgttacaactgatgatggtaatcatacagatactgtaactgtagttgttaaagataaagtacctgaagctcctacaaatgtaactgtagaccctaaagaaacaacagctgatattacagcataatcggaggtaaaaataaatggaaaaaacattaaaagtatataaagatggtgaagtagtaggtactaagactgctgaacaaacaggtaaaacaactatttctattagtggtttaacagcagatactacgtatcctaaaggaacttataaagtagcatactctaatgagtctggtgaatcagaaaaggtagatgtacctgaatttaagacagcaccacatagtgagctataataaattaaagtccaacttaattgttgggcttttttttattgacttatatttatttatatgctatagttaaacaagaattaataaaggaggtactatatggatattaaaacagtacacttaacttcagataacgaccatttgaaagttaaaaaattaatggaagataaggaaaagtatattgcagttgcgtatgataataaatcagtaagtaaggtcaatatcattatggttattaatgttattaaagacctagtgcatatgtataggtataaaattgttgagtatggattatctaataatgataaggaaaatgaaaaggtagggtatttaatagaaaggattgaggattgatgttatttattttaaatgaagtagcttcacacacagaacatttatccgaaggtatagatatgttcgacgttgaaagtgtagtatcttttgactcggcattacatttagctagtgataatacgtatgattctgttatttttttaggttttatatacaataatgaggattacttaaagtataaagaattattaaaggatacaaaattatactttgtttctaatattaacttacccgataacaaagattttattaccgtaggtgatgatttaaaatacctatccttattagagcaacttgtggttctagatgacaaaggtttaaatattaatttcagtagttatacaaaagacattgctcacaagtacactgagttgtttagctttaaatctacttatgaggaggcaaaagaaaagggtctagtaggttaccctgtagatatagtattacctattatgactgacaagttttacaacgtaaatgatttgtatgtactatatgaagtagcacctgagtataaattacctttagcatatgaatgccttaaggataaagaaaaaggtattgttgttataggttcacaaacaagaggtactagtgatatccttactttctatgtaaaagggtatgatgtcagtagtgttgctaaaacgtttggagcatcctataatgaaaattctaatatatttagtatctttattgatagtcatattagtgtattaggtgagaatatgaacaagtatttaataacggaaggatgtatttatgagtaattataaaacaatagaagagttacaagcaattattgtaggtattttatttaaagatgaaggtaaggtagttacatctaaatttaataagattctaacaagttttggtattgatagagtaaaccgtaatgagttaaaagacattgtagaaaatattagaagtgatgagtaccttaaccaactaaagaataaagctgttaaaggtgaagtgttattagaagacttaagagatgtagaagataaacaagtatttgagggtaatgactatcatcaagaagtgtcatcttatgtagtagcacatgaaaaagaactatctagactaagggaattacgaaagattaatagacaaactgcataccctactattttgtttgatgaattaaagcgtactatggttaatgagctaaaagggaataagttactagaccataaagtaagtaagtatacttctactgaggaagaagaagagctggttatcttactgtcggacttccatgtaggttgtgcttttcaagatttaactaatgagtataactttgaggtactaaaaagaagattaaatcagctactacaggaaactattaaggatattagtaaacgtggtattagtaatgttacagtatactttgtaggtgatttaatagagcatattaatatgagagatgttaaccaagcttttgatactgagttcactatggcagagcaggtagcaaaaggaactagactattaatcgacttcttaacagaattatctccatatgtatggggtaatttacgtttcggtatgatagcaggtaaccacgaccgtgtacagggtaataagaaccaaaaagtatacaatgatagtgtagcttatattgtattggattctttactgctgttaaaagaaaacggtgtattagaaggtattgagattatcgacaatagaaaagatgtatatactattaaagacacagtatgtaacttaaatattattataaaccatggagatggacttaaaggtaaaggtaagcatattcctaaatttattgagaacactcacattgatttgttaattacaggtcatgtacaccacttctcagtaacacaagaagattacaatagaatgcatattgtagcaagtagtcctatgggatataataattatgctaaagaactacatctatctagaacaaaaccatcacaacaactactatttttaaataaaaaaaataaagacattgacattaaaactgtattcttagattagggagagataatatggacattctatcaattatagccttaacattgcttatcattattattgttaataccacaatgaactttgtaggtatgttacgtggtgagcgtgaattagtaaaaaaaggaggtaaccctctacccaattggcagtactataatgtgttattacctaatttatgtggtattatcctcttaggtattgttgtttattttggagattcgatttataaaattacaacaaggttagaagttctttttgctattattgcgcttattgtaatagatgtattgttaacagcgttagtattattagtattgagtttcgtaacaaaaaataaagaatagtaaagaagggagtctggtttatacttgactctctttgttatttatggtatattagatatgaggtgatttttgttgcaattaaattttggtaaatttgataatgaaattattaagacaaaagtaagtgaaggtgtttactcttttagaggtgttccttattactacatagaacatgtccaagatgaaacttctgagtatgtattagtatataacatacatagtgttgatgatgaggtaccacaaaagaaatataaaatagaaacagtaagtaaaactattagaggtggtacgatattaagtaatacaattaagtcaatgctacctaataataagaagtataaaaaagtatatgaacctcctatctttttagctaatattatacctttaggtacagatacaactacaggttctgtaggtaaagggtttttcgaaagagaaaaagatagagtaactattacacaaaaagaaggcactaaagttatacatggtgagtatacaggagtatttatatgtttatctaatgttaaatggattaagtcttatacacctttagatagtatattacaatattatcaacgtattaaaggggatagaataaatgtctgagataactaaattctatgagcaggatattaaagatttaattagaacgaagagacacatgtttaaagatgatgagataacaagtaatatacaagatattagaatatttaatgaaaaagctatttgtcaaggtaaatgtagaactgactgtttaattcttgatagaaatggtactgttatgggtatagagataaagacagaacgagactctacacaaagacttaataaacagttatactattatagtctagtatgtaagtatgtttatgtgatgtgtcatgataagcatgtacctaaagtagagcaaatactaaaaaggtataaacaccatcatgtaggtattatgagctatattaactttaaaggcagtccagtagtaggtaagtataaggaagctacgccttcaccactaagaagtccttatcatacactaaatattttatggaagcaacatttaatgactatacttaaacagttaagagattataatacatacctaactggatataactatagggagcacggtaaaaatactaacaatgagggtagttatacagagggaacccaacgtatgagaatgaaaaagtcagctattattaaccaagttattcattacttaggtgtagagaactcttataagatatttactagaggtgttatctatggttacagtaatagatggcagatattagaagatgatttctttaaagttatacagaacggagtgagggtattatatggcaaagaggaaacctgatacatttaaacctaaatctaacggatataaacatcaacctgttaactttgctcctacgggtaacttatcaggtagaagtacttctttttttaataaaaaaagaaaagatatatcagatgaaagtattgttgttaaatacaaacctttgtttgttaaaaggtttgataatattactgctacagacattaaaatacaaaagaagtatgcgttagacttaattagtgaagctgtgaatataaagaagaaatatcttgtaatgaagcaaaaaggtaaattaacacaaactattttacatacggatagagtttattatgtttatagaggtaagaaattaataggtaaatgtagtattcgtgaacaacgtacatttagaggtacacatttaatctacatttttagtactagacatagaatatctatacgcaaaaatagtagattagataagaaaagaacaccaaagaaaatgatatataaaggtggtaaataatggttaaatatttaagaacaaaagctaaaaaaacaaatataagtactttatttaagaaactacaaagtaaagatattacactattaggagtgtcctatgatagtgattatttccctagcggtgtaacaataataccttatttagaggacatatcacaagtagaagatggtatagaatttacgaataaagtaatagttactgagaacttaaaacctgctattgtaggtatgaataatatgataagtgattcaggactaggttatgttaaaacagagcagttaaataaaagattagaaaatactggattaatgacagatttattatctaaaggaacagaattaacttcaacaaagaaagtagacattgtatcaacttttattgaacctactattatgtaccaagatactactattaataaagaattaaaacttagattatacactatcgaagatgtatcccctttaaatgattacacacatgttgtatacttactagtaactgataagcaatatgacggtcaatcattcataggtacattatgtaaccaaggtacattaaacaaattagatactctaaaagttcttacattcttcaagggtaataatttaatcaatagaagtgtgttttctgttaagttagatacgaataaatatcattatagtttatataatacacatgagaccggtattttctttttagatgatgataaagacttaattattgcttgtggtcaatcctatgtaaaagttagatataaagatattgttagtagtaaagtagagaaagttagtgataataactataaaatgattgttaatcttgttggtaatgatgagctaacaattctcttatagtataatactcccttaaatatgtatactaaggtataaatatacgtatataagggattttttatacatatttttttcaagtagttagttaatttctaaaaggtattattcttattttaaaattatatttataaaacattaaaataccttttaggtatagggttttagaaaaaagatttatattgcttaaaatagttacactatagtgtaaaaaattagaatatcctttatgaaaataaaatagatgcagaaattgtgatatattatatagtgtaaagtataaaacagttgatttgataaggttatgtgaatacacatactccttattttagttcacacatatgtaaagacacttgagacaagaagggaatatataatggctagaaaaaagaatttaagaaataaaaaatctgatataaaggtagttccagataaggaaagtattctatctaaactatataataataaactattaaggtctaaagtggataatgctatagatgaagatgttagttatgatgatattatagacctatgtaaagagtatgacttagagttatccaaatctgccattacaagatataagagtaaacgtaaagaagctattgaaaatggatgggatttaggggaacttattgataaaagaaagaaagttagcgctactcaaatacagaaaaaagaaaatcctatgaaagaattagatagtgagaattatacaccttttgaactggctacacagaatgtacaaacaatatatgatgatattcaaatactagatatgattattcaaaaaggtgctaaaggtttaaactttgtagaaacattagaccctgctttaatgattaaagctatggaaacaaaagatagaattacgggtaaccaactaaaaggtatgtcattcataggactaagagaactaatgattaaacaacaagcacaagatactgctatgacagaagttatgcttgagtttattcctgaagataaacatgaagaagttcttcaaagaatggagcaactacaagaagagttctataaaaacttagatttagatgaagaaggaagaaagttaaaagatacattagatagagtaggctatacaatatagataatgaggtaatacatatgacagaagaaattagtttaattccaataaaagatgttaaaccacttacagatatagtagatattataacacacctatataaaggtgatgtacttagagttaaacaagagaaccaaggagatatattacttagactaagtactggaaaacataagtttactgaagtgtcaagagacttagataaagaaaccatgttctataaaaggtattgggtactatacaatgtatctattaattctttactaacttttgatgtttatttagaagacgattatgtagaatctacaaaagttaagttccctaagaacacaattgtagaatatgtaagagataatcaagaatcagatgtagctaaggttaaagatattttagtagatagtaacggtaattatttctatgcactttcaggagaaacatctttatataatgaagataaacttaataaaattaaaagttaatgattgacacactcatatgggtgtgttattcttttattacaaacaataaaaggagcaattgattatgattattacattaaatgaagaagagaaagcattattagaaaataaaagcaactatacgccattaagtaagaataaagagttcaatacaccaaaagaagaatatattgttacaagttataataaaggtgtacctgtaactgatattgccaaagaagctaaagtaagtatgggtttgatttatacagtgttaaacttctataaagtacctaagaaacataagaaatctagagtagcaaaacgtgtaaagcatattactaatgataagaataaagtacaagccttaattaaagattatcaatatatggacttacaaagtatctataaaaagtataacattcacaagaatggtttatactatattttagatttatataatattgaaagaaaatcagatttaaaagagaaagccttagaagatactatcgaggtagaataagaggtgtcatatgcgcaaagaacgaattgtagattacttgtataatgtaaagaacattgataataatttaactatacaaggaatagaaaatagtttatttaataaagaatctctacaagacttagtacctgattttgatacttctagtaaagagattaatgaaagtaatttatacctatgtacaataccagaagattataactccgaccatgtagaaagtggtcagtatataggtatagatgttgggtatgtatctgaagaccctgcttttgaccatttaataggtcaagttcctagaagtgtatatgagaaagcgcatgttatgcaaccactaattaagatagaaaatactgatatagattatacccaggctgatatgattaatgatattaaagtaggtacaagtattagtgatgtaaagattaaagataaattaagtttaatgtcaaataatcttattacacatttagaaattgttgataagtattactttgatagtagtatagaagaagcactactaaagtattatgagttaaaaaatcctgactattatttaaaacattttcttaaactaaaagaacttgtcagagacaatagaatgatatattgtcctttactgttaaaatgtattaaagttatagactaagttattatattataagtagtaaaggatggtgcgctatgagtaaaaaagcaataattgcttatgtaattatcttagttatgttagctttatctatatctacttattacgtatcctcatacttatatcatgaaaaaaccaaatcacaagttacagaccaattaacacaccacggtaaactaaaaaaagataagaatgtagagtatgtaggggactatacacttaagaaagtaatagacaacaaagcatactttatggaaaaactacctacctatttaccaggaagaacaagtgataaaagtattgatatgagatattataggactagtaaatttagagaaggtgtaaactttaaacttattcgtgtatacacagaggataatgacaataatccagtacacaaatataggtttgaggcagtattaaataaaaagtaaaggtatgatagtctatgaccgtagtaattatactagtatgtattgctattatatactatttatttaacaaggattgagtcaagtttatacttgactctttttttatgctatgttatagtttaaatataaggagatgataaatgatgaaaaaaagtgatttagataatttgtatattgtggtcgtaagtattaagaatggtgttttacctgatgagcgtgtaattccaattggtttagatgtactaagtaaagtattagagggtaaaactacatggacagttaagaaagaaattaaacatacatttggtaaagatgaattaaaaagatttaaagataattacaagttcttgcaacataaaattgtagaccttgctacacaaggatataggtaggtggtataaatggatatagtagataaactcatgttaggttttattttattgttttatgtaggtgtatttatttggtacttatgggaagtctggaaattaacttctttaatatctgatatagttattaagtattatgttcgtaagggatgggttaaaggtttattaccttatttgattcatcaagttattattacgttagtttgggctactctagtattagtaccttttatacttagttatataatgataaaatatttgataggaggttactaaatggaagtagtatatgagaatgaatgtagaggatattatatttgtgtcatagataatgaaggtaagtatgaagtatcatcttttaaaaaatcagtaaacaaatatacccctgaaattatagggtatactacagaaaagttactcttggaattaaaaataccatgtacatatgtaaatgctagtgaagtagaaagttttatgcaagaattaaaagtagcacgtattgcgcttgacaagtttactaaagatttatataataaaggcataacaaatattatattctaaagggtggttagtatgtatacagaaaaatatagaaatttaatccatattacacatagtatcttggttagtattcaagaattagcaagtaatttattaaataaggaagtaaaactagttacaaaagaaaataagtacttatgtgttgtagatggtattattatgttaattgttgaagaagggttagtatacacctattttgataaggtaggtttaaaagtgcctgaacgtattgcacttggtcatttagcaaaaagtttacagtttacaatatcagaatcaaataaagatttagaaaatcaaaaataagtgttgacacctttaacatgttgctttatactaagagtaacaaaacgttagaggtgattttttatgtgtaaagaaaaagttcatagacagctatctaatgctatagaaatattagcaacttacaaagagtggtggtcttttccaaaagaattaagtaccacagaaacatttaagattattaactggaaagaagatacattagtatttgaagttagagataacggtaattatataggtaggtttagtgtagcaactcctattattgactttcattatgatagaattaaaaaaacgacagcacaatacatgataaattattttgctaaactagtatgtaaagatatgtatacgtattataaattacagagaggacagtaaaaatgaaaaatttaaataaacagaaacgcttgacgttggaattgattagtgataatctaaatacacaacataacttaaatacatcggttagtcttgtagattatagtataaaagactttacaaatagaaaagtgtatgtaagaactaaaaaaggtttagaacttactaatatgaaaagtttaaaaagtactgagtcattagatttattatttaagtatctagaattaattttaaaaggagatgtataacatgctaacaccagaacaaagaacacaattaaaagaatatcaaaataaaatgacaaaaaagaaaaaataaatgttgacttactaaataacaagttgtataattaacttaacaaagaaaaaggagattgataaatatgaaaaacttatataaaattttaacaggagtattctttagcactacactattattaggaggttgtgcttactactcacaagaagatacaccgacgcacaattataaggaagacaagaaagaaaataagaaagatgaaacttcctcattagaagaacaagagaataaacaaattaaagaagacaacactgttcaaaataaccaacaagataaccaaacacaaggacaagataagactcaagctcaacaaaatgaacaaatgcaagaagcaagtagaagtgaacataatggtttatctaatgctgagtatgcagacaagtataatcaatatagggaagctcaacgagcacaagcagaattagagaatagaactgaggcggataaagctagcggtggtggaggcggaggagcctcatggaactatgcaggagctaatgaaagttttgaacaatggttacaacgttcacaacaagaaagagcagaggcaggattacaataaaattaataaaagatacaggagaataagtatgaaaacaataacacaggaagaactaaataaaattctagtagaacataagctagcgtaagtagatacaaggatgattaaatatgaacattgtagaacaacgaaaacctataaaaagtaagcatattaacctaactaaacacgcgtatgaacgttataccttaagagttagtaacgatagtaaagaacgtgcttgtcagtggtttgctagtgctttaatgcaagctacttttgaacatacacaaaaaggcggtaatgaagtatatgtatttaaaaattatagaattattattgataagcatctaactattattaccgttatagatactgaagatgataattgggaaggcataaaggaagcaagagaacaaatagatacgtatattaaaggtaagctaaaaagaactgttacacctctatttactaagttagatagaatagatattagtatccatgaagcacatatcagcatgattaaaacaagaaaccctaagacaaaagatataattaacaaaaatattgttgaacttgaaagaaataaatctttactattaaatagtatagaaggtgtaaagaaaacagcacataagtattatgtagaattaaaagacattcataaaagattaaaggactgataataatgtgtttaaaagatttaattgaagattatataaatcaagataaagaaataattataacagttacaagtactaaagtaattattgaggcagactatgatgttatttgtttacctaaaagtaaaattgatattgacgatttaaaagcttatgtatatggatttaatagatttaatatagagcaaattgtattaaaggacgtggaataatgcaagacttaaaaaggataacacaacctgaattagatttattgatagaaaagcatgaacaatggctagctacaaatgaaaaggaaggagaaaagctttttttacagtgtgtagatttaagaaatcttgactttcattccgctgaccttcaatattcaactattgtaatgtctagtttaagtgatgctaacctaagttattgtgatttaagttattctgatttaagttacagtgagtgtatatctacgagatttacaaatgctcgaatgaaagatattgagttgcatcatactaacttagagtgggctaatttagtaaaagcaaacttagaaaatgcctgcttagctgaagctaatgtaagtgaggcagacgcaaggtgggctaacatgaaaaatgctaacttaagtatggctaatttaaaaggtgcagattttggtggtacaaacctaactaatacaacattatattatgctaacatgcttgacgtgtttgggataagtatctatactataagtaatgtacttgattttgacgagcatgtaacttatttacctgattatgatagaattattagtaattataatgaatatacccttgaagagtttggtaaactagaacctacacttgagtacgaagaagatagaataaaagaagtctcaaagaaaattaaattagcttacgatttctttaaaggagtgaaagaacttgataaaatttaattcagacgaacataaacatatacttaatgttatggatagattaccatatatttgttggttagatactgtagatggtgagtcaggtgctgactttttatgcgtatcgcatagaacacactatgaacttactgaactaggtataatgatttatagttatggtagttggtatatgataccttatgacaaagaagatttaattgaatatgcaataaaaagagatatagaggaggaaggggaatggtaggtatggtgattacatttttaattgttttaggtattatattgctaagtgtactaattattgttataccagatttaagcaaagcaaaaaaacgtaaagaagactcagatattaactataatcaatatactaaagacatggcttctgagtactatgaaaagtatataacacctaccgaacacattattcaaactaaagtagaagaggtaattgaaaatataaagaaagaatataatgtctctactgtaattattgactcttctagtaatgaagacaaaattatcttacatgttaacgataagtacttaaaaaaggtagtgtataaagatacgtatgaatacatctacgattctaatacaaaataccaatacattattaataatgtaacatatgagtcgagtatcttattaaaacaagatgaagtaaataaattaaaagatatacttaataaagtacctgtcacagctaagcatacatttaaaaatgagttgcttaatcgtaggaatgaatatgtgttaggttatattacaatatatatttatggtgacttaactgctaaaattacaaagaatggttatgtacaagactttagtacactagaagatttttgtgataagtatgaagcttactgtagatttgttagaattgatttatatgactgggtaaaagaagtaagaactattaatgggtataaattaaaggtagatattaaaccttattttaaaattactgctactaaaacaagtaaatacgaatttgtagtaagtgatgatgtatctaatgttaaggtatatagagatgatatactactaacaaacaggtatgtgcaaaaagcaattgatatacaggaaatagtagaggaaacattgaataataatactgtggtattttaaaaaaagggtatgaataatgaaaaatttaggtaaaattataaatattattgaaagagaatgggatattaacatagatagagaatatttggcagatatttattataatgaagttgaacaaggtagaaataaagcaattagtacgctaaaacgtatactagaaagagataaagagttatcaaagtactattatgacacttttctagaggcattaaaaaagagagacattaaaagagttgtatcttattttaaataaattttataaggtgggagaataatgataattcaaaagacatatacttgtaaagaattaatagaaaaatactttaatggttatttaaaagaaggtacttacacaggtacatccgatggcttagatacttattaccttctgttagaaaataaccgtatgactattggatatcaagtacctttggacgctagttttatagtagaagaagaaattactaatgacacaaaactagaaaactgctgggtattattaaatagtcatgcaaaagaactattcatggataacacaggtaatgtgagtattaatgattgtatagaaatatacggagatgaattattaactgtcagtatcttaatcgatggtacctttattaatgtatggagaaatgatgattattttaatagaggaggagaataatgaaaacaataacaaaagaagaattaaataaagttctagaagagcataaattatggatagaaagcaatgaggaagaaggtaaaagagcagatttaagttttacagacttacgtgacctagatttaaaggatgttgatttaagagaagctaccttaataggtgctgatttaagttatgtagatttaacaggagcagatttaagatatgctgatttagcgtgtgctaaactaatagaagttaatttaacagaagctatcttagcatatgcagatttaagttctgttgatttaataaggtctaatttaaaaaatacccatttaacagaagctaacttaaaaggtgctaatttagtagatagtgatttaacaaatgctactttaatagaggctgacttaaagcatgcctccttatcaggtattaatttaataagtgcatatttacaaaatgctgatttaagattttcttatttaagaaatactggtttaataggtgctaacttatctcaagcatctactcaaggtattagaggtttagaaatgtattctatagataatattggtagttttaaaaataaagtaacgtacctacctaaatataataaagtatttgtaggatgttgggaaggaaatttagaagaatttctaaaaaaaggattagaaatgaatgaaggagataataaagagcgtaccaacattgtactagcttatcagttctttaaaaaccagctataataatttaagtatgagtatttcttagtaagtaataagtaggataaaatatcctacttatttcatatataagtgttgacatgaataagtgtaaggtttataataagtatataagttaaataattatagaggagacattaatcatgaaaaaaatattaagtttagttttatccgtattaattgtactaacaataatcaatgtgttgaaaaaccaagagcaagtagaacaaagtaaccaagaacctaacaaccaagagcaagtagaacaaagtaaccaagagtctcaaaataatgatgcagatgaccatacttcatacgtacctgagaataacggagagcataaagacgttagtgaagaacgtaaaagattatggggtacagagtatcaagaaggtgaaacacataacccaaatgctaaagtgtttaatcaagatacaggagaacaggttcaataaagagtaagaggagcacttattatggagtactggaaagaacaagacatatattattcaaaaacaaaacaaggtgttataagctatatagacttatcttccttacctaaaaagaagtctcgtattgattgggtaaatgtaaaccattatataccctttgagtatcatagcacgaaaggtaagctctatattaaataccttacaccaactaaaaaagaatctagagtagaaataacttacaacaataaaatccaagctacccaagtagctaatgtctataaggttaaattcaactttattacccaaggtcagaagtcttatactgttcataatataaaacaaggagatattattaaaaattgcgaagtactaaaagtagtagacactcatttaggtacacaactttatgtatataacataaaggaagaagtctatggtaaagtagtaaacaaaatttactaaaatataaaactgataactatataccatatggtactattcctcaatctaagatgttataccataagaaacatattcataagtatgctaaatataaagacttatttaaatataagtatacaactaagaaaaagatagaaacgatttgccctaattgtaaaagaaaaaaagatattagaattacagatttaattaaagataactatagctgtcaatgctctaaacaatactctagcttcggagaaagattaacacaaagttatttagatattgcaaacatagagtatattagagaatataaatttaaagaattaggtaaaaaaagatttgattttttcttacctaagtataataccgttatagaagtacacggtgttcaacattaccaagaagtcagtggatacatgaaccaccagaaaacaaaattatcagacatagaaaagagagaatattgtaaagaaaatagtatacaatatatagagattgatgctagaaaatctacctttaaacatatattaagtaacctacaaagaacatttaaatactctaatatagatagtaaagaagtaattaaaagatatagacatgtatataggtaagtgtagaagagtaaacaaccaaacagacaaacaaataggtatagagattgatgtaggtagaagttatataagtaagtaataggtaaaagaaagcaatatatatatataagtagataaagaaatcagataagtaacacataaagaaacaactgatattataagtacctatatatcaaggaaataaataatagataaaaaaacaataattgatagcacatagataagcagtaggtagtaggtagtgtataagtaatagctaaatagataagtaaataaatactgtataacagtttgaagatatgaataataagaagaaagaattattaagtaacttaagtattaataaatattctaatagtaagtaatagatattaagtaataggtaagtaactaaaagaaagtaactaaaagaaagtagctaccactatcacttaggttaggtaagaacagttacctagtaatttctttacatcttctagatagataaataagtagatactaagtaatagctagtaagtaattaaaagaaaacaggtacatggtatacctatatactaagtagcaagtatcaagtatcaagtatcaagtatcaagtatcaagtagcaagtatcaagtatcaagtatcaagtagtaagtagtaagtagtaagtagtaagtatcaagtatcaagtagtaagtagtaagtagtaagtatcaagtatcaagtagtaagtatcaagtatcaagtagtaagtaagaacagttacctaggaatttctttgcttcccaccttcatagtgtataaatagttaggtatctaactgttagatacctaactatttatttttatatagttttaactactgtgaactatgcttatgtagtattaaactatgctgattatatgctaaactattctaatgtatagttaaactaagctgaagtagtatttactgtgtataaaggtatatttagtattagatattgtgtatatggtatactataattctgaacattacttatttaatatatatatatagtttgtttctatggtagtattgacttaattttccctagagtaaaagtttaatatatttagtaatttccgaacaataaagaaattttaactattttacacatatgtgttgacagtatattacttatggtttatactaagtatataaagttaaaggagatgatttatcatgaagattaaaagagaagtttcgtttatagaattaatgtatatgtattataaaggtgatgtaggtaaaggtcagtactattctactaatagtcagtttattgttgttataactgttgatgatatatcgttttataatgcattgtaccctaataaatctattactattactatacctaatgacactaagtttattgtagaagcggttatagatgcttctactgagttaagtagtatagtagttacttataagcaaggtaacagtgtattaaactcagtatggtataggattagtattaatgaagttattgactactatgtcgaaggtaaaaatacatggttactatctgttagtactgttgtagataatgagttagttactatatggaaagatggtaacgtattgtgatttggtatatagtaatattcagtatacttatatatgtacatcacttgtattattatgtgtagtactagtatatagaggggttaaataaccgcttcaataaagacttagtaacgaggagttgtattatgagtttatcagatatttatgtacatttaaatcagttaggatataaaatagaagttttaggggacatgagtacattaaaagttaatgactactacatcgaaaatagatgtatagatagtggttttagggttacaggggataatgtatttttatctgtatattatggtgaagacgtagtaagattctttaaacaaaggttattaaaatagtattttgttaagttatacttgagaataattataaaggagggattagtttatgagtatatttataagtatagtttctatatttttagctactattagtattgtactaagtataaggtttggtaagaagtattataagcatgtacttgataattatgatagtgttagtgatttagaatcagctagtgtatttggtttattatttatgagtatttgtattggtactatttctattgttttgagtttaggttacatagtaagtagagtattttattatataggagggttgtagatgatgagttcaactttaatgtatattgcggttagtattttaggtattagtattatagttggtatgcttagtgctttaattactgatatagttaaatctattaagcagaaagaagtgttttttagtatatatcttattgtttcttttgttttaactgttttagtttttgcttctataatactacatatgttaggtatgtaatagaaagagggtattagtataggttactttgtagtaagtttatcattaatgtatttagttatagatatagatatatgttatagaatatatagtaaagaaggtaatagtaatagatttaagttagcagtgtttttaactatacttatattgattatgtttatatatggagtaggtgttttattaacacaatagaaagaggcttattatggaagaattagatatactagatagagtgttacataataaatcaggtataggtgagtttacgtatacaaattgtataagaacaagtgatgataagtatatttataggttctttctggaagatacttattttggttgtgctattatggtatatgatgatatgagtgagtatgaagtagtagtacaaggtacaggtaaggtgtatgtttccttattgaaagatgtagttagtaatatgtataagatactagtagatgagtatgataagtatttaaaagatagtgcatatcaaaaatatatgtataatatatttgaagaacaagtagaacagtataactaaagtttttctctagggaaaagcagtcaaaggtaaaaatagggcttgaacccgcattctatggttgacatttaggtaaaattagataggttttccctagaagaaaagtttaaaaggtaaaaaaggagttggttactatgtcaaaattagctaagttttgtaggaagtatggagaagttactggcataatggagagtattacagattatgaggtgtacttaggtaggggttataaagtaaggtgtagagtaggtaatagccttatttatattcttgtatataaagacaatcatatcattgctaggtttgaggaagcggataatatagtatttcctttgagtgatacgcttgaggtgggaacaattaaagacttagaggagctagaaggtttgtatactaatatctatatattactagggtatttatatacagaatatgaaaataaagttatgtatgaatatgggaggttagaataatggatattatggtaagtgaattatttgagtatatattaggtcataacactcacattcttgggaattggcatacttattacttagataaaggttactgtattaaagttaatgatgataaacattataataacatgaagtttatcaaaaatggtagagtagtaaaagaggttattaacagtgacaccttactacctaaaggattcagtagtatctatagtcttattgattactatgaggaagctttagatgaaaagtgcagaaaagaaaaagagcgtctgaaagaaattcgtacgttcctaggtaagtaagtattatatataaccgcttctatatgaaactttatacctatagctattataactataggtatttttttatatttcttacataaatgtgttgattttatgtgtgttaaggtttatacttaatatataattaaaggagtgaatataatatggatatgaacaaggaagtattagaatgattagatagtttaggtggtaagttagaacaagcaggtacacatagttatgaatctttaattaaatacacaattactcaaggtattatagatttatgttcaataagtatattattatctataacagctatcttatgggtggctttgtataagtcagataaaaagtctaatgaaggtcatagtaccctattatttgagtttgtttataagaatgaaaaagagctttctgctgcaggatgcacagtaattatatttgctgttactctgtctattatttcttttttagtgctattaataggcttgcctattgctattcaagagatttttaatccagaaggatatttgattaagagtacgatagataatttaaaataattgtaaaaataaggaggaaaatataatgttagaacaagctaaaggcataactacagattatattatggaatttattgatgaacatgagcattatttctatgaagacttacacaagttagcaggggagttagtgttgaaagatacacatgacacaaaccctattgttattaatgatggtgatggttactcaggtactattatatctaaatttaaatcaaaggtagagggtgttccagatatcactgttagtgatttttacggttcatgttctttctgtgatactttagctaatatctatgaaggtagtgatgaacttgagataattagagatttagctactatggtactacatacaattcaatctatggaagagaaagtagaataatgtataagggagtgatatagtgtttagtaagagaaaagaaattaaagagtataagaaacaaggttactctgttgattatatgggtggttttttaggattatttggtcttggcgatgttttatactatgtagaaactaataagaaatacttaaagtcaattagcgtaggtaaagatgacaaccaatatgtatatgactttaatggttatgaatacttatcaagacataagttaaatatcaaagaagaagaactaatgtctaaagctattgctcagttaagtatcaaaggtttaaagaattttaatggagttaagcatatttgggatgtaggtttcttagagcaatcttttaatatacatcgtaaagatattgatacggttaaacttaaactaagttttgatgaagagggtaacctatattatgaattagaattaggttggattaatatttataaagagtatacaggtgtaaaagactttcttaatacagtagataactatctcaacttcttctataaagatatagtcactagtggtcttgagttaaaacaagtactatataatacttttatgggaggagattatgagtttgtagtatctccatataatgttgtagtatatagggaagttgagttaattgatataaagaaattttactctgatacttatggaatttattcctatggttatggcactaatgtacataaacctttacaaacaattaagtattatctggaataccctatgaatgaagtataaccacttcaaatattatataaaaggagtgcttaatatgagtaacggaaaagtagcgttaattggtagtaaggtattagaagattataatatttatacaatcatgagtgagaaacatatatacttaagagatagtgttgatgtgacttttaatgatgggaaggtattacctatatacattgatagtgtattaaaacaattaagtaaagatgataaaaatctatatttttctgaagaagatatagatattagtaaacttactaatgacgagtattatacagttactaaaaaataaaaggagaatagataatgagtagtaataaaaaaatagcagaaagagttattgagaaatttttagatgagactaataaaggtgtagattttactaatcaagatgaatttaaccgcttcttagataatacagaagacttgtatgaagtatctggtactcaaagagataagattattaagtgtttacaaaacattgtagttactgtatttatggataaggggaaataacaatgaaaacaataaaaagaaaaatagtacaagaaggtatgaacaaagaagaaactatgatggaagacgattctgttgtaataaaaaaaattcaaaaaataacaggcggagctattcttcctttagccaatggcaaggaatatgtagtagtgtttagtaaagtagaccctactaaattaattgtatttaatgtggaacgtaatcaaatctatttaagtggttctggtttcgatagtgaactgtcttatgtagacagtaaaagcaaccctattaccttagaaacagttaggttaatcactaatttattataaaggttattaggcttttaaagattaagttattttatagaataaatactactagaaaatgattactgtttaatacagaattttaacgtacctagcttaagggtattaaatataaaggagggtattagaatgactaaagttgtaagtagaggatatagttttgaagaacaagaagtaagtatgaactataatcaaggacaatgggtgatttatgcaagtagaaaaccttatattactgatattatgaaaaagtaccctgataaggtggaagtattagaacaattagaaaatgaaacaccagtattggttaaagttgtattggatgaagatttaattacattgagaaagccaataagtaacgagcgtaaagaacaaatgaaaaagattgctaaagaaagatttggtaaataatagagaggctgactttaatgaacatcagtatgtacactaacacctgtagggactagattaatattacaagatatcttatagggttatagtgtcattgagaatgagtatgctttattaaggttagttaaagaagataaacagtatttataatatcatattgaagaagtagaagcggtattaaaccgcttcttttttatattatatttatattctgagtaattatgttgtatttaatcttggctaataattgtatatcaatttgagcatagtatgcccgtttcaacatagttaatctaataatttgtatatttatctgttagttatgtataaatatttgttattacttatttatatatttgtatttcgtactaaccgtacgacttatctttttaaaaagataagaacaaaacagcgtataccttgttgttttacgctacgctacaacaaggtaacgcctgtaataagttattcgtattaaaatactaacaactaattacttgttgtttagattataacatatgtttatgagaattagatgagaatttagttacaacagcattataataatattacagttatgttacaagttgctaagtatgttttgttactacaatatattgaatatgaaagctttagaccttttctgcagacaagtaattattacaggtatgtatgataaaagataagtatcagggattaaattaaggttggattaatgaagcggataagttagatactggttaaaataaaatactttacattacctaaaacttatgatacacttaggataggataaaggaggaatatcaatgaatattcaagaagcaaaggataaattatacgatgaattactttatacagtaagtctttgtgagggaacagaagaagatttactagaacttattgagaatttattttatatttacaatgacgagcctgcatttttaagtaaagacggtatgcaatatactattaaagattttatggagagaagaaaaacattagaacctatcttaactgaattaggtatagggtacgcaatgatagaatatcacacacatacagaatacgagttagttacttattcagtacttaaagttatacagtatgatgtagataactatactgtaattattgatactaatagtgataagaatattaagttatcagatttaacattagaagctgttaaggatattttgaatcaaattaagtagctagggttgttttactctagctttttcatatgcttgtgttgcctatttaactaccttatataagcaagtaagtattataggataaatactacctaaaaagtattaccgttgaatatagacctataagcttaatgctctcttactgttataaacatcataagtataaatctatgattttcctaagtagcaggcgctgttttccctttatattaaccgcttcaaacaacataaaaaaagattaggttaatagcctaatctataactcttttatataattcttgtaattctttttctaacttttttattgtttctatatcattatttcttttaatagactgacatataaatgcaatacctttaatcaaattaaatggttagttttacactgcattgtatacaattaaatcgatcgattataataattaatttaatctgtactttctagtttcttctatataatagtaagaaatcatttcttttgaaaaactttcgtataacctattgacattaaatctaggatagtgtaatgtaataagcgtagtaaacaaaccgaaaggaattgataagatatgaacgtaaatcaaattggagatatactaatggaattattagatatagatggttacaaaagcaataactactatagaaatgacttaaaatactcaacacattatcaagttaaatatttagcttacattgatggtagttatgaaacattagaactatttaaagaaacgaaagtctttagcgtatcacattcaaatagattaccaattaacattaatgttaaggtagtgaacaaacacttatttatagagctagataatacgttatacctagtgaatgagaatgcagaaattattaaagaaaaaaagttaaaataatagttgacactatcattaagatagtgtaatataatagatgtagtaaacaaacagaaaaggaattgattaattatgaaaacactaattactaacagtaaaccatataagaactacggctatgaaatggaacaattcgagagagaaaacaacgtaattatcacacgagcataccacgaaccaacaaagcaaggttatagtcttgtaatacactatacggaggttaaataatatgaaatatttatattacattaaaggaacttataacagaattaaagcacaaggtacaatatcaaatgactatagattatggtcaggtattgatgaatgtgatgacttatactaccatattaataaagcttgtcatagtctttataataatggtgtaaaggaaattacagttacagacaaatgcgttaaactaacacttaataataataaaacaaaacttattaaaaaagtttaagaaaagtgttgacacttactaaaatacatggtaatatataaatataaagaaaacacaataaacgaaaaggattgattaattatgaaaaacacagtaaaagaaatggcaaacggaaacactaaacttgaaagattattagttttagcaattgaggaagcggttatgacagagcttgttgaatatgatgcattagataaatacaaccacttagaacaacagttattaaacaatgaagcattaacacaagcagaacgtaaaccatctaacattgattatagcacttacgagggagctatattagcacaagacaaagcagaacactttactaacgttatcttaaaccacatgaacaaagaacaatttgaacaaatgattatggatattgaaacactagaatttaatgcagaacaaatgctattctaaggagacattacacatgaacttatctgataaagaacttatagatataatggacaacgttaatagcttcactgatatcagtgaagctattaacgaaacagtaagcaaacaaacaattaaaggcagtgacaaagacagcggaacgattaaacagtacaatagtttagatgagttaagacaagactatgacataacaacactagcaccagacgtaataaaagaattactgaataatcattagtgtaaaacttcccaaaataattaactaaaactattgcaattaactgtatatttgatataatgtaattaaggaaatacaaataaaagaaaagggattgataaactatgaaattacttaaccaaatggaacaaacagcagaacttgaaggaattgaatttgacttattactaacagctatctatgaaacaatagaggtagatgttgctgaacacgatttactatgcaaatacattcatattgtaaaccaattaaaagagaataaagacataacaccattagaatatataccgtctaacctagactttacagacgatacaacaataagactagttgataataaagtgtttgagttagcagattatatattggaacatatgcgtgttgaagagtttaataaacacattaaagactatgaacaaattaaagcagaagctatagaaatgatgtaaaacaaattgaaagggaataaactccctttctttttttttattatttattaggattgtatacaataggttaatataaatatattatacgggggcaggggtaaaatgccactaaagtgggtgggtctgacagggctatgg

SEQ ID NO: 2 - DNA, 145838 bp, Bacteriophage ARSA0002
  source 1..145838: mol_type = genomic DNA
ccttcatagtgtataaatagttaggtgtctaactatttatttttatatagttttaactattgtgaactatgcttatgtagtattaaactatgctgaacctatagtaaactgttctaatgtatagttaaactaagctgaagtagtatttactgtgtacaaaggtatatttagtatcagatattgtgtatatggtatagtataattatgaacattattattattattcatatatattgtttgtttttacagtactattgactcaattttccctagagtaaaagtttaatatatttagcagtttccgaacattaaagaaattgtaagcactttgtatatatgtgttgacactgtgttgcttatagtgtatacttaatgcataaaggtaaaggagatgatttataatgaagattaaaacagaagtttcgtttacagagctaatgaatatgtattacaaaggcagtatagacaaaggtcactactattctactaatagtcagtttattgttagtataactgatggtgaggtatcgttctataatgtgtttttccctaatactacggttggtacacctaatagtactactatacctaatgatactagatttattgtagaagcggttgtagataccactactgaattatttagtctagtaactacttttaagcaaggtagtaacgtattaaactcagtatggtatagagttagtattgatgagttatataatcattataaaagtaaaaataaagagttattatcagttagtactgttgtagatgatgagttagttactatatggaaagatggtaagatattgtgatttggtatatagtactatttagtatacttatatgtatagttagtgttttaattctatacgtatcttacaaagaaggttttactattaggatactaggttactgatactactatttatactaacagaaattacaagatttagtatcaatacaagtaatgctattctatgtacagtacttgtattgttatgtgtagtactagtatatagaggagttaaataaccgcttctatatgaaactttatacctatatctattataactataggtatttttttatatttcttacataaatgtgttgattttatgtgtgttaaggtttatacttaatatataattaaaggagtgaagttaatatggatatgaataaagaagtattagaacgtttagatagtttaggtggtaagttagagcaagcaggtacacatggttatgaatccttaattaaatacacagttactcaaggtattatagatttatgttcaataagtatattattatctataacagctatcttataggtggctttgtataagtcagataaaaagtctaatgaaggtcatagtaccttattatttgagtttgtttgtaagaatgaaaaagagatttcttctgcaggatgcacagtaattatatttgctgttactctgtctattatttcttttttagtgctattaataggcttgcctattgctattcaagaaatttttaatccagaaggatatttgattaagagcactatagatagtttaaagtagagtaatgtataaaggagtggtatagtgtctaaaaaaagaaaagaaattaaagagtataagaaacagggttactctgttgagtatataggaagtattttaggtttatttggtattggcgatgttttatactatgtagaaactaataagaaatacttaaagtcaattagcgtaggtaaagatgacaaccaatatgtatatgactttaatggttatgaatatctatcaaaatataagttaaatatcaaagaagaagaactaatgtctaaagctattgctcagttaagtatcaaaggcttaaagaactttaacggagtgaagcatgcttgggatgtaggttttttagagcaagcttttaatgtacatcgtaaagctattgatatggttaagcttaaactaagctttgatgatgagggtaacctatattatgaattagaattaggttggattaatatttataaagagtatacaggtgtaaaagactttcttaatacagtagataactatctcaacttcttctataaagatatagtcactagtggtcttgagttaaaacaagtactatataatacttttatgggaggagattatgagtttgtagtatctccatataaggttgaagtatatagggaagttgagttaattgatataaagaaattttactctgatacttatggaatttattcctatggttatggcactaatgtacataaacctttacaaacaattaagtattatctggaataccctatgaatgaagtataaccacttcaaatattatataaaaggagtgcttaatatgagtaacggaaaagtagcgttaattgggagtaaggtattggaagactataatatttatacaattatgagtgagaaacatatatacttaagagatagtgttgatgtggcttttaatgatgggaaggtattacctatatacattgatagtgtattaaaacaattaagtaaagatgataaaaatttatatttttctgaacaagatatagatattagtaaacttactaatgacgagtattatacagttactaaaaaataaaaggagaatagataatgagtagtaataaaaaaaaatagcagaaagagttattgagaaatttttagatgagactaataaaggtgtagattttactaatcaagatgaatttaaccgcttcttagataatacagaagacttgtatgaagtatctggtactcaaagagataagattattaagtgtttacaaaacattgtagttactgtatttatggataaggagaaataacaatgaaaacaataaaaagaaaaatagtacaagaaggtatgaacaaagaagaaactatgatggaagacgattctgttgtaatcaaaaaaattcaaaaaataacaggcggagctattcttcctttagccaatggcaaggaatatgtagtagtgtttagtaaagtagaccctactaaattaattgtatttaatgtggaaagtaatcaaatctatttaagtggctctggtttcgatagtgaactgtcttatgtagacagtaaaagcaaccctattaccttagaaacagttaggttaatcactaatttattataacggttattaggctcttaaagattaagttattttataggataaatactactagaaaatgattatcgtttaatatagaaatataacgtccctagtttaagggtcttaaatataaaggagggtattagaatgactaaggttgtaagtagagggtatagttttgaagaacaagaagtaagtatgaactataatcaaggacaatgggtgatttatgcaagtagaaaaccttatattactgatattatgaaaaagtaccctgataaggtggaagtattagaacaattagaaaatgaaacaccagtattggttaaagttgtattggatgaagatttaattacattaagaaaaccggtaagtaatgagcgcaaagagcaaatgaaaaagattgctaaagaaagatttggtaaataatagagaggctgactttaatgaacatcagtatgtacactaacacctgtagggactagattaatattacaagatatcttatagggttatagtgtcattgagaatgagtatgctttattaaggttagttaaagaagataaacagtatttataatatcatattgaagaagtagaagcggtttaataccacttcttttttatattatatttatattctgagtaattatgttgtgtttaatgttggctaataattgtataccaatttgagcatagtatgtccgtttcaacatagttaatctattaatttgtatatttatctgttagttatgtataaatatttgttattacttatttatatatttgtatttcgtactaaccgtacgacttatctttttaaaaagataagaacaaaacagcgtataccttgttgttttacgctacgctacaacaaggtaacgcctgtaataagttattcgtattaaaatactaacaactaattacttgttgtttagattataacatatgtttatgagaattagatgataatttagttacaacagcattataataatattacagttatgttacaagttgctaagtatgttttgttactacaatatattgaatatgaaagctttagaccttttctgcagacaagtaattattacagttatgtatgataaaaggtaagtatcagggattaaattaaggttggattaatgaagcggataagttagatactgattaaaataaaatactttacattacctaaaacttatgatacacttaggataggataaaggaggaatatcaatgaatattcaagaagcaaaggataaattatacgatgaattactttatacagtaagtctttgtgagggaacagaagaagatttactagaacttattgagaatttattttatatttacaatgacgagcctgcatttttaagtaaagacggtatgcaatatactattaaagagtttatggagagaagaaaaacattagaacctatcttaactgaattaggtatagggtacgcaatgatagaatatcacacacatacagaatacgagttagttacttattcagtacttaaagttatacagtatgatgtagataactatactgtaattattgatactaatagtgataagaatattaagttatcagatttaacattagaagctgttaaggatattttgaatcaaattaagtagctagggttgttttaccctagctttttcatatgcttgtattgcctatttaactaccttatataagcaagtaagtattataggataaatactacctaaaaagtattaccgttgaatatagacctataagattaagtctctcttactgttataaacatcataagtgtaaatctatgattttcctaagtatcaggcgctgttttccctttatattaaccgcttcaaacaacataaaaaaagattaggctattaacctaatctataattcttttatataattcttttatataattcttttatataattcttgtaattctctttctaacttttttattgtttctatatcattatttcttttaatagactgacatataaatgcaatacctttaatcaaattaaatggttatttttacactgcattgtatacaattaaatcgatcgattataataattaatttaatctgtactttctagtttcttctatataatagtaagaaatcatttctttaaaaaactttcgtataacctattgacaatcaatataagatactgtaatataataagcgtagtaaacaaaccgaaaggaattgataggatatgaacgtaaatcaaattggagatatactaatagaacttttagacatggacggttacaaaagcaataactactatagaaatgatttaaaatattcaacacattatcaagttaaatatttagcttacattgatgatagctatgaaacactagaactatttaaagaaacgaaagtctttagcgtatcacattcaaatagattaccaattaacattaatgttaaggtagtgaacaaacacttatttatagagctagataatacgttatacctagtgaatgagaatgcagaaattattaaagaaaaaaagttaaaataatagttgacactagaactaagatagtgtaatataatagatgtagtaaacaaacagaaaaggaattgattaattatgaaaacactaattactaacagtaaaccatataagaactacggctatgaaatggaacaattcgagagagaaaacaacgtaattatcacacgagcataccacgaaccaacaaaacaaggctatgaccttgtagtacactacacggaggttaaataatatgaaatatctagaatacattgaaggaacttataacagaattaaagcacaaggttctataataaatgactatagactatggtcagatatggaagaatgtgatgatttatactatcatattaataaagcttgtcatgctctttacaatagcaatgtaaaagaaattacaacaacagacaaatgcgtgaaactaacacttgaaaatggaaaaacaaaacttgttaaaaaaggttaaaataatgtttgacattacatttaagatactgtaatataatagatgtaatacaaaaacaaacaaaagggagctaataacatgagagatataagaatagttttaagagaaattgacgaggtacagcaagccttaatatatggtaggaatagcagagagttagcacgttttttacgtagacaactggtaaaactagagaaagaaagacaagatataaattggtcatagccaaaagcctattacataggcttaaaaaataaaaataagggattgatacacaatgattaaagttaaatataaaggcaagaaataccgctcaacacaaaatacagatgctattgtaactattataggtggtacagcagtaatcacattgatagactggtttattagattacaagagaatattaatatatttattagataagggctaaccacccttatttttttatgtctatataaccgcttcacattagtgtaaaacttcccttatttctttgatataatactttacaaatacgagcttatactgtaatatattaattgtagtaaggaacacaacaaacaaaaaaagaagagggattgattacttatgaaaaactataaaactttagaagaattaaaccaagattatacagttattagaacacaaaacgaagcttcaagcaaacgtaataacttagatgttactgatatttatgttaatgattggggtttgttacaatatggtatttactttaacaaattcgataacgtgtacaatgtagtttcaatgacatcaggaaacaagaaatataatattactgcttatgaacttgatagctttattatggatgagttaaacaaacagttagagttatttattaatgcaggaagactataaaggaacacaacaaagaacagcaaacaaataaaaaaaacttaaaaaagttttaaaaaagtgttgacactaacatcaacatagtgtaatataatagatgtagtaaacaaaaacaaacagaaaaggattgattaattatgaaaaacacagtaaaagaaatggcaaacggaaacactaaacttgaaagattattagttttagcaattgaggaagcggttatgacagagcttgttgaatatgatgcattagataaatacaaccacttagaacaacagttattaaacaatgaaacattaacacaagcagaacgtaaaccagctaacattgattatagcacttacgagggagctatattagcacaagacaaagcagaacactttactaacgttatcttaaaccacatgaacaaagaacaatttgaacaaatgattatggatattgaaacactagaatttaatgcagaacaaatgctattctaaggagacattacacatgaacttatctgagaaagaacttatagatataatagacaacgttaatagcttcactggtattgatgaagctattaacgaaatggaaaacaaacaaacaactaaaggcagtgacaaagacagcggaacgattaaacagtacaatagtttagatgagttaagacaagactatgacataacaacattagcaccagatgtaataaaagaattactgaataatcattagtgtaaaacttcccaaaataattaactaaaactattgcaattaactgtagatttgatataatgtaattaaggaaaaacaaataaaagaaaaaggattgataaactatgaaattacttaaccaaatgaaacaaacagcagaacttgaaggaattgaatttgacttattattaacagctatctatgaaacaatagagatagaagctgttgaacaggatttactatgtaaatacattcatattgtaaaccaattaaaagagaataaagacataacaccattagaatatataccttctaacctagactttacagacaatacaacaataagactagttgataataaagtgtttgagttagcagattatatagtggaacatatgagtgttgaagagtttaataaacacattaaagactatgaacaaattaaagcagaagatatagaaatgatgtaaaacaaattgaaagggagttaattccctttcttttttttattattaattaggattgtatacaataggttaatataaatatattatacgggggcaggggtaaaatgccactaataccacacattttatatgtatatacccgtctacccaggtactatgtaagtactataactatacagtatcaatacaaaagtcaactaaataaataaacaaaaacttatttattttattataagtcaacaatttttttttatacctcttgttagctttacatgtattatattataggtagataataaaaaaatgaggtattaaaaatatggataaaaatattataaaagttaataagagtaattatgaggtatggatagacttatctaaccttcctttaaagaaaactaaatatataaaatataactataatattatagaaaaaatgaaaaacattgaggttccttttagttacaaaggtttagaagacacaatgtacttaactttaaagtctagaactcctgaggtaacgtatgactatgagtataaaggtgaaaaaggaacaataactatgagttctcttaatagagtagcattagatagagtaacatacaatgaacgtaaaagaaaaggcttaattaaaagatttaatggtaaacgttctgtagacttaaataatctaagaaaacttgataaagaaaagtattatggtagagtatcttatgatatagattttaaaaatagtataggtctttcttttacctattgttttgaagataagaaaggaaaacttaaaattatagatgctttttctaaaaaaaataacaaaggtaaagacgttgaatatgttagattagaaaatagtaatggagatactttcttatataatgctcttaatattatcataggaaatttacaacgggttgctgacccttatgtttatccttatgatgtaggaaatactttaggtaaatttaatgtaaaagaaaatataacagaagaaaagatagctaaggatggtaaagtagagacaacacgttttagtatagtagagtgtaatgaatgtggtatgataaagaaaataaaaacaagccacataaatagttattctagggattattgtgcggactgttcttctatgaataaatccattaacaaggttaataaacatagaagccgaggggagtctatggtggagcaggtattaaaacatcaaaatattacttatattgcggaaaaatcatttgactggtctaatcgtaagcgttatgacttttacttacctgagtataacaccgttatagaggtacatggtgttcaacattacctagaacaaaaaaagggtcaaaggtttcatttaacattagaagaaaacaagaaaaatgataaatggaagagaagtcaagcactttcccatggattaaagtatatagaaatagatgtaagagaacatagctttgactatattaaaaaaagtattataagtagtgacttacctacagaatgcgtagactggaaagaagtatataggtatatggaaaatggatttattcaagaaattatagatttatacagtaaaggttatagtattaaaaaaattagtgaagcattaaatacttctacttcagtaattacggataggttaaaaaaggcatctagtcttggtcttgtagactatacaccttttgaccataatattaagcaagtagaagaaaaactatcaagaatgaaaaaagaaagagacaagtatttaaaaaatataaggaacctttagtttagattccttaccttaacacttatatacctctattatgatatgaaaaaaagcacctactaaataccataggtgcttactatacatactcattaattagacatcaaggtcatatctatattcaaacaattctctatctacttgttcgaaacctttctttttatcttttataatatagttaccgcgtacaactttctcaatacctgaaggtgttttaataaacaggctattgtctccttgtttatctactgctctaaatgtttgaccttctgtccactgattaataacatgcttattatatcctgtatattgaatatactctactacgtcttccatatcgattatgacttttagcatagactctcttactttagcataatcatctgtaggggtatctaagtctaatggttttaattttgctatattgcttatattacattctcttacaggtgtttttgaatattctaccattaaatctagtaattcttcacttgcacctagactcctaaaagaatattgttcgttgagaaacacccaagcaataggttgttggttttcttttttaataagtattttttcttttcttttggtagtatttttatttgctatggtaacaatataacctaactgttttacattttcaataaattcattggtatacataggtggtacttcctttgtttaattattatccactatgtctatttcatggattgtaatcatgtcatctctatactcatctatatgcttaaggtaactgacgccatcatgttttctagacattccttgtaattctctatatccttcggctttaatatctttaacaagctgttctttgttagtgtatactttatcttctctatatgagtagctatcctcataaggctcacaatagtcatgttctacttgatatagtttcatattatttttactcctttttaattttatcttctaattctggtaatacattttttgaaatatatttatttaagtcatttattgttttaactctataagtacgcatgtttctgttactaatatataaattatataaatactctaccttactttcaatataatagaaaggatagcttatacaactcattaatagagatatactatacagtatattttcaggtaattctctagtaaattttttaattttatttacccttttttctcttcttaattttttatatagctttcttaggttgtgctcatgttttttaattacaagtagttgattttggttaagtaagctatcgtcgtttttgtttatatagtttaatattcctttatataaagttagtaattctctatcatccatattaatattatctccttatgttctacatttaaccagactaaagtataaatgacttgaggctctatatctcatttattctatgtccttcttttactgattggttccatgattttagtttttcaaagaacttggagtctactttaccttctaatttttggttagtataatcatcatagaaactaaatgttccgcctttaaaaaagcaataatacgtgtctgtcgtatagttaatattaatgtactctatatagtcttttgttacttctggtttgtcataacgtaagactggtatttcttgacgtaacaattttattccttctttacttaaacctgtatatctttttagcttgaaaccgaacctcaaccaaaagtctatttcattgaggttaaaaggttcatttacttttttacctaaaatactatgcataaatagcatataaacaagttctatatactctatacatgttgttaaaaaatgttgtttgtcttcaaagtgatggtaagccaacaaatacaaaaacgtaaaagggaaaagaacaatggacattaaaaaatataaaaatgattttaatcttgtgttcattactcagacaccttctctacatcatatagtattgggtatagattgaagatatgaactctatatccaatagttttaacttttactttatctcctacttttaatctagcttgtatgtctgcgctatcaaactttcttttgaagaataagtctgagttttcaatgacttgtttgttgtctaatacaatatagaacttgtcttctttatcttgtcttttgttatatttatctgtaattgttccttggtgcgtttctttgtgttgatagctagccactgtatagataggtaaagtgataacaagtagcaatacggttataccgaataatgacagtattccaaaaataaagatatcgaaccaattcatatttttaagttttttaatcattgttacagcaatctcccatcttcccaaattagtcttaacttactttgatgcataatatagaagcgctcagatttactagatttaacttcatttattgacatattatgatgagtgagtatattttcgttatctagatactcgtatagcttagtaaataccatgtcttcggtatagtcaataactgatagctctttgattaaaacatggtcttccttataatcatttatatttttgtaatacccatatccccttactgcatcccatacctttcgtacccttggtctaaaagtgagtttactacttgttctttatccctatagacttttgtttctgtatatgtttcttgatattcacgaggtttacaattatcatgttctagttgatacagtttcatagctaccccatcctttccaataagtatggattttcatatatatttcctttaacttgtaactctaaagtatctgtgtatggttttatggagaaatcactaagtaaagtaagtttacaccctgcatacctcactatatagaaaaatcccttacccttcttaactaaacctaggtcactataattacctttggtatttagtatttctataatatctccttcatatatatcattaccgtttacatctttaacttctgtgctttgcataatttcaaggttttctcctaaataaccttcacaagcaaatagatgtgaatatactgtgtaatctgcaaaatcaaatctatcaatattaaacattctttgtgattcttcatcccatgctctaaatttcaacatcatattacctccactttttcgatttctatgcttacagttttgaataggggctttttacgagtcagttttaataccgtattcgtgtcttcttcctcattcgtactttgcacaaaataatgcttttttaatttataattacatttagacgctaagaacttgatacaaagacttactttataggtttgcatcatactatcaactccccatctttccagattaacgtcatggttaggtcatcgtttaagatgtagaatgctttggtagggaaagacgtgttctctaaacgttctttgatactggtatttgtgtgcagcgctgacatataggctctttctcgaaactcatatacttcaaacaacctatcaaacttagtatcttctgtgatttcctcttcaactttgacttcgaaaagagtatcgatagaaataaaacctcctagagtaaaacatttatttgtatctttttgaaaacataccgcttcataactttcacctgttgtacaatattttttgccttgtgatagctctggattttctctagcccatttaattaactcgtctaatggcatttcttttttagtttttattttcatttttatatctcctaattatttaatttttctatcattttcataatcttttttgattctctaatgtcagcattttcacagttttttaatataaagttaattttgttttttacatcattaacgatactgtctacttctttttttgttggctcggtagggaaaagcaaccctacaactatctgaatttgatttttatttagtgagtaaacactaatgttacatttctcattagagtcaatatgtggtttacttgaattaaatacctcatctacaattgtatttaattcttggataggtttaaaataatgttgttttctttgtatcattacctgtgacattagtttatatagtttctgttctttatcaagcatttcttttgtaagtcttttaacttcccctacctttatagtattacctcgtgcatcataattatttttaatatactcagcttcttctatgttagtagtatatcctattacttctgaggtgggttcctcatcgtagtaacagtcctcttctacatatatagcatatatcttattcatatctatcaaacctttctttaagttaaattaactataacatataagcattagggtgtcaactactaatatacactttttacagtaaacaaaaaaaagaacctttttacaggttctttactaggttattaattttttaaatcttgatttccttttattagcttcttatcatcaataggatgaccattgtataaatattttacttccatatatacatgattatccgtagccgtaagggcatctaggatatccggatttgtgttaattgcatttgtatcaggaaacgaaaattttcttttatcagactggattgtatctacatgaaatttttcttgttttggtaaagagtaattaaggtcttctaaattttgtaggaatttagtaagagaaaagggtttgaactttccttgtaaattattatctatataatcatttaatagtcttgtcatttcctctttatttaaagtcattgtttcataatcgttaacatcataaaaaacaacaatattaggttgctcaatacttgtatctccatctttgtacttagcaatgtaatagcctatttcacctttatgagctaaataatcttctttcatgatattaaatacttctctagcaataggttttgtatatagttcttcccattcatttgtttctgtgttgtaaaaatgtggtaataagtttgccattatagtttatcctttcttattccgatttaagccatattttgttagtgtctttaggtggttcattgctgattgtgtacccgtcggtacccatatctaatgtgtttacaatgacaggctgagctacagcttctttttcactctctaatgttttgaaactaggcacatctgttttattagattcttgtccatcgactaaccaagagactttaaagtcacctttattataagtagtattaggttttaaatctgttaatgttacttttactgtgctacctgtaacttcatcagaagttaaaataagctgttcctctttgttgtagacctttaattttttagtcataatgttatccctttctgttgtgtaatacactaataatatagcatataaaaaaacaggtaagggctttcaaaacctttacctgcttattctttactgataatcataccaatcaagaaaatcatctgaacagcttaactctatagttatatcatttataagttcagtagcttcatctttacttaatgactctatatcaaaagtaaactcatcatataaacaatctatatgcttataaataaaatctttttgtttttccgtagctttgtttaattcattgctttttttacttataaattgactaaagttatcactcatttaaatcttcctctttaataaaggtaccattaattgttttacctttacgtcctttaatttcaccataagcaaactctaaacactcttgtaatgacatgtcgtgttgctgagctaaaataattaaagtaactacagtgtccccaataccatctttaagagcttctaggttaccacgtgataatgccgatgctacttcacctgcttcttcgtagaatttaagagcttgtctatcagggttgttattgttcaatcctttatctttactccattcctctacttggctaactaaaccttctagcgtgcggtcttctgtttcaccttcattaaagttaggtttttcatcatttggtacatgttcttcttgttctctatcctctattgtaataaataaagtgtctatttctttttcgtacttatttctctgcatataattatactgaccatactcaggataaggtacatatacagtatcgtctgtaccatcctcataattcaaacaaacacaaacaatatctttgccacttaatctatcatataatgtcatgttgtagggctcatctaagaaaccatgaaactcttcaaagtattcatccttcataccacttacctcaatagaaactttttttgctgatttacttgtatctacttcactatttccatatatcatagttttttcttcactaataccttctagtatcaactctacattttctggttctaacattgcgctatctaagttttcaaaaacaatttcaatatttttaatttttttagtcattaataatcattccttttttttagtatatagtaaatataacacataagtaaatagaagtcaataaaaaaaaacacctactttaaaaataagtaggtgtaagatattttgaaggtagtttgtaggaaaaaatcctgagagaggactcgaacctcaacgactaggtagctacaactagccaataccattactcaggattgctagtaacgataaatagagttataacgttaccgtagaccttttctacgcttgttagataggtagaatataatgattttacagtactcatatagttaggctgttactctcattataaggttaaaaaatgctaactatgtttcgcattgttaagaggcgttagttaactactataataatatagcatatattttattgtttgtcaagttctttattccatttacttttttcttttagtaatccttttttaattaattcttcactaatacttttatactctttattttctttatagaacactttagctaaatatctaccaaaagaatcatctttgtatgtttgaattaaaacatcttttccttctaacatagttgttgtaaattccttagcttcttggtaattattttgtttcttttctggtgtgtcgacacctagtaatcttattctttgctcagaatatgttctcattccgtggtcaatagttacatgtatagtatctccatccactactttgtccacgtgtgctttaaataaatataaattgtcttctatcattattaaatcccctttttactaatttaaaaatatccaatcagtattgcttgatgtacctttagctatatatacagatgagcctactacagcaatttgacctttatataaaggtgtgttgttaatactttctgttactcctatttgacttacaacttcaaaattagcttttgtagctataaattcccaaaatatagaaccatctgatataacaccgttattatgagtaggttgtgttccaccacttgtacctgaagctactgctttatatacataattcccattataaacaatttgaccttgtgtataatccgtgttagatgtccacttcatattagtagcaactccatcgctaatacatctccatttctcaggactacctgatgttaaggttctactagtattaacaacaatatctcccaatctccaacttcctgtcgttggtattgcagtaccgttcagtactttaggtaaattataagctatgttatccttacttataacattaaatgcattactagaaatacttaagtttttattcatattgttatttaatacgaataaattactacaagaaccttgaacacttaatgaatcattacctttatctgtttttatatcattattagtaattgtactatccataacaaaatttaagaacatagctttatccaaagaagtatttgtacttcttaatgtattatttttaaagttaatttctgacacaggtttactagttgttcctgaggcataaataatacttgtttttaaacttgttgtatcttgttttaatacattattttgtatatctatgttattaactcctggataaaggtacaactcgtaaccctcattacctgatacattattgttactaataattcctttactacctcctgtaatagcaataccattagatggtttattacttaactctcctttattactattgccataaattacgttattagtaattgtatagttagtacctgatatactcagtcctgagtacttattgtttgtatgtatgttattacttataataacattttctgttcctgattcaaaaccgcttgaaccatttcctgtagatatattattaacataatatccatttttagtaccgcccataaagttaaaaccattctctaaacattgtgtagctgagcaactgtctatagtaatgttatctgagtaaccataaacaccatctaccgcttgcatagtgtttactgcatttacaacgttacccataatattataaccatgtacattatatgctttacagtttctacttcctttaaatgctattccaatagaacgaggtgacgcatatctaaattcacctttaccatcccattcccctttaaatgttaatccttctacaagaatgttatcacagttgataaatgagaatcctgtaaacatatctctaccactagaagaaacatcatctatagagtctagatagtttttagtgattcctgacataataaatgtaggattaccttcacctttaatgtatatattacttcctgttacttttacacgtgatgggttttgtacagtaggtgttttagttttgaataaatagtcctctgttcctttaggtactataagagttccacccttagtacttattaaataatctaaagctctttggaatctagggtaatcattatcttcatcattaattcttgggtaatcttctatattaactattactttactcttagaattagtatctttacctagttctttccaatcattaccttttagtacccaaccatatgcttgtgttgtaccattgttgtataccttttcatcatatttaaaatcttcaacactagaaggtttaacatgagaataaaaaacatcagttttattcttgctactatcgtttgaagacttaataatattagatggattagttgaagctacaagtaaaccttgcatagttccagatattaaaataacgatagacttaacatcttcactattaacagtaaaagtatctttttgggtgttattgaaagtgaacgctttatatttatcgttataataaccatccagtaatattgtatcataattattagatattttgtctaatatattaccgtctttatcaaaacatttaataaatataccactagaattacctataaatgataacttgttaataattaactcgtcaccaacactaacattattaacatataaagccatagggtacgagcctgtaaaggtaagtaatcctttattatctactaaactaggtgtagtactgtagtcataagatacttcatctgttagagaagttgactgtaatggttttctaaatacatttttgactgtatgatagttacctcctaatcttctatagtctttgctaaaatcattatttttatacaataacgttgttttgtattcgtctactattttagcaatactacttctaggtattttagcagtattaatatttgtaaacgttagtttatgtccattacctaaaacaaattcaggaataaacttaatatctttactatgtgttacataagattttgtcatatcttgagtttctaaatcaacaaccgcaaaagtagctttgttattaaattcaaatctgtaattcttaaatataaatcctactgctttcttaagataaaccataacataattttcagatgttgtggaagcagtttcaaatttaagtgaatcgaatacattgctatttcctccataggtattaccatttgttagagtttgtttgatgttatagtatgtgttagggtgtttattaaactcagtgccactatatgaaaaagctgatttatagaagtaattactattcatccaagattgctcaccattagaacctataccatctgtatttaattctatatgtgtttcattattaataaagaaattaataatagtattattaaaccaatgacctctaccaccagtagctttatgtttattacctactgtaaaaccaataatttcattaatattagtattaacatttctacagttaactaattctagtcctatatagttttgactcttaaaaccatgatagcctccaccataagttggataagaactatcatcataaatacctttaattgtaacattagaatatgataaactatctagtttaatagctggtttatccttactaccattataaacaatcttagcatcagatacaatattaatattcttatctattatcaaagtatcatctattttaataggtgattcactagaaggaatgtgtagggtattaccttcacttgtatctttaatagcacgtcttaacctttgaacatccgaagtatcatctttttgctttatatactcgttagagtatacataaggacttttacctgttttaagccaatctgtagctgaacctgtaaaaccgttatttacagctacttcataagcacttaaaccttgcttaccttgttcccctttctcccctttttctcctttaatagacttaacccattcttcttcagaaccgacaaaaccattatctaccgctacatcataagcagatttagcatttacaagtatatggtctttaaaatataatgttatttctttgaatgatgattctagggttgtaaaaccaggaactactactttatcggtttcatatttttcagcttcccatgatacatagaactcaccttgagggtaatgggtatgaggagctaagttaggtatgattacagaccctacttcttcatgtaactctacattgtaacctattgccaatatattaccgtatttatcatatgcttttaaatgaggcattgaaaatctcctttattattagtcaatgttaatatatcataaaacacaaaaaagggcaaattgccctttagttacagatatcatttagatgaatatggtcttgtactgaatagtctacaatgacatgtgtatgctttttcttttttgttaacactttatatgtgataaagaatgatataatagttaaacatatatttaattttttcataaaatttctcctttaaagaaggtatttaccgtctcagtagttaattctttgcttttaaagatacaagattctgtatttttattcaatatttctaacatattttgcatactacttgctttttgtaaatccgttaagtctataacaactgttatttctgcgttatctattagaatattacctatgtaataatttttaactccgttctccgtagcatgagaggtaacattatgatttaaaacaactataccttttgatggtaagtaatctgaaaagtataggtaacaaaagtatttatgtatcttttttttatcctcttccattactattaccatattctatgaagtttaacaacctttcggtataggttttaattttctctacttcttttctttcatcgtcttttcttcctaatcttgttgtatatttaacaatattaaatagcatgcagtctataaagccaataatgtagaaatattcttctaaaaatgatataacatcttttttacctttataatggttcggtgtattagtggtagagttgacattatagtataactccagttgttctacatttttcataagctttttagcataatataatgtgtcatctaaagaatgtgtcacatctaaagatgcttcaacaatgtttatctttaattcttttaattctgttgcctctttgttacttccaatttgtgaatcaataaattctctagtatccatattggtgtcctttcgtatctaaaataaaaaccctatctagaatatagatagggaaaaggaaagatattatggaagtgtactataaagtacacatataatataacagttcctttgccttttaatctatatcatttattttaacttcttgtactttaacataatctgataaactattaacgacgatattatcttctactaattcgtttctattatttgtaacatcatatgagtctctataaggtgtttgtactaatgctctaactgtaatgtctatatctacatattctttactatacctttccattactcaacgtagtttcctttagcaagctgatactttttgtgtaattcttcatataatttctccatatctttatattttcgtacatcttcttcaaaatgacttaaatactctattaatgtcttaacttctttaggttctaaagttataactggttttaccattacgcaacatcctcactaaattgtttatttatatttttaatatcttttattaattcgtcttttaattctttaggtaactcatgactttttaaagattgttgttgacctaacgtatatccatattttgttatccttacttctacagtataccatgatttttctaaatcgtcaacttttctagctagtaatattaaacatctgtcatttataatattcgaagcataactgccgacacaatgagatagctgttcaccttctctttttaatttttcagtagtatccgcaggtaaaaatttaactttagagccatcgtttaacttatattctttatttgtgatattatttaatttattatcatatttttgttgtaactttaagtcatcgacttcatctttaactgttaaatactcatcagtaacaatatcatgctctagttttaaagaatatggtgttaagttaatactttctttagttctatatccaagaagtactaggtctgaagcaaagtctatataatctctatataaattattaacagcgtatgaatgtatacgttgtctgtttctgacatcatagtttatataattcaccatttttctaaagtcagaaaatgaaaagttaagaacttctttcgtagtacctttaaaagataaagcatttcctattgaatatgaattagtcaccattataaagtcgtcatcgaatagtttgtgtaagttagactcattatataaactaggaaatttatttatgataactcgaagtatattaaggtaattttcttgctcttctttactaagagtaataaacttattatatgtttgtttacttaaatttaaagcttcatgtaatttccatttatttttgtttggtattactagaaactctttgtgttctttaaatatatcacaatcttcgttgttataaactttatcaatattagacacaactaattttaacagattgtttaaattatcagtagcattttcacctttactaactgtgcgataaccatcaggtctatctatcatagagtatacatcaaagttttttacattaagtctacttagatttaagtaataatcattattctttagtgtttggattattatgttaaaagctaaattttcctttccatatttacttaaaataatatttaaaaggacaggtaaagtaatgttattttttagtttacccttattaaaatcatcttcatacatattaaatttaaattctttatgttgtagtgtatttgataggtgtaggtatacatttccatttttattattaaagtcaccgtttactatactccaatctgttgttactatacttatgtcttttaaataacaagtcttaattaaaaacggagtattattatcttttataggtgttaatttgttaagccctatttttatattttttaaaacttcattgtagtattcgtagttattatcataagatttaccaaatgcataagggtcaatattaataccaaatatactatagtcacccattaataatacatcaagtatatcattagttgtagtatttatttgtctttttaaatctcttattttttttgtaaggtagttattactgctcttattttgcatactgtcaaattgttttaattcattttcatagtttgcacggcagtatatcttattacgatagtgtcttttgttatataaaggaaattcagacctatcgtcactttctatatactcactatatacagtatttacatctgtaactgttttacttaactcttgtacttttttatatgggtctcctttaaacgtagtaagttttttattcccacataatgctataaataaaagcaaatttaaagcactagaataacaaaaggtattattaatatctcttaataattctttagagccaatatctacaatatttttattatttatttttaaatatactctttcttcacctataaatatactatttctatttactttagataatttaagaatttcttcttcagttaatcctgtttctttatattctactgacatgtgtcatcacctcatttattattatataagtatgataacatagttaaacttaatagtcaacaaaaaagaagaacttttttacagttcttctagataagtttcatatttaatttttttagttcttcttacgggtacaatatcgggattactcattaagtctttgtacttcttagaatttttaaagttaatagtattaggcattgctaatacttcctctaactcatctgtgtacatagtaactttagatattatatctatgtcactaatatataaacttttagatttacttttaaaaataatctcatcggtactagttacttctttattatctattaaatccatatagaagtcctttctagtctacataaacaagtatacaatctttgtctgataaagacaccgaggtcatatcattaagttcttgtagttgtttaatagacacatcatatttcttacttatagtccataaggtatcaccatttctaaccttataataaaatttattacctttttgtattaagtctttcattgctatctacgcctttatattcataataagatgcctgtaggtaacatatcataataagaataaagttagctacttctgtatatattacatgtggtgtcgtatttgttattaacatactaataattaaacaagctaatgcaatacctataataagaaataaatatttgctagttccatttgtacttttagttttataaaatttatttatttgggataaataagctacgataatactaatagtagccattatctgtccatattcaacactatagttagctactgaatagccaatataaataacaattataaacataaccatttctaaaagactaacttctgttgttctaaataagtatataataaaacaaataatacctagtaataaattaatacttactgatataatttgaaatagaggtgcatgtgttacaaataagcttgttaagctaatacttactgtaacaacaatataaaaccaaaaatagctatctacacctttaacattcttatctcttatcaaacctactaaagcaggtatatatcctacagtaactaatatagcatataggatgcttaatatatgagctaaattagacattagatgcacctaacttttctaattcccttaaaacttcttcccatgaaataaaacctctaccgttggttagtgataaaaccattccatagacaaactggttcgttgaaaactccgctctgtctgggttagtgtatggcttcccgtgcccttgacgaatatcagagcaataaattaaaacaggtttattgacgatatgatacgcttctaatatgtcatcttgtgtgtttttatctaactccctaaaatctatatcttcatatttattgataatttgttgagcttggtgtttcatacctaataaaatactaagttctgctattgtacctagcccctcattcagaacatcaaacacgaatatatctgactcttgcatagccttaaagtcattatttaggatacgctcagccaaaccctcctgtctagcatttgctttgtcgttaattgagccatcttgatgaggagagtatggagatacacctacaattccttctacttgattgtgttgtttttctctatattctatcattgcttgattaagtagatgacctcccatatagcaaatcttatctttgattttatttaccattgtttttatctcctttttatttatttaaaagttactttacccattttcaatacttgtattctcttatttctctgattgtagttctatttacaccatacttttttgctaattctgtatgacttaaatcactcgctctcacttcttttattttttcttcatttaacttagcaacagggttttttcttttaccacttatataagaattaagtttgtgcgtagaagttaaataaagggtaaaatacctttccatcaacatttttaatatttataggctcttgatttctattaacaaaaccatatttatttttcttcttgtatgctgagtaacaaccacaacttctaatagttttatttttatactggtcttttcttacctctactatattaccacacttacataagcatttaacatatttacgctttctatttttacattgtttaaatagtgtttttattgggattaaacatgtttcttgtaattccatcatacaactccctcatatcttataatttctccttttctttttcaaactcatcttttagtgtgatgatatctttattcagttcttctgagtctttcattctttttttactaacacatatttcatcaaatattttggtcattttttattttccttctcttggtttcatttgccataatttattttactttataaaactgtttttaatttatctttttcttctttttttcttcctaagtattcatcgttagctttttgctctcgttttctatgtacattcaaataatgttccattgtcttgataaaaccttttatatttacctaggtctctagttgttactattactttatcagctctgtcgtatttagtattacggaatccttgttcatatatttttacgtctattgattctttagtgtaagttgtgctaaaagcatcatcaaaataaacagttacatataacttgttgttttttaaactcaaatcttatatctttatttcgtcactgttaaaagtgtcgaacattatatttatttcattttttttaattacatataaatcattaatagtgtagtttttgcagtacttccctgtcatcttattttctttgtattttttaaaaatatttttaaatataattagtaccttcctttattttaattatcaagtactcttaagtcacttttaagaacatccatgctattgctatgtttacctataccatctagattcccatcttttaacttattatctttcatgatttcttttttaatttgtttttctgtctttttatttgtcatagtgttttcaatatcaaaagacatctcttcaataataagtgttttaataaaatcaataaattccttaattgtatatctactaaaataatctttatcctcaaagtacgtagcacaaaaacttttacgtcttgttagtacacctattgacttattggaagtagcaatataaacatgatatgtttttccttttacttttattttaaatcttgtattatctaacaaatggttatagtctttaaataagtaattgttcaaaattagccctcctttgcgttaacccaaatttatagtattttattgtagttttctctccgctgtagtaaaggtgtcattattccaaccaccatcatgattaataaactctaaatcacctgtcacagtacataagacatcaaaactaatattttctcttctaccttttgaaatacctgtgtaaactattttaggtatacaactaagggtctgtaatcctaaaccatctatatattctctaaaagatacatgaggtaataaataagcactcaattcactactgatacttctactaaaataaatgataatgtctccaacaggtagttcatcattatacacatctacgatatcatcttcttcaatagtccacgttgtatgttctatatctgcaggtgttacatgaacaagttgtttaacttttccttgtataatagactccttataaaaacctacagcatttgctttttctaaatactgtgcaataaaagtaccatcatcactaattaaatacggtgttctattttgaaaatctatagattgttccatatctagtttttcgtaggtatcctttaagtactcactaattttattaacatcttttgttttaactttatttgttcctaccttaacttttaattctatcattattttagtctccttataagtactttctaactggttccgagtaatcattaatatcctttactcttggatactttaaaatactaaaagcaataatacctagagctacaataggtgctaataaaaccaagccccagaatgttaatactgtttgtaagaaaaacattacactatctactaaaaaccatgtagctactactagtaatgttgtaactgtaaataaaaataacttttcccaaaatccacctttttgaaacttttttgataattttttcatcataatatttctccttatattccaattgctttttttgctaatgcatctacatattcattccatttgttattatcatgagcctttacttttataaaatttatatctataaactttgagtattcccttagcatattaatatatgttttacttaaattgtttttagttttccacgaaccttcataccattgaataagtcctatataatctacatgaataatagcctgcttataacctagttttatagcctcttcaataccataacaacaagctagtatttcacctgcaacattattatacttaattaaactactattttcaataggtttactgatttcttttacaatgttctcatttttatctaaaataatagctcctgctccaacaagacctttattataagatgagctaccatctgtatatattaatgcagtcttatgcatatttatgcccttctacaaagcaagggaacatctcacttgtataaatctctttgtagtcttgtaattttatagtagagaaatcaaaaaacttataacaatgttcttctgcttcgatatagtcatctgcaataacattaacttcaaaaccatcaactaaaataacgaacaatttcttaaacatttaaaaaacccctttataatttaataaggtacttaattaaatcaatttcagtgtacttttctgagtgttctagctcttctaatttttcttttacttcttcaagtctgtatgactcttctaaaagttcaaactgctctaataatgtgatactatcaaattcactactattaatgatgttgtgtatatattctagatgtgtgtctttaataatgtttgttaagtcatcaccagtataataatcctcgatatacgataggtctcctaataactcagccgtacttctaataatactatatttagatatattagggtaagattgatataaacaattatctaatttattacttaaatgctttactactttttgcattgtattacctttttcttcagtactatatgaaattctaatattagggtttttagaatcataatcaactaaagaactcctattagtgtttaataacttatctaaagaagttaaaataatagctctatcttctatattatctaaggttaagatagtatctataatagtataaaatgctttgtctcctattgctttattaacatcttgaagttcaaaaatattaatcatatcctcaatctcatgacgctcaacaaagttaataatagattttgtaaatttattcatcaacatcaacctctttaaataattctacactattagtatttaaagctttttctaatttcataagattagcactcttaggttttcttttaccactctcccaataagaaataagtgagtagtgaactccaatctcttgtgctaaacttctaactgtatgacctttttcttctcttactttttttaaatttaatactttagtctcttccataataaaccactacctatctttcttcaatttttaaaatactatagttatttttattataagcatacttatttaaaacgtcaagcgttttttctttagcctctactttacctttagctttcatattaacatagaaaacattacttttatttgaacgtgtgtgcttaatatatactttgtactgcgtaacctcactttttaatacattacgtttaaaccatttctttaatttttccatttattataaccacctttaaaacttatatatgtattgtagcatatgataaatacattgtcaacttaaaaataaaaaaaatactagggaaaattccctagtacattatttccagttaatacgaccccaatacttctcttttagcatacgttcccacttgtcagtgattttacatactgccatatagaagttaccagctgacgggtttgtaggatatttaaacctaatccaccagtagccgtctgctttaataacttggtcaaagtttacatattgaccactataaataaatgattcagcacctactattgaaccccttaaacctggtgtgcgtcttaccttaatcgttctatcagcagtaaatttacctccccaattccaagttgtttgtacctcgtctttgaaattaggttctataaaccacataggaaagtcgtaaccatgggctctaacggtagctttctcccagccaccgcctccttgagcatctccccaagtccaacctccacctaaccaattattttcaattactataatttggttttggtttgcacttagtacccatgcgacatgaccatgaccgcatccgtatgaattattaaagataaccatagtgcctggtttaggaatgtaggatagtgtattttctactactctagctttatcttttaacatagctttattatcccatggaatgttacatgcacttgctccacctaaagaataacctgggaagagcttgttaaatccatagttagctaagtcgtaacattgctgaccataccaaccatcaaagtctattgctttgccttctagactttttaaaaatcgaacaaattcacttctagtcatcgatgctttcaaaagttttcactccttatttccaatctatttttccaaaatatttttctttcttaatacgctcttgtttatctgttatctcacatacagcacaatagaaataatttttactagagcctggttttggatatttaaatcgaatccaccaatagccatctgctttaataacttggtcaaactcaacccaatcatccttatcgtatagccatgaatctctatttactatagtgcctttaagtcctggagattttctaactttaatagctctatctggataaaaagtacctttccagctccacatagttttagtagattgtgtttttactctaggctcagcattacctgatttctttacatctactgacttaataactttcttgtaatacttagctacttccttatctaagttactagtgtttctacctaatccacatgcttctaataagttacccgggtcttgtttgtcgttttggatgtcttggtgacctggcatatttgttttgtaatcaacaccccagtattcagctaaataagctaaaacacgagctccattgtctaatgattttttagaacgttctttatcactaaagtaacaaatttctacaccaatagcaccatcattagcgttaattccgtaccataggtcatccataggagcgttatacaacacatgccaagctttttctgtagtaggaacacatatgatagcttctttatcatctacaaagatatgtgcggatgcagtagaagcccaatcaatgttatatgtatttttgtaataatctacattattttgagctgtagtattaatattccctgtatcatgagctacaataaaaacgggttttttaaccgccatagcttgacctgtgcgtcttgttccaataggcagtaaatcatatctaacacttacacctttccaaatttctatcattactgttcacctacttcttttgggttatcttctttatctaattcagacactaagtctttttctactttaccatgttctaatggttctgcatgacttgtatctatgtcttcattattaactccttcaggagccatcggcatctttgtaggaggtaactctgaggtactattcattgcttcactcttgtatgttaaagctttatctgggtcattataatctctaggtttgttaaagtcctgtacaatagggctatccgctatacctttagtgtcatgacttacaagaacacctaaaccagtcaagaaactaataactacacctgcaattgcagtgccttggtttacaatatcttctgcacctttgattcctaataaactacatacactgacaataaacgtagctaaaacaggtacaaaacctgtccaaaatgtagcgctttttgcttttgcagtaatattgttacctgctattaataatggttttttatctttttttgacataaaaaaataccatcctttctgttattgtaaagctaataataatataacaaaaagaatggttttagttattaaattttaagttattgttcgttaatttgagaaagtgttttgattacttctgacatttgtttattatctaagtcttctaacttagtgtgtggaagtaaatctaatttatcccaaatatcttttttagttgtgaatttttcattaataattgctctaccgattaaactttcagtataataaatttgttttgcagatgccatattagtatctacccttctatagtataattaaacatacatacataaaccgtctcactggtaagtaacatactagacctattgttaagttaatcactacgcttgactctattaggtagctaacctaagtccatctcacgtaacctattaaagcagtacaaccgtagggttgattctgtttaatattgtaatgtgatactaacatattaattagtaactttcacatggtatgaagaattttagcggtattctataatttgaccaattattattttgcaggtttatgctacttgtactagtatcgtatgttatcaaacatttttcaatgctccaactgcatatacttgttaaggtgtatttgtaaaggattccaccaagaccctttacaaaaattaagcccatcagatactatactttataaacctaatatgttactgctgagtattccgattgtaataagcattactattgacgaattatctcaccgatataaaccgacaagttttaattcgttcgacaatctcacgccttacaggtaccaccctgtttaaatcagccattatgtatgtatgtttaataatactacttttaaagtataacatatatttattcggttgtcaagaaaaatttataactttttatagtttttttatactagggtaaaaaagttttaaaagacctagtttcgacaaataaaaataaggagaaataacacctcaatttgacaacaaattaatctctatgcaagtttaactataatataacataatattctaagtttgtcaagttcttttttacttttttatcttgataattcttcttgtattttatcccaatatttttctgattctgttctattatcctcaatgtgagcagtggtacatttacatctgatacaatgtaaatgttttatatgtccttcttctcttctttgagctctttttcttggtactgtaaaagtattaccacattctgaacaaactaaattactgtggaacattttctgtctttttttcatgattatcaatccttttaaaattttttgtatagctagtatatcatataattattttgatgtcaatacaaatactgttgttttgataaaagtttattactagttatatttaattattctgaatatttagatactttacctatttggaaaataaggaggactccttatatatataataaataattataaatactaaaataataatactagttataataattaataatatatataggaaagtcaacaaagagtaataactggttcttgataaactattcaatattcttacttatatttctgagtaaaaaataagaaatccttttatatcaagggttttaaagatttttttacctatttggaaaataaggaggactccttatatatataataaataattataaatactaaaataataatactagttataataattaataatatatataggaaagtcaacaaagagtaataaaaaagtgacttacttaaaagtcactaattttcaaatactaacttaaatgattctatgaataaagggttaatgaatactgtatcagaattaatatcctcagagactttaactctaactgatgcttgtccatgtcctctaattgttccatttatttctctttgtgtatactcaatactatcatcagagattaaataatatttttttccactaatcattgttacctcagcatgttttaccatatcaaaactcctaactaaatttattgaaaatgaaagttataatagaacctaacactagtagtagtactttctcggtagtatccttctttcttatgtccttaacaactgtattgttagtgagttcttttatcttttcgtctaatctatctagctgtatgtagatagacgattgtttctcactattaacagctacttctttgtctaaactaactaccatttttcttagttcatcaacttcattttctaattccttatacgtatttttatcaatatagctaccatcttgtattttatcttttatcttatctacttgtccgacaaggctttttagttcttcattcaactaaagtcacctcttaatgtctaccttttcagattctgaatggacgtcatagttttcgaaaaagtcattaataatttgcatataatcctcagtaactatctcaccatatgatgttttagtatttatttcaaacctaagcttaccaaactcatggaggtctatttctttaataacggtattaggattatcagttggtatgtaataataatcaaaatatataatagagccgttgataagcaacctatcaatcctataaacgtggaagaattttctatctcgtctaaagtgagctaaggcatctttaaactctaattttaatatatctttatacttagtcaaatcaacttacccccttataattaattttgagctacactagttttgtgttataatagttattgtagtagcagatatgagtattacgtttaataaaattagcaaccaccaactacctgtacttattaaccataatgaaaaacaagataatatataaacaaccaccaagtagcctatatatttcataatgtttttacactcttttaatactactacgtatgctagtgatttagataagctaaacagaaaataaaaataaaaaataaaaagaggtatagctataatgcctgttagtataaccactaattacacctaatttcttttagctagataataaaatcttatataatacaactttaatataacattgaaacctgtttataaatcaacaatgttacattaatagtgtacggtatattttaaattaggaggactgaactaatagggtgaataataacatagctgtatttatatttaaaactctaataattattatttttattttgtttattttgtctattattaactcactgtcattaatatactctattcgacctagtgcagttatgacttactttatattcggtgggatagtttctaatgtggcattaacaataactgataaattattattacacaaagaagaccccttacctgattatattctaaaagaagtagagctaaataacaatgaattacaagtactaaagaaaattgtagagtcacaaactaatgtttctgctgaggaagtaagagtaagagcaaaagcacaacgtcgaatactaaaagataaaaagagagatgctgtcaatgaaaacgaagaaggacattaaagaacaacgtaaagagctgaaagataatgctactgctgtttccttaacaaaaaaaggagataaacgtattgcaagtcctagtagagtgtgttgtttatgtggtcaatctctttcagggatgaattatactaaaggtaaagctatagctaaagttaatcattttcatttacagtactctaagtacttatattttgacatgtgcgcagatattaataactgttacaagaatttaaagagagtaggtgaattgatagagtgagcgctgaaagtttaagagatatgttatctaagaaaaaaattgaagacgaagataaacgtaaatatatagctgatggatttatgtcaggtattactaaacttatgtatgactttaataaaaagattgatagaggggaaatagaagtaaaagaccctaatgatttgtataaagtgtttgttatcttccaacaaatgcaaaacttaattacagatggttctgatggtggcggtgctataccgcaattatcaagaccacaacaagagctctttgatgaaataacaaatgagaatagtaaaggtgagaaagaagtagatttagagaagttatctcaactatctgctgatgacattactgctatgattattgataaagaaaaggttatgaacgaagagaactcaaatacattctaaagaaaggtgtatatgaatggacggtaaagaattattaaaaataacacaagaaacattcggtacacaaagtgttacacaagaacaagttgaccatattatcaatatgttaaatcccagtacgtatatgttaaaatatcatacgttgagaggacaccccattacatttagtattcctaatagggatagaagtaaagcacaagcacatagaccttggcaggttaaaatcgtataatcatttgcgaccttacatagtaatatgtattgaataactctgttaaacgggcaaagctgaataatgaataagctggtaagagaacctaagtccaaacggatagaggcaatcccgtgctaaattcttaatttaattgttgactacctagactatgtagtttataattaaattaagataaatgcctaacgactaaatttctaggtagctatataaatggatacagtgagaactagataagaggtttctataaggctaaataaaagaaaggtaggtgtaataatgactagagtactatcacaaaaatatgtagataaaaacaaagataaattcataaaacgtttagaagatgagaaaaataatgagtaccaacttattggtgaatataaaacaatgagaacacctacaacatttagacataaatgtggatatgaatggactacacagccaaaaactctaatagatgctgaagggctattaggttgcccacattgtcaatacagaatgaaatcaaaaacaacagaacagtttaaaaaagacataagaattaaaacaaacggagaatatatactattagaaggacaaaaatataaaacaaatagagaaaaattaaaatttaaacacattaaatgtggtacggaattttacatgattgcaggcagtattttaagagaacaagagagttatccttactgtagaagcttaaaaccttccccaagtaaaagaagtaccaaatctttcaaaaaggaattattaagagagaaaggtaaagactacacactagtatcagaatatacacatgctttagagaaggtaaaaattaaacatacatgtggcaatacatggtgggttagaccttatcatattctgtatgacaatagttgtccttattgttatggctcaacaggtgaattattaatagctggtttattggcaaataaaggtatagattataaaagagaatatatttttaaagactgtaagcatattgggtatttaaggtttgattttatgtttattattaacaataatatcattcttattgagtatgacggagagcaacattttaaacctgttgaatactttggagggcaagaaacttttgaaaaaactaaagaaagagataaaattaaaaataagtattgtgatgacaaaggatatatgttactaagaataccttattattatagtaatgaggaagttaaaattaaaataaataactttatagaaaccgtaaagcagagaacccttaaccaaaactaaggaatatgatatagtctaaaccgattcttaaatgaatgttaaagtattacgaaagtaacggtagtaaatgaaatgataaccaccctaataaagctgtaataaagtcgagacaattagggctcagtgaaatgtcagtaatggaaatggttcattttgctgacatgtacagttataggaatgtaaaatgtttgtatacatttccaacacaggaacaacttaagaaatttgtacagtcacgtcttaatcctgtattagataaaacttattttagagatattgtgaattgggataaagactccttaggctataaacaaattagaaactcaagtttgttttttagaactagtagtaaaccaagttcagttgagggtgttgacatagacttcctcgcaatggacgaatttgagagggtgaactcactagccgaaagttcggctttagagtctatgtcaagttcaccttttaaagtagtaagaagattctcaactccgtcagcacctggggtaggtatacacaaattatatcaacaatctgaccaatggtactatgctcatgtgtgtcaacattgtcaatatgaaaacgaaatgaaatatgctgattatgaccctaatgacttaaataagagtggtaatcttttacttttaaacccagatggtattgatgaacaagctaaaacagtacaagaaggtacatatcaatacgtttgtcagaaatgtggaaaacccctagaccgttggtataatggcatttggaaagtaaggtatccagaaagaacaaaaaataattcaggtattagaggttactacatttcacagatgaatgctgtatggataagtgcaagccaattagtagagaaagaaatgaatacagattctaagcaagcattttataactattctttaggtatgccttatgaagatgttaaaatgcgtgttttaccagaagacatctttgacaataagtcagaaattgcaaaaacacagttatttaatagagataaatacaaatttatttcagtaggtatagactggggtaacttccactgggtaactgtacatggtatgactgaggagggtaaagtagatttaatacgtttattctctattaagaaaaacagtagaccagatatggtagaagcagatttagaagctataatattagagatatctaaatatgaccctgatattattgttgcagataatggtgattcgggtaacaacgtacttaaattaattaatttctttggtaaggataaagtatttggttgtacttataagtctagcccacgctcatcaggtcaattatatgcacaatttaatgaaaacagtaatacagtaactgtagataaattaatgcataataaacgatatatacaagatttaaaagctaaacgtataagaatgtaccaacaggtagatgatgaattaaaaacttatcttaagcactggcaaaatgtcctaatcatggacgaagaagatgaaaaaacaggagaactttatcaagttattaaacgtaaaggcgacgaccattattctcagtctgcctgtattggttacatcggtcttactcgtctaaaagaattatatgacaaaggcaacggtacagctttcacatctacttttataccaacggattataatagtacacaaaacaactcgtctaatgagttctattttgatgactaagaaaggactgacgacttgacaagtatattaaattatgatacaatacaagaagatgatataagtaaagagacatttaatttattaattgaagagcctttaactttatcttatgtttctaaacatagtgattctttagatagtaaatatgactacatatataaatcactaggctttgataacttcatggactgttatttatacatagcaagagaaccagaacgtatatacaaaggtggagctaaagacataggctcattgaataaagtaaaaagaactgtagtacgtaatgggaaagaaattgaaatgacagtctatgaagataataaaaatgatgataaacctaaagagaataatagtgaagaatcaaaagaacctaaaactgctaaaggttctagggttcttactcatggtgatgctgaaaaacctaatcctaaaaagattgctagttctctttctacattaaagaataaaggtgtaaatactgaccatatagatggtaatgcatcgatatataaagaatttgtagatgatagtaataatacaataggattagcttcttttaaagagacagattctgacattatattacatggatacgtatcagactttgaaactaatggtgtaggtgctaggtctatattagagcttatttctttagcaataaagaagaataagaacgctgtagtttatgacatagaactacctcaagctgtagagttcttaaaatctttagggtttatgaaaaataaagattactatgtattaaaaaaacaagatataaagttatttttaggtgagtacagtgagcttacttagcactgtactcatttatacaattatatatataagtattattatattagtagttgtaaatgtagtaataagagagaaaagaatgtacaatactaggaagttgcttatacaagtaaaaaaggatattataaacaacttaaaaaagggagatatgaatgatcaaagataataatacaaaaacattggaagagttaattacacctatttctacaaaagaaaaagaaaataaagttaaagaatttagtaaaacattatgtgagatgattaataagctatatagttcttacaatgtattaaggcaagaccctattgatgaaacacaaagattagacggttctctaatggtgtttcaaagtagattagatgatacattaacaggtgaggaacaccaaaaactttttaaattagcttttgattataatattgatatttttgaagctaacaagcagtttaaagaagatgtacaaaatggaaaagctattaggttaggtgatgttgttgtaaccgacacagctttaagtaatgttttatctagtaatgagcttaatgttagtattacatttatgttaagcaaagactatgaagaaaagaaacgtattgaagaagaagaaaaggaaaaactagataagttataatagatattatgagacctttatggataggtaggaataaataatatgaaacgtaagtttagtagtaatgagattactataacacttataactattgtagttgctctatttataatattaattacagtagcatttaacaagtaccaaattgctaaagaagataaggacagatatcaaaagctagtagaaatttatcaaaaagcagatgatgatgacggtaaaactaaaaaaaggtatgttgaaaagttaaatagggctgaagaagaacttaaaaaggttaaagaagaaactaattataaaggttataatgataaaacagaaaaagaaagagataaagaggataagagtgtaagagaacgtatttatggtaatgataaggataaagatttagtattagtaaataataaggttgttgtaagtaatgaagttactagacctaagataatagaagataatggtgtcagtacagttgtagtacctcctgttgtaagtcctgttgagccaccaagtgaacctagtataccttctcctactatacctaaacctattattcctaatcctattaaccctaaccctattctacctaaacctaacatccctacccctaacccactacctcctatagaacctgaaaaacctatagaacctgaaaaacctatagaacctgaaaaacctatagaacctgaaaaacctatagaacctgaaaaacctagctcagatactacgatacctttagttccattggaacctagcacacctgtagatgaaagtaatgatactaatggggagggataacctctcctttttttgctatattaaaagagtaataaaataaacattatagttggtgattttttgataggattaaacgtacttacagctatatccgtagtttttgcttgtgtaagtctactagcactaatggtatttgcgtatattaaaactgaaaaaagtagtaagttggttctatacttgatatatgctataatttctataggttcttatgtagtattaacaatgtttcaagccacgtcaatcattataaaaaatgatgttatagactctattgagaatacagagcagtatgtagggtttaataaccctttaattgtttttggtataagtttctttggagctgtaataggcagtttttggtttttagttatgaagattgtaaatactaggaatttaaaaaataaaaaattaaaggattgaagcatttgctattttcaaaagacgataaatgggatgaagcaaaagactttataaaaggtcaaggattagcagataattggatagaagtagtggattattatagacagataggtggtaaacatgtagctatgtttattgctattgataaagttaaatatatggtactagaagctacaaaggataataaagtaatattggtagataaggataataatattgtgttagaggattatgatattgttatggaaagtaagaaaatgttttactatatagaagaaccattcgaagtaaagataaatattcctaaacatatccaagacattacctataataacactgttatactaactactattcgaggaggtaaataagatagcaataatctataaaaccttataaatttatttgcatttttagataaagtatgataacgaaaaaagttagattaaaaccaaatcccacacaagaaaagaaactatataaatcctctggaacagcaaggtttatttacaattgggttttaaggaaacaaaaagaaaattataaacgtggtaataagtttatatctgattctgtattaagaaaagaaattacccaacttaaaaaaactgaagaatttaattggttaaatgaagtaagtaacaatatccctaagcaagcagttaaagatgcttgtaactcttacagaaggttttttaaaggacaagctaagtttcctaaacttaagtctaaaaagaaaacaagacctagtttttataatgatacacaaaaactaaaagtaaaagaaaaagtagtcttactagaaaaagtaggttgggttaggacttctgaacaattacctgtagatactaagtattataatcctagaattacttttgatggtaagtattggtatctaagtgtttctattgaagaagaaaataaatatgaagagttaaccaatgaatctttaggaattgacttaggaataaaggagctagctatatgctcagatggaattatatatgggaatattaataaaataataaaggttaaaaaactagaaaaacgattacgtagattgcaacgtaaggtatctcgtaaataccaaaataataaagaagggagtacgtttgtcaaaacttgcaatattataaagcttgaaaaaacaaattaaacacatacaacgaagattgtctaatattcgtaaaaactatttacatcaaacaacgacttctatcgtgaaaaccaaacctagaagaatagttattgaagacttgaatgtaagaggtatgatgaaaaatagacatttagctaaatcagtacaaaaacaaggtttttatgagtttcgtagacaattagaatataaatgtaaaagaaaaggtattgaacttactattgctgataggttctttccttctagtaaaacatgttctaattgtggtaacataaacaaaaacttaaaactatcagatagaatatttaattgtgaatgtggttttagtatcgatagagactttaacgctagtattaatttagctaactataatatttagattatcactatcatgatactaaatatatgtaccattcgttgtatgggaatttacgcctttggagtatcatacaaacctaagtagagtatataaaagtaatcagtaatggtgaaaatactcaaaaagggatacgctgaaaaaggaataaggtttataaagttttataaatttttgacaacggtaaatgataagttgcctgatttatttaaaagtctaaggttaggtagtatgtataaagaagacactgaagaccttatggttcctatagatgacggaatacaagctaacattagacaaatagaacaagatacaaaagagatgcaagaaattactaaatctttgtatggtaaacagcaagcttatgcagaaccatttttagaaatgatggatacaaatcctgattatagagataagaagagttatatgagaaatgctcataatttacatgaagtgttaaagaaatttggtaataactctatactaaatgctataattattacaagagctaatcaagtatctacatactgtaaacctgctaggtattcagaaagaggagtcggttttgaagttaaattaaaagacttagacgctacacctggtattaaagaaaaagaacagatgaaacgtattgaagaatttattcttaatacaggtacagataaagatatagacagggattcattccaagagttttgtaaaaaaatagtaagagatacttacacgtacgaccaagttaacttcgaaaaagtttttagtcctaaaaacaaaactaagatggagaagtttattgcagtagaccctagcaccattttttatgctactgataaaaacggtaagataattaaaggtggcaatcgctttgtacaggtcatagataaacaagtagtagctagttttacaagcagagaacttgtcatgggtattaggaaccctagatcagacttaaattctagtgggtatggattgtcagaagtagagattgcaatgaaagaatttatagcttacaataataccgaaagttttaatgatagattctttagccacggtggaacaacaagaggtatcttacagattagagctgaccaacagcaatctcaacatgcgttagagaactttaaaagagaatggaaatctagtttttcaggtataaacggtagctggcaaatttgcctctatataaagtaatttgtatagatgaagggttaactgctaagaacacctaaagctcaatacactacaaagtaacccgaaagggtaggcttgaatgttacgaaagtagaaaaaagatattgagatacgttaaggttaaatcctaagacgaagaacaatggttgtttagcatcgaaagtcctaaagaactgaaaggtctatggaatacgttcaacgactatcccgttacaagacgggagtaaagctccaagctattgggagaagaaaaatccttaccctacacaatagggataacaaatagtctagtctcgtactgaaaggtagagcatgtgaaatgacacaggtttacaaagtagcgtttgtaaataaataaaactaaggtttataaaaaaaaattattcctttactagttgacaattaatcttttctatgatatacttaaaccatagaaaagaggtgaaagtcaatgaagaaactaaaattaagattattaccaactccagaacaagaagacttaatgtgggttcatgtaaatcatacacgtaatgttaaaaatatatttttagctaaatgttttgaactaaaagaaacaggagaatttataagtaaaaccacatcaaataaactaagattagaattaacggaaatgttgaaaacagataaatataagttatttaaaaagtgtatcaagagatacactagatagagctgtaaatagtgtagttcaatcttatgttgattggtgtagaggtactcatggtaaacctaaattcaaaaagaaaaataaatgtacaaaatactttgatgttagggaaaatagattaaaaaggtatggacatacttttcatatacccagtataggaaaagttaaagtatctaaaaatactttaaagaataattttagtattataaattctatagaaagtttagataaatttagaaaaacatctattaagtatgatggtaagtattggattatgtttattacttatgatgacacaatagttacccctcgaaaaccaagagaattaactgatgaagttataggtatagatttaggtattaaaacgttagccacttgttcaaatggtaaatcttataaaaatattaataaaagttctaaagttaaaaaactagagaaacgtttaaaacgattacaaagacaagttagtagaaagtatgaaatgaataagcaaggtaaaaagtttattaaaaccaataatattattaagttagaaaaagaaataaaactcttacatagaaaacttagtaatattagaaataatcatattcatactatgactaaagaaatagtagaacaatatcctagtgaaatagtaatagaagacttaaaggtaagtaacttaagaaaaaataaacatttatctaatagtatttctaaagctaattggtatacaattagagaataccttacttataaatgtgaagatagaggaatattacttactattgctaaccagtactacccaagttctcgaacttgtagtaactgtggtaatcgtttaactaaacaatataaactatctttatcccaaagaacatataaatgttcttgtggaattaatcttgatagagacttaaacgctagtattaacttaaagaactatagatactccaaatggtatcacaatcatgttttaactaaataaaacagtaaacatgatatgtagggttccgtaaacccgaattaacgcctttggagtattataacaaaccttagtagagtatataaaagtaactagtaatagtgaaggtactcaaaaagggatacgttgaaaaaggaataaaattttttataaattttagattacgaagtccccgtgatgatggctgatgatgttaagtttgttaatatgacaccaacagcaaatgatatgcaatttgagaaatggttaaactttttaatcaatattatttctgctttgtatggtatagacccttctgaaataggattccctaatagaggaggaggagccacagggtcaaaaggcggttctacattgaatgagtcagaccctgctaagaagcaacaacaatctatgaataaaggtttacaacctctattaagatttatagaagaattaattaacagacacattatatctgaatttggtgataagtatactttccaattcgttggaggagatactaagtctgagttagataagcttagtattttacaaaaagaagtacaagtatataaaactgtaaatgaagcaagagaagaacagggattgaaaccaatagatggtggagatgtaattcttgactcagcatttttacaaagtacagcacaagaaatacaaaaagaccaatacctagatactaaacaaaaagaaagacttcaattacttatgagttatacacaaccacaaactgatgagcctacagatgatagtaaacaagaaactactaatcaagaagtagggacagataaccagttaaaaggtgatgataacgtatatagaacacaaacatcaaataaaggacaaggaagaaaaggtgagaaatcttccgactttatgtagataatattagcaacctaattaatacttaggttgttattttttaatttggagctacatttatactatgttatactatgtgtgtagtaatgaataatgttgatgatatactttttgttatattatgtatgtacatataaaataacaattaagtgaggtgtataatcactttggaagaaatgaaatttaatgcttttgttcctatggacttaaagaaatccatagatacggaagatgatacaaataaatattctatcgtatctggttgggcttccacaccagctgttgatttgcagaatgacattataaatccaaaaggtattgatatagaatactttaaaaaaaatggatatattaactacgaacatcaacaagataatgtagtaggtatacctacagataactgttatgttgatttagaaaaaggtttatttatagaagctaagctatggaaagatgatgaaaatgttataaaaatgttagacctagctgagaaactagaaaagtcaggtagtggtagacgtttagggttttctatagaaggcgctgttaagaaacgtaacataaatgacaatagaattgttgatgaacttatgattacaggggtagcactagtaaaaaacccagctaatcctgaagctacatgggagagttttgttaaatcattccttactggaacggaaacaacaccagacacacaagtagatgcaggagccttacgtaaagagtcattagcaagctctattactaatttaacctatgtaacaaaaattaaagatgttaaagagtataatgatatttggaataatgtagtagaggacttaactaaatctaataatatgggttatgaagaatcagttattacattacaattagccaaaggactatcaagaaaagatgctgagatagcagttatgagtattaataaaaaaaatttagaataggggattatattatatgcctacggaattacaaaatattctacaagaatatgataacttagacaaagaagaagtatcaaaatctgtagaagagtctaaagaagaagtaaaagaagaagtaaaagaagataaaacagaagacactaaaatagaagacactaaaacagaagacactaaaacagaagaaaaagaagaaattaaaaatgaagtagaagctacagaagagcaagtagcagaagctaaagaagaaccacaagaacctgcaaaggttactgaagaagatgctaaagaagcacatgaacaagaagaaaatctagaagaagaagtatctaagtcagctaaggagtcgaaagaccctgtagataaaaaagatactaaaacagaagacaaagacaacgagaaacgaaaaaataaaaaagacaaaaaagaagaaaaagatgaagaagaggaagaaaaaacatctaaatctatttctgataaagatattgtagatggtttccgtactatcttgaaatcattccaagacattaaaaaagaaaatgaacaatatgtgactaaatcagatttagaagaagtaactaaatctattcaagctttaactgataaaattaattctaatgaagaagtttctaaatcagttgagagcaaccaagaagataaagaagaagcagtagaaaaatccgtaacgactaataacacttcgaatgaacaagatgtagttggttatgtagctaaatcagtacaaacagaagaacaagcagaaactacagaagataaagaagaacaagaagtagaagttaaagaagaaagtagagacttatctgtagaagaccgtgaaaaattcatgagtacctataaagaaaaatctcaaaacccacatacgaatcgttcagaagtattgaatgcttatcaagcttacttaaatatccgcaacaatccagagcaagcaagtgaaaaagactttaatattgtaaaagaatttgctaatctttaattaagaaacaaagttgtgttatattatagtgtgtaaaataaaacaataaaaaaaagagcatatatgaaaggtgatatatataaatgaaccaaaatgaactttcaaaaaagcaacatgcagttgctgatgaattacaagaaaaattatcgaaatccttccaaactggatatggtatcactcctgaaacacaagtggatgcaggtgctttacgtcgtgaaattttagacgaccaaatcactatgcttacatggggaaataatgatttagtattttaccgtgatgttgcacgtagaccagctgaatcaacagtaattaaatatgatgttttcttacgtcacggtaaagtaggtcattctcgttttgtacgtgaaatcggagtagcgtcagtttcagaccctaatatccgtcaaaagaccgttacaatgaaatatatttctgatactaaaaatatgtcattggcatctagtttagtaaataacattgctgacccaggtcaaatcttgactgaagatgctatctcagttgttgctaaaacaattgaatgggcttcattctatggtgacgcaagcttaacttctgaagttggtggagaaggtttagagttcgatggattagctaaattaattgatgctgataacgttatcgacgctaaaggtgctcacttagatgagaaattattaaacttagcttcagttaaaatcggtaaaggttttggtacagctacagatgcttacatgcctatcggtgtacattctgactttgttactaatatcttaggtcgacaaatgcaattaatgcaagacaatagtggtaatgtaaatactggtttcagtgttaacggattctattcatctcgtggattcattagattacatggttctactgtaatggaaaatgaattaatcttagatgaaactttaatcccacaaccaaatgcaccacaaccagctacagtaaaagctgaagtaaaaacaaaccaaaaaggtaaatttactaaagaagaagaccgtgcaggtttatcttataaagtagttgttcactctgatgaagctgaatcagcaccatcagaagcacaagttgctacagtaacaaatgctacagatggagtagaattaaagattactgttaactctatgtaccaacaaagcccacaattcgtatctatttaccgtcaaggtaaagaaacaggtatgtactacttaattaaacgtgtagcacttaaagatgcacaagaagatggttctttagtattcgttgataaaaatgaaacattacctgaaacagcagacgtattcgttggtgaaatgtcaccacaagtattacacttgttcgagttattaccaatgatgaaattaccattagctcaaattaatgctagtattacatttgcagtattatggtatggtgctttagcgttacgtgctcctaaaaaatgggctcgtattaaaaacgtaagttatcttgcattaaaatagtaaatacaattaaaaattgaatacaatagaataaggagtatacctaagggttgctccttttttattattacttatagtatacaagaaaggaatttaaaaatcaatgttaacttataaattaaaaaatattaaaatggctactgtacatggtcaagtagaagtagatgataaaggcgtagttaaaggactaactgctaaacaagaaaaagattttgctaatttaccaggttttacacatatggaagacaagaaaacaacaaagaaagaagtaaaacctaaggaagaaaaagaagagaagaaatcaattaaaaaaacaactaaaaaagaagataagtaggttgatagaatggtaaacagtatgtttggtggtagtttagacccttacgaaagctctttatcatatgagtatccttatcacccatctggaaacccaaaacatatagataaaagagaattagatagtattactctagctgattatggttggtcagcagatgctgtgaaagcctatatgtttggtataacagtacaaaaccccgatacaggacaacctatgggtgatgatttttataatcatatattagaaagagctataggtaaagcggaaagagctttagatatatctattttacctgacttacaacatgaaatgagagattactacgaaacagaatttaatagttatatgtttgtacacgcatataaaaaacctatattacaggtagaaaatttacaactacaatttaatggtagacctatgtatgattatccagctaactggtggaaagtggagcatttagcaggtcacgtacagttattccctacagctttaatgcagacgggtcaatctatgtcttacgatgctgtattcagtggatatcctcaattagcaggaatgtaccccccaagtggagcaacttttgcacctcaaatgattacattagattatattagtggtatgttacctagacaaagagcaggtagaactaaaccatgggaagtaccaccagaattagagcaattagttattaaatatgcactaaaagaaatttaccaaatatggggttacgatgatattaccctgtgtgtagcgtaagttgcacattttcaaaggttaactgctaagaaatcctaaagctaactaaactacaaagtaatctgaaaggataggcttgaatgttgagaaatcagaaaaaattagttagatactctaaggttaaatcctaaagagaagagcaatggaagtttagcatcgaaagtcctaagtcaatagataaggaatacgttcaacgactattctgtatagacgacagaagtaaagctccaagtgattgggagaagaaaaatctttaccctagtcttagggataacaaatagtctgcacacgtcttgaaaaggagtgctaggaattgacctagacttatttagtagcgtaagtaaggaaacaatgttaattaaatgaaaacttcctattgaaattaccccaagtatatgttatattatagatatagtatataaggtggtgaaagtaaatgaaaaaaacggaaaatacattttatagaggtataacactaaaagtaaaccctacccaagaacaagaagagttaatgtggaaacatgtaaaccattctagatttatttataattatatgttagaaagatattggaaagctctcgataacggtaaatatatagaccataaaaatatgatttctatattaaaagaaataaagaatgatgataagtatagtttcttaaatgaagtatctagaaaaaccttagttggtaaaatagatgacttaagagaaacattaataaaatatcataatcatgaaattagaaaacctaaatttaagtctaagaaaagggaatctaagaggtttcctattagatatgaccgaatgcactttaacaaagaaaaacatgttaaagttgaaaaattaggttacttaagaatttctaatggtacatataaaaataataaatcagtactagagaatgaaagtattaaacctttaaaccctaaatgttactatgatggtaaatattggtatatatccttttcaatagaagtagataatcaatacaagaaagaaaaggataatactaacgaagtcataggtatagacttaggtagtggtaaaaaacacgtaatttgttctaacagaaaaatatatggtaatattatttattccaagaaaatgaaaaggttagaaaaaaaattatctgacctacagaaaagattaagtaataaatatgatattaatcacaccaaaacaaataatgttttaaagctagaaaataagatagctaaagtttatagaaaaatgtcaaatatccgtaaaaatcatgtacatgaaataacaaaagaaattgttgaaaagtatcctaaagagattattgtagaggatataagagttcgtaatttaataaaagacaaaggatatacaggacaaaaaagaaaacaaatacagttttctaatttctatatgattagacagcaattacaatataaatgtgaagatagaggtataaaatttacagtagctagtacttattatccatcatcacaaatatgcagtaattgcggagaaagaaaaagaaatgctttaaaattacagttaggtgataaagtctttaaatgtaatgcttgtggatatgaagaagatagggatataaatgcaagtattaatttaaaaaattaccataattcccaatggttaaaagaacaaaaccaataatatgtttgcttttagtagggttccataacccgaatgtataatgccttttattttacaaaggaagttaatttaattaacatatggtaatctcattatcggtgcaggaatagcaaataaaactctagatgtagatggtattacagaaacaataggtactacacaatctgctatgtatggtggagcaagtgctcaaatactacaaattaatgaagatataaaagagctattagctgggttgaaatcttactttggctccaatatgataggcatatagtaggaaggagatattaaatgcaaaaaccttttatgataggtactaataataataatacttctaatataattaataagtctactatgtatatatctacaacacaagcagatgaacaagaacaaaagttatctacagctagactagagtttgacactaaggatatgagaagatttgtcaatgatagaggtataaaggtactgtgggaaagggcttacttttgtccttgccttaaccctgacacaggacatcctagagtagattgtcctaggtgtcacggtaaaggtattgcctatttacctcctaaagagacaataatggctatccaatctcaagaaaaaggaactaacaatttagatataggaattttagatactggtacagctataggaactacacaattagaaaaacgagtatcttatagagacaggtttactgttcctgaagtattgatgccacaacaacttatataccatgttactaaagagcgtatacaaaaaggtatacctctatactatgatgtcaaagaagtaacctttattacatctaacgatggtaatgtttatgaagatgactataatatagagaacaatagattattcatggacgctaagtttgaaaacaaaactatatccttaaatattttaatggtacttagatatgtagtatcagatatattaaaagaaagtcgataccaatatactaaatttaatcaacctaatacaaagtttgaaaatttacctcagaagctattattaaaaagggaagacgttattgtactacctgagccatttaaagttaatgatggtatagaagaggacttagagatacaggtagaagaccctaaaaaagaatctaaagataatcattcaggtggtttctttgggggaatgctataatgcctataaaagcaactagacctaagttatttagagacaagagctataaaaatgtaggtaagagaacagtagatgctatgcgttctgatattttagatagattacaagctacagcattacaagtagataatgtgagtgtcaaacgtatgcctacctacttacaaataactgagcagaaactagagaagaaaggtgtaatagaccttaaaaaagcttttgcacgttcatctaagaaaaaaagagcaaaagatggtggttggtatctaacggttccaattagaataaaaacaagtaaaatgaataataaaacataccaagatttaaggtctttaaaaataggcagttcaagtaattcagtatctactcttactgattatctagaaggtagacgtaataacataagccatccttctttaaaagtacctagcagtaatggcagaataacaaaagtaagaaatggtaaaaaatctagttattttatgtttagaacagtttctagtaagtcacctgcaagttcatggatattaaatagagacaaagttaacgagaataatttctctaaaacaacattaaaatatgttaggaatttaatgaattggaaaattaaaaatttaatgtagaaaggttatggaagtaggatggcaataacatcagtagactcatatttattagagcaaataaaacccagattacatacagtactaagtaattgttatattatagatgaagttttaaaagactttgattatcaaactagggaaatgttcaaagaggctttttgcggtgagaacgcaaagcacgaagtaacagtagggttcaatttccctagctttaaaaacaactacgaagctcattacctaatacaactagggcaaggtcaagaggttaaaaactctttaggtagtatacaggggtcttatctagaagcttcgggtaacacatatagtgaacaatcagtagctaaaagagaaggtaatcgtttagtatttcatgtatctaaacctatttctaatgtaataagagtagaagacatagcgtttgattcttacgatgatgttaaagtatcagataacactgtatcttttaattatactaacaatgagacttatgaaggctacaacgcaaatattgtatatgtagaaaaaacaaatgatagtaaagggttagtaaaaggtattaccatagaagaacaagtaacaattgtgggtatgtcgtataatgtagatgttgctagatgtttagatgccgtattaaaaatgatattgatatctatgagagatagtatagaagaacaacaaacattccagctacaaaatcttgcatttggagatatcgcacctatattccaagacggtgagtcatctatctttggtagacctactataattaaatacacaagttcattagatttagattatacaataacacaagatattaataagattactttcaagaataaaggagcaaaaagataatggctaagaaaacacctaaaggtaaaacctttacaggttatgtacatatagatacatttttaaaaacagcacagaccttgtttaatatgaaagagtcacaggttgcaggttttaaagcctatatggaaggtaaacattacctattcaatgaaaaagactttattccatttttagaaaagtatttaggtagagaattagaaatttaatatagagataggagaattatacatggcagttgaaccgtttccaagaagacctgtcactcgtccacatgcagtcattaatgtagatagtacaggtatcggtaaatcagctagctccagtgaaaagattttatgtttaattggacaagctgaaggtggtaaacctgataccgtatatgaattaagaaactacgcacaagctaaaagactattccgctcaggtgaattacttgatgcaattgaattagcttggggttctaatccacaatacacagcaggtaagatttttgctatgcgtgtagaggatgctaaaccagctacagctgaagtaggcggattgaaagttacttcacagatttatggtaacgtagctaacaacattcaagtaggattagaaaaaaatacaatcagtaattcattacgtttacatttaatcttccaagatgatagatttaatgaaacatatgataatattggtaatatcttcactatcaaatataaaggtagtgaaagtacagctactttttctgtagagcatgataaagaaacacaaaaagcaaaaagattagtattaaaagctaatagcacagaagttaagtcttatgacttaacaggaggagcttacgctactactaatgccatcatcaacgatattaaccaattacctgattttgaagctaagttatcaccttttggagacaagaacttagagtcttacttattagaccctatgacagatgttgacattaaggataaagctgagtatgtaaaagcagtatttggtgacttagagaaacaaacagcttataacggacttgtttcatttgaacgattagtatctgaacaagcacctaaaaacgtagaagtagacgcacacgaagagtctgccactgtaacagctgtatcaccaattcaagaaatcacaccttttgaattaacaaaactttcagggggtacaaatggtgaacctcctgctacatgggcggataaaatcgaaaaatttgcacatgaaggtggatactacatggtaccactatcttctaaacaatctgttcatgcagaagtagcttcttttgttaaagaacgttctgatgcaggtgaacctatgcgtgctattctaggtggaggatttaatgaaacgaaagaacagttgtttggtcgccaatcatcattatctagcccacgtgttgctttagtagctaactcaggtatattcacaatggacaatggtcgtaagttacatgtaccagcttatatggtagcttctgctatcggaggtttggtaagtggtttagctataggtgaatctattacatttaagcaattacatattaataatgtagaccaattatatgagtcattagacttagatgagttaaatgaaaatggtatcattaccattcaatatgttcgtaatagagctaatacattctttagattaacagacgatgttacgacatttaatgataaaaatgaccctgttaagtcagaaatggctgttggggaagcaaatgacttcttatgttctgagttaaaacttgagcttgaaaataactttattggtactcgtactatcaatacaagtgcttcaattatcaaagactttgtacagtcattcttaggacgcaagaaacgagataacgaaattcaagacttcccacctgaagacgtacaagttattgtcgagggtaatgaagctagaatctcaatggttgtataccctatcagaagcttcaagaaaatttctgttagcttggtataccaacaacaaacattaagagcctaaactaggacaaggagtacctagtttaggtactcctattaaatttaataggagagtgaattaatatggcttcagaggctaagcagtctgtccatactggtaataccgttatgctaatggtaaaaggtaaaccagttggaagagcacaatctgctaaaggtcaacgtgaatacggaactactggtgtatacgaaattggtagtattatgccacaagagcatgtttacttaaaatatgctggtacaattacactagaacgtttacgtatgaaaaaagaaaacttcgcagatttaggatatgcttcacttggtgaagaaattcttaagaaagatattattgatatcttagtagtagataacttaacgaaacaagttattattagttaccatggatgcagtgccaataactatgaatcttgtagcttctagaagtaattctagtcgaaaaacttttctaaacagggaaccctaagttacaatgtaatatgggaatcttgtgctaaattcagtagatactgataaatgccaaacgactaaccgaaacctacttacagtagtaaggggagtaggtgtacagccaagtggtcagggtgttgggtaattaccatataaaaccttgctaagtggaaaagaaaagagtcctcgattgaggattgtgatatagtctaacctttatggaaacataaagcagttcataagagaacggataaagattaacgacctttattgaatatgagtgaatgagacttggcaaacaaatgaaattgtaactgaagaaattgagtagaagaatgctcctttaactagaaatagttatagcaaatccctttaattcatgggaacccttaacagataatgctgaaggcaatcatgagccaagcctagtaaattactaggaaggtgcaacgactagtcataatgacgtacatctaagcagatggaaatgggggacaccttcaatattaaggtgaagatatagtctgaactatatagaaatatatagaaggttcgagagtagcgttttgaatcgtaacaaatttgtttcttatttaacagctagtgataaagcccgtacctaaagggtttaaagacctaacaataaaagttaggtctttttttattgacatataactaacgatatggtaacattaaaatataatattagagaggtgttaagaatggataaaaaaataataggattaaatacaaaaacattagaagagaaggtttttactactataaaagaagcagaaaaagaaattgaattaagaggtaccgttaaaagatgtttaaataatgaaattaagtctactaaaggatggttattcaggtatgaaaatgaagattttccggagaaggcaagaacaataacagacaaaagaattaaggttgttgttttaagtaaagagcaggaacaaataaagttacctcttgagatggttaaaaatttatatggaatacctaaacctatgattattgaagctattcaatataaaaggaaaattaatgactactctgttagaagagctacaaaagaagagcaaaagattacattatctaacattagttatatagactatataaaaacagttaataacccaaacaatattaaaattattaaaaataaaggttcttttggtgaacagttagttaagatagtattagactataataatatagaatataaaagagaatattcttttaagaatgaattagggacagtacagagaatggattttttagcagtagtagataataaaaaatactgtattgaatatatgggtgagcaacattatgtagaaaaaggtaataagtggggctcgttagaaaagagacaatttctagattctataaagaaaaagtactgttacgaaaatggaatatattatattgatatcagttataaaaatagtgatttagagagtgtatttaatgttattaataaacacataccaggtattaaaaaaccaaataaatctattattgatattggtgtgacaataaatatagaagaaatgttagaatactataaggagcatactaaaaaagaaacagcttataagtatggagtaaaggaacataatattgataacttattagtaaatttaaactatgaaagcaaaagaaaacctaaagatataatacaagtatataagagagatgaaaaaatatttgaaggtacttataacgaaattaaggatgaatttaattttactatgagtaatgttttgaaatgcttaaatggagaactagataaaacatcacaacattactttaaatataaagattcagaaaaagataaagttagacaactaaaaactaaagaaagacatgctaaaagaggaacagtaagaggaagtagtaacaaagatgtcgttctaaaggacatatactcaggagaggaaactatatattcttctattaaggagtgtcatgaaataacaggtctaaacaaagacaaattgtacaagttatttaatccaaataaaagtaaacaaattgtagacaaatatataggtaaaatctacggtggtgagtaccctataactgtagaagaggctttaaacataattaacccttgaccgatatcaagggttatgctatcataataaataaggaggtattaccttgacaaacagaaaaacaattggtaagatgagtaatacaagagcaacatggaagataaaacctgtaactaaaataacaaaagataaaacaaaatattctagaaaaaagaaacataaaggtattgacacttataaataactgtagtactatagttatataaggagatgataaatcatgaaaactttttattcaggtagaagaacaacaagtaaaaataggcaagtaaagaagcattataaacaaatgactaaccaagaaataactgtgtgtagaaacttattaaaagaatcttataataaaaataattactttaagttaacaaatcactttaaaggtaaatgtaagaaaccagtaaattttaaagcgtttgttcattacatacataataagaacttagatattatagaatataatgaaacattatataataacaaaatacaacgtagggtagttgttagacatccttatgtagtaaaagtagataataaattatcttatcaatatctagttatagagatagaaacaggtgaagttgtaactatgtattataacagagttacagacaaccacagaacattaaatttaaatgactattatgataaaaatttaaaaataaagtattgacataagtaaatacataatatatactaaggttaaattaataaagaggagaataaaaaatgacaaataaattaagagctatccatgaagaaatgaaaattgagttacataaattcccacagaatgtagatttaacaagtaagacaactgctattgcaatcaatcaaattcttgacaagtttaaaaccttaacggaacaagcaggtaaaatcactaaaaagtattttgagggtcaagagattttgacgattgattttgaatattatgactcacttcaagattactatatttacttacttaaaaatagtgaaacaatcgaacaaagattacaagaaattgaaaaaagagtagctgaatatgttaagtaatttggagttataaccacattatgttatactgtatgtgtataaaaaacataagttagctgttatattaattatgagaaaataataaaggatggataaagaattggaaaaagaacaaaaagatattaaagatatgacacctgaagaaattgatgagttaaaatatcagcaacgaaaagataaagagcgtgttattaacaaagtaattaaaggtgttaatgatgtatgggagaaagaatacaactttgaagaattagatttaaaattcaaagtaaaaattagattacctaatgctagagaacaaggtaatatcatggctttacgttctgcatacttaggtggaatggatgcttaccaatctgaccaagttattaatgcttatcagatgttagctactttacaagaagtaggggtagaagtacctaaagaatttagagaccctgaggagatttataatctaactccgcttgctattatgtgggatgattggttagactttatgacgtcctttcgttactagtaacgtaaagcagttaaatgaagatatagaacggttaggtggaatttcacatatagctaaagagcctctatcaagaaacctatgggctattatgcgtgagtttaatgtattacctactgaacaacgttttaaagatttagatgattaccaaatagagtttattatagctaacatgaatcttgatacaaaagaaatgatggaagctagagaaggtaaaaactatgatatgaaagcggaagacgtagacacatcatggttcgaagcgccgacagatgaatttgatgttgttcctgatttcttagacttagatgatttaaatacacaagtagataaaagtttaagtgagaaagaaaagatagaaagggatagacgagttgaagctgagttagaagatgaaactgaaggattaacaactcaacatttggctatgatggaacacataagacaaaaacaaaaagagcttgatgaaagtttaggtattgacagtaaagaaataacacaagaagatataaacaaagcaataaaaaatgtagatgattggtacatgtagggtaatggtataataccaatacccttattttttgattaggtggtgaatactgtttggcaatgaatgacgattataggttggtcttgaccggtgatagttctagtttagaacaaagtttaaaagctatagagatgtatatggatgcgttagagtctaagaacatagatgcaccattaacaaacttcttagagaaactaaaagtaatagcaaaagaagtaaaatctgtacaaaatattatggataagcaaagtgataaatctttaatatcaccaaaagctatggacgaggctattagttctacacagaatgtaactaagaacataaatgacctaaagagagctttaaatgacatacaaacagataatattacaaagggtatagctcctgaccctgaagtagagaaagtatacagtaaacttaataagacactaaacaatacacaaacagaactagagaaagtagcatctcaaaagattggttcagactcagatgtaacaaacagaataaaagaaatgaaaacactgaaccaagttacagaagagtataataaattagtaaaagatgccagttcagctaaggagtacacaaagcaactaagagctaatcgtaatatggttaggggtcatatatctaggtctgaaggctctaatagaatgtcctatgaccaaggtgctagagtacgttctgaattaggtaaagtagacacttttgaaaagcaacgagaagctaacaatagaagaataaaagaaaaccaagatagatatagaggttatagacatcaacaacaagatttagtaggtaaaagagctcgtggtgaaatcagttctgaagactataaaaaacaatcagcatctattaaaatgatgattgatgaaagtgaaaagctttctgaagtctatagaaaaacaggtgctgaattagacaagtcaatcaactactataaaaatagtgctaaaaaacagtttgctcagcgtgagatagaacagcaaagaggaacaatgggtaaattgtttcaagataggttaccttctattggtgcacatgctactatggcagtaactgctcttgcaggcggtatgtatatgcgtggtgcttccctatctgaagcaaatagacctatggtaacatcattaggacagaattcagataatatggacatagatgcagtaagaaatacttatggtgatttatccatagataataagcttggttacaattcaactgacgtgttaaaaatggctacatcatatgaaagttctataggtcataaaagtgatgcagatacatacaaaggagctaaacagttagctgtaggtggacgctcattaggtatacaagaccaagaaatgtaccaacagtctatgtcacaacttatgcatacaggtggcgtaaattcagataatatgaaagctatgcaagatgctttcttaggtggtatacgtgaatctgatatggttggtagacaagatgaacagttaaaagctttaagtactattgccgagcagtcaggacaaggtagaacattatctaaaggagacatgtctaatcttacttctttacaagctcaagtagcgggtacaggtagtaaaggtttacaaggagctcaaggggctcaagcgttaagtagtattgatcaaggtattaaaaatggtatgggtaattcttatactaggttatccatgggttggggtactaagtaccaaggattagaaggtatgcatgacttacaagcacaaatggataaaggtatatcagaccctaataacttagttaatatatttgaccaagctaataatataggaagtacaacaaaagaaaaacaagccatagctaaaaaaggctttgaaagcatgggagctaatttaacacaagagcaaacagatgagttgtacaatctttacgcctcaggtaaactatctaaagaagagttagctagtaaagctaaaaagatggagcaagaaggttccaaagcaggagataagaatagggataagtattccgaatctaaagcaggtaggaatgaccagaacaaagctaagacagatgataaagccgaggatatttatgacttagctcaacctattagagatgttcatagtgctatggcaagcttgccagctcctttatacctagcttcaggagcagtacttgcatttgtagcatctttagctaaatctactgccatgatggctggtggctctttattaggtaaaggtcaaaaaggactaaaagattggtttaataaccgtaaaggtggttcaataggtaagactggtggtgggcgttttggtggacttaaaaacttagctgacactatactaggagataatcctaaaggtggagaccgaccatttaaagaccaagtaaaaggtgcaggacaaactgctaaaggtatgggtaaaaccctatttgataccttcggaaaaagagactttacatttggtgaatatatgggaagaaccaaagacttaggtaaaggtgcatggaacaaaggtaaaggagcttttggtaaagctaaaggtaaatttagtggtaaaggttccaatttaggttttatgtcacaagcacctactgctaacgcaggtggtcttggtaaactaggtggtcttacaagtaagctaggttggattggtgtaggtctatcggcgtttgatataggtagttctttaatacaaggagatactaaagaagcttcttctaaatttggtaacactataggaagtgtcatagacccattaaacctaggttatggagacggattaagagactatgctacgaaaacagcagaaggtgcttcgacaggtgatggttggaaattatgggctaatggagataaagatggtaagaataaattccaagactcacctgtaggtaaactatggggtggcataacagatttcttcacaccagatgacccttctaaattagatgaaacagtgaaaaataaaaaaggtaacataattagttatatactaattgccttgcttatagagcaatctgtaagtaccgagtgttaactgctatgaaactactaaagctaactaaactacaaagtaagtcgaaagactaagcttgaatgttgagaaatcagaaaaaattagttagatactctaaggttaaatcctaaggagaagtacaatgggtgtatagcatcgaaactcctttaacatggagaacgttcaacgactattccgtataggtgacggaagtaaagccacaagtgattggtggaagaaaaatactcaccctataaactagggataacaaatagtctgcacacgtagggaaacctagtgtctagaaataactagagaatatacagtagcgtgtatatttaaacattgttaaaatatataaataattaaaatttgtattctacttctgcatataaacctttcttattttgtaaggtaaacttgttattaaataaatatacataactaatacgataggttatatatgaaagtatactattgtttatatgttttaacttatggtattaatgataaatcacaaagtaaagacaggcaaaaagcagaagaaaaaagagaaaagaataatgattctagagaaggaaatgtaacagtatactcacatttcttagaccatttagcagacaatctaacaaataaaggcagtagttccgacaatagttcatcaggaggaacacttgactatcttgatggtaagaactttacagataaagacgtaactaagcatgacttaggtaaaactgctaaaggtgtaaatgccgaaatgttaaataaatggattgagtctcaagcacctagtaattctaaaatgcgtggtttaggtgagctttatatgaaagcaggtaaagagtcaggacttgacccaaggtacttagtggcacatagtgctgttgagacaggctggggaacctcgaacctttccaaaggtggagaccctaacaaaggtaattggttcggtataggtgcttttgataacaatcctaataatggttttaactatggattaggtattgttggtggtgctaaatggattagagataacttctacaacaaaggacaaaagaacttacatgatatgagacataacaatggtgttcatgagtatgcaacagcaggtaattgggatactatgattgccagtattatgaaaggttcagacaagttcataggtggtggaggtggaggcagtgatgttatgcctagacatgctctaaaaaatgaccatcaaccaaagaatcttaaatataacatagaagtacctaaaagtaaaaaccctaatgaaacagggaaagcaataggtagaaaattaaaacaattattagaatctaatttttagaatacaattataatatttatatactttaacaaagggtacaaacaataaaaaacagaaaatgtctacagaacaactaagagaaaagaataataaatcagaaactaaaaacttaaaaacttacagtaacctattagatagggcacaacaaattatagaagatgctaaagcgctagactcaggaggttcagacagtagttctgattcaggaggttcagcttccgatgtagatggagaaggtgcgggtaagatttacaaattccttaaaggtaaaggtttatccgataaccaagtaggtgccgttatgggtaaccttaagcaagaatcagggcttgaccctaatgcaaagaacgcctctagcggtgcttttggtattgcacaatggttaggtagtaggaaatcagaccttgatagctttgctaaatctaaaggtaagaagtctaatgacctagacgcacaattagatttcttatggaaagaaatgagttctggtcaaaacagtgatatgcttaaaaatgcaggatggagtaaaagtggaagcttagaaagaaatacaaaagcttttgcacaaggttttgagcgtatgggtgctggagaagctatgatgagtaccagagttaacaatgccaaaagctatgttagtaaatacggtaagtcaggtggtggcggtggcggagttataccatcgtcatatacttcagtcatgagaaatcctatacttagtagtacatctggaagtagtaaatctcaaaacaatgtttctgtgagtgttaacatcacaggagtaaacgataaagaagatgcggaaggcataagcaaaggtattaagtctacctttggtgacgatttagacatcttctcaaacacgtataaacgtaattattagggtagtattattaagtactaccctttatataaaaataaaggaagtacatatatgtatagaattagacgacctaagatacgaatagaaatagtaacagatgataatacatttacattaaagtttgaagatactaaagattacaacggcaatcaatttgcttcaaagttacttagttttcaaactaagaatgctatggaagatgatagtgctgtattccaaataactatgtcaggggatacgtattgggataagctagtaatggcaaatgatattattaagatatatattacgcctaataatgaccctaaagataaagagggaaaccaagagaaacttatacaagtgggtatggtatctcaagtatctaaagtaggtagttatggcaatgaccaagtacaatttagaataacaggtcaatcatttgtaaaaccttttatgaagtttggtttaggtgttatacaagaagtacaagctgtattaccaactgttggttggttagttgatggcgatggagagaatcaggttaaatttacaggtagttccgctaaagatgtaatgcaaggtattatacaacgatttattccttacatgaagtacaattatacggataaaacatacaacacattagagagttacttagattatgatgatttatctagttgggatgagtatgaaaagttaactgaagtttccgcttttactaactttgatggtacattaaaacagctaatggacttagtaactgctagaccttttaatgagttattctttagaaattcagataaacataaaggtaaagcacagctagtattaagaaaaactccttttaatccaactgaatggaaagcattagaatatatagttgtaccaactgatgattttatagaggaggatgtaggtaagagtgatgtagaaacctactctatctttacagtaaaaccagcaggtatgttaaaagaattaacaggggatgtattctctaaaccacaattccaccaagaattagtagatagatacgggtattctaaatatgaaacagaaaacctatatataggaactaaaagtggtagtgctacagaagatagtgaaactacaggtggagataatggacacgaacgagtaacttatgacagactacttaaagatttaaataactatggtagagagtctatatctaaaggattagataaatactctagtaagatatcatcaaaatacaacaatattactaaacctgaagtaaagagtattttagaatcttatataaaaaaaggtaatctaagtaaggaagaatatgaaaaaataacaaagaacaatacttcaagagagtcagtatctgatactagaccaaaactaacaaaagaaaaattaaagtctatattagcggaaaagtttaataagaaagaaacttttgatgataaagatttaaataagaaaacaacaaaagctgtaatagatgatattacaaagaattataagtatggtaatcctacacatgctaagaaactactagagtattacactagatttaaaggcaacccaccgacaagtaccacaggtgatatttatgataagtatcttaaagccgtagaaggagtagctaatgttgctagagatacaggttctgacgcaagtgatgactctttaattatatttactaaaatgctatttaattggtatcactcaaaccctaatttttatgcaggtaatattgttgtattaggtgaccctaagtatgacctaggtaaacggttatttataaaagataaacaacgaggggatatatgggagttttacatagagtccgtagaacataagtttgattataaacaaggatactttactacaataggagtaacaagaggactaaaagatgctattataaaagatggtgaaggaagcaaacataggtttaaaggactatggaatcagtcatctgactttatgggtggtcttatgggtgaagagacatctaaagaattaaaagaaaaaggtgtagccgagaagaaaataaaaggaaataaagatggagactccgaaggtggtgcacaagatggaggcactttagcttcattagaaaaatataatggtaaattacctaaacatgaccctaactttgttcaaccaggcaataggcattataagtaccaatgtacatggtatgcctataatagacgtggtgagttaggtattcctgttccgttatggggagacgcggcggattggattggtggtgctaaaagtgctggttatagtgtaggtagaacacctaaacaaggagcttgtgtcatatggcagagaggcgctcctggcggaagcccgcagtatggacatgttgcttttgtagaaaaagtattagacggtggagccagtatatttatatcagaacataactttgcaactcctaacggttacggtacaagaacaatagatatgagttcagcaataggtaaaggtgctcaatttatatatgataaaggataattgatatggcaacggataaacaagttaaagatgttatagataaatttatagataatcttttcaattttgaagtattaaccaaagaaaaggttgaagaaaaagataaggaagttaaaaagataactacagaagacgtatacaataaagttgtttatattagaccatatgtaggtgttatacaaagtgtaacacctagacaggtaacatatgatagttttactaataatggtacagatatagaagctaaactcacatttagaaaaataagttatactactgataatctcacagtacctactgatgcattatcaacttgtatggtgcatatggtagaaagaaatggtgtacttgttatagattactttgatgagttacagaacatactgtatggtagctacatggagaatgagcatatatttgatgaagatgttccagttagtacattagtaacattagacttgaaagagaatttaaataattataaacatatacagtatatgtttaaagggaatacggataaaaacccttttgaaagtaaggatatagtacctttagatacatacaacttattatattggatgtataaaaagagaaatgtagagcttaaaaatccattaacagttaactacttcttcacaggtaaaagcttctatactatatttggtaagggacacaaatataaagttgatattgacaaattcaaaataggtgatatactattctttggacgtagtgatgataatatagggatatacataggagataagaagtttatttctcttataggtacatttcctaaagataacacctctattagtacctatgagttagacgattattgggatgactttaatggtagggttatgaggtttgatgaggatgatataacatggtggtaagattacagtcttctttaggtagaagtttaaaaaagatagactctgaagaacttaatgttaagggtctattattaggtacagtaagcaaaataaattataagtaccaaactgtagaagttaaaataaatagtttaacactaggacgtaaccctagtgatgacggtaagttagctgttccttatcctaaagattatgtagggagaacacctgaaggtagtatttatggtagtaatactttaataacggaaggaactactgtactgttaggatttattaatgatgacattaatcatcctattatactatctatctatggtaatacagaagtaaatcagctcataaataccaatcctttagaaggaggtaaaatgaggaatgacgacgtgtataaatacagtagtgctttatactctatattacctgacctaacttatacatataaagatggtgaaggaacatccattaaaacatttaatggtaaatctttcttatctattacttcgggagataaagaaaaaccattagcgtcagacttttatacaggcactgaatatcaagacctattcccatcatattatagtaacaaaaatttaatagaacctagaatacaaaaggcacctaacatgctatttaaacaccaaggactatactttgatgatggttctgaagacaatcatgttacaactttatttatatccgaaaaaggtgatgtgagagcttcagtattagacaaaagtacacaaaaaagaacaacacaagagatgtctcgtgatggttcttatagagttgtaaaacaagatgatgacctattattagactcagcacaagtatgggtagagtttggtataaatgataataatgtgttttatataaaaaaccctaagcatatgtttgaatttaatgataaaggtatatatttagatgataagccaattttagatacgttagatgctagtattacagagtctatagatagacttaaggaagtacagaaagaattagatgacattaactacttacttgagggtgtaggtaaagaaaacttagaggagttaatccaagagactaaaaaatctatagaagtttctagtaaagcaagtagcgatgttacgactatgaaatctcaaatatccaatgtaagtagtagagctgagggcattattacacagtttcaagagtttagagacaaaacatttaaaggcttatatgaagatgcttctactatgataaataaatacaaccaagaaatacctgagttaaaaagtagattgaatactctagatacaattacattacctaacattaataataccttattagagaaatcaagtaagacctatgttaataatagttctcaaagttttagtagtaattatctaggttcattactgcaactagatagtaaccttactatttatccatacacacctacacgtattacatggaataagatagtttttaataattctgagtttgtaaataagactaaggatgcttttgttattccattaggtgtatctaaagttaaagtttctattaatcttaaatgggatatattacaagataaagtaaaaactattacagtcaagaagaatagtaatacatacaaaactataactaatggtaatacacacgataatataattacaaatgttatacctgttaaacaaggagatacattcacagtagaagtacaacaagataatgatattaaccttacattactacaagatgatacaatgtttagtatagaagtattagaaactactttagcacctactaatactgagttattaggtacagtatccgaacaaacagataagcaactacaaacacttgtaaattctaatgttgatttactaagtttaacaacacaagttactaaagataatcagttagtttgtcttaatgattccaaattagacaatataacaacaggtaaaggtaatgtatctgactatactctacaacaaataaagtcattttcagctaagaatggtgagagtattttatctttagaagatgtattagctaaatataaaaattatattagatatagtattaatttaccaactactgcaaacatagtagaactactgaaaaaatataatttaattggtataagagcacctaggaatcaggtgtatttgaaagttaaagatttaacaatcacacaagaagtattaaataatttttctaacttaccagtattatataatacttcctatctaacaagtacagatattaacaaaggtatatatacaaatatatatggagtactactaccatatactaatatcactcaagatacagtaaatacagcacataataataacctatctatatatgctagtggtgatatgacaagtgatattataaataagcttattttatatggtatagatggtgtagaaacaaataaaatattagagtttaaaaaagtttaaaagtcctattaatagggcttttttatatatgtcatgttatattaaacttgagaaagtagttgtatttaaatcctatatatgatataatacgatatagaaaggtggtaagttatgcctcaatcagatggaaagaatacaataagacgtattgcattacgttttccaacagcaagcggtaaatataatatgtaccgtttcaaagtaaaccctgaaagttatgaaatagatgcaccacaaagaacatctataactaaaactaaatcagacattattgttgaagattacggtaaagatatagagacaataactttttctggtactactggatttagacctgtaaaagaagtagatggtactaaaacaggaaaacaaaaaatagaggaactacaagaacaagtagaagcatacgctaagcagggtggagatggtagcgtagcaggagcttatatagagttctataactttactgatgataaatactacaaagtacatttagcaccgcaaggtttaaaaataacaaggtctaaagatgaatccttgttatatagatacgaaataactttagttgttataggtaaccttgctgaagcagacagaggttccgtaacatctgctgagtttggtaatgtaaaacctaatgctaaacaaagagttgatgagggtgttaatgatttagatagtaacgcacgaaaaacaagaaacaagaataatcaaatagtatcacaatttgaaaataaaagtagtaaaagctcttctaattctagtagtagtggttcacgatatggaacggcaggaacattaagaggaggtagtggcataagaacaggtggttctgaaggtaatagagacggtatgagtaatataggtatatataaccctagacagaatactaacggtcttagtagtactgttgatgatatggcacttaaaataggatacggtgatggaggtgtatctaggtaatgaataattatatacctcaaccacaaggactattaaggtttttaaatggattagagttagacatgtcttacacacatataaacttattagatgaggaagtaccttttgtttctaaattttatacaccacaaatacaactaagtgaacttgcaagagtaaaattaaaagagattaagtctactgatatccctactttagaaagaacatttaacaacaatacaatagttaataaagtagaacaaacaagtttaaagttacaatcacctagaatgtatttaattatgcaatctatcgtaatggaagcttacgctattgtaaactgctttgtagaaagtcctgaatacttaaaatatttaacagaaaaagatgtatctatagtgagaagtaatgttaactatgtagctgactacttaagtgattatgaagaatacaatagtattgttctcgatttaagagacttagacttatgttttggctctttggaattacaactaccgctaattaagagtgaggtggtataatgagatttagaaagcatgaagtccaaccagaagaaaccttacaagctatatcacaaaggtattatggtgatgttagttattggttagatttaatagaacataacaacttacaatatccttatattgtagatacagatgaagaaaagaaaaggaaccctgaacacttaataactaagggtgatattattattatacctttagatgctgagttaacagactcggtttctaaagaaataacatcaagagataaagatgtattagtagaattagctttaggtagagacctaaatatgacggataataaagagtatatagacgctcatggaacagatgatgaaatattagccctatcttcagatggtagaggagaccttgatatagttaaaggcatagataacattaagcaatctttaagagctagactattgacacctaaaggttctttattattacatcctgaatatggttctaacattcataatttatttggtttaaatatacctgaacaagctacgttaattgaagtagatgtattaaagactctaacatcagatagtagagttaaatcagctaacttaaaaggttgggagataaaaggtaatgaatactatgggtcatttaatgtagaaataaagtccgtagaagaatctatagactttgtattaggtcaagatgattcaggtgtctttgctttatttgactaagaaaggaaatttatatgagaacaagaaagttaacacagatattatctaggttaatagataagacgatacaaggtacaagcaaaataacagactttacacctggttcagctgtacgctctcttttagaagcagtagctttagaaatagaacagtattatatattaacaagagaaaacattaattggggcattcaagaagggattattgaagcttttgattttcaaaaaagaaaagctaagcgtgcttatggtaatgtaaacattcaattttaccaacctttagacatgagaatgtatatacctgcaggtactacattctcatctactaggcaagaataccctcaacagtttgaaacattagttgattactatgctgaagaaggaaccactgaaattacagtagaagtttattgtaaagaagttggtattgtaggtaatatacctgaagatatcattaatacaatagcctcaggctctagtttaattcgtacaattacaaacccgacttcctttaatacaggaacaaaagaagaatctcaagaggattttaaacgtaggttccatatgtttgtagaatcgagaggaagagctactaataaatctattagatatggtacattacaagtacctgatgttgatggtgtttatgtttatgaagaagtaggtcatgttactgtatatgcgcatgataaaaacggaaatttatctgctactctaaaaaatgacataataacggcattagaagattacagacctagtggtataaagttagatgttgtaggtgtagagaaagaggaaatagatgtgtctgcagtagtaacaatatctaataaagctaggataggtgatacattacaaagacatattgaaagtgttatcagagggtatctaaatagtctgactacttctgatgatttaattattaacgatttaattcaagctattatgaatatagatgataatttaatttatgatgtagcatttaagaatttatctacaaatatagaagtatcttcgaaaggtattattagagcaggggatattaaggtagaattaatatagggggtaaacaatggctaattttttaagaaatttacaccctttattaagaagaaataagaataataaagataaccaagaccctaactatgctttaataacagcacttaatgatgagatgaacaatatagagttagacaccattaaaagcaaacttcaatcatcattaaaaactgccacaggagattacttagataagtttggggattggtttggtgtatacagaagaataaatgagaatgatgacagctatagaaaaagaattataaaatacttattattaaagagaggtactaataatgctatcattagtgctattaaagattacttagataatgagaatataaatgttagtgtatatgaaccttttaaaaatatattctataccaataaatcaaaattaaatggtgaagattatttaatgggttattattatagatttgctgtgataaatgtttctgttgatggttactttcctttagagattatagatgtaataaatgaatttaaacccgcaggtgttaagttatatttaacttatgatggtgctactaccataaagggtgatggtgttgttaaatggcttaaaaatctacctaaaattgaaacatatacggatttagatagattattaggttatgatgaaactttctatgggcatttaaacatgagtgaagcaacagctgaacaatttgataaaacaaggttcagaacaaataagagtatattaaatagtgaagatattctatcaggctcttctaatattggacgtagatttataaattatgcttatgttacgacatatgcatatgaacctactagtacatcagccgtcacacatatagcttctttaaatgataaaggaaaagaagcacctttagattattacttatacacatccttaaagaatactaatggtataaatatatcaatggaaactgcttatggtgtatcttatttatacaataactttaatgtaagtgagtttatcagcagatacaaaccgaatatagatataactagagataatgctaaaaaacaaatatccgattttataggagaactaacaattgatttctttgcaaaagctttagtttcacctgatgaatcattacctattaagttacaaatatttgattttcgtactaatgaatggttaactgtatcagaaacagaactatctttttacgagaaaaacataggtgtaaacataggttatataagagattacttaaatgatgaccttaatttatttacaagattagaggttaatataggtaaagatgagcaaaaagatataagtattaactacatggatttacatttttataattataaaaaagatgtgtatactatcaaaccttttaaggcactagttgaaaactacatagatttgactaaagatacttatattgaaggctttaaagtagcctcattattaaatggagacataattaataaaaatggatatcaacccataaaatatttaaggttacatgctacttatgattcaactaaacctattgtaagtattacaggtaaaaacagcaataatgaggtacaaaataatacacctatagatttatataaaaatacaactaatagaaacttattgcaatcatataaaggtgatagtaatatattcaagggtgttgtaagcacaaaagagttttatatctcatcatgggctgacaccttatataattcaacgtatttatctaaagtactagaaccacaaaaattatatactttgtcattcgagatggaaatcacagatgctaatatggacttaaaaacatattctgacaatcatggtatatatttatatagtagtactaaaggtattgttgctaatggtgttaaaagcatgcaaagaaaaataggtaataagattgaggtatctatcacatttacagcacctacaattacagaccatagacttgttgtttacacaggtagatatacagcagacggtactgcttctaaaacacctattgcttataatacggtaaaagtaacaaaccttcaattaaaagaaggagctactaagaaggactacacggtagcacctgaagatgtaactaatgttatagacaaagctattgtgtttgataacatattaacagatatacaaacaataggaataaatacaacaacacctcttaaaaatattgagctaagttattcttactatggggataattggacaagcctaaaaacaataagtaatttaagtcaaggtgaaacaataacacctaataacttagtagatttatatgtcttagaaacaatagattattcaagtattacacctttctctagggttatacttaaaactatatggaatgctaacctgaataatttaacaaataaaaaaggtagtatttctaacatgagtaataactatttcaatgctgtgtggcaagaggtagatatgatatataacactacattaagtagcatgagcatacttactgatagtgaaggtaatgtatttgatagttcaacaggacaggtaataaaattagcacctaagatataccaaggatatactgatgtagatagattgctaacgtctactactctatataacgagaatattacattaggttcaaatagatttatttatgacttacgggatatgttactatcaagtgatacattcacagtagataatagaattaaagtaatagatacgtacacagaagtacagtaaagatattgacaataaagtattaatatgatatagtaaggtatgcgtattatagtataccttgttatattaatacagaatactagacatgaaaggaataaaaaatatggctatagcaacaaatgcttcacatgtagaattagctaaatacattgtaagtaaggctgattctatttatttaacaattggtaaaagtacagcgtgggctaatgaaacaagaccacctcaacctgaagaaacagcaacatcattacaagaagttatcggatataaaaaagcaactaaggtagcattagttagacctgcaaaaacaacgggtgatgaaagtaaaaaagaaattacatatggtaataaacgatgggtagaaattgactcagctaatgcagttgctgaaggtgctaagtgggtgtacttagaaagtagtattgttggggatgagttacccttaggtacatatagacaagtaggatttgttatggatttgttagctaaagatagtatttctaaatttaacttactacctagtgaagtgcaatctacaggaactttattattttatgataataaacagttccaaaatagaagtgaacaaacaactgtaaaagagcgctttatcattgaagtttaaaaaggagaattgaatatatatggcaattaattttaaaagttctccatacttagatagatttgaccctacaaaagatagaacaagagtactatttaatccagatagaccactacaacaatcagaactaaatgaaatgcagtctattgaacaatattacttaaaaaatttaggggattctatttttaaagatggggataaacaatcaggattaggttttacattaactaaagataatgtactacaagttaacccaggttatgtgtatataaatggtaagatacgttactatagtagtgaagacactgttaaaattacgggtgtaggtaaagaaacagtaggtattaaactaacagaaagaattattacacctgatgaagataatagcttattagaccaaacaagcggagttcctagttatttttctaagggtgcagaccgtctagaagagaagttatctttaacagtcaatgaccctacttctgctactatttatacttttatggatggagacctctatatccaatctactaatgctgagatggataaaatcaacaaagtattagcagagcgtacttatgatgagtcgggttcatataaagttagtggatttgagctattttcagaaggaaacgccgaagatgataatcatgtatctgttgttgtagatgctggtaaggcatatgtaaaaggttataaagtagataaaccagtttctaccagaattagtgtagagaagtcaagggaattaggtaaagcagagaatgaaagtactgtatttaataaatcatcaaataatattactttagctaatacccctgtaaaagatgttcaacgtgtaacagcgcaagtattagtagataaagagcgtgttacaagaagttcaacaggagattctcaagactatttatctaataaaacagcttttgaagtagttagagtatggacagaaacaagtccaggtcagacaacaaaagaatataaacaaggagaagactatagacttactgatggtcaaacaatagattggtctccttcaggtcaagaacctaatggaggtacatcttattatgtttcgtataaatacaacagaaagatggaagtaggaaaagactatgaagtaactactactggtgaaggtgtaggtaaacgctggtctattaactttacgcccgttaatggtactaaacctattgaccaaagtgttgttcttgtagactatacttattacttagcacgaaaagatgctgtatttattaataagtttggtgatatagctatccttaaaggtgaacctaatattatgagattagtaacaccacctctaaacacagaccctgagaatttgcagttaggtacagtaacagtattgcctgattctaacgaagcagtatgtatttcatatgctattactagattatctatggaagacctacaaaaagtaaaaactagggtagataacttagagtataaccaagctgttaatgctttggatgatggtgcaatggagaaacaaaacccattaacattacgttctgtatttagtgaaggttttatatctttagataaagccgacattacacaccctgactttggtattgtatttagttttgatgatgcagaggctactttagcgtataaagaagctgtaaatcaacctaagattatccaaggagacactacagctcatatatggggtcgattaatttctgctccatttacagaagaacgtacaatttaccaagggcaagcatcagaaaccttaaatgtaaacccatacaacataccaaataaacaaggtgtattaaaattaactcctagtgaagataactggatagatactcagaatgtaacaatcacagaacagaaaactaaaaaagtaacaatgaaaagattctggagacataatgaaagttattatggagaaacagaacattacttatattctaacttacagctagataaaggacaagagtggaaaggtaagtcatacgcatatgatagagaacatggtagaacaggtactctattagaggctggaggacagcgtacacttgaagaaatgattgaatttattcgtgttcgtgacgtatctttcgaagttaatggtttaaatcctaatgataataatttgtatattttatttgatggtgttcgttgtgctattacaccagaaacaggctatagaaaaggttctgaagatggtacaattatgtctgatgctaaaggcacagctaaaggtaaatttacaattccagcaggtattcgttgtggtaatagagaagtaacattgaaaaatgctaactctacaagtgctacaacatatacagctcaaggacgtaagaagactgtacaagatgttattatcaaaacacgtgttacagtaaacttagtagacccattagcacaatcattccaatatgatgaaagtagaactattagttctttaggtttatactttgctagtaaaggagataagtctagtaatgttacaattcaaattagaggaatgggggacacaggataccctaacaaaacagtttatgctgagacagtaatcaatgcagatgatattaaagtatctaataatgcaagtgctgagacacgtgtttactttgatgaccctatgatggctgaagctggtaaagaatatgctattgttattattactgaaaatagtgattatactatgtgggtaggaacaagaacaaagcctaaaatagataaaccaaatgaagttattagtggtaatccatatgtacagggtgtattatttagttcttcaaacgctagtacatggacaccacaccaaaattcagatttgaaatttggtatatacacatctaagtttaatgaaacagctacaattgagtttgaacctattaaaaatgtatcagcagacagattagttttaatgtctacatatttaacacctgaaagaacaggttgtacttgggaaattaagatgattttagatgatatggcaacatctactacatttgaccagttaaaatgggagcctattggtaactatcaagacttagaagtattaggagtagctaagcaagttaaactaaaagctacatttgagtctaataagtatatttcacctttattaagttctaatgacttgacttttactacattcttaactgagttaaaaggttcatacatcggtagagctattgatatgacggaagcaccatataatacaattagatttagttatgaagcatttttacctaaaggtacaaatgttattcctaaatactcagatgatgatggtagaacttggaaaacatttactacatctgctcaagtagttaaggctaatagagactttaacagatatgttattgatgagaaagtaaataaatctactaagaatagtaaattacaagtaagattagacttatcaactgaaaacagcttcttaagacctcgagtacgtagacttatggttacaacaagagatgagtaagtttaaagagggtttaataccctctttttatttaattaaggaagtgatatcatgcctacagaatacagagacccttattcacaagcaaaactctttattcctactgtagaagagaaggctataaaagaaatggaacaaacatataaaaataaattacaagaagttgatgagcttatcaatgaacttaaaaaattgaaaggtgattatacacatggcgtttaactacacacccttaacagaatcacaaagattaaaagatatgtaccctaaagtaaatgagatgggtagatatttacaagaagaagttaatctagcggatgtaaaaacaataagctaccctgacttaagtaatattctaacttacatacaagagtcaggagattactttgttacacaagcaagaaacacaccaggaggagtaagtaacacaggttttttacacttagataaaaaaggagatacttattataagttatattatgctccttctaatacaaataaattatttgtaaagacatactataatggtacggtatatgattgggttaactttaaattagatgaaggtaacctatatgatgcaggtaatacaattaatgtaaaatacttaaatgaatctactacgcagtttgctactttagtaaatccacctaaaagcaacttgaatacagggtggataaattacaaagagagtaagaatggtatgtctgctttagctgaatttaaccctattaactctacttctacttttaaaatgattagaaagttacctaaccaagaccaacaacctaacttactgagagatagtttgtttaccttacctatggctgatatatctaatgtaagaacggattacatagataatacatctttttggggttttaccgataatgcttctatatcaacagttaagtatatgggtgaaaatacggtacagttagatgatggtaaggagacgtatcctacagttatgtcaaaacgattcagaataggaaacgaactatatgtaggagatactgttacaatgtctgtgtatcttaaagtaaatgaccctaatttgttaaatggtaatttcccttactttgagattgctgggtatgattatgtttatcaagacaataatccatatacaggaggtaggagagaggttaactattatgatttatctacggagtggaaaaagttctcttttacctttactatacagtcctataatcaagagtacggcaatggaagagttaattatatatcagctttattaagatttaactgttctaactctactaataatggtgcaattgtgtattatgcactaccgaaattagaaaaatcatactcagtaactccttttattacaaatacttatgataatagaaagtatgatgagatatggagtaattggattgaaacaacaagtaaagatgagcttcaatcacatagtattgtagacacatctaataatgattattacaagtatatgtggaattaccaaaacgcaaacggtaaaaatttacaacagttagcaatgtctgtacctcaaggtttttataccttttactgcgatggttctataagtggaactcctagtaataaaacaatacaaggtacaatacaagtagcatataaaaataataatataaacactacaaataaagtaatacaaatggattttaatgacactaaaggcaagaaatattctctaacttataataaagatgataatggttggcaagacatgaaagttcaaatggctagtaacagtctttgggaaggaacgctcgacctatcttcaggaaatatagtaaacttatcagattcgttagataattatgattttattgacgtagtatattggactagaagttctggacataatgcaactagaagattccctgctactacatctagtattgtaattagagactttaacctagtaaatgatggctctaactctagtgtagatttctttgaagggtattgtgatattactgatagggtaaacgtcactgctaaaatgtcaaaaacagttaacctagacggaaatagaaatactacatcagtagcacaattcaatcaaacaggtcacattgaagtactaagagtaatagggttaacatatatttaataaaaggggttttatagatgataaagtatgatattggtaaagatgaaattgtattacatttaaaagataataaatatattatggggtacactattgtaggaggttataatgagagcaatggtgatgtaaaaatatcaaggtctattttacccgatggattctttgctgactttgttagtaataagtatgtgtactataagaacaccaacgaagtaatatataataaaaactttgaagataatacaaagagtgtagaaaacacagaataccaaccgccagcagagtttgtacctaaagaagattacaaaaaactagagagtgaagtagaagaactaaaaagaatgttacaagaattgttatctaagaaaggataaatagtatggcactaaattttacacctattacggaaaatagtacaatagcagacctaacaaaacaagttaataatataggtggtgtattaactgaggatagaaatatcttcgaagtaactgcagacttacaattagatgtaagtagaacacagaaagttaagttaacaacagataaagggttagctaaagatatagactataccaattacttaagagatattaaaaaagtaggtttatattacataggggcacgtacattagctactatgtatgataaaccagatatggaaaatatagatgtattactgcaagtattaacactagatactgaggatagagtaatacaaatattgtatacattatcaacggcggatagtaaatcaaaaatgatgcatcgattcgttactaactatcaaacatcacaatggcaagtagtacaaagtttacctaataataaatatacatctcatataggtggaagtccttttgatattaatacacaaggtacatattacgtgtctaatatgtcggatatgcctagtggtgtttatgagggattcttacaagtttctatagataataatgataatagaatgttgagattaacagatagtagtacaggaaaagaatatcttaatgttaagaaagagtatggcaattggggttcttggagaactgatttagatgtacaaaagataagcaaatatcttgtatctaatgtaaatggagatgcggaaaacaataattactcattatccgtatatacagcagataatataacattccaacaagctatagctaaacatatcgaagaaacaagtaaaactgtttttactttttatgtacaaggaggtgttgtagggtctccatcttcagttagttgtagaggcgtattcatatcagattcaactaactttactaatctatatggtgtttatactgctataggtactgatggcagaacgattaatggttctgtaaatggtaacacatggaatgtagcaaaaacattacctggatttaaagagctgtggaatggtgcacataactttaaagatacaaataaaaaagaaaccatgtctgattctatatctaactaccagtatgtagaagtatacacaagatacagagccttacaaaacacaaaaggtacggataaaacaggtactttatgtcataagttttatgtagatggagatggtatttacacatgttcaggttcatatgtatcaggtgactctaatattggagtagagtactatcgagtaactttatctattactggtgatacatggacagttaaagatagtgcagtaaataataataaagaccaatatgttacacgtgttgtaggtatctctttaccatagactaggataattcctagtcttttttttttcttgactaactttaataaatatggtaagataataaaggactaaaaagaaaggagaatatatgcagttaagacaacagaatatatatacatatgtagattttgatgaaagtgacgggtatattaaagatatcatacttaagcgtgttcataaaacgttaggagctaagaaagatggctatcaacatagcttagcttttaaacgaggtgtgtgggatggttatgtagatttctatgattatgctgagaacaaattccctagtgggttattacctaagatgataacattactaggtgagttacaatcaagacataactttcaatacgaaattattgatgaaaaatctgaaagctttttagctgaagaagatattgatgatgaaatacagctactagataataacgtaggtaagattacattacgtgactaccagtatgaagcggtgtttaatagtttagtcaattataatggtatatgtaagcaagctaccaatgcaggtaaattttgccccttatagtagaaatattataagtgtaactcctttaattcacggaaactctctcatttgagacaatcatgagccaagcttagtaaatatcaggaaggtgcaacgactattatgtacatccaagtggatggaaacagggagaacctaagtctcattagatatggttaagatatagtctgaactttgtaggtgactacaagaaggttaggaataacgaacctaatcgtaacataattgaaaacagaggttgctagcggtatcatagaccaactattaccacaactagaaaaaggagagcgtgtggctttctttacaagctctaccgagatttttaatcaatctgctgaccgtttaagtgaacgattgaatataccagtaggtaaagtaggttctggtaagtttgatgttaaacaagtaactgtagttatgattcctactgttaactctaacttaaaagaccctacagaaggagtgaaagtatctgctaaagtaaacttaagtaaaaagatagctaaagagatactccctaaatttgaaggtggtaaaaatcaaaaaaggttacttaagttattattagaaagtacgacacctaaaactaaagtagagcagaatgtgcttgatatactgcaagatatttatcataattcaaagacggatgctgaagtattaatggcattaaacaatcataatgttattttccaaaaagaagtaagaaagaaaaatcaaaagagttatgacaagtatcataaaatgcgtgaatttctagactctatagctgtaatgatagttgatgaagctcaccactctaaatctgattcgtggtatacaagtttaatgtcctgtgagaatgcactatatagaatagcactaacaggctccatagacatgaaagatgagctactatggatgcgtatgcaagcattattcggagatattattgtaagaacatctaatgagtttttaatcgagaatggctattcagctagacctacaataaatattattcctgttgctaatcctgatgatatagataatattaaagattatagagatgcatataataaaggtattgtacagaatgagtttagaaataaacttattgcaaaactaacagctaagtggtatagtcaagataaagctacgcttattattattaattttgtcgagcatggtgaaatattatccaatatgttgaatgatatgggcgtagagaatttctttttacatggtgaaatagagtcagatatgcgtaaacagaagttagatgaaatgcgtaatggtaaacttaaagttatgattgctacaagccttattgatgaaggtgttgatatcagtggtattcgttcattaatcctaggtgcgggtggtaaatctttacgacaaacactacaacgtgtaggtcgtgcgttacgtaaaaagaaagaagataatacaacacagatatttgattttaatgatatgacacataagtttttatataaacattctaatgagcgtagaaaaatatatgaagaagaaaaatttgaaattaaagaattaggaaagtaggtatgagtagtgactaagacagaaagaagattatatgattacattgaaaaaaaatcaaaaaataatacttatcaattagctactaaaaaagagttagcagaagagctaggtgtctcagtctctactttatctaaaaaccttaaaagattagagcaagataataaaatcaatgttgtttctaaaagaggaaataaaggcggtattattatatcactagtaagagattatgatacagatagtctcttacattttaatgacacagatgataatgttattacttctaatctagaatacgctactgaattacgaaatagattttttcctagctacgtatatgaaagaaaagaaaataaacgtagaacaaaactagaaatggttcaatataatgctattaaagataaaaacaggaaaataatagcagatatgaatttttataataatagtttaccttatcctactaaagatatttttaatatgtcttatgaccctgagggtttttataaagcatatatcttatgtaagttatatgaccaatacgctatagcacatatgaatgctaagtacatacaacatttaacattagcaaatggtaatgaagatgcagatagacataagcatttatctgagtactacagaaagaaaatgttacagaatttatcacgagatagtatttgtaaagacttctttggtagtaagacatttaatacattctacaacttttaccttaagataaaggataaaaatattaatgtatttaagtatatgcaaaatgtttttaaaaatacaacatttggttttgagaataaaacacaaccttaccctatacctgcaccaaatttttttagttccgataaatacctaactaattatgaaaattatgtaaaaggtattaaacgtggagtaaataaaacaaatagacaagtaggagaagtagagtctttaattaagtcatctgattacctattaaatcctgctgtagcacaattacatcaattatatacaacacctttaaatgaagagatacatgatattgatacaatgtttaaaacagctttagatatagaagatactatttatggtatatttaatggcatgaaacatattatacttttatcatataataaatatatagaacatctaatagaaaaattacctagtaaacataaaaacttattaaataaatttattaaacaatgtattgtaaatgagtattcacctactacaatacctaacaatgctagactatctatgtttttaatgcaaaaagagtataaagcaaataccgcagaattaaatggtggtatagatagaagagacttaatagatataagtttagtaaacactacagacttaagtaaacaagatattgtaaatatagaacaaacaacaatgaattatctacatatgagaagatttacttcaacatcatatattctacaaatgtatagtaactacttaggatatgaggttaatttaacagaagttaagactattattgaaaagtataatttatttgatgaaataccattgacaaaagaaggtatgatagaatacaataaggttattgatatagtaaaggatgacacatatgagtaaacgaattaaggagattatcctacataaatctatgaaggatatacattttgcaagagaagtactagataagttacctaaaagtctatttgctacagaatcggaagacatgggttacttattcacagctataaaaagaacagcacacattgctgataaaatgtcaaatgaatctttagctattaaagtagaacagttaatgggtaatgataaacaagatgacgagaaagtaacacaaacattaaaatatttagatagtttatatacagtagacatgaataataaagacgattctgtaaactatgaagtagaaaagtatattaaaacagaaatgtctaaagaagtattagttaagttcatagctgagaataagcaagaggactcagataacttacccgaattagtagagaaactaaaacaaatagaagttagtgacattaatggtactgatggtgaatttattgacttctttgaagacgtagataagaagagggaactattaagtaacttaagtattaataaatattctacaggtttccattctatagaccaacaaattgaaggtggtataggtagaggggaagtaggattagttattgcacctacaggtagaggtaagtccttaatggctagtaatttaggtaaaaattatgttaaacaaggcttaaatgttttatatattgcactagaggaaaagatggacagaatggtattaagagcggaacaacaaatgataggtgtacaaaagaatcaattacttaatggtgacttatctttaaacacagatgcttataataagatacaagaacactataaaaagaacagacagctactaggtaatttttatatagttaagcatatgcctggtgaggtaacacctaaccagctagaacaaattatagttaatacaacaattaagaaagataaacatatagatgttgttattattgactatcctaagcttatgcgtaatccttacctcaagtatcaaagtgaatcagatgcaggtggtcgtatttttgaagatattcgaaaattatcacaagaatataattttgtttgttggactttagcacaaacaaacagaacagcttatggttcagaaattattacaagtgaacatgttgaagggagtagaaagattgtcaatgctgtagaggttgcttttgccgttaaccaaaaagatgaagaatttaaaaatggtttcttaagattgtacttagacaaagtaagaaatagttcaaacacaggtgagagatttgttaatcttaaagtagaacctactaaaatggttgttagagatgaaacaccagaagaggcagaagaacataaacaaatactagcagaacttaacggtgaagataaaagtagatttaaagaaaaacctaataaagctgaagctattaataattcttttggaggtattagtttctaatggacgattttactaagctttataaaaaagtacgtagaacatttacacgtaataaagcagaatgggattatacaaatacagggttaagaactttccaattaaaaggaactaatgtatgtgttacattactagattctatagcaaggaacaatacaacaggatataagtataacgttatgtcgaataaagtacctgctataccttgcaatacacatcaagaagtaatcaaagcaattgaatataaattaaaaaattgagaaaaagccttgacaatatgtttgtcagggcttatacttttattataaacaataaagaggtggtatactttgaacttaaatgaagcaaaagtaaaattagaaaactttaaagttattaataaaaatggatttatagaaactgcgagcacaagatatactgaaaaaaaattagaagatgtttcaactaacatatttaatacattaaataattacgtaacagataaatatggttgtaaattgtttaatattaaacctaaaaggagtattatagagttagaattaacaaacaataaaattaaacacttaagaaagtaggatattaatatggaattagaattaaataaaatatataacgaagattgtttagaaggtatgaaaaagatacctgacaatagtgtagacttaattgttacagaccctccatatttaattaattatagaaccaagcggagaaaaaataaagaacatagatttggaaaagttattttaaacgataataacgaacaacttattatagattatataaatgagtgctacaggttactaaaaaacaatagtgccatgtatatgttttgctcatcagacaaagtagatttttttaaacaacaattagagaagaaatttaaaataaaaaatatgattatataggtaaaaaacaatcatatagcaggagatttaaaaggttcttttggtagaaaatacgaaatcttatttttagtagtaaagggcaaaaaacattttaatgggagaagattaactgatgtatgggaatttgatagggtaagtggtaaaaaacaattacaccaaaatgaaaaaccattagatttaataaaacaatgtattatgaagcatagtaataaaggagatgtggttttagatgggtttattggtagtggcactactgctattgcttgcatgagtactaaccgcaattacattggttttgaattagacgaagaatactataatttagccagtgaaagaatagaaaaacataaggagatactaaacaatgaaaatgacaatacaggaactacaagataagactgaatttttaagacatgtaaaggtagatacggaagaagcaatggatatccttgaagatatattaagaggagagccttacacagatgaggtatgtgaattgatacatttttattcaagtaagctactaaatgagattaatgacaatcatatgtttaaattacaggagtttgctaatcatgatgttatagaaattagtgaagagggagtaaaataatgataaaattattacagggggattgcttagagttattagatacaattgaagatagtagtatcgatgctattatcacagaccttccatacaatattgcaaaagaaaataggttcacaactatgggcagagctggtattgattttggggattgggataaaaattttgaaactaccttatggattaataaagctataccaaaagttaaaaaagggggtaatatcgttgtgttttgtgacatgagacaatttacacctattattgatactatggaagcattaggctgtgaatttaaagatgttatacgtatggaaaaatctaatcctatgcctcgtaatagagatagacgatttatttttgatactgaatttgctctttattttgttaaaaaaggagctaaatggacttttaaccgtttagatggaaaatatgaaagaccttgcattaaaacaagtgttacccctagaagtgaaaaattaggtaaaggacacccgacacagaagcctttatatgctatggagtggttgttagagcgtttaactaatgaaggagatattattctagactgcttcatgggaagtggaacaacaggtgtcgcatgtaaaaatttaaatagggattttattggttgtgaactatcaaaagaatatttcagtatggcacaagacagattattaagtactacaaattaaaacaaaacccttgacaacatgtttgttaggggttataattttgttataacgtactaaaggagggtactaatgaaatttatattttttacagatagtcattttcatatgtttacaaattatagtaaacctgatgaagaatatggtaatgatagatttaaagaacaaattgagacattacaaaaagtatttgatttagctagagaaaacaaagctaaagttatctttggaggagacttattccataaaagaaacgcagtagacacaagagtatataataaggtatttgaaacctttgctaagaatcaagatgttaaagtatatatggttagaggaaatcacgatgctgtgtctaattctttatatacggaatccagtattgatatatttgaaacattacctaatgtggaagtaactaaagatttacgtagtgactcactttctagtaaagtacaattaactatgtgtgcttacggagatgaaacagaagaaatcaaagagtatattaaaaaatcttatcaaaaagataaagtaaatatattagtagcacatttaggcgtagagaatagtttaacaggtaaaggctctcacagattagaaggtgcgtttggctatcaagacctattaccagaacaatatgattttattcttcttggacattaccatagacgccaatacttaaacaataatcctaaacatatgtatggtggttctctaatgcaacaatccttttcagatgaacaagaagctaacggtgtgcatttaatagatatagataagctaacatcaacgttcattccattagatactagaaagtttattacagtacaaggtgacaatgtaccagataacatgaatgatttagtaaacaagggtcactttattaggtttattggtactccagaacaagctaaagtaattgaattagataaaacagaagatgaatctaatgtacaagtacaaatgaagaaagaatacacggtagaaaagagagtcgacgctgacgtgtctgacagccctataacgattacttctagttatgcagacaaatattatccagaagcaaaagatgagcttctagagtgtttaaaggcggtgctgtagtatgatagatattattataaagaatattaaaccaagtatagataaatctaaacctattttacagttcttacataataaaagaacatatgatgtatgtaatgttaaaaatattgatgatgcgatgtaccttattaacaaagtattagaggaagctaactacgctcattattatacgagggttattgaacaagaagattgttatattctagattatggttcacatcaatgtttctttaaattgttaaaagaaggtggtacaaatggttaagtttaattatgtagagatgaataactttttagctattgagcatatcaagctaaatctggataagcaaggtcttgtacttattgaaggtattaataaaacaaatgatagttttgaaagtaatggaacaagtaaaacaagcatgatatcctctataacatatgcactgttcggaaagactgagaaaggtttaaaggcagatgatgttgttaataagtataagaagaaagacacctacgttaagctttctttcaacataggtaaagacgagtatttaatcgaaagataccgtaaacataaagaaaacaagaataaggttaagctgttctgtaataacaaagagataacaggctctacaaatgatgttacggatactcaaatacaagagttatttggtattccttttaatacatatgtaaatgctattatatatggtcaaggagacattccaatgttttcacaagctaccgacaaaggtaaaaaagaaatattagaatctattactaagactgatatttataaacaagcccaagaagtagctaaagaaaaagttaaagaagtagaagagaaacaaagtaaagaacaacaagaaatagaaaaactagaatataaaagaaacctaaaacaagaacaatataataatgaagtaaataaatataataacttattagagcgtaagaagcaagaagaggaacagtttaatcaaaagaagcaagaatatgaggataaattaaaagatttagataatcaaatcagtgaatataagaatgatattcctagtattgaatcttctgaatttgtatttagtgaaagctatacaaaagccaagcaaggtatcgaaaagattaatacaaatatcaatgataaactgttacctttacttaaccaagaaacaacaaataagaatacaactctaaatatgattaatcaactaaaacaatctattaataaattagatacaaatgaccattgtcctgtttgtggttcccctatagataatacacacaaaatcaaagaaaaagaaaatttagagttacaaattcaagatgagcaagctaaaatagcacaatatgaacataatgagcaggctatagttaacaagaaagaagagttactagctaaatctaaagaattacaacaatttatacaacaagaagatatagaaaagaaaaaacatgatgatgacattcaatggcaatatagagaacaacaagaggtgtataataagattagtcaattagaaaatactagagctaacttaaaagaaccaacattaaatgattattcttatatagaagaacctaatgaagaattacacaataaagaactaaaagatattgacaatactattgacaaacataaggaaaatatagtacaattagagagtaagaaaacaaaatataaacaagctgtagatgcatttagtaataaaggattacgttctgtaatattagactttattacaccgttcttaaatgagaaagctaatgagtatttacaaatattagcgggttcggatatcgaaattgaatttcaaacacaaatagaaaatgctaaaggagaacttaaagataagttcgatgttattgttaagaataacaatggaggggagtcttacaaatctaattcagctggggtaatgaaacagctaggaaataattacaaaaatatataaactgtttgccctgcttatagagtaatctgtaagtaccagaggttaaatgctatgaaatcctaaagctagttaaactgaaacataatctgaaaagataaatggaagagttacgaaagtagaaaaaattaactagatgtactaaggttaaatcctaaggtatagaacaatggaagtatagcagggaaagtcctaaatgagcttatagctcacatggaagaccctcaacgactattccgtataggtgacggaagtaaagccacaagtgattggtggaagaaaaatctctatcctagtaaaactaggaataacaaatagtctgcgcacgtaggaaaacctagtgtctagaattaactagagaatatacagtagcgtgtatatttaaacattgttaaaatttataaataattaaattaaattttatcctaaattcctgttgacatattcatttctttatagtatattagtactataaggaggtgaaatgaatgtcaaaggaaaaagaaacgtacaaatatttaaaattaagattagtaccaacgaaagaacaagaatctaaaatattattgcatataaaccattcaagaggattaagaaatattttagttgaagaaggaaatagaatattagatactgataacgagtatatttatagagatacatttagaaagtatcttaaatatctacttaatttagaagaataccaatggttgagagaggtatctaaaggctctatatcttatttatgtgataagttataccaacagtataaaaggtattttaataaagtttctaacaaacctaagtttaaatctaaaagaaatgctaaaatgtattattatccaagaagtgatagtaaagataaattttacttagataaaaataataaattgcaaattgaaaaattaggtcgagttaaaatatctaaaggtactttaaaatctaatttaactaaattaaaacttatagagaaaaatgatagattaagacaatctattattacttttgatggaaaatactggtatatatctatttcatataaagttagtgctttaagtgagaaccaagctaaaaaagaattaactaatgaagttataggtatagacttaggtattaagacattagcaacttgttcaaatggtaaatcttataaaaatattaataaaacaaaaagagttaagacacttgaaagaagattaaaaagaaagcaaagacaacttttacgtaagtatgaaataaataaggattactttactatttgtttacttgatgagaagggtaaacctaaacgtaataaaagtaatggagaagttattcaaaagaacataagaaaaacacaaaatattattaagttagaacaagaaattaaactcttacacagaaaacttactaatatacgtaaaacttatgtacatactgtaacaaaagaacttgttgaagaattaccacaagaaataatcatagaagatttgaaagtaagtaacttgatgaagaataaatatttggcaccaagtattgctaaagtaacttggtatcgatttagagaatatcttacctataaatgtaaagatagaggtatattacttacagtagcaaatactttctacccttcaagtcaattatgttcagtgtgtggtaatcgtaaaaataagcaaaacaaacttaatcttaaacagagaatttataattgtgagtgtggtaatacacttgatagggattataatgcaagtcttaacttgaaaaactatagatactccaattggtatcatgaaaatgttaatctcaagctaactaaaacagattaaccttatgtacctgtccgtaacagggaattgaaagcctttggagtgctatagcaactttagtagaagtcataaaaagtaattctatttatagaacgaaagatttcgaggaagagtacgtagaagaaggaataaaatttatagtaattaagttaattatttataagttttaacttacggaacaaaaacgtattgatttagctattagttttgctatccaagacttaattatgagtaaagaagatatatctacaaatatagccctttacgatgaatgctttgacggactagacactattggttgtgagaacgtagttaaactactaaaagatagactaaaaacagtaggtactatttttgttattactcataataagagtctagcacctttatttgagaatacaattacggtagtaaaagaaaatggtgtagctacactaagaaaggaattaaataatgaaattaaagattaaagataaccagtttgcatctttaacagtaaattatacaaacaatgcaaagttacatatagataatattcctgtttctactttagtagactggtaccctttaagtaatgcatacgagtataaagctagtaatgactttggatatattgaactaaagagattacgctctagcttacctgtgtcttatgggttaactcaccaaaccttatataaatgtgaaactgttaaatgtaaattaggattatggtataatgaaaaaataaaagaagataatgagaaaattattgagaaagctaagttgtacggtttacctactatagatgaacccttcacaagtaaagatgtaaagcaaggatttagtgacttaggtgttattttccaaacgctaaaaacaatcagtacaaacgagtacttaaaagataaaacaattgaagagattaacacctttagtaagaaatcagaggattatcaattaaatgaagtactcaagtacagtacaacactggtagatgatacttatagtgacttaagtcaaatctataatatgttactattaatgaagaaaattgtttctatataggaagtgactgaaaatgaggtttgaagactttttaagtcaagagattggaacacctaaagagaatactgtaggagaattacgatattgttgtcctttttgtagggaacagaaatacaagttttatgttaagcagtctttagactctactaatggtatgtatcattgtaagaagtgtggagaacatggtaaccctattacttttatgaagtcttattatagtattacaggtagacaagcttttgagttactagaaacaaagaatattgatatacaattctcaccaacattagaaatatataacacagatttaactgaaagtgagaaattattactaaggcttaatggatatacaaaagataatagtagtataaaatcaaaagcacctgaactacctatagggtttaaatcgattaaagataacttaaataataaagaagtagtaccgtacttaagatatctcaagaatagaggtattacactacagcaaatcctagattacaacataggttatattacagatggttattgttattcttatggtattaataatgagaaaaagaaaatagtacttagaaatagtgttatattttttacttatgataatgaaggtaagtatatatattggaacacaagaagtattgaaactaatccatatattaaatctataaatgcaccttctaaacctaatgaatatggtaagagtgatgttatatttaatttaaatatagctagtaaacaaaagtttgtcgttataactgaaggtgtttttgatgcgcttacctttgacagatacggtatagccacttttggcaaacaagtttcaaagacacaagttaacaacctcatttcaagtattcctaaagaaacacctatatatattatgttagacacagacgctacagactttagtattaatttagctagtaaacttatcatgcattttaatgtagtgtacttagtacctcatggtaatgaagacgctaatgatatgggtatggaaaaagcttttagtactttgaaacacaatagatttttagttacacctgaaagtactcaaagttacaaactacaacaaaagttaaaactttaatcctgtactataatgtataatgtgttataatacatatataaaaataaaggaagtgttagtgtggaagagaaagagaaagatattctacaatttgtagatgaatatgttacagcattacgtgtaggtaatgaacagcgtatgcaccaattagaagagttaggcaaagaacaaacagcaactttaacagatgttatgaaagctattactaacttattgttaggtgttaataaacaaatggttgacctagagcaaaatacagagttaaacttaaacattctaattgatgctttatacaaagctgagttagttaacgatgacgtattaaaatatattgaggaagcaattgaagaatcaaaagaaaaggatgaagaataatggaaaaaaatattagtacacatacaaaaggtatttcacaaaaagatatggacatgtggttagaagctattacacaaggaacagtagaaggcaaggaattaactgaggacactgctaagcaattacaccgtattggagcacgtagtgtatcattagatgaagctactagaattgcacgagcaattaataccattacagttcaagagtatgcatctgcttttaatgatgcttttaatgctattgatttgttaatgattgttatggaagataacctagatgtaactactgagcaagtagagaaggcgcaacttaaattaaaagaaaaacgtgaaaagtatttagaagaaaaacaagaagaagtaagaaaaaaacaagaagagcataaagcaaaagaagataatgaaaaagtagtgcagttgaagaaacatgacaaatagtaaaaaaaaaggtgatgtattcgaaagaaagattgccaaagaactttctgattggtggggttatccttttagtaggtctccacaatcaggaggtgcatcatggggagctaacaataatgctgtcggtgacattgtagcacctttagagtctaattttccattagttgtagagtgcaaacatagagaaaattggattatggacaatgttttacttaataataaagaacctcatacatggtgggaacaagttataggagatagcttacaagttaataaaactccttgtttaatatttacaagaaatagagcacagtcatatgtagcattaccttatatagaagatgtatatgtaaagttaagagatggagagtaccctattatgagaacggattttattgtagaaaatgttagaaaagataaacatttttatgatgttcttataactacgatagatgggttgactaacttaacaccttcttatattatatcttgctacaacaaaatagagattaaaccatatagagttgtcaagtctaaaatatctaaagttagtaaaaaagaagataagttgattgatgatttacttgatggtatataaggaagggatactagttgattacacaaataaaaaaacgtgatggttccgttgtagattatgatttaagtaaagttactaatgctattttaagagccaactcagaaacagaagaatatattgaagatgttattgattctcttgtagatgacgtagactctttactagaagaacaagacactatcattactgtagaattaatacaagatatagtggaggaagttttatttagtagtatgtataaagatactgctaaagcttttattttatatagggataaaaagaaaagagagcgtaaaagagacctattcaaaccaagaaaagaattaaaaccctatgaataccctgaattattagaatataaagatgctatacgccaaagttactgggtacacaccgagtttaactatacatcagatattcaagattataagaataacgttaaatacaatgagcaaatgaccattaaaaaagctatgctagcaattgctcaagtagaagtagcagtaaagactttctggggtgacctatatcatagaatgcctaaatgggaaacaggagctgtaggtgcaacttttagtgagagtgaagcacgtcatgcagacgcttattcacatttattagagattttaggtttaaatgaagagtttaaaacaatcgataatatacctgctcttaaagaacgtgttgatgagttagcaatgcatgtaaaactaagtaagagtgaggatgatagagactacgtattatctgttttattattttctatatttattgaacatgtatctttatttagtcaatttttaattatgatgagttttaataaatataaaaacttatttagaggtttatcaaatgctattgaagctacatctaaagaagagcaaattcatggtttatttggaacagaaatcattaatattcttagagaagaaagacctgagtggttcaacaaagaaatggaagatgcagtttacacagcgtgtaaaaaatcttatgaatcggaaacacgtttacttgattggatgtatgaagatggggaactagagttcttacctaagaaagtagtacaagaatttattaaaaatagacttaataactctctagtagctgttggatataataaactttttgatgttgatgaagagttactaagtgaagtgtcatggtttgatgatgaagttattagtactaagttaacggactttttatctaaacgttctgtaaactatacaaaatttagtaattctgtaacggaagatactattttttcaacggaatctgattttgaaaatactggtaaacgagttactaaagataaagtaaacgctattttaagactaaaaatgatataaaggcagtgtatatagatgagtaattttgaatggttgaataatgattctagagtttttctagaacgtggttatttaagtgatggtgaaacaccagaacaaagaattaaagatattgcagattatgctgaagatatcttagatattgatggtttttctgacaagttttatgattatatgagtaaaggttattattccttatctagtcctatatggtctaatttcgggaaagatagaggatttagtattagttgttttggtagttgggtagatgatactataccatcgatacttaatactgctagtgaagtaggtatgatgagtaaatacggtggtggtacaagtggttacttcggtaacatcagacctagaggctcagaaatcactgataatggactgacaagtggttccgttcattttatgaagctatttgagcaaatgacagatacgataagtcagggtagccaaagaagaggaagatttagcccttatctacctatagaacacaaagatattgacgagtttttagagattggtaccgaaggtaatccaattcaaaacttaacacatgcggtaacagttactgatgattggttattagacatgattaatggggacaaagaaaaaagaaaaacatgggctaaagtattaacaagaagaacacaaataggatacccctatatcttctttcatgataatgcaaatagtaatacagtagatgtatataaagacaaagggttaactattaataattctaacctttgtgttgcaccatacactaaaatactgactgacgagggatacaaagaaatagataaacttaaagaccaaaaagttaatgtatggaatggtgaagaatggtctgaagtaacagttaaacaaactggagaaaaccaagaaatgttaaaagtaaaaactaatagtggttttgagttagactctacaccttatcataaatattacgtattagaaacaactaaatcaggtaaaagagcaagaaaaaataaatataaagaagttagagctaaagaccttaaagtaggagataaattaattaaatttgatttacctgttattgaaggtcaaaaagaactaaattttgcttatgataatggtttctttactggtgatggaacactaaatggtaaaaatcaagttatctatttgtacggtgaaaagcaaaaacttattgattattttaaatctatagacaaatggtcagaaagtaaagaacaggatagacatgtaggtcttactaaaaaattagaagataaatattttgttccaatgaacgagtacactattgatagtagattaaaatggttagcaggtttcttagatgcagatggtacagtaacaaataataatggttctcagtctattcaagcaggttcaattaacaaagagtttttaaaagatgtacaattaatgttgcaaacattaggagtagatagtaaagtaactctaggtaaaaaagagggaacttctatgatgccagctaatgatggtactggagaatataaagaatttaagacaaaagagctatatagaatattaataaatggtaattctttatataaattaaatcaattaggtcttgaatgtaatagacttacatgggaaacaaaagaacctaacagagaatgttctcaattcataaaagtaacaggaattgaagaaattgaaaaaacaatggatactttctgtttcacagaacctaagagacatatgggaatgtttaatggtattttaacaggtcaatgttctgaaattatgttacctaataaagaagattggtcatttgtatgtgacctatctagtatgaaccttgttaagtatgatgagtggaaacacacggatgctgtagagacacttgtttactttttagatgctgttatgtctgagtttattagagacttagaatatctaagagactcagatgaaaaagataagcaagaagcatttaaatttatggagagagcttataacttttctgttgagaacagagccctaggaataggcgtgcttggatatcattcatacctacaatcaaaactcataccttttgaaagtattagagctagccaaattaatgaagagatatttaaattattaaaagaaaaaacttataaagctagtgagcagttagctaaactttatggagagccttctattttaaaaggttatggtagaaggaattcgtgtttgatggcggtagcaccttaatttatagggcgtatatggattaacatatatgataacactcttgaatttctgaaaagcctaagtgtgtaaacataaggtaatcagaagctaagatttatttatagttaacttgacaaaaacaaatatatacattatactagttactaagaggtgataataaatgtggaaaaatagtattagttttcctaggctcgaagtaactaaggatggaagagtaagaatgtggaataaatcatggaaaagatatgtagaaaaaagacataggtatgataaagatggttactcaataatttctacaagaaaagaagatggtacatccacatcagcaagagttcatagattagtagcagaagcgtatataccaaacccatacaataaacctgttgtaaatcataagaatgggattaaagatgataatagggttgaaaatttagaatggactactatatcagaaaatactaaacatggttatgatgttttaggtgttaaacacgctaacgctcaactaattttagtctacttagatggcaaacctttttctagttatgactccttaactttaatgggtaaaacattaggtataaatagaaatcttatacgtagagtagaaaaagaaactgatgggtacatctcatttaaatggatagaaaaaccatataaagtacacaataaagatttttggaaaacatcaagaaaattaaatacgagaggctcttgttatgttgtaaataatgtatactataatagtgcaaaaaaagctatggaggctcttaaagtaagacaagatgttttctataatagacttaaaaaagataaaaatgtatatagtgtaagtattaaagagtacttaaaaaattgtcaagatataaatagataaagttcaacgactatcctgttgcggggagtacactcaagtgagtggaagtgggagtggtcttacatttagtaagatgtgatatagtctgctctctatggaaacatagagctgttattaaattaacgcatgtaaagtaacgaactacatggaacataaaggacaacaagttcatcatttatcctaggtcaagtttctaaatcaattgagccttttatgagtaattactacgttgtagacactgctaaagtaaagaaaactatgattaatccttaccttaaaaaattattaaaagaaaaaggaaaagacaataaaaaagttattgagagtatcagggataatgatggttccgtacaacacctaaccttcttaagtgattatgaaaaagaagtatttaaaacatatggtgaaattaatcaatataatatattagaccaagcttctacaagacaaaaatatattgaccaagggcaatctataaacattatgattaatcctaagcatgttacagcagaggagttaaatgaactttatctctttgcatggtctaatggtattaagtcattgtattatcagcatggtacaaatgcatcacagcaattcaatttatctaaaatttgtattaattgtgaagcctaaagaggtgactaaatgaataaaaaagaagcattagaactactaaataaagtagaagttatatttaaatcaaatgatattttctcacaagtaagtaatgtacaagaccctattaagatgtttaatagggaggacgatactaaagcagatagtaaatcaagtaaatttcagctagagtttatttataatacatatgtgtatattcttatatacgaaggtacattgaagctatttaaagctgatggtacggagcagttaacacatgtagctgatattgaaacatttaaagatattattgacacactagaaaaggatgatacagaaaatggaaaaagtgaatagtttattagaattaaatacaacaattagacaaaaacaggatgttattgtcatggttacacaagatgagtgtgctaaatgtgaaattttaaaaagtgtgattcctctttttgaagcagaaggggatattactaaaccggtttatgttattaacttagatgataaagatgtagacagagaaaaagcggtcaagttatttgatatcatgagtacacctgtacttattggttataaagatggggaacttgctaagaagttcgaagaccaagtaacacctaaaaacttaatggaattagatttactttaatttgtaattacctactacttatgctatacttatattagtaaaaggtagtaggttttttcatgaaaggatgaatagttatcgcaaaaaataaaacattaactatatataatagtgatagatattttaatatacatgtaaaaaacaaagatgaattatctaaagctattaaagttacaactttaaatgaggatgatatagaaaaagatatggatatcttagctaataaatcaactaggtatatactaagagacgataaacattacatgctatttaatgagaagtataacaatgataagttgattgaaaaaatatgtaagcatggtggacaagtaacatactataccgattcagtagttccttactatgtgcttaaagacttatcagcccaccctaaatcctcagttgtatacagaatgcgtagtaaattcagtaacaaagagatggataatatagctttgagtttcatgggaacaaaagtcattattgatatttcagttgtatttccttacgtcaatccttacgatattattagaagtttatacgatgttaaaacgaatgtagacgaggttcatctatcattccctcgtattagttctgtagacgctaaacaaggtaagtactatacgtatgataaagaagcttataaactaaatcctgagtatatacttgattttgcagataaattaagagtttctttgtccgtgtggaaaatgtatatctatattcttgctagtaaagaaaataaagattatgaaataatagaagacctacttactgaattaaaaagacaaagaaagattaagatttaggtgattttgtgagtacagcaaatagaagagatatagcaagaaagatatctgagagtacagggtactatatccaagacgtagaagagatactagaggcagaaacaaaagctatttctgaattgttacttgaagattatgataaggtaaagaatcataagtactttcaaatagaagtagtagagcgtaaaggtaaaaaagcatgggatggattaaataaaacatactttacattacctaatagaaaagcactaaagtttaaacctttgtctgaactagataaagttatagacgtactaaacaaagaggattaaattcctctttttttattgactattattaatagtcatgttatactgtatttaagttaaagaagggattggtaatgatatgaaagttttaattttatacgactatatcagggaggaacacttttcagttggtagtgatggtagtgttaagaatgtattactaaatacacctaatggtagagtattaaaacaactattatctaatatttctggtattaatcgagatagaagtacaaaagattatgatattgagtttttatatcctatggtgcctacacctattaagaataattatggtaaaacaattaagtaccaagacgttaagttatctgaagttaaaccttactatgaaagaatgaatcaaataatcataaacaataagtacgatattattattcctttaggtaagctaggggttaaatatttattaaatgtatcagctattagtaaagttagaggtgtacctaataaagtaactattactagtgaagataaaaaacatgatgtttgggtattacctacttatagcatagagtacacaaacgttaataagaatagtgagcgtcatgtagtagtagaccttaagttattaggtaagtttgtagaacaaggtgaagacgtatttaaacctaaagaagttaagtatgaacttgttacaagtattgagcgtgtaagagaaatatttaataaagaagtaaagaatgataatcatgacggtgtagacattaccgcatgggatttagaaactaactcacttagtcctgatagagaaggaagtaaacccttagtactatcaatgagttgggagaatggtcaaggtgttacaatacctttatataaatcagactttacttgggagaatggacagcaagatattgatgaaattctttccttactaaaagaatgggtagctagcaaagaagacattaaggtactacataatgctacttatgatataaatttccttatggcaacgcaaggtttcacaaactttgaaaataatcaagacactaaggtaggttggtatttagctgtaacacaagaacaagcagagtctctaagattatcagaccttgcttacgaagtaacagatgtaggaggatatgacaaaccccttgaagattttaaagagtggtatgtattgaagctactacgatttttatcagataaactaaaagaaattaaaaaagaaaataaaaaagttactaagaaagaatataacataaaagcaaatgagtatgatacttggttgaaaaataaactcgataacatcgatgtagaactaacagatgaagataggtattatggtatcacagaagagcaaaaaagatatttagagttaaaactaacacctgaggttattacaaagaatatgcttatggattctgaatttaaagaagtagccgaatcatcacctgagtatatgagtttatctaacaaagctaaagattatgtattaggtacatctattaatttaattaacacgtataaagacaatactaaagttattaatgaggtagatggaggtgactttaattatgattggattccattagagcttatgcatccttatgccagtggtgataccgatgtctgtagaagaatatactgtgatgttattgaaaagttaaaagaacaaaatagacctaaagcattggacttaatgtcaataagttaccctagattaattagaacattagctagaatacaatcaaatggtttacattgtgacctagaatacatgcataaaaatgatgaattttatattaatgagatggaaaaaacccaccaagaaattagagaacattgggctatccaagaatttgaggagactaggtataatttataccaattagccttagcagaacatgagaagaaaccatctgatagaaataaagaaatacatgagtatagagctaaatttaaagatgaaggttggaagtttaaaccaagctcaggtgaccataaaggtgaagtattgtatagcattttaggtatacagttaccttatagtaaagagacagttaaggataaacctttcagtaatggtactaaagaagatgagttatcatggaaagattacaaaacagatactaagtctataaagatggcattatctttggttgagaatgaagataataaaaagctactagacttacttatttattatgcctctttgcaaactaaaagaaattcttttactaagaaattacctaaaagagtaaataagaatacacataatttacatggtaactataatagtacaggaactgcatgtattacaggtgattctttagttattactgataaaggtataaaaaaaatagaagacttatcaaataatagaaaagaaaaagtatttagtagtattgatgtaggtattgttaatagacagggaaatttagaaaaagcttcacacttttattatagtggtgtaagaaatggattaaagattactttagaagatggtactacattaactactactttaaaccaccccttattacgaaataattattatagtaatactggtagggttctaaataaaacaaaacaaacaactaagcatttactagataatgattgggttgtagctgaagatttaaaaataggtgattatataaaaatgtcttataattctaatctttataataatagttatattgatttagatactaaagattttatatatagtaaagaaaaaagtattacaaacacaaaaagctatacactacctaattatgtatctgaagattttgctgaatggtacggtatgtatacagctgatgggtctttttctacaaataacggttcattcagtataagattaactaatagtaatcttgaagttaggaatagattctttaacctaactaaagatttatttgatattacaccatactacattagtaataaagatagaagtgattctattgaattttcttcaattggtttaggtagatggttagaacatatttttaatatgcaaagtaaagcattaaataaagaaatcccacaacaaatattagatagccctaaaagtgtacaacaagcatttcttaagggtttaagtttagatactgcaacagaaaagaaaaagtatcctagtttgtactataatactgtatctaataaaatggctttacaaataagaacaatgctaatgaatatgggtatctattgtaggtattctataggtaaagtttataaaaatattaatagaaaagtacaaaatgaatgctattctatacaaataacttatgatgcattagataaattttacgatgaaatagggtttattgaaagtattaagcgtgatagagtaaagtataaatcagaaaatctaaaggcattaggcagaagaaacggtattcttttatatgataatgttttattaggtagaataaagaaaattgaaaaaataaaagatatggaattttttgatttacatgttcctagtagccattcatttgtagttaataatattgtaaaccataatactagcaggctttctagtaacaaccctaaccttcagaatcttcctgctcatacatctgatgtaaataagtttgattatcaccaccctattaaacgttcttttatctctagatttaaagatggtgtaattttacaagccgattacagtgccttagagatgcgtattacggcactatatacagatgataaagaaatgttagagatgtttttaacaggacaagatattcataaaaatacggcaagtattatgtatggtaaaagtatgaaggacgtaacagcagaagaaagacaagccagtaaagctgtagcctttggtttaatttacggttaacaaaatatagccgtagtaaaactaggtaaacctattgagtatagggtgtatagttcacgttaggagctacaggaaatggtagttaagaactatgctaacagggaaggctttaaaatgctaatcctgtgtagaataatttaagtgtaatttaataagtaaggatgatttaaatagaatttaaaataggagaaagatataatcacaataagctatattctaaattagctcatgtatggtctcatatgatagatagatgttataatcctaaatgtaaggaatataaatattatggattactaggaataacagtagacgatagatggcataattttaataactttcttaaggatgtagagttaattgatggttggagtttagagaagtataaaaatggtgagttagtgttagataaggatattaaagaactaggtaataaagtatatagcttagataaatgtaaatgggctactagagaagagaacttaggatttgtaccttcaagaagtaaaaagatattaggtatagaccctacaggtaaagaatatacattcttcaatcagtctaaatttgctaaggaacataacttaattcaaagccatatttctgcagttcttagaggggaaagaaaacatcataagaactgggtttttaaaacactacaagaatataattatcaacctgtagaaaacccttctaaactttatgcacctcactatgttgcttataagaatcatgtggaaattgattatgatagagtaaaaactaagctactaaagagactaaatatacctacgtcaggcactaactttagaaaaatggaaaccggtgaagattgttatgggattacattcaaaatagagaaaagagaacgatttgattacacttaaacacttaaattattttatcgaacgactatcaaaaggtgagctttacgccgagagggcttattgcaatataaccgagtagagtacatgtagctcatacctacatggaatagcctagaagtagatagtaacgtatttacttaagatatagtctagtcccctaataaatatcgggaaaccgagggtataaacggagtcagagttttcgtttgcaggtaaaaataatatgacagttgatgaagctactgaaatatttgataagttttattctaataaacctgctattaagaagtctatagatgaaactcatgagtttgttcaaaaacatggatatgtagaaacaatgaatggacacagaagatatatacactcagcacaatctagagataaaaagattaaaaatgagggtcttagacagtcctttaacacgattattcaaggtacaggtggttaccttactaacatggctattacttatattgacgactttattcaaaataagaatatgaaatcgaagttagtagctactgttcatgatagtattgtagtagactcacctcctgaagaagttaatattatggctaaagttattgtccatgtaatggagaaccttccttatgatttcttaaaaataaaaattaatggtgagttaagacagtaccctatagatgcagatatagagataggattaacatataatgatatggtagaatacaacgaagagttaataaagcaatttaattcgtataaaggatatattaagtataaattagccttacaacaaatcaaagactactatgaatcagataaactaactgaggagcaatacaaacaaaaaacggaatatattaaaaataatattgacagttttaaagttatatagtacagtaaaatagaggagggtattatggagatacatttagatacattagatttcaatgagttaaccttaaaagataacaatggaaacactcagacttttaatatccatgatgaactaaaacttagtgagtatacaattcaagacgaaatgtaccaacagtcgtctaaatttgcatggtgggcttcattaaaagagcgtgtgagtaattatgccgaagcagagcaacgtaagctagaaaaaataggagctcaacttaatctacaaattagagctcaatatgaacaacagggaaagaaacctacaaaagaccaagtagagtctgctgtatttctttctgatgaataccaacagcaagcacaggttgtagaagcatggaactatagagaaaaacaattgcattacattgtaaaagcttttgaaacacgtacaactatgttagtacagattagtgcggaactaagacaaacaaataaaaatggtggtattacaaatccatttacacattaaaaaaggagaagtatgctaaatatggcaagtgaagttaagaaatattttttagaattagctgataaaaaaagactagcatacatggaagagcacggtatagactttatcgttaatagagaaaggtattcttggaaaacaaataaaaaaatgtttaccatcttatgaagaattgataaaagaaaaataaagtaaatggtacggttgacagcaaaggaaaaatatgttatactaattaagtaatttttaaaataaaaggagaaaagatattatggattttaatcaatttattaatcagcaatcagaaaagctacaatcaagtggttttgacaatgaggtagaagtatacaagccaaaacaacccgttttgcgcttaggtaaagtaaaagatgctaacggtaatcaaatcaataaagagtctgcacttgtacgtgtactaccgcctgtagaaggttctaatgagttctttaaggaatttagaacattaggtattaactatgttaaaaaagacggggcacaaaagttctctatgcttactttacccgagaaagttaactcatctgttgtagacctttatgtaaatacatggttaaagcaaggtgtacaatttagtaacttccctaataaacctgcacgacgagcatatattcatgtgattgaatactttaatcaaaatggtcaattagttcctaacacagatgaacaaggtaatgttattattcaacctatggagctatctaatacgggtctgtctcaattgattgaccgattaaaagataaaatgttatcaccatcacctactgctacacatagttttatttcagcagatgatgcattccctattaatattgctaaagctaaaaaaggtgaaaaatcatggaatgtaactgtttatcctacagtaaaattaggtgctttacctcaaggttgggaacagcagttatctgatttagataagttagctacaccaacagaggaaaataaccctaactttgtaaactggttgattaacaatgttaataacacagaagtttctcatgataattttaaattcaatagagaaacaaacacattaggtgaagaaccatcaacgcaagcacctacgcaacaaagcatagaacagcagttgccagataatttaggagggcaaaataacacacaaccaaactttaataatgtacaacaacctgtaccacaggtaccaccacaacaaaacacacaatttggtcaaggaacacctgtacaacaagaaccacaaacacaaagtaatccatttgagaactttgatgcaaacaatatcgatgattctcaaattccattcaacacaaatgaatcaacaccagaaccccctaaacaaaatcaacctaaaagtgtagatgatgtattagcaggtttagatttataataattttatagagtgctaggatagcactcttttatttaaatttcacataaaaggatgatataatggttagagcaaaaaaaggtaaagaagtagacattacaaatttaaatactattgacttaggtaaagagttaggtttaactttactatcagatagtaatagagcagatattaagaatatcattcctactatgattccacaatatgataggattttaggtggaggtattcctcttggtagattaaccgaggtctacggattacaagcctcaggtaaatcgacctttgcagtgcacttgtctaaaatatcaacacaactaggagttattacagtatggattgatgttgaaggtaccgctgataatcagcgtatggaacaactaggtgtagatgtatctaaattgttctcagtacaagctggtgaaggtagacttaaaaatattactgaactatcagtagaaactgtaggtaaagagttagaatactggattgatacatttaatgagaaagcaccaggtgttcctattttatttatttgggactcattaggagctacacgtacgcaacgtgaaattgaaggcggtattgacgaaaaacagcttggctcgaaagcgacagctactcaaaaagtagtaaatgctatctcacctaaacttaatgatacaaacacaggtttaattatcattaatcaagcacgtgataacttaaatatgagtaacccttatgatgaccctattaaatctacaggaggaagggcttttcaacatggtgctagtttacgtattaaagtaactaggggtaaagagtcggagttaaaacagaatgatgttatgacaggtaaacctacgtataaaggtcatgttatgcgtattgaaactaagaagtctaagcttactagacctggtcaaaaagcggaagctgatttattatcagagtatgaagtaggttctgatgaagatactatccaattaaatggtattgacccttaccatattatctataaagaggctgttgagcgtggtttaattactaaaggaacatggcgtaattatgttacgttaaatggtgaagaaattaaaaagtatgataaagattgggtaccgacattaatggacaaccatgagttatacttagaattatttaatagagtttatcatgaaaacttccctaataagtatgcacctttaagcaatacaaaagtagacattacccaactagaagagtttaaagcattacaagattactacgaagagcttagctctactaataaagagactaaggaagatgtaaatgaataatctaatagatagaaatattaatagtgttaaagaagcattaggcagggctaatacaaatgacgtattgcccttaccttatattgaaatagcagaacgttttaaaaaagctagagatactaaggaagctattattgtagaagaagcaggctttccttacactgattctacagttatgtatattgagcatgtagagtcaaggtgggcagggggttattctttagtacgttataatggttctatggttaaagtacctaaaacaatacattattccgatatctatgtatcaaatgatgtacacaggattaagattatatttgagggagcacatccttatgaagaaagttaataatgggaatagatatattatagacttagatggtattcctactgactttggtagagacttagacatgcttttaaagagatataaaaaccttagatggtcattatttcataggtactctagaggtatgagtcatgactttgaaaaacaagagttacgggaatacatagatgagcagtttattaagttagttaaggagtatgatatacaaagtaaagtagattttccaggatatattaaaacgaaactaacactacgagtacagaacagttatgttaagaagaacaataaatataaacgtactgaacttgtaggtaaaacagattatacagtagagtccttaacacaggagttaaatgtgggattagaagaaaatgaattacttaattatgtctttgatgacacacaatttactgaagtacaaagtgaactgctaaaagaactccttattaatacagataaagaagacaacgcatttattgtttcaactgtagctaataggttagaggtagagcgtccagaagtatctagagagcttacagaacttaaagaatatgttaaatttaaaataaatgcttaccacgaacaaaacagtagaagatacattagggacaacaaaatagatacacaaaatcatgtatgggaataacacaaacaagccttccttttgttatattattaatgaaaataataacatagggaggttttttctatgttataatgtaaaaaataatgattggagcttaattattatggctaagaaaaatattaatgaagtattaaaacaagctaccgctactgtagcagataagtatttacaagttcaagtagagcaagatggttatacacgtacacaccgtggtcagtataactacaaagtagtagataataagggagaattattcttatacccgattgaaacagatggtagaggaaatattaaaattatgaaaaaggctcctattgtctatacagatggtgaccaaattcattttgtagttaatactgttaaagacccttacaaccatgcttttattagaactgaaaatattaaaggtaaagataaaggtaaacaattaattcaagcgtttttagcattcgttgaagaccgtttcagctttggagtatacaatgtattccttgctaataacaagaaggatgtttttgctttagttgatgctgaaagtaaagatgctaaaaaagtagtagatagtaatgagaaacctcatgaagacgtgagtgctgagtttcctgctagcccattacgtaaagacgttaagggtgtagattcaggagaaggtcaaggagacacttcagagccttcagtacctaaaaatgtaacagtaaccgctaaagaaacaacagctgatattacagctgaataaacatacaaaggatgacttagtttggataaattaaatttatacaaaggtgaccagctattaaaaagtgaagagaaacaagcaggtaaaacatcaattacaattgataacttaactgctaacacagactacccacaaggtacatataaagtatctttctctaatgaatcaggagaatcagaaaaagtagatgttcctgcgtttaagactaaagatattaaagtagtatctgttactttagatgttgaaagtttagatttaaaagtaggagaaacacataatttagtagctacgattgaacctagtaacgcaacaaaagctacttatacatttacttcagaacatgatgatattgctagtgtatcaagtaaaggtttaatagaagctaaagcaaaaggtcaaactacaatcactgttactactgatgatggtaatcatacagatactgtaactgtagttgttaaagataaagtacctgaagctcctacaaatgtaactgtagaccctaaagaaacaacagctgatattacagcataatcggaggtaaaaataaatggaaaaaacattaaaagtatataaagatggtgaagtagtaggtactaagactgctgaacaaacaggtaaaacaactatttctattagtggtttaacagcagatactacgtatcctaaaggaacttataaagtagcatactctaatgagtctggtgaatcagaaaaggtagatgtacctgaatttaagacagcaccacatagtgagctataataaattaaagtccaacttaattgttgggcttttttttattgacttatatttatttatatgctatagttaaacaagaattaataaaggaggtactatatggatattaaaacagtatacttaacttcagataacgaccatttgaaagttaaaaaattaatggaaagtaaggaaaagtatattgcagttacgtatgataataaatcagtaagtaaggtcaatgtcgttatggttattaatgttattaaagacctagtgcatatgtataggtataaaattgttgagtatgggttatctaataatgataaggataaggaaatggtagggtatttattagaaaggattgaggattgatgttatttattttaaatgaagtagcttcacacacagaacatttatccgaaggtatagatatattcgacgttgaaagtgtagtatcttttgactcggcattacatttagctagtgataatacatatgattctgttatttttttaggttttatatacaataatgaggattacttaaagtataaagaattattaaaggatacaaaattatactttgtttctaatataaacttacctgacaataaggagtttattacagtaggtgatgatttaaaatatttatccttattagagcaacttgtagtattagaagataaagacctagacattaactttagtcagtatactaaagacacggcttctaagtacactgagttgtttagctttaaatctacttatgaggaggcaaaagaaatgggtctagtaggttaccccgcagatatagtattacctattatgactgacaagttttacaacgtaaatgatttgtatgtactatacgaagtagcacctgagtataaattacctttagcatatgaatgccttaaggataaagaaaaaggtattgttgttataggttcacaaacaagaggtactagtgatatccttactttctatgtaaaaggttatgatgttaatagtgttgctaaaacattcggagcatcttataatgaaaattctaatatattcagtatatttattgatagtcatatccatgtgttaggtgagaatatgaataagtatttaataacggaaggaagtatttatgagtaattataaaacagtagaagagttacaagcagttattgtaggtgtgtttcttaaagatgaaggtaaagtagttacctctaaatttaataaagtattagcaagttttggtattgatagagtaaatcgcaatgaattaaaagatattgtagataatattaggaaagacacatacttaaatgacttgaaaaataaagcagttaaaggtgaagtgttattagaggacttaagagatgtagaagataaacaagtatttgagggtaatgattatcatgaagaagtatcatcttatgtagtagcacatgaaaaagaactatctagactaagagaattacgaaagattaatagacaaactgcataccctactattttgtttgatgaattaaagcgtactatggttaatgagctcaaaggtaataagttactagaccatagagtaagtaagtatacttctactgaggaagaagaagagctggttatcttactgtcggacttccatgtaggttgtgcttttcaagatttaactaatgagtataactttgaggtactaaaaagaagattaaatcagctactacaggaaactattaaggatattagtaaacgtggtattagtaatgttacagtatactttgtaggtgatttaatagagcatattaatatgagagatgttaaccaagcttttgatactgagttcactatggcagagcaggtagcaaaaggaactagactattaatcgacttcttaacagaattatctccatatgtagagggtaatttacgtttcggtatgatagcaggtaaccacgaccgtgtacagggtaataagaaccaaaaagtatacaatgatagtgtagcttatattgtattggattctttactgctgttaaaagaaaacggtgtattagaaggtattgagattatcgacaatagaaaagatgtatatactattaaagacacagtatgtaacttaaatattattataaaccatggagatggacttaaaggtaaaggtaagcatattcctaaatttattgagaacactcacattgatttgttaattacaggtcatgtacaccacttctcagtaacacaagaagattacaatagaatgcatattgtagcaagtagtcctatgggatataataattatgctaaagaactacatctatctagaacaaaaccatcacaacaactactatttttaaacaaaaaaaataaagacattgatattaaaactgtattcttagattagggagagataatatggacattctatcaattatagccttaacattgcttatcattattattgttaataccacaatgaactttgtaggtatgttacgtggtgagcgtgaattagtaaaaaaaggaggtaaccctctacccaattggcagtactataatgtgttattacctaatttatgtggtattatcctcttaggtattgttgtttattttggagattcaatttataaaattacaacaaggttagaagttctttttgctattattgcgcttattgtaatagatgtattgttaacagcgttagtattattagtattgagtttcgtaacaaaaaataaagaatagtaaagaagggagtctggtttatacttgactctctttgttatttatggtatattagatatgaggtgatttttgttgcaattaaattttggtaaatttgataatgaaattattaagacaaaagtaagtgaaggtgtctactcttttagaggtgttccttattactacatagaacatgtccaagatgaaacttctgagtatgtattagtatataacatacatagtgttgatgatgaagtaccacaaaagaaatataaaatagaaacagtaagtaaaactattagaggtggtacgatattaagtaatacaattaagtcaatgctacctaataataagaagtataaaaaggtatatgaacctcctatctttttagctaatattatacctttaggtacagatacaactacaggttctgtaggtaaagggtttttcgaaagagaaaaagatagagtaactattacacaaaaagaaggcactaaagttatacatggtgaatatacaggagtatttatatgtttatctaatattaaatggattaagtcctatactcctttagacagtatattacaatattatcaacgtattaaaggggatagaataaatgtctgagataactaaattctatgagcaggacattaaagatttaattagaacgaagagacatatgtttaaagatgatgagataacaagtaatatacaagatattagaatatttaatgaaaaagctatttgtcaaggtaaatgtagaactgactgtttaattcttgatagaaacggtactgttatgggtatagagataaaaacagaacgagactctacacaaagacttaataaacagttatactattatagtctagtatgtaagtatgtttatgtgatgtgtcatgataagcatgtacctaaagtagagcaaatactaaaaaggtataaacaccatcatgtaggtattatgagctatattaactttaaaagcagtccagtagtaggtaagtataaggaagctacgccttcaccactaagaagtccttatcatacactaaatattttatggaagcaacatttaatgactatacttaaacagttaagagattataatacatatctaactggatataactatagggagcacggtaaaaatactaacaatgaaggcagttatatagaaggaacacaacgtatgagaatgaaaaagtcagctattattaaccaagttattcattatttaggtgtagagaactcttataagatatttactagaggtgttatctatggttatagtaataggtggcagatattagaagatgatttctttaaagttatacagaacggagtgagggtattatatggcaaagaggaaacctgatacattaaaacctaaatctaacggatataaacatcaacctgttaactttgctcctacaggtaacttatcaggtagaagtacttctttttttaataaaaaaagaaaagatatatcagatgaaagtattgttgttaaatacaaacctttgtttgttaaaaggtttgataatattactgctacagacattaaaatacaaaagaagtatgcgttagacttaatcagtgaagctgtgaatataaagaagaaatatcttgtaatgaagcaaaaaggtaaattaacacaaactattttacatacagatagagtttattatgtttatagaggtaagaaattaataggtaaatgtagcattcgtgaacaacgtacatttagaggtacacatttaatctacatttttagtactagacatagaatatctatacgcaaaaatagtagattagataagaaaagaacaccaaagaaaatgatatataaaggtggtaaataatggttaaatatttaagaacaaaagctaaaaaaacaaatataagtactttatttaagaaactacaaagtaaagatattacactattaggagtgtcctatgatagtgattatttccctagcggtgtaacaataataccttatttagaggatatatcacaagtagaagatggtatagaatttacgaataaagtaatagttactgagaacttaaaacctgctattgtaggtatgaataatatgataagtgattcaggactaggttatgttaaaacagagcagttaaataaaagattagaaaatactggattaatgacagatttattatctaaaggaacagaattaacttcaacaaagaaagtagacattgtatcaacttttattgaacctactattatgtaccaagatactactattaataaagaattaaaacttagattatacactatcgaagatgtatcccctttaaatgactacacacatgttgtatacttactagtaactgataaacaatatgacggtcaatcattcataggtacattatgtaaccaaggtacattaaacaaattagataccctaaaagttcttacattattcaaaggtaataatttaatcaatagaagtgtgttttctgttaagttagatacgaataaatatcattatagtttatataatacacatgagaccggtattttctttttagatgatgataaagacttaattattgcttgtggtcaatcctatgtaaaagttagatataaagatattgttagtagtaaagtagagaaagttagtgataataactataaaatgattgttaatcttgttggtaatgatgagctaacaattctcttatagtataatactcccttaaatatgtatactaaggtataagtatacgtatataagggattttttatatatatttttttcaagtagttagttaatttttaaaaggtattattcttattttaaaattatatttataaaacattaaaataccttttaggtatagggttttagaaaaaagatttatattgcttaaaatagttacactatagtgtaaaaaattagaatatcctttatgaaaataaaatagatgcagaaattgtgatatattatatagtgtaaagtataaaacagttgatttgataaggttatgtgaatacacatactccttattttagttcacacatatgtaaagacacttgagacaagaagggaatatataatggctagaaaaaagaatttaagaaataaaaaatctgatataaaggtagttccagataaggaaagtattctatctaaactatataataataaactattaaggtctaaagtggataatgctatagatgaagatgttagttatgatgatattatagacctatgtaaagagtatgacttagagttatccaaatctgccattacaagatataagagtaaacgtaaagaagctattgaaaatggatgggatttaggggaacttattgataaaagaaagaaagttagcgctactcaaatacagaaaaaagaaaaccctatgaaagaattagatagtgaaaattatacaccttttgaactggctacacagaatgtacaaacaatatatgatgacattcaaatactagatatgattattcaaaaaggtgctaaaggtttaaactttgtagaaacattagaccctgctttaatgattaaagctatggaaacaaaagatagaattacgggtaaccaactaaaaggtatgtcattcataggactaagagaactaatgattaaacaacaagcacaagatactgctatgacagaagttatgcttgagtttattcctgaagataaacatgaagaagttcttcaaagaatggagcaattacaagaagagttctataaaaacttagatttagatgaagaaggaagaaagttaaaagatacattagatagagtaggctatacaatatagataatgaggtaatacatatgacagaagaaattagtttaattccaataaaagatgttaaaccacttacagatatagtagatattataacacacctacataaaggtgatgtacttagagttaaacaagagaaccaaggagatatattactcagactaagtactggaaaacataagtttactgaagtgtcaagagacttagataaagaaaccatgttctataaaaggtattgggtactatacaatgtatctattaattctttactaacttttgatgtttatttagaaaacgattatgtagagtctacaaaagttaagttccctaagaacacaattgtagaatatgtaagagataatcaagaatcagatgtagctaaggttaaagatattttagtagatagtaacggtaattatttctatgcactttcaggagaaacatctttatataatgaagataaacttaataaaattaaaaattaatgattgacacactcatatgggtgtgttattcttttattacaaacaataaaaggagcaattgattatgattattacattaaatgaagaagagaaagcattattagaaaataaaagcaactatacgccattaagtaagaataaagagttcaatacaccaaaagaagaatatattgttacaagttataataaaggtgtacctgtgactgatattgctaaggaagctaaagtaagtatgggtttgatttatacagtgttaaacttctataaagtacctaagaaacataagaaatctagagtagcaaaacgtgtaaaacatattactaatgataagaataaagtacaagccttaattaaagattatcaatatatggacttacaaagtatctataaaaagtataacattcataagaatggtttatattatattttagatttgtataatattgaaagaaaatcagatttaaaagagaaagccttagaagatactatcgaggtagaataagaggtgtcatatgcgcaaagaacgaattgtagattacttgtataatgtaaagaacattgataataatttaactatacaaggaatagaaaatagtttatttaacaaagaatctttacaagacttagtacctgattttgatacttctagtaaagatattaatgaaagtaatttatacctatgtacaataccagaagattataactccgaccatgtagaaagtggtcagtatataggtatagatgttggatatgtatctgaagaccctgcttttgaccatttaataggtcaagttcctagaagtgtatatgagaaagcgcatgttatgcaaccactaattaagatagaaaatactgatatagattatactcaggctgatatgattaatgatattaaagtaggtacaagtattagtgatgtaaagattaaagataaattaagtttaatgtcaaataatcttattacacatttagaaattgttgataagtattactttgatagtagtatagaagaaacattattaaagtattatgagttaaagaatcctgactattatttaaaacattttcttaaactaaaagaacttgtcggagacaatagaataatatattgtcctttactgttaaaatgtattaaagttatagactaagttattatattataagtagtaaaggatggtgcgctatgagtaaaaaagcaataattgcttatgtaattatcttagttatgttagccttatctatatctacttattacgtatcctcatacttatatcatgaaaaaaccaaatcacaagttacagaccaattaacacatcacggtaaactaaaaaaagataagaatgtagagtatgtaggggactatacacttaagaaagtaatagacaacaaagcatactttatggaaaaactacctacctatttaccaggaagaacaagtgataaaagtattgatatgagatattataggactagtaaatttagagaaggtgtaaactttaaacttattcgtatatacacagaggataatgacaataatccagtacataaatataggtttgaagcagtattaaataaaaagtaaaggtatgatagtctatgacagtagtaattatactagtatgttttgctattatatactacttatttaacaaagattgagtcaagtttatgcttgactctttttttgcgttatgttatagtttatttataaggagatgataaatgatgaaaaaaagtgatttagataatttgtatattgtggtcgtaagtattaagaacggtgttttacctgatgagcgtataattccaattggtttagatgtactaagtaaagtattagaaggtaaaactacatggacagttaagaaagaaattaaacatacatttggtaaagatgaattaaaaagatttaaagataattacaagttcttgcaacataaaattgtagaccttgctacaaaaggatataggtaggtggtataaatggatatagtaaataaactcatgttaggttttattttattgttttatgtaggtgtatttatttggtacttatgggaagtctggaaattaacttctttaatatctgatatagttattaagtattatgttcgtaaggagtgggttaaaggtttattaccttatttgattcatcaagttattattacgttagtttgggctactctagtattagtaccttttatacttagttatataatgataaaatatttgataggaggttactaaatggaagtagtatatgagaatgaatgtagagggtattatatttgtgttatagataatgaaggtaagtatgaagtatcatcttttaaaaaatcagtaaacaaatatacccctgaaattatagggtatactacagaaaagttaatcttggaattaaaaataccatgtacatatgtaaatgctagtgaagtagaaagttttatgcaagaattaaaagtagcacgtattgcgcttgacaagtttactaaagatttatataataaaggcataacaaatattatattctaaagggtggttagtatgtatacagaaaaatatagaaatttaatccatattacacatagtatcttggttagtattcaagaattagcaagtaatttattaaataaagaagtaaaactagttacaaaagaaaataagtatttatgtgttgtagatggtattattatgttaattgttgaggaaggattagtatacacctattttgataaggtaggtttaaaagtacctgaacgtattgcacttggtcatttagcaaagagtttacagtttacaatatcggaatcaaataaagatttagaaaatcaaaaataagtgttgacacctttaacatgttgctttatactaagagtaacaaaacgttagaggtgattttttatgtgtaaagaaaaagttcatagacagctatctaatgctatagaaatattagcaacttacaaagagtggtggtcttttccaaaagaattaagtaccacagaaacatttaagattattaactggaaagaagatacattagtatttgaagttagagaatatggtaagtatataggtaggtttagtgtagcaactcctattattgattttcattatgatagaattaaaaatacaacaacacaatatatgataaattattttgctaaaatagtatgtaaagatatgtatgcgtattataaattacagagaggacagtaaaaaatgaaaaatttaaataaacaaaaacgcttgacgttggaattgattagtgataatctaaatacacaacataacttagatacatcggttagtcttgtagattatagtataaaagactttacaaatagaaaagtctatgtaagaactaaaaaaggtttagaacttactaatatgaaaggattaaaaaatactgattcactggatttactatttaagtacttagaattaattttaaaaggggatatgtagtatgctaacaccagaacaaagaacacaattaaaagagtatcaaaataaaatgacaaaaaagaaaaaataaatgttgacttactaaataacaagttgtataattaacttaacaaagaaaaaggagattgataaatatgaaaaacttatataaaattttaacaggagtattctttagcactacactattattaggaggttgtgcttactactcacaagaagatacaccaacacatgattataaagaagataagaaagaaaataagaaaaatgattcatctaaagtagaagaacaagaaaataaacaaattcaagaagacaacactgttcaaaataaccaacaagataaccaagtacaaggtaaccaagcacagcagaacgaacaaatgcaaaaagcaagcagaagtgaacataatggactatctaatgctgagtatgcggacaaatacaatcagtatagagaagctcaacaagcacaagcagaattagaaaacagaactgaagcagataaagctagtggtggtggaggcggaggagcctcatggaattatgcaggtgctaatgagagctttgagcaatggttacaacgctcgcaacaagaaaagactgaggctggagtacaagcaaattaataaaaggtataggagaataacaatgaaaacaataacaaaagaagaactacataaagttttagaagagcataaattatggttagatagtaatagtgaagaaggagctaaagcagatttaagttatactatcttaagaaatgctaatttagtagatgctgacttaaggagtgctaatttaagaggtgctgtcttaaggtatgctgatttagaggatactaatttaagaggtgctatcttaaaaggtgcagatttaacaagagctaacttaacatgtgctaacttaagctgtgttgatttaggtgatgctaacttagaaggttctgttttatatggaactgatttatattatgctgatttaagaaaagctaatttaacagattccagattaaatgaggctaatttagaatatgcagacttaacaaataccaatttaacagatgctaaattatcccaagcaagtactcaaaatatcaaaggactaaaagtatactctatagataatattggaacttttaatggtaaagtgacttatattcctagcttagatagagtatacgcagggtgttggataggtagtttagaagcgtttttagaaaaggggttagagatgaatgaaggaaataatagtaaacttgaggaaataagagatgcttactgcttttttagtaaacgtaaatagatataaggatgattaattatgaacattgtagaacaacgaaaacccataaagagtaagcatattaacttaactaaacacgcatatgaacgttatactttaagagttaataaagatagtaaagaacgtgcttgtcagtggtttgctagtgctttaatgcaagctacttttgagcatgcacaagaagacggtaatgaagtatatgtatttaagaattacagaattattattgatagacatctaactattattacagttatagatactgaagatgataattgggaaggtataaaggaagcaagagaacaaatagatacgtatattaaaggtaagctaaaaaaaactgttacacctctatttactaagttagatagaatagatattagtatccatgaagcgcatatcagcaggattaaaacaagaaaccctaagacaaaagatataattaacaaaaatattattgaacttgaaagaaataaatctctactactaaatagtatagaaggtgtaaagaaaacagcacataagtattacatagaattaaaggacatccataaaagattaaaggactgataatagtgtatttaaaagatttaattgaagattatgtgaatcaagataaagaagtaattataacaattacaagtaataaagtaattattgaagcagactatgatgttatttgtttgcctaagagcaaggttgatattaatgatttaaaagcttatgtatatggatttaatagatttaatactgagcaaattatgttaaaggatgtggaataatacaagacttaaaaagaataacacagcctgaattagattcattgatagggaagcatgaacaatggctagctacaaatgaaaaagaaggagaaaagctctttttacagtgtgtagatttaagaaatcttgattttcattccgctaaccttcaatattcaactattgtaatgtctagtttatgtggtgctaacctaagttattgtgatttaagttatggtgatttaagttacaatgagtgtatatctacgagatttacacatgctcgaatgaaggatattgagttgcctcatactaacttagagtgggctaatttaataaaagcaaacttagaaaatgcttgtttagctgaagctaatataagtgaggtagacgctaggtgggctaatatgaaaaatgctaacttaaatatggctaatttaaaaggtgcagattttggtggtacaaacctaactaatacatcattatattatgctaacacgcttgacgtgtttgggataagtatttatactataagtaatgtgcttgattttgacgagcacgtaacttatttacctgattatgatagaattattagtaattataatgaatatacccttgaagagtttggtaaactagaacctacgcttgagtataaagaagatagaataaaagaagtataaaagaaaattaaattagcttacaatttctttaaaggagtgaaagaacttgataaaatttaattcagacgaacataaacatatacttaatgttatggataaattaccatatacttgttggttagacactgtagatggtgagtcaggtgctgacttcttatgtgtattgcatagaacacactatgaacttactgaactaggtataatgatttatagttatggtagttggtacatgataccttatgacaaagaagatttaattgaatatgcaataaaaagagatatagaggaggaggaagaggaatggtaagtacggttattatatttttaattgttttaggtattatattgctaagtgtactaattattgttataccagatttaagcaaagcaaaaaaacgtaaaaaagatgcagatattaactatagtcaatatattaataaagacatggcttctgagtactatgaaaagtatatagtacctactgaacacattgttcaaaccaaagtaacagagataattgaaaatataaagaaagagtatattgtctctactgtaattattgactcttctagtaatgaagacaaaattatcttacatgttgatgataagtatttaaaaaaggtagtgtatgaagataggtatgaatgcatctacgattctaatacaaaatatcaatatgttgttaatcacttaacatatgaatcaagtactttattaaaacaagatgaagtaaataaattaaaagatatacttaataaagtacctgtcacagctaagcatacatttaaaaatgagatgcttaatcataggaatgaatatgtgttagattatattacaatatatatttgtggtgatttaactgctaaaattacaaagaatggttatgtacaaaaatttagtacactagaagatttttgtgacaagtatgaagcttactgtagatttgttagaattgatttatatgactgggtaaaagaagtaagaactattaatggatatagattagaggtagatattaaaccttattttaaaattactgctactaaaacaagtaagtatgaatttgtagtaagtgatgatgtatctaaaattaaaatatatagggatgatatgttactaactagtaggtatatacgaaaagcaattgatgtacaggaaataatagaggaaacattgaacaataatactattgtattttaaaaagtagaaagtaggtaaattccttacttttttcatatataagtgttgacattaataagcgtaaggtttataatatgtatataagttaaataattatagaggagataataatcatgaaaaaaatattaagtttagttttactcgtactaattgtactaacaataatcaatgtagtgaaaaaccaagagcaagtagaacaaagtaaccaagaatctcaaaataatgaagatgtagatgaccatacttcatacgtacctgataatgatggagagcataaagacgttagtgaagaacgtaaaagattatggggtacagagtatcaagaaggtgaaacacataacccaaatgctaaagtgtttaatcaagatacaggagaacaggttcaataagagtagaaggagcacttaatatagagattgatgctaggaaatatatattaagtaacctacaaaaaacatttaaatactctaatatagatagtaaagaggtaattaaaagatatagacatgtatataggtaaatggatagtttataagtagtaggtaagtaattaaaagaaagtcatatataagtaataagtaacacataaagaaacaacgtttgatagtatacagataagtaataggtaagtaaataattaaaagaaaacatctactactactatcacttatattgtaagtacctatatatatatataccaagtaataggtaataggtaagtagtaacaacagctaccgatattacagataccttgtatatagatgcatagtaagtagtaaataattaaaagaaaatagctatatggtatacctatatatattaagtagtagataagtattaagtagtagatacgtattaagtattaacaacagctacctgggaattttatatataataaggaatagataataggtaagtagtaagtaacaagtagtaagtaacaagtagtaagtaacaagtagtaagtaacaagtagtaagtaacaagtagtaagtaacaagtagtaagtaacaagtagtaagtaacaacaactacctgggaatttctttacatcccaccttcatagtgtataaatagttaggtgtctaactatttatttttatatagttttaactattgtgaactatgcttatgtagtattaaactatgctgaacctatagtaaactgttctaatgtatagttaaactaagctgaagtagtatttactgtgtacaaaggtatatttagtatcagatattgtgtatatggtatagtataattatgaacattattattattattcatatatattgtttgtttttacagtactattgactcaattttccctagagtaaaagtttaatatatttagcagtttccgaacattaaagaaattgtaagcactttgtatatatgtgttgacactgtgttgcttatagtgtatacttaatgcataaaggtaaaggagatgatttataatgaagattaaaacagaagtttcgtttacagagctaatgaatatgtattacaaaggcagtatagacaaaggtcactactattctactaatagtcagtttattgttagtataactgatggtgaggtatcgttctataatgtgtttttccctaatactacggttggtacacctaatagtactactatacctaatgatactagatttattgtagaagcggttgtagataccactactgaattatttagtctagtaactacttttaagcaaggtagtaacgtattaaactcagtatggtatagagttagtattgatgagttatataatcattataaaagtaaaaataaagagttattatcagttagtactgttgtagatgatgagttagttactatatggaaagatggtaagatattgtgatttggtatatagtactatttagtatacttatatgtatagttagtgttttaattctatacgtatcttacaaagaaggttttactattaggatactaggttactgatactactatttatactaacagaaattacaagatttagtatcaatacaagtaatgctattctatgtacagtacttgtattgttatgtgtagtactagtatatagaggagttaaataaccgcttctatatgaaactttatacctatatctattataactataggtatttttttatatttcttacataaatgtgttgattttatgtgtgttaaggtttatacttaatatataattaaaggagtgaagttaatatggatatgaataaagaagtattagaacgtttagatagtttaggtggtaagttagagcaagcaggtacacatggttatgaatccttaattaaatacacagttactcaaggtattatagatttatgttcaataagtatattattatctataacagctatcttataggtggctttgtataagtcagataaaaagtctaatgaaggtcatagtaccttattatttgagtttgtttgtaagaatgaaaaagagatttcttctgcaggatgcacagtaattatatttgctgttactctgtctattatttcttttttagtgctattaataggcttgcctattgctattcaagaaatttttaatccagaaggatatttgattaagagcactatagatagtttaaagtagagtaatgtataaaggagtggtatagtgtctaaaaaaagaaaagaaattaaagagtataagaaacagggttactctgttgagtatataggaagtattttaggtttatttggtattggcgatgttttatactatgtagaaactaataagaaatacttaaagtcaattagcgtaggtaaagatgacaaccaatatgtatatgactttaatggttatgaatatctatcaaaatataagttaaatatcaaagaagaagaactaatgtctaaagctattgctcagttaagtatcaaaggcttaaagaactttaacggagtgaagcatgcttgggatgtaggttttttagagcaagcttttaatgtacatcgtaaagctattgatatggttaagcttaaactaagctttgatgatgagggtaacctatattatgaattagaattaggttggattaatatttataaagagtatacaggtgtaaaagactttcttaatacagtagataactatctcaacttcttctataaagatatagtcactagtggtcttgagttaaaacaagtactatataatacttttatgggaggagattatgagtttgtagtatctccatataaggttgaagtatatagggaagttgagttaattgatataaagaaattttactctgatacttatggaatttattcctatggttatggcactaatgtacataaacctttacaaacaattaagtattatctggaataccctatgaatgaagtataaccacttcaaatattatataaaaggagtgcttaatatgagtaacggaaaagtagcgttaattgggagtaaggtattggaagactataatatttatacaattatgagtgagaaacatatatacttaagagatagtgttgatgtggcttttaatgatgggaaggtattacctatatacattgatagtgtattaaaacaattaagtaaagatgataaaaatttatatttttctgaacaagatatagatattagtaaacttactaatgacgagtattatacagttactaaaaaataaaaggagaatagataatgagtagtaataaaaaaaaatagcagaaagagttattgagaaatttttagatgagactaataaaggtgtagattttactaatcaagatgaatttaaccgcttcttagataatacagaagacttgtatgaagtatctggtactcaaagagataagattattaagtgtttacaaaacattgtagttactgtatttatggataaggagaaataacaatgaaaacaataaaaagaaaaatagtacaagaaggtatgaacaaagaagaaactatgatggaagacgattctgttgtaatcaaaaaaattcaaaaaataacaggcggagctattcttcctttagccaatggcaaggaatatgtagtagtgtttagtaaagtagaccctactaaattaattgtatttaatgtggaaagtaatcaaatctatttaagtggctctggtttcgatagtgaactgtcttatgtagacagtaaaagcaaccctattaccttagaaacagttaggttaatcactaatttattataacggttattaggctcttaaagattaagttattttataggataaatactactagaaaatgattatcgtttaatatagaaatataacgtccctagtttaagggtcttaaatataaaggagggtattagaatgactaaggttgtaagtagagggtatagttttgaagaacaagaagtaagtatgaactataatcaaggacaatgggtgatttatgcaagtagaaaaccttatattactgatattatgaaaaagtaccctgataaggtggaagtattagaacaattagaaaatgaaacaccagtattggttaaagttgtattggatgaagatttaattacattaagaaaaccggtaagtaatgagcgcaaagagcaaatgaaaaagattgctaaagaaagatttggtaaataatagagaggctgactttaatgaacatcagtatgtacactaacacctgtagggactagattaatattacaagatatcttatagggttatagtgtcattgagaatgagtatgctttattaaggttagttaaagaagataaacagtatttataatatcatattgaagaagtagaagcggtttaataccacttcttttttatattatatttatattctgagtaattatgttgtgtttaatgttggctaataattgtataccaatttgagcatagtatgtccgtttcaacatagttaatctattaatttgtatatttatctgttagttatgtataaatatttgttattacttatttatatatttgtatttcgtactaaccgtacgacttatctttttaaaaagataagaacaaaacagcgtataccttgttgttttacgctacgctacaacaaggtaacgcctgtaataagttattcgtattaaaatactaacaactaattacttgttgtttagattataacatatgtttatgagaattagatgataatttagttacaacagcattataataatattacagttatgttacaagttgctaagtatgttttgttactacaatatattgaatatgaaagctttagaccttttctgcagacaagtaattattacagttatgtatgataaaaggtaagtatcagggattaaattaaggttggattaatgaagcggataagttagatactgattaaaataaaatactttacattacctaaaacttatgatacacttaggataggataaaggaggaatatcaatgaatattcaagaagcaaaggataaattatacgatgaattactttatacagtaagtctttgtgagggaacagaagaagatttactagaacttattgagaatttattttatatttacaatgacgagcctgcatttttaagtaaagacggtatgcaatatactattaaagagtttatggagagaagaaaaacattagaacctatcttaactgaattaggtatagggtacgcaatgatagaatatcacacacatacagaatacgagttagttacttattcagtacttaaagttatacagtatgatgtagataactatactgtaattattgatactaatagtgataagaatattaagttatcagatttaacattagaagctgttaaggatattttgaatcaaattaagtagctagggttgttttaccctagctttttcatatgcttgtattgcctatttaactaccttatataagcaagtaagtattataggataaatactacctaaaaagtattaccgttgaatatagacctataagattaagtctctcttactgttataaacatcataagtgtaaatctatgattttcctaagtatcaggcgctgttttccctttatattaaccgcttcaaacaacataaaaaaagattaggctattaacctaatctataattcttttatataattcttttatataattcttttatataattcttgtaattctctttctaacttttttattgtttctatatcattatttcttttaatagactgacatataaatgcaatacctttaatcaaattaaatggttatttttacactgcattgtatacaattaaatcgatcgattataataattaatttaatctgtactttctagtttcttctatataatagtaagaaatcatttctttaaaaaactttcgtataacctattgacaatcaatataagatactgtaatataataagcgtagtaaacaaaccgaaaggaattgataggatatgaacgtaaatcaaattggagatatactaatagaacttttagacatggacggttacaaaagcaataactactatagaaatgatttaaaatattcaacacattatcaagttaaatatttagcttacattgatgatagctatgaaacactagaactatttaaagaaacgaaagtctttagcgtatcacattcaaatagattaccaattaacattaatgttaaggtagtgaacaaacacttatttatagagctagataatacgttatacctagtgaatgagaatgcagaaattattaaagaaaaaaagttaaaataatagttgacactagaactaagatagtgtaatataatagatgtagtaaacaaacagaaaaggaattgattaattatgaaaacactaattactaacagtaaaccatataagaactacggctatgaaatggaacaattcgagagagaaaacaacgtaattatcacacgagcataccacgaaccaacaaaacaaggctatgaccttgtagtacactacacggaggttaaataatatgaaatatctagaatacattgaaggaacttataacagaattaaagcacaaggttctataataaatgactatagactatggtcagatatggaagaatgtgatgatttatactatcatattaataaagcttgtcatgctctttacaatagcaatgtaaaagaaattacaacaacagacaaatgcgtgaaactaacacttgaaaatggaaaaacaaaacttgttaaaaaaggttaaaataatgtttgacattacatttaagatactgtaatataatagatgtaatacaaaaacaaacaaaagggagctaataacatgagagatataagaatagttttaagagaaattgacgaggtacagcaagccttaatatatggtaggaatagcagagagttagcacgttttttacgtagacaactggtaaaactagagaaagaaagacaagatataaattggtcatagccaaaagcctattacataggcttaaaaaataaaaataagggattgatacacaatgattaaagttaaatataaaggcaagaaataccgctcaacacaaaatacagatgctattgtaactattataggtggtacagcagtaatcacattgatagactggtttattagattacaagagaatattaatatatttattagataagggctaaccacccttatttttttatgtctatataaccgcttcacattagtgtaaaacttcccttatttctttgatataatactttacaaatacgagcttatactgtaatatattaattgtagtaaggaacacaacaaacaaaaaaagaagagggattgattacttatgaaaaactataaaactttagaagaattaaaccaagattatacagttattagaacacaaaacgaagcttcaagcaaacgtaataacttagatgttactgatatttatgttaatgattggggtttgttacaatatggtatttactttaacaaattcgataacgtgtacaatgtagtttcaatgacatcaggaaacaagaaatataatattactgcttatgaacttgatagctttattatggatgagttaaacaaacagttagagttatttattaatgcaggaagactataaaggaacacaacaaagaacagcaaacaaataaaaaaaacttaaaaaagttttaaaaaagtgttgacactaacatcaacatagtgtaatataatagatgtagtaaacaaaaacaaacagaaaaggattgattaattatgaaaaacacagtaaaagaaatggcaaacggaaacactaaacttgaaagattattagttttagcaattgaggaagcggttatgacagagcttgttgaatatgatgcattagataaatacaaccacttagaacaacagttattaaacaatgaaacattaacacaagcagaacgtaaaccagctaacattgattatagcacttacgagggagctatattagcacaagacaaagcagaacactttactaacgttatcttaaaccacatgaacaaagaacaatttgaacaaatgattatggatattgaaacactagaatttaatgcagaacaaatgctattctaaggagacattacacatgaacttatctgagaaagaacttatagatataatagacaacgttaatagcttcactggtattgatgaagctattaacgaaatggaaaacaaacaaacaactaaaggcagtgacaaagacagcggaacgattaaacagtacaatagtttagatgagttaagacaagactatgacataacaacattagcaccagatgtaataaaagaattactgaataatcattagtgtaaaacttcccaaaataattaactaaaactattgcaattaactgtagatttgatataatgtaattaaggaaaaacaaataaaagaaaaaggattgataaactatgaaattacttaaccaaatgaaacaaacagcagaacttgaaggaattgaatttgacttattattaacagctatctatgaaacaatagagatagaagctgttgaacaggatttactatgtaaatacattcatattgtaaaccaattaaaagagaataaagacataacaccattagaatatataccttctaacctagactttacagacaatacaacaataagactagttgataataaagtgtttgagttagcagattatatagtggaacatatgagtgttgaagagtttaataaacacattaaagactatgaacaaattaaagcagaagatatagaaatgatgtaaaacaaattgaaagggagttaattccctttcttttttttattattaattaggattgtatacaataggttaatataaatatattatacgggggcaggggtaaaatgccactaataccacacattttatatgtatatacc